(19) United States
(12) Patent Application Publication
(10) Pub. No.: US 2024/0360113 A1
(43) Pub. Date: Oct. 31, 2024

(54) AMINOARYL-INTEGRIN AGONISTS COMPOUNDS

(71) Applicant: Texas Heart Institute, Houston, TX (US)

(72) Inventors: Robert V. MARKET, Houston, TX (US); Ronald J. BIEDIGER, Houston, TX (US); Frederick MEECE, Houston, TX (US)

(73) Assignee: Texas Heart Institute, Houston, TX (US)

(21) Appl. No.: 18/682,750

(22) PCT Filed: Jun. 9, 2022
(86) PCT No.: PCT/US2022/032900

§ 371 Date: Feb. 9, 2024

Related U.S. Application Data
(60) Provisional application No. 63/231,558, filed on Aug. 10, 2021.

(51) Int. Cl. C07D 409/12; A61K 31/137; A61K 31/381; A61K 31/422; C07D 213/89; C07D 277/56; C07D 413/14; C07D 513/04

(52) U.S. Cl. CPC C07D 409/12; A61K 31/137; A61K 31/381; A61K 31/422; C07D 213/89; C07D 277/56; C07D 413/14; C07D 513/04

(57) ABSTRACT

N,N-disubstituted arylamine compounds and to their use as integrin agonists for enhancing binding of integrin-expressing cells to integrin-binding ligands or receptors.

### CROSS-REFERENCE TO RELATED APPLICATIONS

This application is a National Phase Entry into the U.S. under 35 U.S.C. § 371 of and claims priority to PCT Application No. PCT/US2022/032900, filed Jun. 9, 2022, entitled “AMINOARYL-INTEGRIN AGONISTS COMPOUNDS,” which claims priority to U.S. Provisional Application Ser. No. 63/231,558 filed Aug. 10, 2021 and entitled “AMINOARYL-INTEGRIN AGONISTS COMPOUNDS” by Market et al., which is incorporated herein by reference in its entirety.

### STATEMENT REGARDING FEDERALLY SPONSORED RESEARCH OR DEVELOPMENT

This invention was made with government support under grant numbers R42 CA203456, R41 AI145507, and R33 HL161778, and each awarded by the National Institutes of Health (National Cancer Institute, the National Institute of Allergy and Infectious Diseases, and the National Heart, Lung, and Blood Institute, respectively). The government has certain rights in the invention.

### FIELD OF DISCLOSURE

The present disclosure generally relates to N,N-disubstituted arylamine compounds as a new class of integrin agonist and to their use as integrin agonists for enhancing binding of integrin-expressing cells to integrin-binding ligands or receptors. These may be useful in cell-based therapies as well as adjuvants for vaccines and cancer therapies. The disclosed class exhibits the potential for lower molecular weights and better water solubility. Potency against VLA-4 is generally in the same range as other more lipophilic classes of previously reported integrin agonist compounds.

### DESCRIPTION OF RELATED ART

Integrin agonists of the prior art are known to activate integrins by augmenting interior cell mechanisms or upregulating integrin gene expression or permanently damaging or chemically altering the integrins. There is however a need in the art for integrin activating compounds that do not permanently damage or chemically alter their target integrins. This lack of permanent damage or chemical alteration of the integrins is critical for stem cell, immune cell, and other cells used in cell based therapies. Hence there is a need in the art for integrin activating compounds that facilitate activating, priming, homing, trafficking, infiltrating, targeting, and/or other movements of cells through the body of a patient, wherein the natural functioning of the cell is not permanently damaged or chemically altered, only enhanced.

### BRIEF SUMMARY OF DISCLOSURE

In one embodiment, a composition comprising a VLA-4 integrin agonist of the formula (I) is disclosed herein:

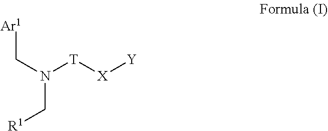

wherein,
R¹ is hydrogen, lower alkyl, or -aryl;
Ar¹ is aryl,
provided that when Ar¹ and R¹ are both phenyl, the 2 and 6 positions of each ring are substituted with hydrogen;
T is aryl;
X is a 1-20 atom linking chain containing any combination of —CH₂—, —C(O)—, —C(O)NR²—, —NR²—, —C(O)O— and —O—, each of which may independently be absent or present one or more times;
provided that when X contains —C(O)N(R²)—, and —C(O)N(R²)— directly attaches to T, then R² is hydrogen;
Y is selected from a group consisting essentially of (C₁-C₄)alkyl, aryl, cycloalkyl, N,N-dialkylamino, heterocyclyl, N,N-di(aralkyl)aminoaryl and (N-aralkyl)(N-alkyl)aminoaryl;
Ar¹ and R¹ may each be independently substituted or multiply substituted and selected at each occurrence from the group consisting essentially of lower alkyl, alkoxy, hydroxyalkyl, —OH, alkoxyalkyl, (C₁-C₃ alkyl)₂amino, alkoxyalkoxy, cycloalkyl, cycloalkylalkyl, aryl, heterocyclyl, alkylaryl, aralkyl, alkylheterocyclyl, and heterocyclylalkyl, groups;
Y may be unsubstituted or substituted with one or more substituents selected from the group consisting essentially of halogen, lower alkyl, alkoxy, hydroxyalkyl, aliphatic acyl, —CF₃, oxo, —CN, —OH, alkoxyalkyl, (C₁-C₃ alkyl)₂amino, alkoxyalkoxy, cycloalkyl, cycloalkylalkyl, aryl, heterocyclyl, alkylaryl, aralkyl, alkylheterocyclyl, heterocyclylalkyl, and aryloxyalkyl groups;
T may be unsubstituted or substituted with one or more substituents selected from the group consisting essentially of lower alkyl, alkoxy, hydroxyalkyl, aliphatic acyl, —CN, —OH, halogen, heterocycloyl, alkoxyalkyl, (C₁-C₃ alkyl)₂amino, alkoxyalkoxy, cycloalkyl, cycloalkylalkyl, heterocyclyl, alkylheterocyclyl, heterocyclylalkyl; provided that when T is thiazolyl group and X and Y taken together form a C₁-C₈ alkyl group, then the 5-position of said thiazole ring is unsubstituted;
substituents of T may be taken together with up to two atoms of X to form ring; and
R² when present, may be selected independently and at each occurrence from the groups consisting essentially of hydrogen, lower alkyl, acetyl or methanesulfonyl.

In some embodiments, a composition comprising a VLA-4 integrin agonist of the formula (I) is disclosed:

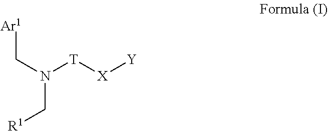

wherein,
R¹ is hydrogen, lower alkyl, or -aryl;
Ar¹ is aryl,
provided that when Ar¹ and R¹ are both phenyl, the 2 and 6 positions of each ring are substituted with hydrogen;
T is aryl;
X is a 1-20 atom linking chain containing any combination of —CH₂—, —C(O)—, —C(O)NR²—, —NR²—, —C(O)O— and —O— each of which may independently be absent or present one or more times;
provided that when X contains —C(O)N(R²)—, and —C(O)N(R²)— directly attaches to T, then R² is hydrogen;
Y is selected from a group comprising hydrogen, (C₁-C₄)alkyl, aryl, cycloalkyl, N,N-dialkylamino, heterocyclyl, N,N-di(aralkyl)aminoaryl and (N-aralkyl)(N-alkyl)aminoaryl;
Ar¹ and R¹ may each be independently substituted or multiply substituted and selected at each occurrence from the group consisting essentially of lower alkyl, alkoxy, hydroxyalkyl, —OH, alkoxyalkyl, (C₁-C₃ alkyl)₂amino, alkoxyalkoxy, cycloalkyl, cycloalkylalkyl, aryl, heterocyclyl, alkylaryl, aralkyl, alkylheterocyclyl, and heterocyclylalkyl, groups;
Y may be unsubstituted or substituted with one or more substituents selected from the group comprising halogen, lower alkyl, alkoxy, hydroxyalkyl, aliphatic acyl, —CF₃, oxo, —CN, —OH, alkoxyalkyl, (C₁-C₃ alkyl)₂amino, alkoxyalkoxy, cycloalkyl, cycloalkylalkyl, aryl, heterocyclyl, alkylaryl, aralkyl, alkylheterocyclyl, heterocyclylalkyl, and aryloxyalkyl groups;
T may be unsubstituted or substituted with one or more substituents selected from the group comprising: lower alkyl, alkoxy, hydroxyalkyl, aliphatic acyl, —CN, —OH, halogen, heterocycloyl, alkoxyalkyl, (C₁-C₃ alkyl)₂amino, alkoxyalkoxy, cycloalkyl, cycloalkylalkyl, heterocyclyl, alkylheterocyclyl, and heterocyclylalkyl; provided that when T is thiazolyl group and X and Y taken together form a C₁-C₈ alkyl group, then the 5-position of said thiazole ring is unsubstituted;
substituents of T may be taken together with up to two atoms of X to form ring; and
R² when present, may be selected independently and at each occurrence from the groups consisting essentially of hydrogen, lower alkyl, acetyl and methanesulfonyl.

In a further embodiment the compositions disclosed comprise a VLA-4 integrin agonist:
wherein,
(Ar¹CH₂)(R¹CH₂)N-T is selected from a group comprising:

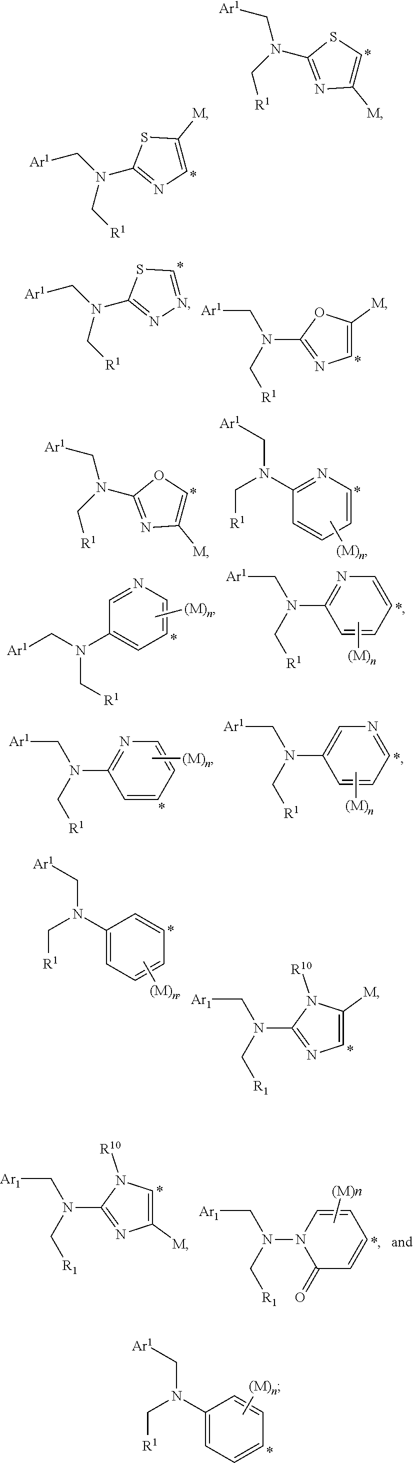

wherein the asterisks of each of the (Ar¹CH₂)(R¹CH₂)N-T groups indicate the attachment point for X;
M, when present, may be selected, independently and at each occurrence, from a group comprising: lower alkyl, alkoxy, hydroxyalkyl, aliphatic acyl, —CF₃, —CN, —OH, halogen, heterocycloyl, alkoxyalkyl, (C₁-C₃ alkyl)₂amino, alkoxyalkoxy, cycloalkyl, cycloalkylalkyl, heterocyclyl, alkylheterocyclyl, and heterocyclylalkyl;
R¹⁰ when present is hydrogen or a C₁-C₄ lower alkyl;
n is an integer from 0-3;
X is further selected from a group of radicals comprising:
—(CH₂)_{z}—, —(CH₂)_{z}O—, —(CH₂)_{z}O(CH₂)_{z}—, —(CH₂CH₂O)ₚ—, —(CH₂)_{z}O(CH₂)_{z}, —(CH₂)_{z}O(CH₂)_{z}O(CH₂)_{z}—, —(CH₂)_{z}O(CH₂CH₂O)ₚ—, —(CH₂)_{z}O(CH₂CH₂O)ₚ—(CH₂)_{z}—, —(CH₂)_{z}NR²—, —(CH₂)_{z}NR²(CH₂)_{z}—, —(CH₂)_{z}NR²(CH₂CH₂O)ₚ—, —(CH₂CH₂O)ₚ(CH₂)_{z}NR²—, —C(O)N(R²)—, —C(O)N(R²)(CH₂)_{z}—, —(CH₂)_{z}C(O)N(R²)(CH₂)_{z}—, —(CH₂)_{z}C(O)N(R²)—, —O(CH₂)_{z}—, —O(CH₂)_{z}O(CH₂)_{z}—, —O(CH₂CH₂O)ₚ—, —O(CH₂)_{z}NR²(CH₂)_{z}—, —O(CH₂)_{z}NR²(CH₂CH₂O)ₚ—, —O(CH₂CH₂O)ₚ(CH₂)_{z}NR²—, —O(CH₂)_{z}C(O)N(R²)(CH₂)_{z}—, and —O(CH₂)_{z}C(O)N(R²)—;
z is independently at each occurrence an integer of 1-20, and
p is an integer from 0-6,
provided that in X for any combination of groups, the integers p and z do not create a chain of greater than 20 atoms; and
a radical of M and 1 or 2 atoms of X may be taken together to form a 5 or 6 membered ring.

In a still further embodiment of the composition Ar¹ is selected from the group comprising substituted phenyl, substituted or unsubstituted thienyl, oxazolyl, isoxazolyl, pyrrolyl or pyridyl; and R¹ is hydrogen, C₁-C₄ alkyl, substituted phenyl, substituted or unsubstituted thienyl, oxazolyl, isoxazolyl, pyrrolyl, quinolinyl, indazolyl, benzimidazolyl, or pyridinyl.

In some embodiments the composition comprises Formula II

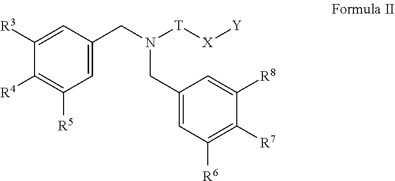

wherein:
R³, R⁴, R⁵, R⁶, R⁷, and R⁸ are each independently selected from a group consisting essentially of hydrogen, lower alkyl, OR⁹, phenyl, pyridiyl, pyrrolidyl, pyrazolyl, imidazolyl, oxazolyl, isoxazolyl, thiazolyl, isothiazolyl, thiadiazolyl, and diazolyl, lower alkyl, hydroxyalkyl, alkoxyalkyl, (C₁-C₃ alkyl)₂amino, alkoxyalkoxy, cycloalkyl, cycloalkylalkyl, aryloxy, heterocyclyl, alkylaryl, aralkyl, alkylheterocyclyl, heterocyclylalkyl, with the proviso that at least one of R³, R⁴, R⁵ and at least one of R⁶, R⁷, and R⁸ are not hydrogen;
R⁹ is hydrogen, —(C₁-C₄)alkyl, —CH₂CH₂OH, —CH₂CH₂OCH₃;
R³ and R⁴ can be taken together to form a fused-bicyclic-rings consisting essentially of alkyl, aryl and heterocyclic fused rings;
R⁶ and R⁷, can be taken together to form a fused bicyclic rings consisting essentially of alkyl, aryl and heterocyclic fused rings; In one or more embodiments, Ar¹CH₂)(R¹CH₂)N-T is selected from a group comprising of

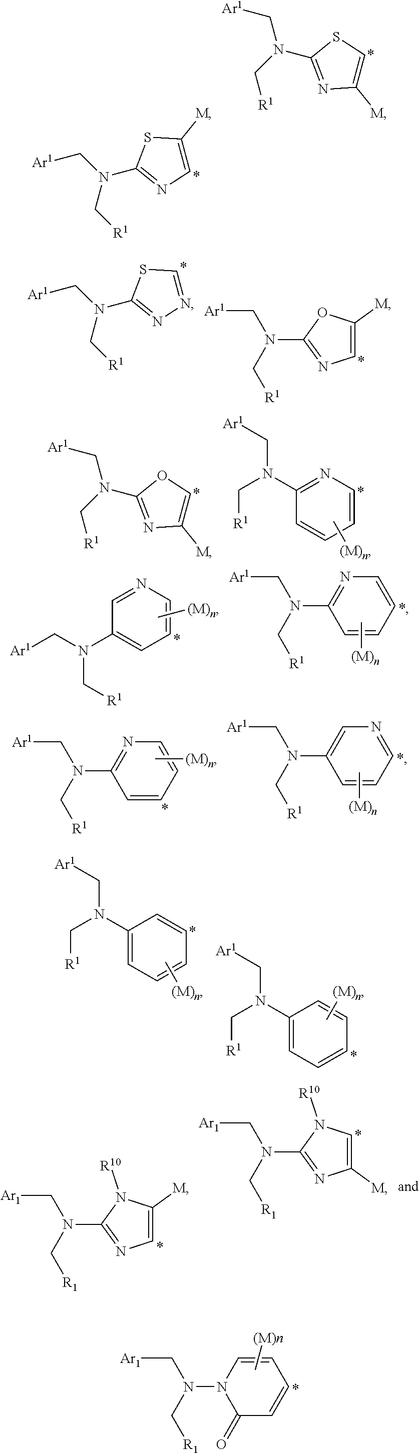

provided that in X for any combination of groups, the integers p and z do not create a chain of greater than 10 atoms; and
M is hydrogen or C₁-C₄ alkyl.

In other embodiments of the composition disclosed herein: Y is selected from a group consisting essentially of: 4-Morpholinyl, 1-piperidinyl, 1-piperazinyl, piperazin-2-one-1-yl, piperazine-2,5-dione-1-yl, 4-piperazin-2-one-4-yl, 1-pyrrolidinyl, 2-oxa-5-azabicyclo[2.2.1]heptan-5-yl, 3-hydroxypyrrolidin-1-yl, 4-hydroxypyrrolidine-2-one-1-yl, 3-hydroxypyrrolidin-2-one-1-yl, morpholin-3-one-4-yl, 6-oxa-1-azaspiro[3.3]heptan-1-yl, 6-oxa-3-azabicyclo[3.1.1]heptan-3-yl, hexahydro-2H-furo[3,2-b]pyrrol-4(5H)-yl, 2-oxa-5-azabicyclo[4.1.0]heptane, phenyl, pyridyl, 2-oxopyridin-1(2H)-yl, tetrahydro-2H-pyran-4-yl, oxetan-3-yl, tetrahydrofuran-2-yl, imidazole-1-yl, di(aralkyl)aminoaryl and (N-aralkyl)(N-alkyl)aminoaryl; including stereo-isomers thereof; and Y may be unsubstituted or substituted with one or more substituents selected from the group of halogen, lower alkyl, alkoxy, —CF₃, —CN, —OH, alkoxyalkyl, (C₁-C₃ alkyl)₂amino, alkoxyalkoxy, heterocyclyl, alkylheterocyclyl, and heterocyclylalkyl.

In other embodiments disclosed herein, the composition comprising a VLA-4 integrin agonist of the formula (I) is selected from the group consisting essentially of: Ethyl 2-(bis(thiophen-2-ylmethyl)amino)oxazole-4-carboxylate; 2-(bis(thiophen-2-ylmethyl)amino)-N-(3-methoxybenzyl)oxazole-4-carboxamide; 2-(bis(3-methoxybenzyl)amino)-N-(3-methoxybenzyl)oxazole-4-carboxamide; 2-(bis(3-methoxybenzyl)amino)-N-(4-methoxyphenethyl)oxazole-4-carboxamide; 2-(bis(3-methoxybenzyl)amino)-N-(3-(4-methoxyphenyl)propyl)oxazole-4-carboxamide; 2-(bis(3-methoxybenzyl)amino)-N-(3-(4-methoxyphenyl)propyl)oxazole-5-carboxamide; 2-(bis(3-methoxybenzyl)amino)-N-(4-methoxyphenethyl)thiazole-4-carboxamide; 2-(bis(3-methoxybenzyl)amino)-N-(3-(4-methoxyphenyl)propyl)thiazole-4-carboxamide; 4-((3-(4-methoxyphenyl)propoxy)methyl)-N,N-bis(thiophen-2-ylmethyl)oxazol-2-amine; ethyl 2-(bis(4-methoxybenzyl)amino)oxazole-4-carboxylate; (2-(bis(thiophen-2-ylmethyl)amino)oxazol-4-yl)methanol; ethyl 2-(bis(3-methoxybenzyl)amino)oxazole-4-carboxylate; ethyl 2-(bis(3-methoxybenzyl)amino)oxazole-4-carboxylate; (2-(bis(4-methoxybenzyl)amino)oxazol-4-yl)methanol; N,N-bis(3-methoxybenzyl)-2-(2-phenoxyethyl)oxazol-5-amine; ethyl 2-(bis(3-(2-methoxyethoxy)benzyl)amino)oxazole-4-carboxylate; 2-(2-((2-(bis(4-methoxybenzyl)amino)thiazol-4-yl)methoxy)ethoxy)ethanol; 4,4′-(2,2′-oxybis(ethane-2,1-diyl)bis(oxy))bis(methylene)bis(N,N-bis(4-methoxybenzyl)thiazol-2-amine); 2-((2-(bis(3-methoxybenzyl)amino)thiazol-4-yl)methoxy)ethanol; N,N-bis(3-methoxybenzyl)-4-((2-(2-(3-methoxyphenoxy)ethoxy)ethoxy)methyl)thiazol-2-amine; 4-((2-(2-(3-(dimethylamino)phenoxy)ethoxy)ethoxy)methyl)-N,N-bis(3-methoxybenzyl)thiazol-2-amine; 4-((2-(2-(benzyloxy)ethoxy)ethoxy)methyl)-N,N-bis(3-methoxybenzyl)thiazol-2-amine; N,N-bis(3-methoxybenzyl)-4-(((3-methoxybenzyl)oxy)methyl)thiazol-2-amine; N,N-bis(3-methoxybenzyl)-4-((3-(4-methoxyphenyl)propoxy)methyl)thiazol-2-amine; N,N-bis(3-methoxybenzyl)-4-((2-(3-methoxyphenoxy)ethoxy)methyl)thiazol-2-amine; N,N-bis(3-methoxybenzyl)-4-(((3-methoxybenzyl)oxy)methyl)thiazol-2-amine; N,N-bis(3-methoxybenzyl)-4-((3-(4-methoxyphenyl)propoxy)methyl)thiazol-2-amine; N,N-bis(3-methoxybenzyl)-4-((2-(3-methoxyphenoxy)ethoxy)methyl)thiazol-2-amine; 4-((2-(3-(dimethylamino)phenoxy)ethoxy)methyl)-N,N-bis(3-methoxybenzyl)thiazol-2-amine; 4-((2-(benzyloxy)ethoxy)methyl)-N,N-bis(3-methoxybenzyl)thiazol-2-amine; 4-((2-((2-chlorobenzyl)oxy)ethoxy)methyl)-N,N-bis(3-methoxybenzyl)thiazol-2-amine; N,N-bis(3-methoxybenzyl)-4-((2-(2-((2-methylbenzyl)oxy)ethoxy)ethoxy)methyl)thiazol-2-amine; N,N-bis(3-methoxybenzyl)-4-((4-methoxyphenethoxy)methyl)thiazol-2-amine; N,N-bis(3-methoxybenzyl)-4-((2-((2-methylbenzyl)oxy)ethoxy)methyl)thiazol-2-amine; N,N-bis(3-methoxybenzyl)-4-((2-(2-((3-methoxybenzyl)oxy)ethoxy)ethoxy)methyl)thiazol-2-amine; N,N-bis(3-methoxybenzyl)-4-((2-((3-methoxybenzyl)oxy)ethoxy)methyl)thiazol-2-amine; N,N-bis(3-methoxybenzyl)-4-((2-(2-phenoxyethoxy)ethoxy)methyl)thiazol-2-amine; 4-((2-(2-(4-chlorophenoxy)ethoxy)ethoxy)methyl)-N,N-bis(3-methoxybenzyl)thiazol-2-amine; 3-(2-(2-((2-(bis(3-methoxybenzyl)amino)thiazol-4-yl)methoxy)ethoxy)ethoxy)benzonitrile; 4-((2-(4-chlorophenoxy)ethoxy)methyl)-N,N-bis(3-methoxybenzyl)thiazol-2-amine; 4-((2-(2-(4-(dimethylamino)phenoxy)ethoxy)ethoxy)methyl)-N,N-bis(3-methoxybenzyl)thiazol-2-amine; 3-(2-((2-(bis(3-methoxybenzyl)amino)thiazol-4-yl)methoxy)ethoxy)benzonitrile; 4-((2-(4-(dimethylamino)phenoxy)ethoxy)methyl)-N,N-bis(3-methoxybenzyl)thiazol-2-amine; N,N-bis(3-methoxybenzyl)-4-(morpholinomethyl)thiazol-2-amine; 4-((2-(2-((2-chlorobenzyl)oxy)ethoxy)ethoxy)methyl)-N,N-bis(3-methoxybenzyl)thiazol-2-amine; N,N-bis(3-methoxybenzyl)-4-((2-phenoxyethoxy)methyl)thiazol-2-amine; N,N-bis(3-methoxybenzyl)-4-methylthiazol-2-amine; N,N-bis(3-(2-methoxyethoxy)benzyl)-4-methylthiazol-2-amine; N,N-bis(3-methoxybenzyl)-4-(piperidin-1-ylmethyl)thiazol-2-amine; N,N-bis(3-methoxybenzyl)-4-((2-(2-morpholinoethoxy)ethoxy)methyl)thiazol-2-amine; N,N-bis(3-methoxybenzyl)-4-((4-methylpiperazin-1-yl)methyl)thiazol-2-amine; N,N-bis(3-methoxybenzyl)-4-((2-morpholinoethoxy)methyl)thiazol-2-amine; N,N-bis(3-methoxybenzyl)-4-((2-(piperidin-1-yl)ethoxy)methyl)thiazol-2-amine; N,N-bis(3-methoxybenzyl)-4-((2-(4-methylpiperazin-1-yl)ethoxy)methyl)thiazol-2-amine; N,N-bis(3-methoxybenzyl)-4-((2-(2-(piperidin-1-yl)ethoxy)ethoxy)methyl)thiazol-2-amine; N,N-bis(3-methoxybenzyl)-4-((2-(2-(3-methoxyphenoxy)ethoxy)ethoxy)methyl)oxazol-2-amine; N-(3-(dimethylamino)benzyl)-N-(3-methoxybenzyl)-4-(morpholinomethyl)thiazol-2-amine; N-(3-methoxybenzyl)-N-(3-(2-methoxyethoxy)benzyl)-4-(morpholinomethyl)thiazol-2-amine; N-(4-(1H-pyrazol-1-yl)benzyl)-N-(3-methoxybenzyl)-4-(morpholinomethyl)thiazol-2-amine; N-(3-methoxybenzyl)-4-(morpholinomethyl)-N-(quinolin-6-ylmethyl)thiazol-2-amine; N-((1H-indazol-5-yl)methyl)-N-(3-methoxybenzyl)-4-(morpholinomethyl)thiazol-2-amine; N-(3-methoxybenzyl)-N-(3-morpholinobenzyl)-4-(morpholinomethyl)thiazol-2-amine; N-(3-(1H-imidazol-1-yl)benzyl)-N-(3-methoxybenzyl)-4-(morpholinomethyl)thiazol-2-amine; N-(3-(1H-pyrazol-1-yl)benzyl)-N-(3-methoxybenzyl)-4-(morpholinomethyl)thiazol-2-amine; N,N-bis(3-methoxybenzyl)-4-(morpholinomethyl)oxazol-2-amine; 4-((2-(dimethylamino)ethoxy)methyl)-N,N-bis(3-methoxybenzyl)thiazol-2-amine; N,N-bis(3-methoxybenzyl)-4-(2-morpholinoethyl)thiazol-2-amine; ethyl 4-(bis(3-methoxybenzyl)amino)benzoate; N,N-bis(3-methoxybenzyl)-4-((2-morpholinoethoxy)methyl)oxazol-2-amine; 4-((2-(indolin-1-yl)ethoxy)methyl)-N,N-bis(3-methoxybenzyl)thiazol-2-amine; N,N-bis(3-methoxybenzyl)-4-((3-methoxybenzyloxy)methyl)aniline; N,N-bis(3-methoxybenzyl)-3-(((3-methoxybenzyl)oxy)methyl)aniline; N,N-bis(3-methoxybenzyl)-4-((3-methoxybenzyl)oxy)aniline; N,N-bis(3-methoxybenzyl)-3-((3-methoxybenzyl)oxy)aniline; 5-ethyl-N,N-bis(3-methoxybenzyl)-1,3,4-thiadiazol-2-amine; ethyl 6-(bis(3-methoxybenzyl)amino)nicotinate; ethyl 2-(bis(3-methoxybenzyl)amino)pyrimidine-5-carboxylate; N-(4-(1H-pyrazol-1-yl)benzyl)-N-(3-methoxybenzyl)-4-methylthiazol-2-amine; methyl 2-(bis(3-methoxybenzyl)amino)-5-methylthiazole-4-carboxylate; ethyl 2-(bis(3-methoxybenzyl)amino)thiazole-4-carboxylate; N-(4-(1H-pyrazol-1-yl)benzyl)-N-(3-methoxybenzyl)-4-((3-methoxybenzyloxy)methyl)thiazol-2-amine; ethyl 2-((3-methoxybenzyl)(methyl)amino)thiazole-4-carboxylate; N-(3-methoxybenzyl)-N,4-dimethylthiazol-2-amine; N-(3-methoxybenzyl)-N-methyl-4-(morpholinomethyl)thiazol-2-amine; N-(3-methoxybenzyl)-4-((3-methoxybenzyloxy)methyl)-N-methylthiazol-2-amine; ethyl 2-((4-(1H-imidazol-1-yl)benzyl)(3-methoxybenzyl)amino)thiazole-4-carboxylate; ethyl 2-((3-methoxybenzyl)(quinolin-6-ylmethyl)amino)thiazole-4-carboxylate; ethyl 2-(((1H-indazol-5-yl)methyl)(3-methoxybenzyl)amino)thiazole-4-carboxylate; ethyl 2-((3-methoxybenzyl)(4-morpholinobenzyl)amino)thiazole-4-carboxylate; ethyl 2-((3-(1H-imidazol-1-yl)benzyl)(3-methoxybenzyl)amino)thiazole-4-carboxylate; ethyl 2-((3-(1H-pyrazol-1-yl)benzyl)(3-methoxybenzyl)amino)thiazole-4-carboxylate; ethyl 2-(((1H-benzo[d]imidazol-6-yl)methyl)(3-methoxybenzyl)amino)thiazole-4-carboxylate; ethyl 2-(isobutyl(3-methoxybenzyl)amino)thiazole-4-carboxylate; N,N-bis(3-methoxybenzyl)-4-((tetrahydro-2H-pyran-4-yloxy)methyl)thiazol-2-amine; N,N-bis(3-methoxybenzyl)-4-((oxetan-3-yloxy)methyl)thiazol-2-amine; N,N-bis(3-methoxybenzyl)-4-(((tetrahydro-2H-pyran-4-yl)methoxy)methyl)thiazol-2-amine; N,N-bis(3-methoxybenzyl)-4-((methyl(tetrahydro-2H-pyran-4-yl)amino)methyl)thiazol-2-amine; N,N-bis(3-methoxybenzyl)-4-((1-methylpiperidin-4-ylamino)methyl)thiazol-2-amine; 1-(bis(3-methoxybenzyl)amino)-4-((3-methoxybenzyl)oxy)-6-methylpyridin-2(1H)-one; and N,N-bis(3-methoxybenzyl)-4-((2-(2-(4-methylpiperazin-1-yl)ethoxy)ethoxy)methyl)thiazol-2-amine.

In other embodiments disclosed herein, the composition comprising a VLA-4 integrin agonist of the formula (I) is selected from the group: N,N-bis(3-methoxybenzyl)-4-(((tetrahydro-2H-pyran-4-yl)amino)methyl)thiazol-2-amine; N,N-bis(3-methoxybenzyl)-4-((oxetan-3-ylmethoxy)methyl)thiazol-2-amine; N,N-bis(3-methoxybenzyl)-4-(((1-methylpiperidin-4-yl)oxy)methyl)thiazol-2-amine; N,N-bis(3-methoxybenzyl)-4-(((4-methylpiperazin-1-yl)amino)methyl)thiazol-2-amine; N,N-bis(3-methoxybenzyl)-4-((piperidin-1-ylamino)methyl)thiazol-2-amine; N,N-bis(3-methoxybenzyl)-4-(((2-morpholinoethyl)amino)methyl)thiazol-2-amine; N-((2-(bis(3-methoxybenzyl)amino)thiazol-4-yl)methyl)morpholin-4-amine; 4-((azetidin-3-ylamino)methyl)-N,N-bis(3-methoxybenzyl)thiazol-2-amine; N,N-bis(3-methoxybenzyl)-4-((2-methylhydrazinyl)methyl)thiazol-2-amine; N1-((2-(bis(3-methoxybenzyl)amino)thiazol-4-yl)methyl)-N2,N2-dimethylethane-1,2-diamine; 1-((2-(bis(3-methoxybenzyl)amino)thiazol-4-yl)methyl)pyrrolidin-3-ol; 4-((1H-imidazol-1-yl)methyl)-N,N-bis(3-methoxybenzyl)thiazol-2-amine; N,N-bis(3-methoxybenzyl)-4-(3-morpholinopropyl)thiazol-2-amine; 4-((2-(1H-imidazol-1-yl)ethoxy)methyl)-N,N-bis(3-methoxybenzyl)thiazol-2-amine; 4-((3-(1H-imidazol-1-yl)propoxy)methyl)-N,N-bis(3-methoxybenzyl)thiazol-2-amine; 4-((2-(2-(1H-imidazol-1-yl)ethoxy)ethoxy)methyl)-N,N-bis(3-methoxybenzyl)thiazol-2-amine; 1-((2-(bis(3-methoxybenzyl)amino)thiazol-4-yl)methyl)pyridin-2(1H)-one; N,N-bis(3-methoxybenzyl)-4-((2-(tetrahydro-2H-pyran-4-yloxy)ethoxy)methyl)thiazol-2-amine; N,N-bis(3-methoxybenzyl)-4-(((1-methylpiperidin-4-yl)methylamino)methyl)thiazol-2-amine; N,N-bis(3-methoxybenzyl)-4-(((1-methylpiperidin-4-yl)methoxy)methyl)thiazol-2-amine; N,N-bis(3-methoxybenzyl)-4-((3-morpholinopropoxy)methyl)thiazol-2-amine; N,N-bis(3-methoxybenzyl)-4-((3-morpholinopropylamino)methyl)thiazol-2-amine; N,N-bis(3-methoxybenzyl)-4-((3-morpholinopropylamino)methyl)thiazol-2-amine; N,N-bis(3-methoxybenzyl)-4-(3-(pyridin-3-yl)propyl)thiazol-2-amine; 1-(2-((2-(bis(3-methoxybenzyl)amino)thiazol-4-yl)methoxy)ethyl)pyridin-2(1H)-one; 1-(2-((2-(bis(3-methoxybenzyl)amino)thiazol-4-yl)methylamino)ethyl)pyridin-2(1H)-one; 1-(2-(2-((2-(bis(3-methoxybenzyl)amino)thiazol-4-yl)methoxy)ethoxy)ethyl)pyridin-2(1H)-one; 4-((2-(bis(3-methoxybenzyl)amino)thiazol-4-yl)methyl)-1-methylpiperazin-2-one; 1-((2-(bis(3-methoxybenzyl)amino)thiazol-4-yl)methyl)-4-methylpiperazine-2,5-dione; 4-(2-((2-(bis(3-methoxybenzyl)amino)thiazol-4-yl)methoxy)ethyl)piperazin-2-one; 4-(2-((2-(bis(3-methoxybenzyl)amino)thiazol-4-yl)methoxy)ethyl)-1-methylpiperazin-2-one; 1-(2-((2-(bis(3-methoxybenzyl)amino)thiazol-4-yl)methoxy)ethyl)piperazine-2,5-dione; 1-(2-((2-(bis(3-methoxybenzyl)amino)thiazol-4-yl)methoxy)ethyl)-4-methylpiperazine-2,5-dione; 1-((2-(bis(3-methoxybenzyl)amino)thiazol-4-yl)methyl)-4-methylpiperazin-2-one; 1-(2-((2-(bis(3-methoxybenzyl)amino)thiazol-4-yl)methoxy)ethyl)-4-methylpiperazin-2-one; 1-(2-((2-(bis(3-methoxybenzyl)amino)thiazol-4-yl)methoxy)ethyl)piperazin-2-one; 4-((2-(2-(dimethylamino)ethoxy)ethoxy)methyl)-N,N-bis(3-methoxybenzyl)thiazol-2-amine; N,N-bis(3-methoxybenzyl)-4-(2-methyl-5,8,11-trioxa-2-azadodecan-12-yl)thizazol-2-amine; N,N-bis(3-methoxybenzyl)-4-((2-(2-(2-morpholinoethoxy)ethoxy)ethoxy)methyl)thiazol-2-amine; 4-((2-(2-(indolin-1-yl)ethoxy)ethoxy)methyl)-N,N-bis(3-methoxybenzyl)thiazol-2-amine; 4-((2-(2-(3,4-dihydroquinolin-1(2H)-yl)ethoxy)ethoxy)methyl)-N,N-bis(3-methoxybenzyl)thiazol-2-amine; 4-((2-(3,4-dihydroquinolin-1(2H)-yl)ethoxy)methyl)-N,N-bis(3-methoxybenzyl)thiazol-2-amine; 1-((2-(bis(3-methoxybenzyl)amino)thiazol-4-yl)methyl)piperazin-2-one; 1-((2-(bis(3-methoxybenzyl)amino)thiazol-4-yl)methyl)piperazine-2,5-dione; 4-((2-(bis(3-methoxybenzyl)amino)thiazol-4-yl)methyl)piperazin-2-one; N,N-bis(3-methoxybenzyl)-4-methyloxazol-2-amine; N-(3-(dimethylamino)benzyl)-N-(3-methoxybenzyl)-4-methyloxazol-2-amine; N-(3-methoxybenzyl)-4-methyl-N-(4-(pyrrolidin-1-yl)benzyl)oxazol-2-amine; N-(3-methoxybenzyl)-4-methyl-N-(3-morpholinobenzyl)oxazol-2-amine; N-(3-methoxybenzyl)-4-methyl-N-(3-(pyrrolidin-1-yl)benzyl)oxazol-2-amine; N-(3-(1H-imidazol-1-yl)benzyl)-N-(3-methoxybenzyl)-4-methyloxazol-2-amine; N-(3-(1H-pyrazol-1-yl)benzyl)-N-(3-methoxybenzyl)-4-methyloxazol-2-amine; N-((1H-indazol-5-yl)methyl)-N-(3-methoxybenzyl)-4-methyloxazol-2-amine; N-((1H-benzo[d]imidazol-5-yl)methyl)-N-(3-methoxybenzyl)-4-methyloxazol-2-amine; N-(3-methoxybenzyl)-4-methyl-N-(quinolin-6-ylmethyl)oxazol-2-amine; N-(3-methoxybenzyl)-4-((2-(3-methoxybenzyloxy)ethoxy)methyl)-N-(quinolin-6-ylmethyl)oxazol-2-amine; 4-((2-(3-(dimethylamino)phenoxy)ethoxy)methyl)-N-(3-methoxybenzyl)-N-(quinolin-6-ylmethyl)oxazol-2-amine; N-(3-(dimethylamino)benzyl)-N-(3-methoxybenzyl)-4-((2-(3-methoxybenzyloxy)ethoxy)methyl)oxazol-2-amine; N-(3-methoxybenzyl)-4-((2-(3-methoxybenzyloxy)ethoxy)methyl)-N-(3-(pyrrolidin-1-yl)benzyl)oxazol-2-amine; N-(3-(1H-imidazol-1-yl)benzyl)-N-(3-methoxybenzyl)-4-((2-(3-methoxybenzyloxy)ethoxy)methyl)oxazol-2-amine; N-(3-(1H-pyrazol-1-yl)benzyl)-N-(3-methoxybenzyl)-4-((2-(3-methoxybenzyloxy)ethoxy)methyl)oxazol-2-amine; N-(3-methoxybenzyl)-4-((2-(3-methoxybenzyloxy)ethoxy)methyl)-N-(3-morpholinobenzyl)oxazol-2-amine; N-(3-methoxybenzyl)-4-((2-(3-methoxybenzyloxy)ethoxy)methyl)-N-(3-(4-methylpiperazin-1-yl)benzyl)oxazol-2-amine; N-((1H-benzo[d]imidazol-5-yl)methyl)-N-(3-methoxybenzyl)-4-((2-(3-methoxybenzyloxy)ethoxy)methyl)oxazol-2-amine; N-((1H-indazol-5-yl)methyl)-N-(3-methoxybenzyl)-4-((2-(3-methoxybenzyloxy)ethoxy)methyl)oxazol-2-amine; N-(3-methoxybenzyl)-4-((2-(3-methoxybenzyloxy)ethoxy)methyl)-N-(quinolin-6-ylmethyl)thiazol-2-amine; N-(3-methoxybenzyl)-4-((2-morpholinoethoxy)methyl)-N-(quinolin-6-ylmethyl)oxazol-2-amine; N-(4-(1H-imidazol-1-yl)benzyl)-N-(3-methoxybenzyl)-4-((2-morpholinoethoxy)methyl)oxazol-2-amine; N-(4-(1H-pyrazol-1-yl)benzyl)-N-(3-methoxybenzyl)-4-((2-morpholinoethoxy)methyl)oxazol-2-amine; N-(3-methoxybenzyl)-N-(4-morpholinobenzyl)-4-((2-morpholinoethoxy)methyl)oxazol-2-amine; N-(3-methoxybenzyl)-4-((2-morpholinoethoxy)methyl)-N-(4-(pyrrolidin-1-yl)benzyl)oxazol-2-amine; N-(3-methoxybenzyl)-N-(4-(4-methylpiperazin-1-yl)benzyl)-4-((2-morpholinoethoxy)methyl)oxazol-2-amine; N-((1H-benzo[d]imidazol-6-yl)methyl)-N-(3-methoxybenzyl)-4-((2-morpholinoethoxy)methyl)oxazol-2-amine; N-((1H-indazol-6-yl)methyl)-N-(3-methoxybenzyl)-4-((2-morpholinoethoxy)methyl)oxazol-2-amine; N-(3-(dimethylamino)benzyl)-N-(3-methoxybenzyl)-4-methylthiazol-2-amine; N-(3-methoxybenzyl)-4-methyl-N-(3-morpholinobenzyl)thiazol-2-amine; N-(3-methoxybenzyl)-4-methyl-N-(3-morpholinobenzyl)oxazol-2-amine; N-(3-(1H-imidazol-1-yl)benzyl)-N-(3-methoxybenzyl)-4-methyloxazol-2-amine; N-(3-(1H-pyrazol-1-yl)benzyl)-N-(3-methoxybenzyl)-4-methyloxazol-2-amine; N-(3-(1H-imidazol-1-yl)benzyl)-N-(3-methoxybenzyl)-4-methylthiazol-2-amine; N-(3-(1H-pyrazol-1-yl)benzyl)-N-(3-methoxybenzyl)-4-methylthiazol-2-amine; N-(3-methoxybenzyl)-4-methyl-N-(4-(pyrrolidin-1-yl)benzyl)thiazol-2-amine; N-(4-(1H-imidazol-1-yl)benzyl)-N-(3-methoxybenzyl)-4-methylthiazol-2-amine; N-(4N-(4-(1H-imidazol-1-yl)benzyl)-N-(3-methoxybenzyl)-4-methyloxazol-2-amine; N-(3-methoxybenzyl)-4-methyl-N-(4-(4-methylpiperazin-1-yl)benzyl)thiazol-2-amine; N-(3-methoxybenzyl)-4-methyl-N-(4-morpholinobenzyl)thiazol-2-amine; N-((1H-benzo[d]imidazol-6-yl)methyl)-N-(3-methoxybenzyl)-4-methylthiazol-2-amine; N-((1H-indazol-6-yl)methyl)-N-(3-methoxybenzyl)-4-methylthiazol-2-amine; N-(3-methoxybenzyl)-4-methyl-N-(quinolin-6-ylmethyl)thiazol-2-amine; 4-((2-(2-(benzyloxy)ethoxy)ethoxy)methyl)-N,N-bis(3-methoxybenzyl)oxazol-2-amine; 4-((2-(3-(dimethylamino)phenoxy)ethoxy)methyl)-N,N-bis(3-methoxybenzyl)oxazol-2-amine; N,N-bis(3-methoxybenzyl)-4-((2-(2-(3-methoxybenzyloxy)ethoxy)ethoxy)methyl)oxazol-2-amine; N,N-bis(3-methoxybenzyl)-4-((2-(3-methoxybenzyloxy)ethoxy)methyl)oxazol-2-amine; N,N-bis(3-(2-methoxyethoxy)benzyl)-4-methyloxazol-2-amine; N,N-bis(3-methoxybenzyl)-4-((2-(2-morpholinoethoxy)ethoxy)methyl)oxazol-2-amine; N-(3-methoxybenzyl)-4-((2-(3-methoxybenzyloxy)ethoxy)methyl)-N-(4-(pyrrolidin-1-yl)benzyl)oxazol-2-amine; N-(4-(1H-imidazol-1-yl)benzyl)-N-(3-methoxybenzyl)-4-((2-(3-methoxybenzyloxy)ethoxy)methyl)oxazol-2-amine; N-(4-(1H-pyrazol-1-yl)benzyl)-N-(3-methoxybenzyl)-4-((2-(3-methoxybenzyloxy)ethoxy)methyl)oxazol-2-amine; N-(3-methoxybenzyl)-4-((2-(3-methoxybenzyloxy)ethoxy)methyl)-N-(4-morpholinobenzyl)oxazol-2-amine; N-(3-methoxybenzyl)-4-((2-(3-methoxybenzyloxy)ethoxy)methyl)-N-(4-(4-methylpiperazin-1-yl)benzyl)oxazol-2-amine; N-(3-methoxybenzyl)-4-((2-(3-methoxybenzyloxy)ethoxy)methyl)-N-(quinolin-6-ylmethyl)thiazol-2-amine; N-(3-methoxybenzyl)-4-((2-(3-methoxybenzyloxy)ethoxy)methyl)-N-(3-(pyrrolidin-1-yl)benzyl)thiazol-2-amine; N-(3-(dimethylamino)benzyl)-N-(3-methoxybenzyl)-4-((2-(3-methoxybenzyloxy)ethoxy)methyl)thiazol-2-amine; N-(3-(1H-imidazol-1-yl)benzyl)-N-(3-methoxybenzyl)-4-((2-(3-methoxybenzyloxy)ethoxy)methyl)thiazol-2-amine; N-(3-(1H-pyrazol-1-yl)benzyl)-N-(3-methoxybenzyl)-4-((2-(3-methoxybenzyloxy)ethoxy)methyl)thiazol-2-amine; N-(3-methoxybenzyl)-4-((2-(3-methoxybenzyloxy)ethoxy)methyl)-N-(3-morpholinobenzyl)thiazol-2-amine; N-(3-methoxybenzyl)-4-((2-(3-methoxybenzyloxy)ethoxy)methyl)-N-(3-(4-methylpiperazin-1-yl)benzyl)thiazol-2-amine; N-((1H-benzo[d]imidazol-5-yl)methyl)-N-(3-methoxybenzyl)-4-((2-(3-methoxybenzyloxy)ethoxy)methyl)thiazol-2-amine; N-((1H-indazol-5-yl)methyl)-N-(3-methoxybenzyl)-4-((2-(3-methoxybenzyloxy)ethoxy)methyl)thiazol-2-amine; N-(3-methoxybenzyl)-4-((2-(3-methoxybenzyloxy)ethoxy)methyl)-N-(4-(pyrrolidin-1-yl)benzyl)thiazol-2-amine; N-(4-(1H-imidazol-1-yl)benzyl)-N-(3-methoxybenzyl)-4-((2-(3-methoxybenzyloxy)ethoxy)methyl)thiazol-2-amine; N-(4-(1H-pyrazol-1-yl)benzyl)-N-(3-methoxybenzyl)-4-((2-(3-methoxybenzyloxy)ethoxy)methyl)thiazol-2-amine; N-(3-methoxybenzyl)-4-((2-(3-methoxybenzyloxy)ethoxy)methyl)-N-(4-morpholinobenzyl)thiazol-2-amine; N-(3-methoxybenzyl)-4-((2-(3-methoxybenzyloxy)ethoxy)methyl)-N-(4-(4-methylpiperazin-1-yl)benzyl)thiazol-2-amine; N-(3-methoxybenzyl)-N-(3-(4-methylpiperazin-1-yl)benzyl)-4-((2-morpholinoethoxy)methyl)oxazol-2-amine; N-(3-methoxybenzyl)-N-(3-morpholinobenzyl)-4-((2-morpholinoethoxy)methyl)oxazol-2-amine; N-(3-methoxybenzyl)-4-((2-morpholinoethoxy)methyl)-N-(3-(pyrrolidin-1-yl)benzyl)oxazol-2-amine; N-(3-(1H-imidazol-1-yl)benzyl)-N-(3-methoxybenzyl)-4-((2-morpholinoethoxy)methyl)oxazol-2-amine; N-(3-(1H-pyrazol-1-yl)benzyl)-N-(3-methoxybenzyl)-4-((2-morpholinoethoxy)methyl)oxazol-2-amine; N-(3-(dimethylamino)benzyl)-N-(3-methoxybenzyl)-4-((2-morpholinoethoxy)methyl)oxazol-2-amine; N-(3-(dimethylamino)benzyl)-N-(3-methoxybenzyl)-4-((2-morpholinoethoxy)methyl)thiazol-2-amine; N-(3-methoxybenzyl)-4-((2-morpholinoethoxy)methyl)-N-(3-(pyrrolidin-1-yl)benzyl)thiazol-2-amine; N-(3-(1H-pyrazol-1-yl)benzyl)-N-(3-methoxybenzyl)-4-((2-morpholinoethoxy)methyl)thiazol-2-amine; N-(3-(1H-imidazol-1-yl)benzyl)-N-(3-methoxybenzyl)-4-((2-morpholinoethoxy)methyl)thiazol-2-amine; N-(3-methoxybenzyl)-4-((2-morpholinoethoxy)methyl)-N-(4-(pyrrolidin-1-yl)benzyl)thiazol-2-amine; N-(3-methoxybenzyl)-N-(4-morpholinobenzyl)-4-((2-morpholinoethoxy)methyl)thiazol-2-amine; N-(3-methoxybenzyl)-N-(4-(4-methylpiperazin-1-yl)benzyl)-4-((2-morpholinoethoxy)methyl)thiazol-2-amine; N-(3-methoxybenzyl)-N-(3-(4-methylpiperazin-1-yl)benzyl)-4-((2-morpholinoethoxy)methyl)thiazol-2-amine; N-(3-methoxybenzyl)-N-(3-morpholinobenzyl)-4-((2-morpholinoethoxy)methyl)thiazol-2-amine; N-(4-(1H-pyrazol-1-yl)benzyl)-N-(3-methoxybenzyl)-4-((2-morpholinoethoxy)methyl)thiazol-2-amine; N-(4-(1H-imidazol-1-yl)benzyl)-N-(3-methoxybenzyl)-4-((2-morpholinoethoxy)methyl)thiazol-2-amine; N-((1H-benzo[d]imidazol-6-yl)methyl)-N-(3-methoxybenzyl)-4-((2-morpholinoethoxy)methyl)thiazol-2-amine; N-((1H-indazol-5-yl)methyl)-N-(3-methoxybenzyl)-4-((2-morpholinoethoxy)methyl)thiazol-2-amine; N-(3-methoxybenzyl)-4-((2-morpholinoethoxy)methyl)-N-(quinolin-6-ylmethyl)thiazol-2-amine; 4-((2-(2-(dimethylamino)ethoxy)ethoxy)methyl)-N,N-bis(3-methoxybenzyl)oxazol-2-amine; 4-((2-(dimethylamino)ethoxy)methyl)-N,N-bis(3-methoxybenzyl)oxazol-2-amine; N,N-bis(3-methoxybenzyl)-4-(2-methyl-5,8,11-trioxa-2-azadodecan-12-yl)oxazol-2-amine; N,N-bis(3-methoxybenzyl)-4-((2-(2-(2-morpholinoethoxy)ethoxy)ethoxy)methyl)thiazol-2-amine; 4-((2-(2-(indolin-1-yl)ethoxy)ethoxy)methyl)-N,N-bis(3-methoxybenzyl)oxazol-2-amine; 4-((2-(2-(3,4-dihydroquinolin-1(2H)-yl)ethoxy)ethoxy)methyl)-N,N-bis(3-methoxybenzyl)oxazol-2-amine; 4-((2-(3,4-dihydroquinolin-1(2H)-yl)ethoxy)methyl)-N,N-bis(3-methoxybenzyl)oxazol-2-amine; 4-((2-(indolin-1-yl)ethoxy)methyl)-N,N-bis(3-methoxybenzyl)oxazol-2-amine; 1-((2-(bis(3-methoxybenzyl)amino)oxazol-4-yl)methyl)piperazin-2-one; 1-((2-(bis(3-methoxybenzyl)amino)oxazol-4-yl)methyl)piperazine-2,5-dione; 4-((2-(bis(3-methoxybenzyl)amino)oxazol-4-yl)methyl)piperazin-2-one; N-(4-(1H-imidazol-1-yl)benzyl)-4-((2-(3-(dimethylamino)phenoxy)ethoxy)methyl)-N-(3-methoxybenzyl)oxazol-2-amine; N-(4-(1H-pyrazol-1-yl)benzyl)-4-((2-(3-(dimethylamino)phenoxy)ethoxy)methyl)-N-(3-methoxybenzyl)oxazol-2-amine; 4-((2-(3-(dimethylamino)phenoxy)ethoxy)methyl)-N-(3-methoxybenzyl)-N-(4-morpholinobenzyl)oxazol-2-amine; 4-((2-(3-(dimethylamino)phenoxy)ethoxy)methyl)-N-(3-methoxybenzyl)-N-(4-(pyrrolidin-1-yl)benzyl)oxazol-2-amine; 4-((2-(3-(dimethylamino)phenoxy)ethoxy)methyl)-N-(3-methoxybenzyl)-N-(4-(4-methylpiperazin-1-yl)benzyl)oxazol-2-amine; N-(3-(dimethylamino)benzyl)-4-((2-(3-(dimethylamino)phenoxy)ethoxy)methyl)-N-(3-methoxybenzyl)oxazol-2-amine; 4-((2-(3-(dimethylamino)phenoxy)ethoxy)methyl)-N-(3-methoxybenzyl)-N-(3-(pyrrolidin-1-yl)benzyl)oxazol-2-amine; N-(3-(1H-imidazol-1-yl)benzyl)-4-((2-(3-(dimethylamino)phenoxy)ethoxy)methyl)-N-(3-methoxybenzyl)oxazol-2-amine; N-(3-(1H-pyrazol-1-yl)benzyl)-4-((2-(3-(dimethylamino)phenoxy)ethoxy)methyl)-N-(3-methoxybenzyl)oxazol-2-amine; 4-((2-(3-(dimethylamino)phenoxy)ethoxy)methyl)-N-(3-methoxybenzyl)-N-(3-morpholinobenzyl)oxazol-2-amine; 4-((2-(3-(dimethylamino)phenoxy)ethoxy)methyl)-N-(3-methoxybenzyl)-N-(3-(4-methylpiperazin-1-yl)benzyl)oxazol-2-amine; N-((1H-benzo[d]imidazol-6-yl)methyl)-4-((2-(3-(dimethylamino)phenoxy)ethoxy)methyl)-N-(3-methoxybenzyl)oxazol-2-amine; N-((1H-indazol-5-yl)methyl)-4-((2-(3-(dimethylamino)phenoxy)ethoxy)methyl)-N-(3-methoxybenzyl)oxazol-2-amine; 4-((2-(3-(dimethylamino)phenoxy)ethoxy)methyl)-N-(3-methoxybenzyl)-N-(quinolin-6-ylmethyl)oxazol-2-amine; N-(4-(1H-imidazol-1-yl)benzyl)-4-((2-(3-(dimethylamino)phenoxy)ethoxy)methyl)-N-(3-methoxybenzyl)thiazol-2-amine; N-(4-(1H-pyrazol-1-yl)benzyl)-4-((2-(3-(dimethylamino)phenoxy)ethoxy)methyl)-N-(3-methoxybenzyl)thiazol-2-amine; 4-((2-(3-(dimethylamino)phenoxy)ethoxy)methyl)-N-(3-methoxybenzyl)-N-(4-morpholinobenzyl)thiazol-2-amine; 4-((2-(3-(dimethylamino)phenoxy)ethoxy)methyl)-N-(3-methoxybenzyl)-N-(4-(pyrrolidin-1-yl)benzyl)thiazol-2-amine; 4-((2-(3-(dimethylamino)phenoxy)ethoxy)methyl)-N-(3-methoxybenzyl)-N-(4-(4-methylpiperazin-1-yl)benzyl)thiazol-2-amine; N-(3-(dimethylamino)benzyl)-4-((2-(3-(dimethylamino)phenoxy)ethoxy)methyl)-N-(3-methoxybenzyl)thiazol-2-amine; 4-((2-(3-(dimethylamino)phenoxy)ethoxy)methyl)-N-(3-methoxybenzyl)-N-(3-(pyrrolidin-1-yl)benzyl)thiazol-2-amine; N-(3-(1H-imidazol-1-yl)benzyl)-4-((2-(3-(dimethylamino)phenoxy)ethoxy)methyl)-N-(3-methoxybenzyl)thiazol-2-amine; N-(3-(1H-pyrazol-1-yl)benzyl)-4-((2-(3-(dimethylamino)phenoxy)ethoxy)methyl)-N-(3-methoxybenzyl)thiazol-2-amine; 4-((2-(3-(dimethylamino)phenoxy)ethoxy)methyl)-N-(3-methoxybenzyl)-N-(3-morpholinobenzyl)thiazol-2-amine; 4-((2-(3-(dimethylamino)phenoxy)ethoxy)methyl)-N-(3-methoxybenzyl)-N-(3-(4-methylpiperazin-1-yl)benzyl)thiazol-2-amine; N-((1H-benzo[d]imidazol-5-yl)methyl)-4-((2-(3-(dimethylamino)phenoxy)ethoxy)methyl)-N-(3-methoxybenzyl)thiazol-2-amine; N-((1H-indazol-5-yl)methyl)-4-((2-(3-(dimethylamino)phenoxy)ethoxy)methyl)-N-(3-methoxybenzyl)thiazol-2-amine; 4-((2-(3-(dimethylamino)phenoxy)ethoxy)methyl)-N-(3-methoxybenzyl)-N-(quinolin-6-ylmethyl)thiazol-2-amine; N-(3-methoxybenzyl)-4-(morpholinomethyl)-N-(3-(pyrrolidin-1-yl)benzyl)oxazol-2-amine; N-(3-(1H-pyrazol-1-yl)benzyl)-N-(3-methoxybenzyl)-4-(morpholinomethyl)oxazol-2-amine; N-(3-(1H-imidazol-1-yl)benzyl)-N-(3-methoxybenzyl)-4-(morpholinomethyl)oxazol-2-amine; N-(3-methoxybenzyl)-N-(3-morpholinobenzyl)-4-(morpholinomethyl)oxazol-2-amine; N-(3-methoxybenzyl)-N-(3-(4-methylpiperazin-1-yl)benzyl)-4-(morpholinomethyl)oxazol-2-amine; N-(3-methoxybenzyl)-4-(morpholinomethyl)-N-(4-(pyrrolidin-1-yl)benzyl)oxazol-2-amine; N-(3-methoxybenzyl)-N-(4-morpholinobenzyl)-4-(morpholinomethyl)oxazol-2-amine; N-(3-methoxybenzyl)-N-(4-(4-methylpiperazin-1-yl)benzyl)-4-(morpholinomethyl)oxazol-2-amine; N-(4-(1H-pyrazol-1-yl)benzyl)-N-(3-methoxybenzyl)-4-(morpholinomethyl)oxazol-2-amine; N-(4-(1H-imidazol-1-yl)benzyl)-N-(3-methoxybenzyl)-4-(morpholinomethyl)oxazol-2-amine; N-((1H-benzo[d]imidazol-5-yl)methyl)-N-(3-methoxybenzyl)-4-(morpholinomethyl)oxazol-2-amine; N-((1H-indazol-6-yl)methyl)-N-(3-methoxybenzyl)-4-(morpholinomethyl)oxazol-2-amine; N-(3-methoxybenzyl)-4-(morpholinomethyl)-N-(quinolin-6-ylmethyl)oxazol-2-amine; N-(3-(dimethylamino)benzyl)-N-(3-methoxybenzyl)-4-(morpholinomethyl)oxazol-2-amine; N-(3-methoxybenzyl)-4-(morpholinomethyl)-N-(3-(pyrrolidin-1-yl)benzyl)thiazol-2-amine; N-(3-methoxybenzyl)-4-(morpholinomethyl)-N-(4-(pyrrolidin-1-yl)benzyl)thiazol-2-amine; N-(3-methoxybenzyl)-N-(4-morpholinobenzyl)-4-(morpholinomethyl)thiazol-2-amine; N-(3-methoxybenzyl)-N-(4-(4-methylpiperazin-1-yl)benzyl)-4-(morpholinomethyl)thiazol-2-amine; N-(4-(1H-imidazol-1-yl)benzyl)-N-(3-methoxybenzyl)-4-(morpholinomethyl)thiazol-2-amine; N-(3-methoxybenzyl)-N-(3-(4-methylpiperazin-1-yl)benzyl)-4-(morpholinomethyl)thiazol-2-amine; N-((1H-benzo[d]imidazol-6-yl)methyl)-N-(3-methoxybenzyl)-4-(morpholinomethyl)thiazol-2-amine; N-((1H-indazol-6-yl)methyl)-N-(3-methoxybenzyl)-4-(morpholinomethyl)thiazol-2-amine; N-(3-methoxybenzyl)-4-(piperidin-1-ylmethyl)-N-(3-(pyrrolidin-1-yl)benzyl)oxazol-2-amine; N-(3-(1H-pyrazol-1-yl)benzyl)-N-(3-methoxybenzyl)-4-(piperidin-1-ylmethyl)oxazol-2-amine; N-(3-(1H-imidazol-1-yl)benzyl)-N-(3-methoxybenzyl)-4-(piperidin-1-ylmethyl)oxazol-2-amine; N-(3-methoxybenzyl)-N-(3-morpholinobenzyl)-4-(piperidin-1-ylmethyl)oxazol-2-amine; N-(3-methoxybenzyl)-N-(3-(4-methylpiperazin-1-yl)benzyl)-4-(piperidin-1-ylmethyl)oxazol-2-amine; N-(3-methoxybenzyl)-4-(piperidin-1-ylmethyl)-N-(4-(pyrrolidin-1-yl)benzyl)oxazol-2-amine; N-(3-methoxybenzyl)-N-(4-morpholinobenzyl)-4-(piperidin-1-ylmethyl)oxazol-2-amine; N-(3-methoxybenzyl)-N-(4-(4-methylpiperazin-1-yl)benzyl)-4-(piperidin-1-ylmethyl)oxazol-2-amine; N-(4-(1H-pyrazol-1-yl)benzyl)-N-(3-methoxybenzyl)-4-(piperidin-1-ylmethyl)oxazol-2-amine; N-(4-(1H-imidazol-1-yl)benzyl)-N-(3-methoxybenzyl)-4-(piperidin-1-ylmethyl)oxazol-2-amine; N-((1H-benzo[d]imidazol-5-yl)methyl)-N-(3-methoxybenzyl)-4-(piperidin-1-ylmethyl)oxazol-2-amine; N-((1H-indazol-6-yl)methyl)-N-(3-methoxybenzyl)-4-(piperidin-1-ylmethyl)oxazol-2-amine; N-(3-methoxybenzyl)-4-(piperidin-1-ylmethyl)-N-(quinolin-6-ylmethyl)oxazol-2-amine; N-(3-(dimethylamino)benzyl)-N-(3-methoxybenzyl)-4-(piperidin-1-ylmethyl)oxazol-2-amine; N-(3-methoxybenzyl)-4-(piperidin-1-ylmethyl)-N-(3-(pyrrolidin-1-yl)benzyl)thiazol-2-amine; N-(3-(1H-pyrazol-1-yl)benzyl)-N-(3-methoxybenzyl)-4-(piperidin-1-ylmethyl)thiazol-2-amine; N-(3-(1H-imidazol-1-yl)benzyl)-N-(3-methoxybenzyl)-4-(piperidin-1-ylmethyl)thiazol-2-amine; N-(3-methoxybenzyl)-4-(piperidin-1-ylmethyl)-N-(4-(pyrrolidin-1-yl)benzyl)thiazol-2-amine; N-(3-methoxybenzyl)-N-(4-morpholinobenzyl)-4-(piperidin-1-ylmethyl)thiazol-2-amine; N-(3-methoxybenzyl)-N-(4-(4-methylpiperazin-1-yl)benzyl)-4-(piperidin-1-ylmethyl)thiazol-2-amine; N-(4-(1H-pyrazol-1-yl)benzyl)-N-(3-methoxybenzyl)-4-(piperidin-1-ylmethyl)thiazol-2-amine; N-(4-(1H-imidazol-1-yl)benzyl)-N-(3-methoxybenzyl)-4-(piperidin-1-ylmethyl)thiazol-2-amine; N-(3-methoxybenzyl)-N-(3-morpholinobenzyl)-4-(piperidin-1-ylmethyl)thiazol-2-amine; N-(3-methoxybenzyl)-N-(3-(4-methylpiperazin-1-yl)benzyl)-4-(piperidin-1-ylmethyl)thiazol-2-amine; N-((1H-benzo[d]imidazol-6-yl)methyl)-N-(3-methoxybenzyl)-4-(piperidin-1-ylmethyl)thiazol-2-amine; N-((1H-indazol-6-yl)methyl)-N-(3-methoxybenzyl)-4-(piperidin-1-ylmethyl)thiazol-2-amine; N-(3-methoxybenzyl)-4-(piperidin-1-ylmethyl)-N-(quinolin-6-ylmethyl)thiazol-2-amine; N-(3-(dimethylamino)benzyl)-N-(3-methoxybenzyl)-4-(piperidin-1-ylmethyl)thiazol-2-amine; N-(3-methoxybenzyl)-4-((4-methylpiperazin-1-yl)methyl)-N-(3-(pyrrolidin-1-yl)benzyl)oxazol-2-amine; N-(3-(1H-pyrazol-1-yl)benzyl)-N-(3-methoxybenzyl)-4-((4-methylpiperazin-1-yl)methyl)oxazol-2-amine; N-(3-(1H-imidazol-1-yl)benzyl)-N-(3-methoxybenzyl)-4-((4-methylpiperazin-1-yl)methyl)oxazol-2-amine; N-(3-methoxybenzyl)-4-((4-methylpiperazin-1-yl)methyl)-N-(3-morpholinobenzyl)oxazol-2-amine; N-(3-methoxybenzyl)-N-(3-(4-methylpiperazin-1-yl)benzyl)-4-(piperidin-1-ylmethyl)oxazol-2-amine; N-(3-methoxybenzyl)-4-((4-methylpiperazin-1-yl)methyl)-N-(4-(pyrrolidin-1-yl)benzyl)oxazol-2-amine; N-(3-methoxybenzyl)-4-((4-methylpiperazin-1-yl)methyl)-N-(4-morpholinobenzyl)oxazol-2-amine; N-(3-methoxybenzyl)-N-(4-(4-methylpiperazin-1-yl)benzyl)-4-((4-methylpiperazin-1-yl)methyl)oxazol-2-amine; N-(4-(1H-pyrazol-1-yl)benzyl)-N-(3-methoxybenzyl)-4-((4-methylpiperazin-1-yl)methyl)oxazol-2-amine; N-(4-(1H-imidazol-1-yl)benzyl)-N-(3-methoxybenzyl)-4-((4-methylpiperazin-1-yl)methyl)oxazol-2-amine; N-((1H-benzo[d]imidazol-5-yl)methyl)-N-(3-methoxybenzyl)-4-((4-methylpiperazin-1-yl)methyl)oxazol-2-amine; N-((1H-indazol-6-yl)methyl)-N-(3-methoxybenzyl)-4-((4-methylpiperazin-1-yl)methyl)oxazol-2-amine; N-(3-methoxybenzyl)-4-((4-methylpiperazin-1-yl)methyl)-N-(quinolin-6-ylmethyl)oxazol-2-amine; N-(3-(dimethylamino)benzyl)-N-(3-methoxybenzyl)-4-((4-methylpiperazin-1-yl)methyl)oxazol-2-amine; N-(3-methoxybenzyl)-4-((4-methylpiperazin-1-yl)methyl)-N-(3-(pyrrolidin-1-yl)benzyl)thiazol-2-amine; N-(3-(1H-pyrazol-1-yl)benzyl)-N-(3-methoxybenzyl)-4-((4-methylpiperazin-1-yl)methyl)thiazol-2-amine; N-(3-(1H-imidazol-1-yl)benzyl)-N-(3-methoxybenzyl)-4-((4-methylpiperazin-1-yl)methyl)thiazol-2-amine; N-(3-methoxybenzyl)-4-((4-methylpiperazin-1-yl)methyl)-N-(4-morpholinobenzyl)thiazol-2-amine; N-(3-methoxybenzyl)-N-(4-(4-methylpiperazin-1-yl)benzyl)-4-((4-methylpiperazin-1-yl)methyl)thiazol-2-amine N-(4-(1H-pyrazol-1-yl)benzyl)-N-(3-methoxybenzyl)-4-((4-methylpiperazin-1-yl)methyl)thiazol-2-amine; N-(4-(1H-imidazol-1-yl)benzyl)-N-(3-methoxybenzyl)-4-((4-methylpiperazin-1-yl)methyl)thiazol-2-amine; N-(3-methoxybenzyl)-4-((4-methylpiperazin-1-yl)methyl)-N-(3-morpholinobenzyl)thiazol-2-amine; N-(3-methoxybenzyl)-N-(3-(4-methylpiperazin-1-yl)benzyl)-4-((4-methylpiperazin-1-yl)methyl)thiazol-2-amine; N-((1H-benzo[d]imidazol-6-yl)methyl)-N-(3-methoxybenzyl)-4-((4-methylpiperazin-1-yl)methyl)thiazol-2-amine; N-((1H-indazol-6-yl)methyl)-N-(3-methoxybenzyl)-4-((4-methylpiperazin-1-yl)methyl)thiazol-2-amine; N-(3-methoxybenzyl)-4-((4-methylpiperazin-1-yl)methyl)-N-(quinolin-6-ylmethyl)thiazol-2-amine; N-(3-(dimethylamino)benzyl)-N-(3-methoxybenzyl)-4-((4-methylpiperazin-1-yl)methyl)thiazol-2-amine; 4-((dimethylamino)methyl)-N-(3-methoxybenzyl)-N-(3-(pyrrolidin-1-yl)benzyl)oxazol-2-amine; N-(3-(1H-pyrazol-1-yl)benzyl)-4-((dimethylamino)methyl)-N-(3-methoxybenzyl)oxazol-2-amine; N-(3-(1H-imidazol-1-yl)benzyl)-4-((dimethylamino)methyl)-N-(3-methoxybenzyl)oxazol-2-amine; 4-((dimethylamino)methyl)-N-(3-methoxybenzyl)-N-(3-morpholinobenzyl)oxazol-2-amine 4-((dimethylamino)methyl)-N-(3-methoxybenzyl)-N-(3-(4-methylpiperazin-1-yl)benzyl)oxazol-2-amine; 4-((dimethylamino)methyl)-N-(3-methoxybenzyl)-N-(4-(pyrrolidin-1-yl)benzyl)oxazol-2-amine; 4-((dimethylamino)methyl)-N-(3-methoxybenzyl)-N-(4-morpholinobenzyl)oxazol-2-amine; 4-((dimethylamino)methyl)-N-(3-methoxybenzyl)-N-(4-(4-methylpiperazin-1-yl)benzyl)oxazol-2-amine; N-(4-(1H-pyrazol-1-yl)benzyl)-4-((dimethylamino)methyl)-N-(3-methoxybenzyl)oxazol-2-amine; N-(4-(1H-imidazol-1-yl)benzyl)-4-((dimethylamino)methyl)-N-(3-methoxybenzyl)oxazol-2-amine; N-((1H-indazol-6-yl)methyl)-4-((dimethylamino)methyl)-N-(3-methoxybenzyl)oxazol-2-amine; N-((1H-benzo[d]imidazol-6-yl)methyl)-4-((dimethylamino)methyl)-N-(3-methoxybenzyl)oxazol-2-amine; 4-((dimethylamino)methyl)-N-(3-methoxybenzyl)-N-(quinolin-6-ylmethyl)oxazol-2-amine; N-(3-(dimethylamino)benzyl)-4-((dimethylamino)methyl)-N-(3-methoxybenzyl)oxazol-2-amine; 4-((dimethylamino)methyl)-N-(3-methoxybenzyl)-N-(3-(pyrrolidin-1-yl)benzyl)thiazol-2-amine; N-(3-(1H-pyrazol-1-yl)benzyl)-4-((dimethylamino)methyl)-N-(3-methoxybenzyl)thiazol-2-amine; N-(3-(1H-imidazol-1-yl)benzyl)-4-((dimethylamino)methyl)-N-(3-methoxybenzyl)thiazol-2-amine 4-((dimethylamino)methyl)-N-(3-methoxybenzyl)-N-(4-(pyrrolidin-1-yl)benzyl)thiazol-2-amine; 4-((dimethylamino)methyl)-N-(3-methoxybenzyl)-N-(4-(4-methylpiperazin-1-yl)benzyl)thiazol-2-amine; N-(4-(1H-pyrazol-1-yl)benzyl)-4-((dimethylamino)methyl)-N-(3-methoxybenzyl)thiazol-2-amine; N-(4-(1H-imidazol-1-yl)benzyl)-4-((dimethylamino)methyl)-N-(3-methoxybenzyl)thiazol-2-amine; 4-((dimethylamino)methyl)-N-(3-methoxybenzyl)-N-(3-morpholinobenzyl)thiazol-2-amine; 4-((dimethylamino)methyl)-N-(3-methoxybenzyl)-N-(3-(4-methylpiperazin-1-yl)benzyl)thiazol-2-amine; N-((1H-indazol-6-yl)methyl)-4-((dimethylamino)methyl)-N-(3-methoxybenzyl)thiazol-2-amine; 4-((dimethylamino)methyl)-N-(3-methoxybenzyl)-N-(quinolin-6-ylmethyl)thiazol-2-amine; N-(3-(dimethylamino)benzyl)-4-((dimethylamino)methyl)-N-(3-methoxybenzyl)thiazol-2-amine; 4-((dimethylamino)methyl)-N,N-bis(3-methoxybenzyl)oxazol-2-amine; 4-((dimethylamino)methyl)-N,N-bis(3-methoxybenzyl)thiazol-2-amine; N,N-bis(3-methoxybenzyl)-4-((2-(2-(3-methoxybenzyloxy)ethoxy)ethoxy)methyl)aniline; 4-((2-(2-(benzyloxy)ethoxy)ethoxy)methyl)-N,N-bis(3-methoxybenzyl)aniline; N,N-bis(3-methoxybenzyl)-4-((2-(2-(3-methoxyphenoxy)ethoxy)ethoxy)methyl)aniline; N,N-bis(3-methoxybenzyl)-4-((2-(2-morpholinoethoxy)ethoxy)methyl)aniline; N,N-bis(3-methoxybenzyl)-4-((2-(3-methoxybenzyloxy)ethoxy)methyl)aniline; 3-(2-(4-(bis(3-methoxybenzyl)amino)benzyloxy)ethoxy)-N,N-dimethylaniline; N,N-bis(3-methoxybenzyl)-4-((2-morpholinoethoxy)methyl)aniline; N,N-bis(3-methoxybenzyl)-4-(morpholinomethyl)aniline; N,N-bis(3-methoxybenzyl)-4-(piperidin-1-ylmethyl)aniline; N,N-bis(3-methoxybenzyl)-4-((4-methylpiperazin-1-yl)methyl)aniline; 4-((dimethylamino)methyl)-N,N-bis(3-methoxybenzyl)aniline; 1-(4-(bis(3-methoxybenzyl)amino)benzyl)piperazin-2-one; 4-(4-(bis(3-methoxybenzyl)amino)benzyl)piperazin-2-one; 1-(4-(bis(3-methoxybenzyl)amino)benzyl)piperazine-2,5-dione; 3-(((3-methoxybenzyl)(4-((2-(2-(3-methoxybenzyloxy)ethoxy)ethoxy)methyl)phenyl)amino)methyl)-N,N-dimethylaniline; 3-(((4-((2-(2-(benzyloxy)ethoxy)ethoxy)methyl)phenyl)(3-methoxybenzyl)amino)methyl)-N,N-dimethylaniline; 3-(((3-methoxybenzyl)(4-((2-(2-(3-methoxyphenoxy)ethoxy)ethoxy)methyl)phenyl)amino)methyl)-N,N-dimethylaniline; 3-(((3-methoxybenzyl)(4-((2-(2-morpholinoethoxy)ethoxy)methyl)phenyl)amino)methyl)-N,N-dimethylaniline; 3-(((3-methoxybenzyl)(4-((2-(3-methoxybenzyloxy)ethoxy)methyl)phenyl)amino)methyl)-N,N-dimethylaniline; 3-(2-(4-((3-(dimethylamino)benzyl)(3-methoxybenzyl)amino)benzyloxy)ethoxy)-N,N-dimethylaniline; 3-(((3-methoxybenzyl)(4-((2-morpholinoethoxy)methyl)phenyl)amino)methyl)-N,N-dimethylaniline; 3-(((3-methoxybenzyl)(4-(morpholinomethyl)phenyl)amino)methyl)-N,N-dimethylaniline; 3-(((3-methoxybenzyl)(4-(piperidin-1-ylmethyl)phenyl)amino)methyl)-N,N-dimethylaniline; 3-(((3-methoxybenzyl)(4-((4-methylpiperazin-1-yl)methyl)phenyl)amino)methyl)-N,N-dimethylaniline; 1-(4-((3-(dimethylamino)benzyl)(3-methoxybenzyl)amino)benzyl)piperazin-2-one; 1-(4-((3-(dimethylamino)benzyl)(3-methoxybenzyl)amino)benzyl)piperazine-2,5-dione; 4-(4-((3-(dimethylamino)benzyl)(3-methoxybenzyl)amino)benzyl)piperazin-2-one; 3-(((4-((dimethylamino)methyl)phenyl)(3-methoxybenzyl)amino)methyl)-N,N-dimethylaniline; N-(3-methoxybenzyl)-4-((2-(2-morpholinoethoxy)ethoxy)methyl)-N-(3-(pyrrolidin-1-yl)benzyl)aniline; N-(3-methoxybenzyl)-4-((2-(3-methoxybenzyloxy)ethoxy)methyl)-N-(3-(pyrrolidin-1-yl)benzyl)aniline; 3-(2-(4-((3-methoxybenzyl)(3-(pyrrolidin-1-yl)benzyl)amino)benzyloxy)ethoxy)-N,N-dimethylaniline; N-(3-methoxybenzyl)-4-((2-morpholinoethoxy)methyl)-N-(3-(pyrrolidin-1-yl)benzyl)aniline; N-(3-methoxybenzyl)-4-(morpholinomethyl)-N-(3-(pyrrolidin-1-yl)benzyl)aniline; N-(3-methoxybenzyl)-4-(piperidin-1-ylmethyl)-N-(3-(pyrrolidin-1-yl)benzyl)aniline; N-(3-methoxybenzyl)-4-((4-methylpiperazin-1-yl)methyl)-N-(3-(pyrrolidin-1-yl)benzyl)aniline; 1-(4-((3-methoxybenzyl)(3-(pyrrolidin-1-yl)benzyl)amino)benzyl)piperazin-2-one; 1-(4-((3-methoxybenzyl)(3-(pyrrolidin-1-yl)benzyl)amino)benzyl)piperazine-2,5-dione; 4-(4-((3-methoxybenzyl)(3-(pyrrolidin-1-yl)benzyl)amino)benzyl)piperazin-2-one; 4-((dimethylamino)methyl)-N-(3-methoxybenzyl)-N-(3-(pyrrolidin-1-yl)benzyl)aniline; N-(3-(1H-pyrazol-1-yl)benzyl)-N-(3-methoxybenzyl)-4-((2-(2-morpholinoethoxy)ethoxy)methyl)aniline; N-(3-(1H-pyrazol-1-yl)benzyl)-N-(3-methoxybenzyl)-4-((2-(3 methoxybenzyloxy)ethoxy)methyl)aniline; 3-(2-(4-((3-(1H-pyrazol-1-yl)benzyl)(3-methoxybenzyl)amino)benzyloxy)ethoxy)-N,N-dimethylaniline; N-(3-(1H-pyrazol-1-yl)benzyl)-N-(3-methoxybenzyl)-4-((2-morpholinoethoxy)methyl)aniline; N-(3-(1H-pyrazol-1-yl)benzyl)-N-(3-methoxybenzyl)-4-(morpholinomethyl)aniline; N-(3-(1H-pyrazol-1-yl)benzyl)-N-(3-methoxybenzyl)-4-(piperidin-1-ylmethyl)aniline; N-(3-(1H-pyrazol-1-yl)benzyl)-N-(3-methoxybenzyl)-4-((4-methylpiperazin-1-yl)methyl)aniline; 1-(4-((3-(1H-pyrazol-1-yl)benzyl)(3-methoxybenzyl)amino)benzyl)piperazin-2-one; 1-(4-((3-(1H-pyrazol-1-yl)benzyl)(3-methoxybenzyl)amino)benzyl)piperazine-2,5-dione; 4-(4-((3-(1H-pyrazol-1-yl)benzyl)(3-methoxybenzyl)amino)benzyl)piperazin-2-one; N-(3-(1H-pyrazol-1-yl)benzyl)-4-((dimethylamino)methyl)-N-(3-methoxybenzyl)aniline N-(3-(1H-imidazol-1-yl)benzyl)-N-(3-methoxybenzyl)-4-((2-(2-morpholinoethoxy)ethoxy)methyl)aniline; N-(3-(1H-imidazol-1-yl)benzyl)-N-(3-methoxybenzyl)-4-((2-(3-methoxybenzyloxy)ethoxy)methyl)aniline; 3-(2-(4-((3-(1H-imidazol-1-yl)benzyl)(3-methoxybenzyl)amino)benzyloxy)ethoxy)-N,N-dimethylaniline; N-(3-(1H-imidazol-1-yl)benzyl)-N-(3-methoxybenzyl)-4-((2-morpholinoethoxy)methyl)aniline; N-(3-(1H-imidazol-1-yl)benzyl)-N-(3-methoxybenzyl)-4-(morpholinomethyl)aniline; N-(3-(1H-imidazol-1-yl)benzyl)-N-(3-methoxybenzyl)-4-(piperidin-1-ylmethyl)aniline; N-(3-(1H-imidazol-1-yl)benzyl)-N-(3-methoxybenzyl)-4-((4-methylpiperazin-1-yl)methyl)aniline; 1-(4-((3-(1H-imidazol-1-yl)benzyl)(3-methoxybenzyl)amino)benzyl)piperazin-2-one; 1-(4-((3-(1H-imidazol-1-yl)benzyl)(3-methoxybenzyl)amino)benzyl)piperazine-2,5-dione; 4-(4-((3-(1H-imidazol-1-yl)benzyl)(3-methoxybenzyl)amino)benzyl)piperazin-2-one; N-(3-(1H-imidazol-1-yl)benzyl)-4-((dimethylamino)methyl)-N-(3-methoxybenzyl)aniline; N-(3-methoxybenzyl)-4-((2-(2-morpholinoethoxy)ethoxy)methyl)-N-(4-(pyrrolidin-1-yl)benzyl)aniline; N-(3-methoxybenzyl)-4-((2-(3-methoxybenzyloxy)ethoxy)methyl)-N-(4-(pyrrolidin-1-yl)benzyl)aniline; 3-(2-(4-((3-methoxybenzyl)(4-(pyrrolidin-1-yl)benzyl)amino)benzyloxy)ethoxy)-N,N-dimethylaniline; N-(3-methoxybenzyl)-4-((2-morpholinoethoxy)methyl)-N-(4-(pyrrolidin-1-yl)benzyl)aniline; N-(3-methoxybenzyl)-4-(morpholinomethyl)-N-(4-(pyrrolidin-1-yl)benzyl)aniline; N-(3-methoxybenzyl)-4-(piperidin-1-ylmethyl)-N-(4-(pyrrolidin-1-yl)benzyl)aniline; N-(3-methoxybenzyl)-4-((4-methylpiperazin-1-yl)methyl)-N-(4-(pyrrolidin-1-yl)benzyl)aniline; 1-(4-((3-methoxybenzyl)(4-(pyrrolidin-1-yl)benzyl)amino)benzyl)piperazin-2-one; 1-(4-((3-methoxybenzyl)(4-(pyrrolidin-1-yl)benzyl)amino)benzyl)piperazine-2,5-dione; 4-(4-((3-methoxybenzyl)(4-(pyrrolidin-1-yl)benzyl)amino)benzyl)piperazin-2-one; 4-((dimethylamino)methyl)-N-(3-methoxybenzyl)-N-(4-(pyrrolidin-1-yl)benzyl)aniline; N-(3-methoxybenzyl)-N-(4-morpholinobenzyl)-4-((2-(2-morpholinoethoxy)ethoxy)methyl)aniline; N-(3-methoxybenzyl)-4-((2-(3-methoxybenzyloxy)ethoxy)methyl)-N-(4-morpholinobenzyl)aniline; 3-(2-(4-((3-methoxybenzyl)(4-morpholinobenzyl)amino)benzyloxy)ethoxy)-N,N-dimethylaniline; N-(3-methoxybenzyl)-N-(4-morpholinobenzyl)-4-((2-morpholinoethoxy)methyl)aniline; N-(3-methoxybenzyl)-N-(4-morpholinobenzyl)-4-(morpholinomethyl)aniline; N-(3-methoxybenzyl)-N-(4-morpholinobenzyl)-4-(piperidin-1-ylmethyl)aniline; N-(3-methoxybenzyl)-4-((4-methylpiperazin-1-yl)methyl)-N-(4-morpholinobenzyl)aniline; 1-(4-((3-methoxybenzyl)(4-morpholinobenzyl)amino)benzyl)piperazin-2-one; 1-(4-((3-methoxybenzyl)(4-morpholinobenzyl)amino)benzyl)piperazine-2,5-dione; 4-(4-((3-methoxybenzyl)(4-morpholinobenzyl)amino)benzyl)piperazin-2-one; 4-((dimethylamino)methyl)-N-(3-methoxybenzyl)-N-(4-morpholinobenzyl)aniline; N-(3-methoxybenzyl)-N-(4-(4-methylpiperazin-1-yl)benzyl)-4-((2-(2-morpholinoethoxy)ethoxy)methyl)aniline; N-(3-methoxybenzyl)-4-((2-(3-methoxybenzyloxy)ethoxy)methyl)-N-(4-(4-methylpiperazin-1-yl)benzyl)aniline; 3-(2-(4-((3-methoxybenzyl)(4-(4-methylpiperazin-1-yl)benzyl)amino)benzyloxy)ethoxy)-N,N-dimethylaniline; N-(3-methoxybenzyl)-N-(4-(4-methylpiperazin-1-yl)benzyl)-4-((2-morpholinoethoxy)methyl)aniline; N-(3-methoxybenzyl)-N-(4-(4-methylpiperazin-1-yl)benzyl)-4-(morpholinomethyl)aniline; N-(3-methoxybenzyl)-N-(4-(4-methylpiperazin-1-yl)benzyl)-4-(piperidin-1-ylmethyl)aniline; N-(3-methoxybenzyl)-N-(4-(4-methylpiperazin-1-yl)benzyl)-4-((4-methylpiperazin-1-yl)methyl)aniline; 1-(4-((3-methoxybenzyl)(4-(4-methylpiperazin-1-yl)benzyl)amino)benzyl)piperazin-2-one; 1-(4-((3-methoxybenzyl)(4-(4-methylpiperazin-1-yl)benzyl)amino)benzyl)piperazine-2,5-dione; 4-(4-((3-methoxybenzyl)(4-(4-methylpiperazin-1-yl)benzyl)amino)benzyl)piperazin-2-one; 4-((dimethylamino)methyl)-N-(3-methoxybenzyl)-N-(4-(4-methylpiperazin-1-yl)benzyl)aniline; N-(3-methoxybenzyl)-N-(3-morpholinobenzyl)-4-((2-(2-morpholinoethoxy)ethoxy)methyl)aniline; N-(3-methoxybenzyl)-4-((2-(3-methoxybenzyloxy)ethoxy)methyl)-N-(3-morpholinobenzyl)aniline; 3-(2-(4-((3-methoxybenzyl)(3-morpholinobenzyl)amino)benzyloxy)ethoxy)-N,N-dimethylaniline; N-(3-methoxybenzyl)-N-(3-morpholinobenzyl)-4-((2-morpholinoethoxy)methyl)aniline; N-(3-methoxybenzyl)-N-(3-morpholinobenzyl)-4-(morpholinomethyl)aniline; N-(3-methoxybenzyl)-N-(3-morpholinobenzyl)-4-(piperidin-1-ylmethyl)aniline; N-(3-methoxybenzyl)-4-((4-methylpiperazin-1-yl)methyl)-N-(3-morpholinobenzyl)aniline; 1-(4-((3-methoxybenzyl)(3-morpholinobenzyl)amino)benzyl)piperazin-2-one; 1-(4-((3-methoxybenzyl)(3-morpholinobenzyl)amino)benzyl)piperazine-2,5-dione; 4-(4-((3-methoxybenzyl)(3-morpholinobenzyl)amino)benzyl)piperazin-2-one; 4-((dimethylamino)methyl)-N-(3-methoxybenzyl)-N-(3-morpholinobenzyl)aniline; N-(3-methoxybenzyl)-N-(3-(4-methylpiperazin-1-yl)benzyl)-4-((2-(2-morpholinoethoxy)ethoxy)methyl)aniline; N-(3-methoxybenzyl)-4-((2-(3-methoxybenzyloxy)ethoxy)methyl)-N-(3-(4-methylpiperazin-1-yl)benzyl)aniline; 3-(2-(4-((3-methoxybenzyl)(3-(4-methylpiperazin-1-yl)benzyl)amino)benzyloxy)ethoxy)-N,N-dimethylaniline; N-(3-methoxybenzyl)-N-(3-(4-methylpiperazin-1-yl)benzyl)-4-((2-morpholinoethoxy)methyl)aniline; N-(3-methoxybenzyl)-N-(3-(4-methylpiperazin-1-yl)benzyl)-4-(morpholinomethyl)aniline; N-(3-methoxybenzyl)-N-(3-(4-methylpiperazin-1-yl)benzyl)-4-(piperidin-1-ylmethyl)aniline; N-(3-methoxybenzyl)-N-(3-(4-methylpiperazin-1-yl)benzyl)-4-((4-methylpiperazin-1-yl)methyl)aniline; 1-(4-((3-methoxybenzyl)(3-(4-methylpiperazin-1-yl)benzyl)amino)benzyl)piperazin-2-one; 1-(4-((3-methoxybenzyl)(3-(4-methylpiperazin-1-yl)benzyl)amino)benzyl)piperazine-2,5-dione; 4-(4-((3-methoxybenzyl)(3-(4-methylpiperazin-1-yl)benzyl)amino)benzyl)piperazin-2-one; 4-((dimethylamino)methyl)-N-(3-methoxybenzyl)-N-(3-(4-methylpiperazin-1-yl)benzyl)aniline; N-(4-(1H-pyrazol-1-yl)benzyl)-N-(3-methoxybenzyl)-4-((2-(2-morpholinoethoxy)ethoxy)methyl)aniline; N-(4-(1H-pyrazol-1-yl)benzyl)-N-(3-methoxybenzyl)-4-((2-(3-methoxybenzyloxy)ethoxy)methyl)aniline; 3-(2-(4-((4-(1H-pyrazol-1-yl)benzyl)(3-methoxybenzyl)amino)benzyloxy)ethoxy)-N,N-dimethylaniline; N-(4-(1H-pyrazol-1-yl)benzyl)-N-(3-methoxybenzyl)-4-((2-morpholinoethoxy)methyl)aniline; N-(4-(1H-pyrazol-1-yl)benzyl)-N-(3-methoxybenzyl)-4-(morpholinomethyl)aniline; N-(4-(1H-pyrazol-1-yl)benzyl)-N-(3-methoxybenzyl)-4-(piperidin-1-ylmethyl)aniline; N-(4-(1H-pyrazol-1-yl)benzyl)-N-(3-methoxybenzyl)-4-((4-methylpiperazin-1-yl)methyl)aniline; 1-(4-((4-(1H-pyrazol-1-yl)benzyl)(3-methoxybenzyl)amino)benzyl)piperazin-2-one; 1-(4-((4-(1H-pyrazol-1-yl)benzyl)(3-methoxybenzyl)amino)benzyl)piperazine-2,5-dione; 4-(4-((4-(1H-pyrazol-1-yl)benzyl)(3-methoxybenzyl)amino)benzyl)piperazin-2-one; N-(4-(1H-pyrazol-1-yl)benzyl)-4-((dimethylamino)methyl)-N-(3-methoxybenzyl)aniline; N-(4-(1H-imidazol-1-yl)benzyl)-N-(3-methoxybenzyl)-4-((2-(2-morpholinoethoxy)ethoxy)methyl)aniline; N-(4-(1H-imidazol-1-yl)benzyl)-N-(3-methoxybenzyl)-4-((2-(3-methoxybenzyloxy)ethoxy)methyl)aniline; 3-(2-(4-((4-(1H-imidazol-1-yl)benzyl)(3-methoxybenzyl)amino)benzyloxy)ethoxy)-N,N-dimethylaniline; N-(4-(1H-imidazol-1-yl)benzyl)-N-(3-methoxybenzyl)-4-((2-morpholinoethoxy)methyl)aniline; N-(4-(1H-imidazol-1-yl)benzyl)-N-(3-methoxybenzyl)-4-(morpholinomethyl)aniline; N-(4-(1H-imidazol-1-yl)benzyl)-N-(3-methoxybenzyl)-4-(piperidin-1-ylmethyl)aniline; N-(4-(1H-imidazol-1-yl)benzyl)-N-(3-methoxybenzyl)-4-((4-methylpiperazin-1-yl)methyl)aniline; 1-(4-((4-(1H-imidazol-1-yl)benzyl)(3-methoxybenzyl)amino)benzyl)piperazin-2-one; 1-(4-((4-(1H-imidazol-1-yl)benzyl)(3-methoxybenzyl)amino)benzyl)piperazine-2,5-dione; 4-(4-((4-(1H-imidazol-1-yl)benzyl)(3-methoxybenzyl)amino)benzyl)piperazin-2-one; N-(4-(1H-imidazol-1-yl)benzyl)-4-((dimethylamino)methyl)-N-(3-methoxybenzyl)aniline; N-((1H-benzo[d]imidazol-6-yl)methyl)-N-(3-methoxybenzyl)-4-((2-(2-morpholinoethoxy)ethoxy)methyl)aniline; N-((1H-benzo[d]imidazol-6-yl)methyl)-N-(3-methoxybenzyl)-4-((2-(3-methoxybenzyloxy)ethoxy)methyl)aniline; 3-(2-(4-(((1H-benzo[d]imidazol-6-yl)methyl)(3-methoxybenzyl)amino)benzyloxy)ethoxy)-N,N-dimethylaniline; N-((1H-benzo[d]imidazol-6-yl)methyl)-N-(3-methoxybenzyl)-4-((2-morpholinoethoxy)methyl)aniline; N-((1H-benzo[d]imidazol-6-yl)methyl)-N-(3-methoxybenzyl)-4-(morpholinomethyl)aniline; N-((1H-benzo[d]imidazol-6-yl)methyl)-N-(3-methoxybenzyl)-4-(piperidin-1-ylmethyl)aniline; N-((1H-benzo[d]imidazol-6-yl)methyl)-N-(3-methoxybenzyl)-4-((4-methylpiperazin-1-yl)methyl)aniline; 1-(4-(((1H-benzo[d]imidazol-6-yl)methyl)(3-methoxybenzyl)amino)benzyl)piperazin-2-one; 1-(4-(((1H-benzo[d]imidazol-6-yl)methyl)(3-methoxybenzyl)amino)benzyl)piperazine-2,5-dione; 4-(4-(((1H-benzo[d]imidazol-6-yl)methyl)(3-methoxybenzyl)amino)benzyl)piperazin-2-one; N-((1H-benzo[d]imidazol-6-yl)methyl)-4-((dimethylamino)methyl)-N-(3-methoxybenzyl)aniline; N-((1H-indazol-6-yl)methyl)-N-(3-methoxybenzyl)-4-((2-(2-morpholinoethoxy)ethoxy)methyl)aniline; N-((1H-indazol-6-yl)methyl)-N-(3-methoxybenzyl)-4-((2-(3-methoxybenzyloxy)ethoxy)methyl)aniline; 3-(2-(4-(((1H-indazol-6-yl)methyl)(3-methoxybenzyl)amino)benzyloxy)ethoxy)-N,N-dimethylaniline; N-((1H-indazol-6-yl)methyl)-N-(3-methoxybenzyl)-4-((2-morpholinoethoxy)methyl)aniline; N-((1H-indazol-6-yl)methyl)-N-(3-methoxybenzyl)-4-(morpholinomethyl)aniline; N-((1H-indazol-6-yl)methyl)-N-(3-methoxybenzyl)-4-(piperidin-1-ylmethyl)aniline; N-((1H-indazol-6-yl)methyl)-N-(3-methoxybenzyl)-4-((4-methylpiperazin-1-yl)methyl)aniline; 1-(4-(((1H-indazol-6-yl)methyl)(3-methoxybenzyl)amino)benzyl)piperazin-2-one; 1-(4-(((1H-indazol-6-yl)methyl)(3-methoxybenzyl)amino)benzyl)piperazine-2,5-dione; 4-(4-(((1H-indazol-6-yl)methyl)(3-methoxybenzyl)amino)benzyl)piperazin-2-one; N-((1H-indazol-6-yl)methyl)-4-((dimethylamino)methyl)-N-(3-methoxybenzyl)aniline; N-(3-methoxybenzyl)-4-((2-(2-morpholinoethoxy)ethoxy)methyl)-N-(quinolin-7-ylmethyl)aniline; N-(3-methoxybenzyl)-4-((2-(3-methoxybenzyloxy)ethoxy)methyl)-N-(quinolin-7-ylmethyl)aniline; 3-(2-(4-((3-methoxybenzyl)(quinolin-7-ylmethyl)amino)benzyloxy)ethoxy)-N,N-dimethylaniline; N-(3-methoxybenzyl)-4-((2-morpholinoethoxy)methyl)-N-(quinolin-7-ylmethyl)aniline; N-(3-methoxybenzyl)-4-(morpholinomethyl)-N-(quinolin-7-ylmethyl)aniline; N-(3-methoxybenzyl)-4-((4-methylpiperazin-1-yl)methyl)-N-(quinolin-7-ylmethyl)aniline; 1-(4-((3-methoxybenzyl)(quinolin-7-ylmethyl)amino)benzyl)piperazin-2-one; 1-(4-((3-methoxybenzyl)(quinolin-7-ylmethyl)amino)benzyl)piperazine-2,5-dione; 4-(4-((3-methoxybenzyl)(quinolin-7-ylmethyl)amino)benzyl)piperazin-2-one; 4-((dimethylamino)methyl)-N-(3-methoxybenzyl)-N-(quinolin-7-ylmethyl)aniline; N,N-bis(3-methoxybenzyl)-3-((2-(2-(3-methoxybenzyloxy)ethoxy)ethoxy)methyl)aniline; 3-((2-(2-(benzyloxy)ethoxy)ethoxy)methyl)-N,N-bis(3-methoxybenzyl)aniline; N,N-bis(3-methoxybenzyl)-3-((2-(2-(3-methoxyphenoxy)ethoxy)ethoxy)methyl)aniline; N,N-bis(3-methoxybenzyl)-3-((2-(2-morpholinoethoxy)ethoxy)methyl)aniline; N,N-bis(3-methoxybenzyl)-3-((2-(3-methoxybenzyloxy)ethoxy)methyl)aniline; 3-(2-(3-(bis(3-methoxybenzyl)amino)benzyloxy)ethoxy)-N,N-dimethylaniline; N,N-bis(3-methoxybenzyl)-3-((2-morpholinoethoxy)methyl)aniline; N,N-bis(3-methoxybenzyl)-3-(morpholinomethyl)aniline; N,N-bis(3-methoxybenzyl)-3-(piperidin-1-ylmethyl)aniline; N,N-bis(3-methoxybenzyl)-3-((4-methylpiperazin-1-yl)methyl)aniline; 1-(3-(bis(3-methoxybenzyl)amino)benzyl)piperazin-2-one; 1-(3-(bis(3-methoxybenzyl)amino)benzyl)piperazine-2,5-dione; 4-(3-(bis(3-methoxybenzyl)amino)benzyl)piperazin-2-one; 3-((dimethylamino)methyl)-N,N-bis(3-methoxybenzyl)aniline; N-(3-(dimethylamino)benzyl)-N-(3-methoxybenzyl)-3-((2-(2-(3-methoxybenzyloxy)ethoxy)ethoxy)methyl)aniline; 3-((2-(2-(benzyloxy)ethoxy)ethoxy)methyl)-N-(3-(dimethylamino)benzyl)-N-(3-methoxybenzyl)aniline; N-(3-(dimethylamino)benzyl)-N-(3-methoxybenzyl)-3-((2-(2-(3-methoxyphenoxy)ethoxy)ethoxy)methyl)aniline; N-(3-(dimethylamino)benzyl)-N-(3-methoxybenzyl)-3-((2-(2-morpholinoethoxy)ethoxy)methyl)aniline; N-(3-(dimethylamino)benzyl)-N-(3-methoxybenzyl)-3-((2-(3-methoxybenzyloxy)ethoxy)methyl)aniline; N-(3-(dimethylamino)benzyl)-3-((2-(3-(dimethylamino)phenoxy)ethoxy)methyl)-N-(3-methoxybenzyl)aniline; N-(3-(dimethylamino)benzyl)-N-(3-methoxybenzyl)-3-((2-morpholinoethoxy)methyl)aniline; N-(3-(dimethylamino)benzyl)-N-(3-methoxybenzyl)-3-(morpholinomethyl)aniline; N-(3-(dimethylamino)benzyl)-N-(3-methoxybenzyl)-3-(piperidin-1-ylmethyl)aniline; N-(3-(dimethylamino)benzyl)-N-(3-methoxybenzyl)-3-((4-methylpiperazin-1-yl)methyl)aniline; 1-(3-((3-(dimethylamino)benzyl)(3-methoxybenzyl)amino)benzyl)piperazin-2-one; 1-(3-((3-(dimethylamino)benzyl)(3-methoxybenzyl)amino)benzyl)piperazine-2,5-dione; 4-(3-((3-(dimethylamino)benzyl)(3-methoxybenzyl)amino)benzyl)piperazin-2-one; N-(3-(dimethylamino)benzyl)-3-((dimethylamino)methyl)-N-(3-methoxybenzyl)aniline; N-(3-methoxybenzyl)-3-((2-(2-morpholinoethoxy)ethoxy)methyl)-N-(3-(pyrrolidin-1-yl)benzyl)aniline; N-(3-methoxybenzyl)-3-((2-(3-methoxybenzyloxy)ethoxy)methyl)-N-(3-(pyrrolidin-1-yl)benzyl)aniline; 3-((2-(3-(dimethylamino)phenoxy)ethoxy)methyl)-N-(3-methoxybenzyl)-N-(3-(pyrrolidin-1-yl)benzyl)aniline; N-(3-methoxybenzyl)-3-((2-morpholinoethoxy)methyl)-N-(3-(pyrrolidin-1-yl)benzyl)aniline; N-(3-methoxybenzyl)-3-(morpholinomethyl)-N-(3-(pyrrolidin-1-yl)benzyl)aniline; N-(3-methoxybenzyl)-3-(piperidin-1-ylmethyl)-N-(3-(pyrrolidin-1-yl)benzyl)aniline; N-(3-methoxybenzyl)-3-((4-methylpiperazin-1-yl)methyl)-N-(3-(pyrrolidin-1-yl)benzyl)aniline; 1-(3-((3-methoxybenzyl)(3-(pyrrolidin-1-yl)benzyl)amino)benzyl)piperazin-2-one; 1-(3-((3-methoxybenzyl)(3-(pyrrolidin-1-yl)benzyl)amino)benzyl)piperazine-2,5-dione; 4-(3-((3-methoxybenzyl)(3-(pyrrolidin-1-yl)benzyl)amino)benzyl)piperazin-2-one; 3-((dimethylamino)methyl)-N-(3-methoxybenzyl)-N-(3-(pyrrolidin-1-yl)benzyl)aniline; N-(3-(1H-pyrazol-1-yl)benzyl)-N-(3-methoxybenzyl)-3-((2-(2-morpholinoethoxy)ethoxy)methyl)aniline; N-(3-(1H-pyrazol-1-yl)benzyl)-N-(3-methoxybenzyl)-3-((2-(3 methoxybenzyloxy)ethoxy)methyl)aniline; N-(3-(1H-pyrazol-1-yl)benzyl)-3-((2-(3-(dimethylamino)phenoxy)ethoxy)methyl)-N-(3-methoxybenzyl)aniline; N-(3-(1H-pyrazol-1-yl)benzyl)-N-(3-methoxybenzyl)-3-((2-morpholinoethoxy)methyl)aniline; N-(3-(1H-pyrazol-1-yl)benzyl)-N-(3-methoxybenzyl)-3-(morpholinomethyl)aniline; N-(3-(1H-pyrazol-1-yl)benzyl)-N-(3-methoxybenzyl)-3-(piperidin-1-ylmethyl)aniline; N-(3-(1H-pyrazol-1-yl)benzyl)-N-(3-methoxybenzyl)-3-((4-methylpiperazin-1-yl)methyl)aniline; 1-(3-((3-(1H-pyrazol-1-yl)benzyl)(3-methoxybenzyl)amino)benzyl)piperazin-2-one; 1-(3-((3-(1H-pyrazol-1-yl)benzyl)(3-methoxybenzyl)amino)benzyl)piperazine-2,5-dione; 4-(3-((3-(1H-pyrazol-1-yl)benzyl)(3-methoxybenzyl)amino)benzyl)piperazin-2-one; N-(3-(1H-pyrazol-1-yl)benzyl)-3-((dimethylamino)methyl)-N-(3-methoxybenzyl)aniline; N-(3-(1H-imidazol-1-yl)benzyl)-N-(3-methoxybenzyl)-3-((2-(2-morpholinoethoxy)ethoxy)methyl)aniline; N-(3-(1H-imidazol-1-yl)benzyl)-N-(3-methoxybenzyl)-3-((2-(3-methoxybenzyloxy)ethoxy)methyl)aniline; N-(3-(1H-imidazol-1-yl)benzyl)-3-((2-(3-(dimethylamino)phenoxy)ethoxy)methyl)-N-(3-methoxybenzyl)aniline; N-(3-(1H-imidazol-1-yl)benzyl)-N-(3-methoxybenzyl)-3-((2-morpholinoethoxy)methyl)aniline; N-(3-(1H-imidazol-1-yl)benzyl)-N-(3-methoxybenzyl)-3-(morpholinomethyl)aniline; N-(3-(1H-imidazol-1-yl)benzyl)-N-(3-methoxybenzyl)-3-(piperidin-1-ylmethyl)aniline; N-(3-(1H-imidazol-1-yl)benzyl)-N-(3-methoxybenzyl)-3-((4-methylpiperazin-1-yl)methyl)aniline; 1-(3-((3-(1H-imidazol-1-yl)benzyl)(3-methoxybenzyl)amino)benzyl)piperazin-2-one; 1-(3-((3-(1H-imidazol-1-yl)benzyl)(3-methoxybenzyl)amino)benzyl)piperazine-2,5-dione; 4-(3-((3-(1H-imidazol-1-yl)benzyl)(3-methoxybenzyl)amino)benzyl)piperazin-2-one; N-(3-(1H-imidazol-1-yl)benzyl)-3-((dimethylamino)methyl)-N-(3-methoxybenzyl)aniline; 3-((2-(3-(dimethylamino)phenoxy)ethoxy)methyl)-N-(3-methoxybenzyl)-N-(4-(pyrrolidin-1-yl)benzyl)aniline; N-(3-methoxybenzyl)-3-((2-(2-morpholinoethoxy)ethoxy)methyl)-N-(4-(pyrrolidin-1-yl)benzyl)aniline; N-(3-methoxybenzyl)-3-((2-(3-methoxybenzyloxy)ethoxy)methyl)-N-(4-(pyrrolidin-1-yl)benzyl)aniline; N-(3-methoxybenzyl)-3-((2-morpholinoethoxy)methyl)-N-(4-(pyrrolidin-1-yl)benzyl)aniline; N-(3-methoxybenzyl)-3-(morpholinomethyl)-N-(4-(pyrrolidin-1-yl)benzyl)aniline; N-(3-methoxybenzyl)-3-(piperidin-1-ylmethyl)-N-(4-(pyrrolidin-1-yl)benzyl)aniline; N-(3-methoxybenzyl)-3-((4-methylpiperazin-1-yl)methyl)-N-(4-(pyrrolidin-1-yl)benzyl)aniline; 1-(3-((3-methoxybenzyl)(4-(pyrrolidin-1-yl)benzyl)amino)benzyl)piperazin-2-one; 1-(3-((3-methoxybenzyl)(4-(pyrrolidin-1-yl)benzyl)amino)benzyl)piperazine-2,5-dione; 4-(3-((3-methoxybenzyl)(4-(pyrrolidin-1-yl)benzyl)amino)benzyl)piperazin-2-one; 3-((dimethylamino)methyl)-N-(3-methoxybenzyl)-N-(4-(pyrrolidin-1-yl)benzyl)aniline; N-(3-methoxybenzyl)-N-(4-morpholinobenzyl)-3-((2-(2-morpholinoethoxy)ethoxy)methyl)aniline; N-(3-methoxybenzyl)-3-((2-(3-methoxybenzyloxy)ethoxy)methyl)-N-(4-morpholinobenzyl)aniline; 3-((2-(3-(dimethylamino)phenoxy)ethoxy)methyl)-N-(3-methoxybenzyl)-N-(4-morpholinobenzyl)aniline; N-(3-methoxybenzyl)-N-(4-morpholinobenzyl)-3-((2-morpholinoethoxy)methyl)aniline; N-(3-methoxybenzyl)-N-(4-morpholinobenzyl)-3-(morpholinomethyl)aniline; N-(3-methoxybenzyl)-N-(4-morpholinobenzyl)-3-(piperidin-1-ylmethyl)aniline; N-(3-methoxybenzyl)-3-((4-methylpiperazin-1-yl)methyl)-N-(4-morpholinobenzyl)aniline; 1-(3-((3-methoxybenzyl)(4-morpholinobenzyl)amino)benzyl)piperazin-2-one; 1-(3-((3-methoxybenzyl)(4-morpholinobenzyl)amino)benzyl)piperazine-2,5-dione; 4-(3-((3-methoxybenzyl)(4-morpholinobenzyl)amino)benzyl)piperazin-2-one; 3-((dimethylamino)methyl)-N-(3-methoxybenzyl)-N-(4-morpholinobenzyl)aniline; N-(3-methoxybenzyl)-N-(4-(4-methylpiperazin-1-yl)benzyl)-3-((2-(2-morpholinoethoxy)ethoxy)methyl)aniline; N-(3-methoxybenzyl)-3-((2-(3-methoxybenzyloxy)ethoxy)methyl)-N-(4-(4-methylpiperazin-1-yl)benzyl)aniline; 3-((2-(3-(dimethylamino)phenoxy)ethoxy)methyl)-N-(3-methoxybenzyl)-N-(4-(4-methylpiperazin-1-yl)benzyl)aniline; N-(3-methoxybenzyl)-N-(4-(4-methylpiperazin-1-yl)benzyl)-3-((2-morpholinoethoxy)methyl)aniline; N-(3-methoxybenzyl)-N-(4-(4-methylpiperazin-1-yl)benzyl)-3-(morpholinomethyl)aniline; N-(3-methoxybenzyl)-N-(4-(4-methylpiperazin-1-yl)benzyl)-3-(piperidin-1-ylmethyl)aniline; N-(3-methoxybenzyl)-N-(4-(4-methylpiperazin-1-yl)benzyl)-3-((4-methylpiperazin-1-yl)methyl)aniline; 1-(3-((3-methoxybenzyl)(4-(4-methylpiperazin-1-yl)benzyl)amino)benzyl)piperazin-2-one; 1-(3-((3-methoxybenzyl)(4-(4-methylpiperazin-1-yl)benzyl)amino)benzyl)piperazine-2,5-dione; 3-((dimethylamino)methyl)-N-(3-methoxybenzyl)-N-(4-(4-methylpiperazin-1-yl)benzyl)aniline; N-(3-methoxybenzyl)-N-(3-morpholinobenzyl)-3-((2-(2-morpholinoethoxy)ethoxy)methyl)aniline; N-(3-methoxybenzyl)-3-((2-(3-methoxybenzyloxy)ethoxy)methyl)-N-(3-morpholinobenzyl)aniline; 3-((2-(3-(dimethylamino)phenoxy)ethoxy)methyl)-N-(3-methoxybenzyl)-N-(3-morpholinobenzyl)aniline; N-(3-methoxybenzyl)-N-(3-morpholinobenzyl)-3-((2-morpholinoethoxy)methyl)aniline; N-(3-methoxybenzyl)-N-(3-morpholinobenzyl)-3-(morpholinomethyl)aniline; N-(3-methoxybenzyl)-N-(3-morpholinobenzyl)-3-(piperidin-1-ylmethyl)aniline; N-(3-methoxybenzyl)-3-((4-methylpiperazin-1-yl)methyl)-N-(3-morpholinobenzyl)aniline; 1-(3-((3-methoxybenzyl)(3-morpholinobenzyl)amino)benzyl)piperazin-2-one; 1-(3-((3-methoxybenzyl)(3-morpholinobenzyl)amino)benzyl)piperazine-2,5-dione; 4-(3-((3-methoxybenzyl)(3-morpholinobenzyl)amino)benzyl)piperazin-2-one; 3-((dimethylamino)methyl)-N-(3-methoxybenzyl)-N-(3-morpholinobenzyl)aniline; N-(3-methoxybenzyl)-N-(3-(4-methylpiperazin-1-yl)benzyl)-3-((2-(2-morpholinoethoxy)ethoxy)methyl)aniline; N-(3-methoxybenzyl)-3-((2-(3-methoxybenzyloxy)ethoxy)methyl)-N-(3-(4-methylpiperazin-1-yl)benzyl)aniline; 3-((2-(3-(dimethylamino)phenoxy)ethoxy)methyl)-N-(3-methoxybenzyl)-N-(3-(4-methylpiperazin-1-yl)benzyl)aniline; N-(3-methoxybenzyl)-N-(3-(4-methylpiperazin-1-yl)benzyl)-3-((2-morpholinoethoxy)methyl)aniline; N-(3-methoxybenzyl)-N-(3-(4-methylpiperazin-1-yl)benzyl)-3-(morpholinomethyl)aniline; N-(3-methoxybenzyl)-N-(3-(4-methylpiperazin-1-yl)benzyl)-3-(piperidin-1-ylmethyl)aniline; N-(3-methoxybenzyl)-N-(3-(4-methylpiperazin-1-yl)benzyl)-3-((4-methylpiperazin-1-yl)methyl)aniline; 1-(3-((3-methoxybenzyl)(3-(4-methylpiperazin-1-yl)benzyl)amino)benzyl)piperazin-2-one; 1-(3-((3-methoxybenzyl)(3-(4-methylpiperazin-1-yl)benzyl)amino)benzyl)piperazine-2,5-dione; 4-(3-((3-methoxybenzyl)(3-(4-methylpiperazin-1-yl)benzyl)amino)benzyl)piperazin-2-one; 3-((dimethylamino)methyl)-N-(3-methoxybenzyl)-N-(3-(4-methylpiperazin-1-yl)benzyl)aniline; N-(4-(1H-pyrazol-1-yl)benzyl)-N-(3-methoxybenzyl)-3-((2-(2-morpholinoethoxy)ethoxy)methyl)aniline; N-(4-(1H-pyrazol-1-yl)benzyl)-N-(3-methoxybenzyl)-3-((2-(3-methoxybenzyloxy)ethoxy)methyl)aniline; N-(4-(1H-pyrazol-1-yl)benzyl)-3-((2-(3-(dimethylamino)phenoxy)ethoxy)methyl)-N-(3-methoxybenzyl)aniline; N-(4-(1H-pyrazol-1-yl)benzyl)-N-(3-methoxybenzyl)-3-((2-morpholinoethoxy)methyl)aniline; N-(4-(1H-pyrazol-1-yl)benzyl)-N-(3-methoxybenzyl)-3-(morpholinomethyl)aniline; N-(4-(1H-pyrazol-1-yl)benzyl)-N-(3-methoxybenzyl)-3-(piperidin-1-ylmethyl)aniline N-(4-(1H-pyrazol-1-yl)benzyl)-N-(3-methoxybenzyl)-3-((4-methylpiperazin-1-yl)methyl)aniline; 1-(3-((4-(1H-pyrazol-1-yl)benzyl)(3-methoxybenzyl)amino)benzyl)piperazin-2-one; 1-(3-((4-(1H-pyrazol-1-yl)benzyl)(3-methoxybenzyl)amino)benzyl)piperazine-2,5-dione; 4-(3-((4-(1H-pyrazol-1-yl)benzyl)(3-methoxybenzyl)amino)benzyl)piperazin-2-one; N-(4-(1H-pyrazol-1-yl)benzyl)-3-((dimethylamino)methyl)-N-(3-methoxybenzyl)aniline; N-(4-(1H-imidazol-1-yl)benzyl)-N-(3-methoxybenzyl)-3-((2-(2-morpholinoethoxy)ethoxy)methyl)aniline; N-(4-(1H-imidazol-1-yl)benzyl)-N-(3-methoxybenzyl)-3-((2-(3-methoxybenzyloxy)ethoxy)methyl)aniline; N-(4-(1H-imidazol-1-yl)benzyl)-3-((2-(3-(dimethylamino)phenoxy)ethoxy)methyl)-N-(3-methoxybenzyl)aniline; N-(4-(1H-imidazol-1-yl)benzyl)-N-(3-methoxybenzyl)-3-((2-morpholinoethoxy)methyl)aniline; N-(4-(1H-imidazol-1-yl)benzyl)-N-(3-methoxybenzyl)-3-(morpholinomethyl)aniline; N-(4-(1H-imidazol-1-yl)benzyl)-N-(3-methoxybenzyl)-3-(piperidin-1-ylmethyl)aniline; N-(4-(1H-imidazol-1-yl)benzyl)-N-(3-methoxybenzyl)-3-((4-methylpiperazin-1-yl)methyl)aniline; 1-(3-((4-(1H-imidazol-1-yl)benzyl)(3-methoxybenzyl)amino)benzyl)piperazin-2-one; 1-(3-((4-(1H-imidazol-1-yl)benzyl)(3-methoxybenzyl)amino)benzyl)piperazine-2,5-dione; 4-(3-((4-(1H-imidazol-1-yl)benzyl)(3-methoxybenzyl)amino)benzyl)piperazin-2-one; N-(4-(1H-imidazol-1-yl)benzyl)-3-((dimethylamino)methyl)-N-(3-methoxybenzyl)aniline; N-((1H-benzo[d]imidazol-6-yl)methyl)-N-(3-methoxybenzyl)-3-((2-(2-morpholinoethoxy)ethoxy)methyl)aniline; N-((1H-benzo[d]imidazol-6-yl)methyl)-N-(3-methoxybenzyl)-3-((2-(3-methoxybenzyloxy)ethoxy)methyl)aniline; N-((1H-benzo[d]imidazol-6-yl)methyl)-3-((2-(3-(dimethylamino)phenoxy)ethoxy)methyl)-N-(3-methoxybenzyl)aniline; N-((1H-benzo[d]imidazol-6-yl)methyl)-N-(3-methoxybenzyl)-3-((2-morpholinoethoxy)methyl)aniline; N-((1H-benzo[d]imidazol-6-yl)methyl)-N-(3-methoxybenzyl)-3-(morpholinomethyl)aniline; N-((1H-benzo[d]imidazol-6-yl)methyl)-N-(3-methoxybenzyl)-3-(piperidin-1-ylmethyl)aniline; N-((1H-benzo[d]imidazol-6-yl)methyl)-N-(3-methoxybenzyl)-3-((4-methylpiperazin-1-yl)methyl)aniline; 1-(3-(((1H-benzo[d]imidazol-6-yl)methyl)(3-methoxybenzyl)amino)benzyl)piperazin-2-one; 1-(3-(((1H-benzo[d]imidazol-6-yl)methyl)(3-methoxybenzyl)amino)benzyl)piperazine-2,5-dione; 4-(3-(((1H-benzo[d]imidazol-6-yl)methyl)(3-methoxybenzyl)amino)benzyl)piperazin-2-one; N-((1H-benzo[d]imidazol-6-yl)methyl)-3-((dimethylamino)methyl)-N-(3-methoxybenzyl)aniline; N-((1H-indazol-6-yl)methyl)-N-(3-methoxybenzyl)-3-((2-(2-morpholinoethoxy)ethoxy)methyl)aniline; N-((1H-indazol-6-yl)methyl)-N-(3-methoxybenzyl)-3-((2-(3-methoxybenzyloxy)ethoxy)methyl)aniline; N-((1H-indazol-6-yl)methyl)-3-((2-(3-(dimethylamino)phenoxy)ethoxy)methyl)-N-(3-methoxybenzyl)aniline; N-((1H-indazol-6-yl)methyl)-N-(3-methoxybenzyl)-3-((2-morpholinoethoxy)methyl)aniline; N-((1H-indazol-6-yl)methyl)-N-(3-methoxybenzyl)-3-(morpholinomethyl)aniline; N-((1H-indazol-6-yl)methyl)-N-(3-methoxybenzyl)-3-(piperidin-1-ylmethyl)aniline; N-((1H-indazol-6-yl)methyl)-N-(3-methoxybenzyl)-3-((4-methylpiperazin-1-yl)methyl)aniline; 1-(3-(((1H-indazol-6-yl)methyl)(3-methoxybenzyl)amino)benzyl)piperazin-2-one; 1-(3-(((1H-indazol-6-yl)methyl)(3-methoxybenzyl)amino)benzyl)piperazine-2,5-dione; 4-(3-(((1H-indazol-6-yl)methyl)(3-methoxybenzyl)amino)benzyl)piperazin-2-one; N-((1H-indazol-6-yl)methyl)-3-((dimethylamino)methyl)-N-(3-methoxybenzyl)aniline; N-(3-methoxybenzyl)-3-((2-(2-morpholinoethoxy)ethoxy)methyl)-N-(quinolin-7-ylmethyl)aniline; N-(3-methoxybenzyl)-3-((2-(3-methoxybenzyloxy)ethoxy)methyl)-N-(quinolin-7-ylmethyl)aniline; 3-((2-(3-(dimethylamino)phenoxy)ethoxy)methyl)-N-(3-methoxybenzyl)-N-(quinolin-7-ylmethyl)aniline; N-(3-methoxybenzyl)-3-((2-morpholinoethoxy)methyl)-N-(quinolin-7-ylmethyl)aniline N-(3-methoxybenzyl)-3-(morpholinomethyl)-N-(quinolin-7-ylmethyl)aniline; N-(3-methoxybenzyl)-3-(piperidin-1-ylmethyl)-N-(quinolin-7-ylmethyl)aniline; N-(3-methoxybenzyl)-3-((4-methylpiperazin-1-yl)methyl)-N-(quinolin-7-ylmethyl)aniline; 1-(3-((3-methoxybenzyl)(quinolin-7-ylmethyl)amino)benzyl)piperazin-2-one; 1-(3-((3-methoxybenzyl)(quinolin-7-ylmethyl)amino)benzyl)piperazine-2,5-dione; 4-(3-((3-methoxybenzyl)(quinolin-7-ylmethyl)amino)benzyl)piperazin-2-one; 3-((dimethylamino)methyl)-N-(3-methoxybenzyl)-N-(quinolin-7-ylmethyl)aniline; N,N-bis(3-methoxybenzyl)-2-((2-(2-(3-methoxybenzyloxy)ethoxy)ethoxy)methyl)pyridin-4-amine; 2-((2-(2-(benzyloxy)ethoxy)ethoxy)methyl)-N,N-bis(3-methoxybenzyl)pyridin-4-amine; N,N-bis(3-methoxybenzyl)-2-((2-(2-(3-methoxyphenoxy)ethoxy)ethoxy)methyl)pyridin-4-amine; N,N-bis(3-methoxybenzyl)-2-((2-(2-morpholinoethoxy)ethoxy)methyl)pyridin-4-amine; N,N-bis(3-methoxybenzyl)-2-((2-(3-methoxybenzyloxy)ethoxy)methyl)pyridin-4-amine; 2-((2-(3-(dimethylamino)phenoxy)ethoxy)methyl)-N,N-bis(3-methoxybenzyl)pyridin-4-amine; N,N-bis(3-methoxybenzyl)-2-((2-morpholinoethoxy)methyl)pyridin-4-amine; N,N-bis(3-methoxybenzyl)-2-(morpholinomethyl)pyridin-4-amine; N,N-bis(3-methoxybenzyl)-2-(piperidin-1-ylmethyl)pyridin-4-amine; N,N-bis(3-methoxybenzyl)-2-((4-methylpiperazin-1-yl)methyl)pyridin-4-amine; 1-((4-(bis(3-methoxybenzyl)amino)pyridin-2-yl)methyl)piperazin-2-one; 1-((4-(bis(3-methoxybenzyl)amino)pyridin-2-yl)methyl)piperazine-2,5-dione; 4-((4-(bis(3-methoxybenzyl)amino)pyridin-2-yl)methyl)piperazin-2-one; 2-((dimethylamino)methyl)-N,N-bis(3-methoxybenzyl)pyridin-4-amine; N-(3-(dimethylamino)benzyl)-N-(3-methoxybenzyl)-2-((2-(2-(3-methoxybenzyloxy)ethoxy)ethoxy)methyl)pyridin-4-amine; 2-((2-(2-(benzyloxy)ethoxy)ethoxy)methyl)-N-(3-(dimethylamino)benzyl)-N-(3-methoxybenzyl)pyridin-4-amine; N-(3-(dimethylamino)benzyl)-N-(3-methoxybenzyl)-2-((2-(2-(3-methoxyphenoxy)ethoxy)ethoxy)methyl)pyridin-4-amine; N-(3-(dimethylamino)benzyl)-N-(3-methoxybenzyl)-2-((2-(2-morpholinoethoxy)ethoxy)methyl)pyridin-4-amine; N-(3-(dimethylamino)benzyl)-N-(3-methoxybenzyl)-2-((2-(3-methoxybenzyloxy)ethoxy)methyl)pyridin-4-amine; N-(3-(dimethylamino)benzyl)-2-((2-(3-(dimethylamino)phenoxy)ethoxy)methyl)-N-(3-methoxybenzyl)pyridin-4-amine; N-(3-(dimethylamino)benzyl)-N-(3-methoxybenzyl)-2-((2-morpholinoethoxy)methyl)pyridin-4-amine; N-(3-(dimethylamino)benzyl)-N-(3-methoxybenzyl)-3-(morpholinomethyl)aniline; N-(3-(dimethylamino)benzyl)-N-(3-methoxybenzyl)-2-(piperidin-1-ylmethyl)pyridin-4-amine; N-(3-(dimethylamino)benzyl)-N-(3-methoxybenzyl)-2-((4-methylpiperazin-1-yl)methyl)pyridin-4-amine; 1-((4-((3-(dimethylamino)benzyl)(3-methoxybenzyl)amino)pyridin-2-yl)methyl)piperazin-2-one; 1-((4-((3-(dimethylamino)benzyl)(3-methoxybenzyl)amino)pyridin-2-yl)methyl)piperazine-2,5-dione; 4-((4-((3-(dimethylamino)benzyl)(3-methoxybenzyl)amino)pyridin-2-yl)methyl)piperazin-2-one; N-(3-(dimethylamino)benzyl)-2-((dimethylamino)methyl)-N-(3-methoxybenzyl)pyridin-4-amine; N-(3-methoxybenzyl)-2-((2-(2-morpholinoethoxy)ethoxy)methyl)-N-(3-(pyrrolidin-1-yl)benzyl)pyridin-4-amine; N-(3-methoxybenzyl)-2-((2-(3-methoxybenzyloxy)ethoxy)methyl)-N-(3-(pyrrolidin-1-yl)benzyl)pyridin-4-amine; 2-((2-(3-(dimethylamino)phenoxy)ethoxy)methyl)-N-(3-methoxybenzyl)-N-(3-(pyrrolidin-1-yl)benzyl)pyridin-4-amine; N-(3-methoxybenzyl)-2-((2-morpholinoethoxy)methyl)-N-(3-(pyrrolidin-1-yl)benzyl)pyridin-4-amine; N-(3-methoxybenzyl)-2-(morpholinomethyl)-N-(3-(pyrrolidin-1-yl)benzyl)pyridin-4-amine; N-(3-methoxybenzyl)-2-(piperidin-1-ylmethyl)-N-(3-(pyrrolidin-1-yl)benzyl)pyridin-4-amine; N-(3-methoxybenzyl)-2-((4-methylpiperazin-1-yl)methyl)-N-(3-(pyrrolidin-1-yl)benzyl)pyridin-4-amine; 1-((4-((3-methoxybenzyl)(3-(pyrrolidin-1-yl)benzyl)amino)pyridin-2-yl)methyl)piperazin-2-one; 1-((4-((3-methoxybenzyl)(3-(pyrrolidin-1-yl)benzyl)amino)pyridin-2-yl)methyl)piperazine-2,5-dione; 4-((4-((3-methoxybenzyl)(3-(pyrrolidin-1-yl)benzyl)amino)pyridin-2-yl)methyl)piperazin-2-one; 2-((dimethylamino)methyl)-N-(3-methoxybenzyl)-N-(3-(pyrrolidin-1-yl)benzyl)pyridin-4-amine; N-(3-(1H-pyrazol-1-yl)benzyl)-N-(3-methoxybenzyl)-2-((2-(2-morpholinoethoxy)ethoxy)methyl)pyridin-4-amine; N-(3-(1H-pyrazol-1-yl)benzyl)-N-(3-methoxybenzyl)-2-((2-(3-methoxybenzyloxy)ethoxy)methyl)pyridin-4-amine; N-(3-(1H-pyrazol-1-yl)benzyl)-2-((2-(3-(dimethylamino)phenoxy)ethoxy)methyl)-N-(3-methoxybenzyl)pyridin-4-amine; N-(3-(1H-pyrazol-1-yl)benzyl)-N-(3-methoxybenzyl)-2-((2-morpholinoethoxy)methyl)pyridin-4-amine; N-(3-(1H-pyrazol-1-yl)benzyl)-N-(3-methoxybenzyl)-2-(morpholinomethyl)pyridin-4-amine; N-(3-(1H-pyrazol-1-yl)benzyl)-N-(3-methoxybenzyl)-2-(piperidin-1-ylmethyl)pyridin-4-amine; N-(3-(1H-pyrazol-1-yl)benzyl)-N-(3-methoxybenzyl)-2-((4-methylpiperazin-1-yl)methyl)pyridin-4-amine; 1-((4-((3-(1H-pyrazol-1-yl)benzyl)(3-methoxybenzyl)amino)pyridin-2-yl)methyl)piperazin-2-one; 1-((4-((3-(1H-pyrazol-1-yl)benzyl)(3-methoxybenzyl)amino)pyridin-2-yl)methyl)piperazine-2,5-dione; 4-((4-((3-(1H-pyrazol-1-yl)benzyl)(3-methoxybenzyl)amino)pyridin-2-yl)methyl)piperazin-2-one; N-(3-(1H-pyrazol-1-yl)benzyl)-2-((dimethylamino)methyl)-N-(3-methoxybenzyl)pyridin-4-amine; N-(3-(1H-imidazol-1-yl)benzyl)-N-(3-methoxybenzyl)-2-((2-(2-morpholinoethoxy)ethoxy)methyl)pyridin-4-amine; N-(3-(1H-imidazol-1-yl)benzyl)-N-(3-methoxybenzyl)-2-((2-(3 methoxybenzyloxy)ethoxy)methyl)pyridin-4-amine; N-(3-(1H-imidazol-1-yl)benzyl)-2-((2-(3-(dimethylamino)phenoxy)ethoxy)methyl)-N-(3-methoxybenzyl)pyridin-4-amine; N-(3-(1H-imidazol-1-yl)benzyl)-N-(3-methoxybenzyl)-2-((2-morpholinoethoxy)methyl)pyridin-4-amine; N-(3-(1H-imidazol-1-yl)benzyl)-N-(3-methoxybenzyl)-2-(morpholinomethyl)pyridin-4-amine; N-(3-(1H-imidazol-1-yl)benzyl)-N-(3-methoxybenzyl)-2-(piperidin-1-ylmethyl)pyridin-4-amine; N-(3-(1H-imidazol-1-yl)benzyl)-N-(3-methoxybenzyl)-2-((4-methylpiperazin-1-yl)methyl)pyridin-4-amine; 1-((4-((3-(1H-imidazol-1-yl)benzyl)(3-methoxybenzyl)amino)pyridin-2-yl)methyl)piperazin-2-one; 1-((4-((3-(1H-imidazol-1-yl)benzyl)(3-methoxybenzyl)amino)pyridin-2-yl)methyl)piperazine-2,5-dione; 4-((4-((3-(1H-imidazol-1-yl)benzyl)(3-methoxybenzyl)amino)pyridin-2-yl)methyl)piperazin-2-one; N-(3-(1H-imidazol-1-yl)benzyl)-2-((dimethylamino)methyl)-N-(3-methoxybenzyl)pyridin-4-amine; 2-((2-(3-(dimethylamino)phenoxy)ethoxy)methyl)-N-(3-methoxybenzyl)-N-(4-(pyrrolidin-1-yl)benzyl)pyridin-4-amine; N-(3-methoxybenzyl)-2-((2-(2-morpholinoethoxy)ethoxy)methyl)-N-(4-(pyrrolidin-1-yl)benzyl)pyridin-4-amine; N-(3-methoxybenzyl)-2-((2-(3-methoxybenzyloxy)ethoxy)methyl)-N-(4-(pyrrolidin-1-yl)benzyl)pyridin-4-amine; N-(3-methoxybenzyl)-2-((2-morpholinoethoxy)methyl)-N-(4-(pyrrolidin-1-yl)benzyl)pyridin-4-amine; N-(3-methoxybenzyl)-2-(morpholinomethyl)-N-(4-(pyrrolidin-1-yl)benzyl)pyridin-4-amine; N-(3-methoxybenzyl)-2-(piperidin-1-ylmethyl)-N-(4-(pyrrolidin-1-yl)benzyl)pyridin-4-amine; N-(3-methoxybenzyl)-2-((4-methylpiperazin-1-yl)methyl)-N-(4-(pyrrolidin-1-yl)benzyl)pyridin-4-amine; 1-((4-((3-methoxybenzyl)(4-(pyrrolidin-1-yl)benzyl)amino)pyridin-2-yl)methyl)piperazin-2-one; 1-((4-((3-methoxybenzyl)(4-(pyrrolidin-1-yl)benzyl)amino)pyridin-2-yl)methyl)piperazine-2,5-dione; 4-((4-((3-methoxybenzyl)(4-(pyrrolidin-1-yl)benzyl)amino)pyridin-2-yl)methyl)piperazin-2-one; 2-((dimethylamino)methyl)-N-(3-methoxybenzyl)-N-(4-(pyrrolidin-1-yl)benzyl)pyridin-4-amine; N-(3-methoxybenzyl)-N-(4-morpholinobenzyl)-2-((2-(2-morpholinoethoxy)ethoxy)methyl)pyridin-4-amine; N-(3-methoxybenzyl)-2-((2-(3-methoxybenzyloxy)ethoxy)methyl)-N-(4-morpholinobenzyl)pyridin-4-amine; 2-((2-(3-(dimethylamino)phenoxy)ethoxy)methyl)-N-(3-methoxybenzyl)-N-(4-morpholinobenzyl)pyridin-4-amine; N-(3-methoxybenzyl)-N-(4-morpholinobenzyl)-2-((2-morpholinoethoxy)methyl)pyridin-4-amine; N-(3-methoxybenzyl)-N-(4-morpholinobenzyl)-2-(morpholinomethyl)pyridin-4-amine; N-(3-methoxybenzyl)-N-(4-morpholinobenzyl)-2-(piperidin-1-ylmethyl)pyridin-4-amine; N-(3-methoxybenzyl)-2-((4-methylpiperazin-1-yl)methyl)-N-(4-morpholinobenzyl)pyridin-4-amine; 1-((4-((3-methoxybenzyl)(4-morpholinobenzyl)amino)pyridin-2-yl)methyl)piperazin-2-one; 1-((4-((3-methoxybenzyl)(4-morpholinobenzyl)amino)pyridin-2-yl)methyl)piperazine-2,5-dione; 4-((4-((3-methoxybenzyl)(4-morpholinobenzyl)amino)pyridin-2-yl)methyl)piperazin-2-one; 2-((dimethylamino)methyl)-N-(3-methoxybenzyl)-N-(4-morpholinobenzyl)pyridin-4-amine; N-(3-methoxybenzyl)-N-(4-(4-methylpiperazin-1-yl)benzyl)-2-((2-(2 morpholinoethoxy)ethoxy)methyl)pyridin-4-amine; N-(3-methoxybenzyl)-2-((2-(3-methoxybenzyloxy)ethoxy)methyl)-N-(4-(4-methylpiperazin-1-yl)benzyl)pyridin-4-amine; 2-((2-(3-(dimethylamino)phenoxy)ethoxy)methyl)-N-(3-methoxybenzyl)-N-(4-(4-methylpiperazin-1-yl)benzyl)pyridin-4-amine; N-(3-methoxybenzyl)-N-(4-(4-methylpiperazin-1-yl)benzyl)-2-((2-morpholinoethoxy)methyl)pyridin-4-amine; N-(3-methoxybenzyl)-N-(4-(4-methylpiperazin-1-yl)benzyl)-2-(morpholinomethyl)pyridin-4-amine; N-(3-methoxybenzyl)-N-(4-(4-methylpiperazin-1-yl)benzyl)-2-(piperidin-1-ylmethyl)pyridin-4-amine; N-(3-methoxybenzyl)-N-(4-(4-methylpiperazin-1-yl)benzyl)-2-((4-methylpiperazin-1-yl)methyl)pyridin-4-amine; 1-((4-((3-methoxybenzyl)(4-(4-methylpiperazin-1-yl)benzyl)amino)pyridin-2-yl)methyl)piperazin-2-one; 1-((4-((3-methoxybenzyl)(4-(4-methylpiperazin-1-yl)benzyl)amino)pyridin-2-yl)methyl)piperazine-2,5-dione; 4-((4-((3-methoxybenzyl)(4-(4-methylpiperazin-1-yl)benzyl)amino)pyridin-2-yl)methyl)piperazin-2-one; 2-((dimethylamino)methyl)-N-(3-methoxybenzyl)-N-(4-(4-methylpiperazin-1-yl)benzyl)pyridin-4-amine; N-(3-methoxybenzyl)-N-(3-morpholinobenzyl)-2-((2-(2-morpholinoethoxy)ethoxy)methyl)pyridin-4-amine; N-(3-methoxybenzyl)-2-((2-(3-methoxybenzyloxy)ethoxy)methyl)-N-(3-morpholinobenzyl)pyridin-4-amine; 2-((2-(3-(dimethylamino)phenoxy)ethoxy)methyl)-N-(3-methoxybenzyl)-N-(3-morpholinobenzyl)pyridin-4-amine; N-(3-methoxybenzyl)-N-(3-morpholinobenzyl)-2-((2-morpholinoethoxy)methyl)pyridin-4-amine; N-(3-methoxybenzyl)-N-(3-morpholinobenzyl)-2-(morpholinomethyl)pyridin-4-amine; N-(3-methoxybenzyl)-N-(3-morpholinobenzyl)-2-(piperidin-1-ylmethyl)pyridin-4-amine; N-(3-methoxybenzyl)-2-((4-methylpiperazin-1-yl)methyl)-N-(3-morpholinobenzyl)pyridin-4-amine; 1-((4-((3-methoxybenzyl)(3-morpholinobenzyl)amino)pyridin-2-yl)methyl)piperazin-2-one; 1-((4-((3-methoxybenzyl)(3-morpholinobenzyl)amino)pyridin-2-yl)methyl)piperazine-2,5-dione; 4-((4-((3-methoxybenzyl)(3-morpholinobenzyl)amino)pyridin-2-yl)methyl)piperazin-2-one; 2-((dimethylamino)methyl)-N-(3-methoxybenzyl)-N-(3-morpholinobenzyl)pyridin-4-amine; N-(3-methoxybenzyl)-N-(3-(4-methylpiperazin-1-yl)benzyl)-2-((2-(2-morpholinoethoxy)ethoxy)methyl)pyridin-4-amine; N-(3-methoxybenzyl)-2-((2-(3-methoxybenzyloxy)ethoxy)methyl)-N-(3-(4-methylpiperazin-1-yl)benzyl)pyridin-4-amine; 2-((2-(3-(dimethylamino)phenoxy)ethoxy)methyl)-N-(3-methoxybenzyl)-N-(3-(4-methylpiperazin-1-yl)benzyl)pyridin-4-amine; N-(3-methoxybenzyl)-N-(3-(4-methylpiperazin-1-yl)benzyl)-2-((2-morpholinoethoxy)methyl)pyridin-4-amine; N-(3-methoxybenzyl)-N-(3-(4-methylpiperazin-1-yl)benzyl)-2-(morpholinomethyl)pyridin-4-amine; N-(3-methoxybenzyl)-N-(3-(4-methylpiperazin-1-yl)benzyl)-2-(piperidin-1-ylmethyl)pyridin-4-amine; N-(3-methoxybenzyl)-N-(3-(4-methylpiperazin-1-yl)benzyl)-2-((4-methylpiperazin-1-yl)methyl)pyridin-4-amine; 1-((4-((3-methoxybenzyl)(3-(4-methylpiperazin-1-yl)benzyl)amino)pyridin-2-yl)methyl)piperazin-2-one; 1-((4-((3-methoxybenzyl)(3-(4-methylpiperazin-1-yl)benzyl)amino)pyridin-2-yl)methyl)piperazine-2,5-dione; 4-((4-((3-methoxybenzyl)(3-(4-methylpiperazin-1-yl)benzyl)amino)pyridin-2-yl)methyl)piperazin-2-one; 2-((dimethylamino)methyl)-N-(3-methoxybenzyl)-N-(3-(4-methylpiperazin-1-yl)benzyl)pyridin-4-amine; N-(4-(1H-pyrazol-1-yl)benzyl)-N-(3-methoxybenzyl)-2-((2-(2-morpholinoethoxy)ethoxy)methyl)pyridin-4-amine; N-(4-(1H-pyrazol-1-yl)benzyl)-N-(3-methoxybenzyl)-2-((2-(3-methoxybenzyloxy)ethoxy)methyl)pyridin-4-amine; N-(4-(1H-pyrazol-1-yl)benzyl)-2-((2-(3-(dimethylamino)phenoxy)ethoxy)methyl)-N-(3-methoxybenzyl)pyridin-4-amine; N-(4-(1H-pyrazol-1-yl)benzyl)-N-(3-methoxybenzyl)-2-((2-morpholinoethoxy)methyl)pyridin-4-amine; N-(4-(1H-pyrazol-1-yl)benzyl)-N-(3-methoxybenzyl)-2-(morpholinomethyl)pyridin-4-amine; N-(4-(1H-pyrazol-1-yl)benzyl)-N-(3-methoxybenzyl)-2-(piperidin-1-ylmethyl)pyridin-4-amine; 1-((4-((4-(1H-pyrazol-1-yl)benzyl)(3-methoxybenzyl)amino)pyridin-2-yl)methyl)piperazin-2-one; 1-((4-((4-(1H-pyrazol-1-yl)benzyl)(3-methoxybenzyl)amino)pyridin-2-yl)methyl)piperazine-2,5-dione; 4-((4-((4-(1H-pyrazol-1-yl)benzyl)(3-methoxybenzyl)amino)pyridin-2-yl)methyl)piperazin-2-one; N-(4-(1H-pyrazol-1-yl)benzyl)-2-((dimethylamino)methyl)-N-(3-methoxybenzyl)pyridin-4-amine; N-(4-(1H-imidazol-1-yl)benzyl)-N-(3-methoxybenzyl)-2-((2-(2-morpholinoethoxy)ethoxy)methyl)pyridin-4-amine; N-(4-(1H-imidazol-1-yl)benzyl)-N-(3-methoxybenzyl)-2-((2-(3-methoxybenzyloxy)ethoxy)methyl)pyridin-4-amine; N-(4-(1H-imidazol-1-yl)benzyl)-2-((2-(3-(dimethylamino)phenoxy)ethoxy)methyl)-N-(3-methoxybenzyl)pyridin-4-amine; N-(4-(1H-imidazol-1-yl)benzyl)-N-(3-methoxybenzyl)-2-((2-morpholinoethoxy)methyl)pyridin-4-amine; N-(4-(1H-imidazol-1-yl)benzyl)-N-(3-methoxybenzyl)-2-(morpholinomethyl)pyridin-4-amine; N-(4-(1H-imidazol-1-yl)benzyl)-N-(3-methoxybenzyl)-2-(piperidin-1-ylmethyl)pyridin-4-amine; N-(4-(1H-imidazol-1-yl)benzyl)-N-(3-methoxybenzyl)-2-((4-methylpiperazin-1-yl)methyl)pyridin-4-amine; 1-((4-((4-(1H-imidazol-1-yl)benzyl)(3-methoxybenzyl)amino)pyridin-2-yl)methyl)piperazin-2-one; 1-((4-((4-(1H-imidazol-1-yl)benzyl)(3-methoxybenzyl)amino)pyridin-2-yl)methyl)piperazine-2,5-dione; 4-((4-((4-(1H-imidazol-1-yl)benzyl)(3-methoxybenzyl)amino)pyridin-2-yl)methyl)piperazin-2-one; N-(4-(1H-imidazol-1-yl)benzyl)-2-((dimethylamino)methyl)-N-(3-methoxybenzyl)pyridin-4-amine; N-((1H-benzo[d]imidazol-6-yl)methyl)-N-(3-methoxybenzyl)-2-((2-(2-morpholinoethoxy)ethoxy)methyl)pyridin-4-amine; N-((1H-benzo[d]imidazol-6-yl)methyl)-N-(3-methoxybenzyl)-2-((2-(3-methoxybenzyloxy)ethoxy)methyl)pyridin-4-amine; N-((1H-benzo[d]imidazol-6-yl)methyl)-2-((2-(3-(dimethylamino)phenoxy)ethoxy)methyl)-N-(3-methoxybenzyl)pyridin-4-amine; N-((1H-benzo[d]imidazol-6-yl)methyl)-N-(3-methoxybenzyl)-2-((2-morpholinoethoxy)methyl)pyridin-4-amine; N-((1H-benzo[d]imidazol-6-yl)methyl)-N-(3-methoxybenzyl)-2-(morpholinomethyl)pyridin-4-amine; N-((1H-benzo[d]imidazol-6-yl)methyl)-N-(3-methoxybenzyl)-2-(piperidin-1-ylmethyl)pyridin-4-amine; N-((1H-benzo[d]imidazol-6-yl)methyl)-N-(3-methoxybenzyl)-2-((4-methylpiperazin-1-yl)methyl)pyridin-4-amine; 1-((4-(((1H-benzo[d]imidazol-6-yl)methyl)(3-methoxybenzyl)amino)pyridin-2-yl)methyl)piperazin-2-one; 1-((4-(((1H-benzo[d]imidazol-6-yl)methyl)(3-methoxybenzyl)amino)pyridin-2-yl)methyl)piperazine-2,5-dione; 4-((4-(((1H-benzo[d]imidazol-6-yl)methyl)(3-methoxybenzyl)amino)pyridin-2-yl)methyl)piperazin-2-one; N-((1H-benzo[d]imidazol-6-yl)methyl)-2-((dimethylamino)methyl)-N-(3-methoxybenzyl)pyridin-4-amine; N-((1H-indazol-6-yl)methyl)-N-(3-methoxybenzyl)-2-((2-(2-morpholinoethoxy)ethoxy)methyl)pyridin-4-amine; N-((1H-indazol-6-yl)methyl)-N-(3-methoxybenzyl)-2-((2-((3-methoxybenzyl)oxy)ethoxy)methyl)pyridin-4-amine; N-((1H-indazol-6-yl)methyl)-2-((2-(3-(dimethylamino)phenoxy)ethoxy)methyl)-N-(3-methoxybenzyl)pyridin-4-amine; N-((1H-indazol-6-yl)methyl)-N-(3-methoxybenzyl)-2-((2-morpholinoethoxy)methyl)pyridin-4-amine; N-((1H-indazol-6-yl)methyl)-N-(3-methoxybenzyl)-2-(morpholinomethyl)pyridin-4-amine; N-((1H-indazol-6-yl)methyl)-N-(3-methoxybenzyl)-2-(piperidin-1-ylmethyl)pyridin-4-amine; N-((1H-indazol-6-yl)methyl)-N-(3-methoxybenzyl)-2-((4-methylpiperazin-1-yl)methyl)pyridin-4-amine; 1-((4-(((1H-indazol-6-yl)methyl)(3-methoxybenzyl)amino)pyridin-2-yl)methyl)piperazin-2-one; 1-((4-(((1H-indazol-6-yl)methyl)(3-methoxybenzyl)amino)pyridin-2-yl)methyl)piperazine-2,5-dione; 4-((4-(((1H-indazol-6-yl)methyl)(3-methoxybenzyl)amino)pyridin-2-yl)methyl)piperazin-2-one; N-((1H-indazol-6-yl)methyl)-2-((dimethylamino)methyl)-N-(3-methoxybenzyl)pyridin-4-amine; N-(3-methoxybenzyl)-2-((2-(2-morpholinoethoxy)ethoxy)methyl)-N-(quinolin-7-ylmethyl)pyridin-4-amine; N-(3-methoxybenzyl)-2-((2-((3-methoxybenzyl)oxy)ethoxy)methyl)-N-(quinolin-7-ylmethyl)pyridin-4-amine; 2-((2-(3-(dimethylamino)phenoxy)ethoxy)methyl)-N-(3-methoxybenzyl)-N-(quinolin-7-ylmethyl)pyridin-4-amine; N-(3-methoxybenzyl)-2-((2-morpholinoethoxy)methyl)-N-(quinolin-7-ylmethyl)pyridin-4-amine; N-(3-methoxybenzyl)-2-(morpholinomethyl)-N-(quinolin-7-ylmethyl)pyridin-4-amine; N-(3-methoxybenzyl)-2-(piperidin-1-ylmethyl)-N-(quinolin-7-ylmethyl)pyridin-4-amine; N-(3-methoxybenzyl)-2-((4-methylpiperazin-1-yl)methyl)-N-(quinolin-7-ylmethyl)pyridin-4-amine; 1-((4-((3-methoxybenzyl)(quinolin-7-ylmethyl)amino)pyridin-2-yl)methyl)piperazin-2-one; 1-((4-((3-methoxybenzyl)(quinolin-7-ylmethyl)amino)pyridin-2-yl)methyl)piperazine-2,5-dione; 4-((4-((3-methoxybenzyl)(quinolin-7-ylmethyl)amino)pyridin-2-yl)methyl)piperazin-2-one; 2-((dimethylamino)methyl)-N-(3-methoxybenzyl)-N-(quinolin-7-ylmethyl)pyridin-4-amine; 4-((2-(2-((3-methoxybenzyl)oxy)ethoxy)ethoxy)methyl)-N,N-bis(3-(2-methoxyethoxy)benzyl)thiazol-2-amine; 4-((2-(2-(benzyloxy)ethoxy)ethoxy)methyl)-N,N-bis(3-(2-methoxyethoxy)benzyl)thiazol-2-amine; N,N-bis(3-(2-methoxyethoxy)benzyl)-4-((2-(2-(3-methoxyphenoxy)ethoxy)ethoxy)methyl)thiazol-2-amine; 4-((2-((3-methoxybenzyl)oxy)ethoxy)methyl)-N,N-bis(3-(2-methoxyethoxy)benzyl)thiazol-2-amine; 4-((2-(3-(dimethylamino)phenoxy)ethoxy)methyl)-N,N-bis(3-(2-methoxyethoxy)benzyl)thiazol-2-amine; N,N-bis(3-(2-methoxyethoxy)benzyl)-4-((2-(2-morpholinoethoxy)ethoxy)methyl)thiazol-2-amine; N,N-bis(3-(2-methoxyethoxy)benzyl)-4-((2-morpholinoethoxy)methyl)thiazol-2-amine; N,N-bis(3-(2-methoxyethoxy)benzyl)-4-(morpholinomethyl)thiazol-2-amine; 4-((2-(2-((3-methoxybenzyl)oxy)ethoxy)ethoxy)methyl)-N,N-bis(3-(2-methoxyethoxy)benzyl)oxazol-2-amine; 4-((2-(2-(benzyloxy)ethoxy)ethoxy)methyl)-N,N-bis(3-(2-methoxyethoxy)benzyl)oxazol-2-amine; N,N-bis(3-(2-methoxyethoxy)benzyl)-4-((2-(2-(3-methoxyphenoxy)ethoxy)ethoxy)methyl)oxazol-2-amine; 4-((2-((3-methoxybenzyl)oxy)ethoxy)methyl)-N,N-bis(3-(2-methoxyethoxy)benzyl)oxazol-2-amine; 4-((2-(3-(dimethylamino)phenoxy)ethoxy)methyl)-N,N-bis(3-(2-methoxyethoxy)benzyl)oxazol-2-amine; N,N-bis(3-(2-methoxyethoxy)benzyl)-4-((2-(2-morpholinoethoxy)ethoxy)methyl)oxazol-2-amine; N,N-bis(3-(2-methoxyethoxy)benzyl)-4-((2-morpholinoethoxy)methyl)oxazol-2-amine; N,N-bis(3-(2-methoxyethoxy)benzyl)-4-(morpholinomethyl)oxazol-2-amine; N-(3-methoxybenzyl)-4-((2-(2-((3-methoxybenzyl)oxy)ethoxy)ethoxy)methyl)-N-(3-(2-methoxyethoxy)benzyl)thiazol-2-amine; 4-((2-(2-(benzyloxy)ethoxy)ethoxy)methyl)-N-(3-methoxybenzyl)-N-(3-(2-methoxyethoxy)benzyl)thiazol-2-amine; N-(3-methoxybenzyl)-N-(3-(2-methoxyethoxy)benzyl)-4-((2-(2-(3-methoxyphenoxy)ethoxy)ethoxy)methyl)thiazol-2-amine; N-(3-methoxybenzyl)-4-((2-((3-methoxybenzyl)oxy)ethoxy)methyl)-N-(3-(2-methoxyethoxy)benzyl)thiazol-2-amine; 4-((2-(3-(dimethylamino)phenoxy)ethoxy)methyl)-N-(3-methoxybenzyl)-N-(3-(2-methoxyethoxy)benzyl)thiazol-2-amine; N-(3-methoxybenzyl)-N-(3-(2-methoxyethoxy)benzyl)-4-((2-(2-morpholinoethoxy)ethoxy)methyl)thiazol-2-amine; N-(3-methoxybenzyl)-N-(3-(2-methoxyethoxy)benzyl)-4-((2-morpholinoethoxy)methyl)thiazol-2-amine; N-(3-methoxybenzyl)-4-((2-(2-((3-methoxybenzyl)oxy)ethoxy)ethoxy)methyl)-N-(3-(2-methoxyethoxy)benzyl)oxazol-2-amine; 4-((2-(2-(benzyloxy)ethoxy)ethoxy)methyl)-N-(3-methoxybenzyl)-N-(3-(2-methoxyethoxy)benzyl)oxazol-2-amine; N-(3-methoxybenzyl)-N-(3-(2-methoxyethoxy)benzyl)-4-((2-(2-(3-methoxyphenoxy)ethoxy)ethoxy)methyl)oxazol-2-amine; 4-((2-(3-(dimethylamino)phenoxy)ethoxy)methyl)-N-(3-methoxybenzyl)-N-(3-(2-methoxyethoxy)benzyl)oxazol-2-amine; N-(3-methoxybenzyl)-N-(3-(2-methoxyethoxy)benzyl)-4-((2-(2-morpholinoethoxy)ethoxy)methyl)oxazol-2-amine; N-(3-methoxybenzyl)-N-(3-(2-methoxyethoxy)benzyl)-4-((2-morpholinoethoxy)methyl)oxazol-2-amine; N-(3-methoxybenzyl)-N-(3-(2-methoxyethoxy)benzyl)-4-(morpholinomethyl)oxazol-2-amine; N,N-bis(3-methoxybenzyl)-4-methyl-5-(morpholinomethyl)thiazol-2-amine; N-(3-(dimethylamino)benzyl)-N-(3-methoxybenzyl)-4-(2-morpholinoethyl)thiazol-2-amine; N-(3-(dimethylamino)benzyl)-N-(3-methoxybenzyl)-4-methyl-5-(morpholinomethyl)thiazol-2-amine; N-(3-methoxybenzyl)-4-(2-morpholinoethyl)-N-(3-(pyrrolidin-1-yl)benzyl)thiazol-2-amine; N-(3-methoxybenzyl)-4-methyl-5-(morpholinomethyl)-N-(3-(pyrrolidin-1-yl)benzyl)thiazol-2-amine; N-(3-(1H-pyrazol-1-yl)benzyl)-N-(3-methoxybenzyl)-4-(2-morpholinoethyl)thiazol-2-amine; N-(3-(1H-pyrazol-1-yl)benzyl)-N-(3-methoxybenzyl)-4-methyl-5-(morpholinomethyl)thiazol-2-amine; N-(3-(1H-imidazol-1-yl)benzyl)-N-(3-methoxybenzyl)-4-(2-morpholinoethyl)thiazol-2-amine; N-(3-(1H-imidazol-1-yl)benzyl)-N-(3-methoxybenzyl)-4-methyl-5-(morpholinomethyl)thiazol-2-amine; N-(3-methoxybenzyl)-4-(2-morpholinoethyl)-N-(4-(pyrrolidin-1-yl)benzyl)thiazol-2-amine; N-(3-methoxybenzyl)-4-methyl-5-(morpholinomethyl)-N-(4-(pyrrolidin-1-yl)benzyl)thiazol-2-amine; N-(3-methoxybenzyl)-N-(4-morpholinobenzyl)-4-(2-morpholinoethyl)thiazol-2-amine; N-(3-methoxybenzyl)-4-methyl-N-(4-morpholinobenzyl)-5-(morpholinomethyl)thiazol-2-amine; N-(3-methoxybenzyl)-N-(4-(4-methylpiperazin-1-yl)benzyl)-4-(2-morpholinoethyl)thiazol-2-amine; N-(3-methoxybenzyl)-4-methyl-N-(4-(4-methylpiperazin-1-yl)benzyl)-5-(morpholinomethyl)thiazol-2-amine; N-(3-methoxybenzyl)-N-(3-morpholinobenzyl)-4-(2-morpholinoethyl)thiazol-2-amine; N-(3-methoxybenzyl)-4-methyl-N-(3-morpholinobenzyl)-5-(morpholinomethyl)thiazol-2-amine; N-(3-methoxybenzyl)-N-(3-(4-methylpiperazin-1-yl)benzyl)-4-(2-morpholinoethyl)thiazol-2-amine; N-(3-methoxybenzyl)-4-methyl-N-(3-(4-methylpiperazin-1-yl)benzyl)-5-(morpholinomethyl)thiazol-2-amine; N-(4-(1H-pyrazol-1-yl)benzyl)-N-(3-methoxybenzyl)-4-(2-morpholinoethyl)thiazol-2-amine; N-(4-(1H-pyrazol-1-yl)benzyl)-N-(3-methoxybenzyl)-4-methyl-5-(morpholinomethyl)thiazol-2-amine; N-(4-(1H-imidazol-1-yl)benzyl)-N-(3-methoxybenzyl)-4-(2-morpholinoethyl)thiazol-2-amine; N-(4-(1H-imidazol-1-yl)benzyl)-N-(3-methoxybenzyl)-4-methyl-5-(morpholinomethyl)thiazol-2-amine; N-((1H-benzo[d]imidazol-6-yl)methyl)-N-(3-methoxybenzyl)-4-(2-morpholinoethyl)thiazol-2-amine; N-((1H-benzo[d]imidazol-6-yl)methyl)-N-(3-methoxybenzyl)-4-methyl-5-(morpholinomethyl)thiazol-2-amine; N-((1H-indazol-6-yl)methyl)-N-(3-methoxybenzyl)-4-(2-morpholinoethyl)thiazol-2-amine; N-((1H-indazol-6-yl)methyl)-N-(3-methoxybenzyl)-4-methyl-5-(morpholinomethyl)thiazol-2-amine; N-(3-methoxybenzyl)-4-(2-morpholinoethyl)-N-(quinolin-7-ylmethyl)thiazol-2-amine; N-(3-methoxybenzyl)-4-methyl-5-(morpholinomethyl)-N-(quinolin-7-ylmethyl)thiazol-2-amine; N,N-bis(3-methoxybenzyl)-4-(2-morpholinoethyl)oxazol-2-amine; N,N-bis(3-methoxybenzyl)-4-methyl-5-(morpholinomethyl)oxazol-2-amine; N-(3-(dimethylamino)benzyl)-N-(3-methoxybenzyl)-4-(2-morpholinoethyl)oxazol-2-amine; N-(3-(dimethylamino)benzyl)-N-(3-methoxybenzyl)-4-methyl-5-(morpholinomethyl)oxazol-2-amine; N-(3-methoxybenzyl)-4-(2-morpholinoethyl)-N-(3-(pyrrolidin-1-yl)benzyl)oxazol-2-amine; N-(3-methoxybenzyl)-4-methyl-5-(morpholinomethyl)-N-(3-(pyrrolidin-1-yl)benzyl)oxazol-2-amine; N-(3-(1H-pyrazol-1-yl)benzyl)-N-(3-methoxybenzyl)-4-(2-morpholinoethyl)oxazol-2-amine; N-(3-(1H-pyrazol-1-yl)benzyl)-N-(3-methoxybenzyl)-4-methyl-5-(morpholinomethyl)oxazol-2-amine; N-(3-(1H-imidazol-1-yl)benzyl)-N-(3-methoxybenzyl)-4-(2-morpholinoethyl)oxazol-2-amine; N-(3-(1H-imidazol-1-yl)benzyl)-N-(3-methoxybenzyl)-4-methyl-5-(morpholinomethyl)oxazol-2-amine; N-(3-methoxybenzyl)-4-(2-morpholinoethyl)-N-(4-(pyrrolidin-1-yl)benzyl)oxazol-2-amine; N-(3-methoxybenzyl)-4-methyl-5-(morpholinomethyl)-N-(4-(pyrrolidin-1-yl)benzyl)oxazol-2-amine; N-(3-methoxybenzyl)-N-(4-morpholinobenzyl)-4-(2-morpholinoethyl)oxazol-2-amine; N-(3-methoxybenzyl)-4-methyl-N-(4-morpholinobenzyl)-5-(morpholinomethyl)oxazol-2-amine; N-(3-methoxybenzyl)-N-(4-(4-methylpiperazin-1-yl)benzyl)-4-(2-morpholinoethyl)oxazol-2-amine; N-(3-methoxybenzyl)-4-methyl-N-(4-(4-methylpiperazin-1-yl)benzyl)-5-(morpholinomethyl)oxazol-2-amine; N-(3-methoxybenzyl)-N-(3-morpholinobenzyl)-4-(2-morpholinoethyl)oxazol-2-amine; N-(3-methoxybenzyl)-4-methyl-N-(3-morpholinobenzyl)-5-(morpholinomethyl)oxazol-2-amine; N-(3-methoxybenzyl)-N-(3-(4-methylpiperazin-1-yl)benzyl)-4-(2-morpholinoethyl)oxazol-2-amine; N-(3-methoxybenzyl)-4-methyl-N-(3-(4-methylpiperazin-1-yl)benzyl)-5-(morpholinomethyl)oxazol-2-amine; N-(4-(1H-imidazol-1-yl)benzyl)-N-(3-methoxybenzyl)-4-(2-morpholinoethyl)oxazol-2-amine; N-(4-(1H-imidazol-1-yl)benzyl)-N-(3-methoxybenzyl)-4-methyl-5-(morpholinomethyl)oxazol-2-amine; N-(4-(1H-pyrazol-1-yl)benzyl)-N-(3-methoxybenzyl)-4-(2-morpholinoethyl)oxazol-2-amine; N-(4-(1H-pyrazol-1-yl)benzyl)-N-(3-methoxybenzyl)-4-methyl-5-(morpholinomethyl)oxazol-2-amine; N-((1H-benzo[d]imidazol-6-yl)methyl)-N-(3-methoxybenzyl)-4-(2-morpholinoethyl)oxazol-2-amine; N-((1H-benzo[d]imidazol-6-yl)methyl)-N-(3-methoxybenzyl)-4-methyl-5-(morpholinomethyl)oxazol-2-amine; N-((1H-indazol-6-yl)methyl)-N-(3-methoxybenzyl)-4-(2-morpholinoethyl)oxazol-2-amine; N-((1H-indazol-6-yl)methyl)-N-(3-methoxybenzyl)-4-methyl-5-(morpholinomethyl)oxazol-2-amine; N-(3-methoxybenzyl)-4-(2-morpholinoethyl)-N-(quinolin-7-ylmethyl)oxazol-2-amine; N-(3-methoxybenzyl)-4-methyl-5-(morpholinomethyl)-N-(quinolin-7-ylmethyl)oxazol-2-amine; N,N-bis(3-methoxybenzyl)-5-((2-(2-(3-methoxyphenoxy)ethoxy)ethoxy)methyl)pyridin-2-amine; 5-((2-(2-(benzyloxy)ethoxy)ethoxy)methyl)-N,N-bis(3-methoxybenzyl)pyridin-2-amine; N,N-bis(3-methoxybenzyl)-5-((2-(2-((3-methoxybenzyl)oxy)ethoxy)ethoxy)methyl)pyridin-2-amine; 5-((2-(3-(dimethylamino)phenoxy)ethoxy)methyl)-N,N-bis(3-methoxybenzyl)pyridin-2-amine; N,N-bis(3-methoxybenzyl)-5-((2-((3-methoxybenzyl)oxy)ethoxy)methyl)pyridin-2-amine; N,N-bis(3-methoxybenzyl)-5-((2-(2-morpholinoethoxy)ethoxy)methyl)pyridin-2-amine; N,N-bis(3-methoxybenzyl)-5-((2-morpholinoethoxy)methyl)pyridin-2-amine; N,N-bis(3-methoxybenzyl)-5-(morpholinomethyl)pyridin-2-amine; N-(3-(dimethylamino)benzyl)-N-(3-methoxybenzyl)-5-((2-(2-(3-methoxyphenoxy)ethoxy)ethoxy)methyl)pyridin-2-amine; 5-((2-(2-(benzyloxy)ethoxy)ethoxy)methyl)-N-(3-(dimethylamino)benzyl)-N-(3-methoxybenzyl)pyridin-2-amine; N-(3-(dimethylamino)benzyl)-N-(3-methoxybenzyl)-5-((2-(2-((3-methoxybenzyl)oxy)ethoxy)ethoxy)methyl)pyridin-2-amine; N-(3-(dimethylamino)benzyl)-5-((2-(3-(dimethylamino)phenoxy)ethoxy)methyl)-N-(3-methoxybenzyl)pyridin-2-amine; N-(3-(dimethylamino)benzyl)-N-(3-methoxybenzyl)-5-((2-((3-methoxybenzyl)oxy)ethoxy)methyl)pyridin-2-amine; N-(3-(dimethylamino)benzyl)-N-(3-methoxybenzyl)-5-((2-(2-morpholinoethoxy)ethoxy)methyl)pyridin-2-amine; N-(3-(dimethylamino)benzyl)-N-(3-methoxybenzyl)-5-((2-morpholinoethoxy)methyl)pyridin-2-amine; N-(3-(dimethylamino)benzyl)-N-(3-methoxybenzyl)-5-(morpholinomethyl)pyridin-2-amine; N-(3-methoxybenzyl)-5-((2-(2-(3-methoxyphenoxy)ethoxy)ethoxy)methyl)-N-(4-morpholinobenzyl)pyridin-2-amine; 5-((2-(2-(benzyloxy)ethoxy)ethoxy)methyl)-N-(3-methoxybenzyl)-N-(4-morpholinobenzyl)pyridin-2-amine; N-(3-methoxybenzyl)-5-((2-(2-((3-methoxybenzyl)oxy)ethoxy)ethoxy)methyl)-N-(4-morpholinobenzyl)pyridin-2-amine; 5-((2-(3-(dimethylamino)phenoxy)ethoxy)methyl)-N-(3-methoxybenzyl)-N-(4-morpholinobenzyl)pyridin-2-amine; N-(3-methoxybenzyl)-5-((2-((3-methoxybenzyl)oxy)ethoxy)methyl)-N-(4-morpholinobenzyl)pyridin-2-amine; N-(3-methoxybenzyl)-N-(4-morpholinobenzyl)-5-((2-(2-morpholinoethoxy)ethoxy)methyl)pyridin-2-amine; N-(3-methoxybenzyl)-N-(4-morpholinobenzyl)-5-((2-morpholinoethoxy)methyl)pyridin-2-amine; N-(3-methoxybenzyl)-N-(4-morpholinobenzyl)-5-(morpholinomethyl)pyridin-2-amine; N-((1H-indazol-6-yl)methyl)-N-(3-methoxybenzyl)-5-((2-(2-(3-methoxyphenoxy)ethoxy)ethoxy)methyl)pyridin-2-amine; N-((1H-indazol-6-yl)methyl)-5-((2-(2-(benzyloxy)ethoxy)ethoxy)methyl)-N-(3-methoxybenzyl)pyridin-2-amine; N-((1H-indazol-6-yl)methyl)-N-(3-methoxybenzyl)-5-((2-(2-((3-methoxybenzyl)oxy)ethoxy)ethoxy)methyl)pyridin-2-amine; N-((1H-indazol-6-yl)methyl)-5-((2-(3-(dimethylamino)phenoxy)ethoxy)methyl)-N-(3-methoxybenzyl)pyridin-2-amine; N-((1H-indazol-6-yl)methyl)-N-(3-methoxybenzyl)-5-((2-((3-methoxybenzyl)oxy)ethoxy)methyl)pyridin-2-amine; N-((1H-indazol-6-yl)methyl)-N-(3-methoxybenzyl)-5-((2-(2 morpholinoethoxy)ethoxy)methyl)pyridin-2-amine; N-((1H-indazol-6-yl)methyl)-N-(3-methoxybenzyl)-5-((2-morpholinoethoxy)methyl)pyridin-2-amine; and N-((1H-indazol-6-yl)methyl)-N-(3-methoxybenzyl)-5-(morpholinomethyl)pyridin-2-amine. N,N-bis(3-methoxybenzyl)-4-(2-(2-(3-methoxyphenoxy)ethoxy)ethoxy)aniline; 4-(2-(2-(benzyloxy)ethoxy)ethoxy)-N,N-bis(3-methoxybenzyl)aniline; N,N-bis(3-methoxybenzyl)-4-(2-(2-morpholinoethoxy)ethoxy)aniline; 3-(2-(4-(bis(3-methoxybenzyl)amino)phenoxy)ethoxy)-N,N-dimethylaniline; N,N-bis(3-methoxybenzyl)-4-(2-morpholinoethoxy)aniline; 3-(((3-methoxybenzyl)(4-(2-(2-(3-methoxyphenoxy)ethoxy)ethoxy)phenyl)amino)methyl)-N,N-dimethylaniline; 3-(((4-(2-(2-(benzyloxy)ethoxy)ethoxy)phenyl)(3-methoxybenzyl)amino)methyl)-N,N-dimethylaniline; 3-(((3-methoxybenzyl)(4-(2-(2-morpholinoethoxy)ethoxy)phenyl)amino)methyl)-N,N-dimethylaniline; 3-(2-(4-((3-(dimethylamino)benzyl)(3-methoxybenzyl)amino)phenoxy)ethoxy)-N,N-dimethylaniline; 3-(((3-methoxybenzyl)(4-(2-morpholinoethoxy)phenyl)amino)methyl)-N,N-dimethylaniline; N-(3-methoxybenzyl)-4-(2-(2-(3-methoxyphenoxy)ethoxy)ethoxy)-N-(3-(pyrrolidin-1-yl)benzyl)aniline; 4-(2-(2-(benzyloxy)ethoxy)ethoxy)-N-(3-methoxybenzyl)-N-(3-(pyrrolidin-1-yl)benzyl)aniline; N-(3-methoxybenzyl)-4-(2-(2-morpholinoethoxy)ethoxy)-N-(3-(pyrrolidin-1-yl)benzyl)aniline; 3-(2-(4-((3-methoxybenzyl)(3-(pyrrolidin-1-yl)benzyl)amino)phenoxy)ethoxy)-N,N-dimethylaniline; N-(3-methoxybenzyl)-4-(2-morpholinoethoxy)-N-(3-(pyrrolidin-1-yl)benzyl)aniline; N-(3-(1H-imidazol-1-yl)benzyl)-N-(3-methoxybenzyl)-4-(2-(2-(3-methoxyphenoxy)ethoxy)ethoxy)aniline; N-(3-(1H-imidazol-1-yl)benzyl)-4-(2-(2-(benzyloxy)ethoxy)ethoxy)-N-(3-methoxybenzyl)aniline; N-(3-(1H-imidazol-1-yl)benzyl)-N-(3-methoxybenzyl)-4-(2-(2-morpholinoethoxy)ethoxy)aniline; 3-(2-(4-((3-(1H-imidazol-1-yl)benzyl)(3-methoxybenzyl)amino)phenoxy)ethoxy)-N,N-dimethylaniline; N-(3-(1H-imidazol-1-yl)benzyl)-N-(3-methoxybenzyl)-4-(2-morpholinoethoxy)aniline; N-(3-methoxybenzyl)-4-(2-(2-(3-methoxyphenoxy)ethoxy)ethoxy)-N-(4-morpholinobenzyl)aniline; 4-(2-(2-(benzyloxy)ethoxy)ethoxy)-N-(3-methoxybenzyl)-N-(4-morpholinobenzyl)aniline; N-(3-methoxybenzyl)-N-(4-morpholinobenzyl)-4-(2-(2-morpholinoethoxy)ethoxy)aniline; 3-(2-(4-((3-methoxybenzyl)(4-morpholinobenzyl)amino)phenoxy)ethoxy)-N,N-dimethylaniline; N-(3-methoxybenzyl)-N-(4-morpholinobenzyl)-4-(2-morpholinoethoxy)aniline; N-(4-(1H-pyrazol-1-yl)benzyl)-N-(3-methoxybenzyl)-4-(2-(2-(3-methoxyphenoxy)ethoxy)ethoxy)aniline; N-(4-(1H-pyrazol-1-yl)benzyl)-4-(2-(2-(benzyloxy)ethoxy)ethoxy)-N-(3-methoxybenzyl)aniline; N-(4-(1H-pyrazol-1-yl)benzyl)-N-(3-methoxybenzyl)-4-(2-(2-morpholinoethoxy)ethoxy)aniline; 3-(2-(4-((4-(1H-pyrazol-1-yl)benzyl)(3-methoxybenzyl)amino)phenoxy)ethoxy)-N,N-dimethylaniline; N-(4-(1H-pyrazol-1-yl)benzyl)-N-(3-methoxybenzyl)-4-(2-morpholinoethoxy)aniline; N-((1H-benzo[d]imidazol-6-yl)methyl)-N-(3-methoxybenzyl)-4-(2-(2-(3-methoxyphenoxy)ethoxy)ethoxy)aniline; N-((1H-benzo[d]imidazol-6-yl)methyl)-4-(2-(2-(benzyloxy)ethoxy)ethoxy)-N-(3-methoxybenzyl)aniline; N-((1H-benzo[d]imidazol-6-yl)methyl)-N-(3-methoxybenzyl)-4-(2-(2-morpholinoethoxy)ethoxy)aniline; 3-(2-(4-(((1H-benzo[d]imidazol-6-yl)methyl)(3-methoxybenzyl)amino)phenoxy)ethoxy)-N,N-dimethylaniline; N-((1H-benzo[d]imidazol-6-yl)methyl)-N-(3-methoxybenzyl)-4-(2-morpholinoethoxy)aniline; N,N-bis(3-methoxybenzyl)-3-(2-(2-(3-methoxyphenoxy)ethoxy)ethoxy)aniline; 3-(2-(2-(benzyloxy)ethoxy)ethoxy)-N,N-bis(3-methoxybenzyl)aniline; N,N-bis(3-methoxybenzyl)-3-(2-(2-morpholinoethoxy)ethoxy)aniline; 3-(2-(3-(bis(3-methoxybenzyl)amino)phenoxy)ethoxy)-N,N-dimethylaniline; N,N-bis(3-methoxybenzyl)-3-(2-morpholinoethoxy)aniline; N-(3-(dimethylamino)benzyl)-N-(3-methoxybenzyl)-3-(2-(2-(3-methoxyphenoxy)ethoxy)ethoxy)aniline; 3-(2-(2-(benzyloxy)ethoxy)ethoxy)-N-(3-(dimethylamino)benzyl)-N-(3-methoxybenzyl)aniline; N-(3-(dimethylamino)benzyl)-N-(3-methoxybenzyl)-3-(2-(2-morpholinoethoxy)ethoxy)aniline; N-(3-(dimethylamino)benzyl)-3-(2-(3-(dimethylamino)phenoxy)ethoxy)-N-(3-methoxybenzyl)aniline; N-(3-(dimethylamino)benzyl)-N-(3-methoxybenzyl)-3-(2-morpholinoethoxy)aniline; N-(3-methoxybenzyl)-3-(2-(2-(3-methoxyphenoxy)ethoxy)ethoxy)-N-(3-(pyrrolidin-1-yl)benzyl)aniline; 3-(2-(2-(benzyloxy)ethoxy)ethoxy)-N-(3-methoxybenzyl)-N-(3-(pyrrolidin-1-yl)benzyl)aniline; N-(3-methoxybenzyl)-3-(2-(2-morpholinoethoxy)ethoxy)-N-(3-(pyrrolidin-1-yl)benzyl)aniline; 3-(2-(3-(dimethylamino)phenoxy)ethoxy)-N-(3-methoxybenzyl)-N-(3-(pyrrolidin-1-yl)benzyl)aniline; N-(3-methoxybenzyl)-3-(2-morpholinoethoxy)-N-(3-(pyrrolidin-1-yl)benzyl)aniline; N-(3-(1H-imidazol-1-yl)benzyl)-N-(3-methoxybenzyl)-3-(2-(2-(3-methoxyphenoxy)ethoxy)ethoxy)aniline; N-(3-(1H-imidazol-1-yl)benzyl)-3-(2-(2-(benzyloxy)ethoxy)ethoxy)-N-(3-methoxybenzyl)aniline; N-(3-(1H-imidazol-1-yl)benzyl)-N-(3-methoxybenzyl)-3-(2-(2-morpholinoethoxy)ethoxy)aniline; N-(3-(1H-imidazol-1-yl)benzyl)-3-(2-(3-(dimethylamino)phenoxy)ethoxy)-N-(3-methoxybenzyl)aniline; N-(3-(1H-imidazol-1-yl)benzyl)-N-(3-methoxybenzyl)-3-(2-morpholinoethoxy)aniline; N-(3-methoxybenzyl)-3-(2-(2-(3-methoxyphenoxy)ethoxy)ethoxy)-N-(4-morpholinobenzyl)aniline; N-(3-methoxybenzyl)-3-(2-(2-(3-methoxyphenoxy)ethoxy)ethoxy)-N-(4-morpholinobenzyl)aniline; N-(3-methoxybenzyl)-N-(4-morpholinobenzyl)-3-(2-(2-morpholinoethoxy)ethoxy)aniline; 3-(2-(3-(dimethylamino)phenoxy)ethoxy)-N-(3-methoxybenzyl)-N-(4-morpholinobenzyl)aniline; N-(3-methoxybenzyl)-N-(4-morpholinobenzyl)-3-(2-morpholinoethoxy)aniline; N-(4-(1H-pyrazol-1-yl)benzyl)-N-(3-methoxybenzyl)-3-(2-(2-(3-methoxyphenoxy)ethoxy)ethoxy)aniline; N-(4-(1H-pyrazol-1-yl)benzyl)-3-(2-(2-(benzyloxy)ethoxy)ethoxy)-N-(3-methoxybenzyl)aniline; N-(4-(1H-pyrazol-1-yl)benzyl)-N-(3-methoxybenzyl)-3-(2-(2-morpholinoethoxy)ethoxy)aniline; N-(4-(1H-pyrazol-1-yl)benzyl)-3-(2-(3-(dimethylamino)phenoxy)ethoxy)-N-(3-methoxybenzyl)aniline; N-(4-(1H-pyrazol-1-yl)benzyl)-N-(3-methoxybenzyl)-3-(2-morpholinoethoxy)aniline; N-((1H-benzo[d]imidazol-6-yl)methyl)-N-(3-methoxybenzyl)-3-(2-(2-(3-methoxyphenoxy)ethoxy)ethoxy)aniline; N-((1H-benzo[d]imidazol-6-yl)methyl)-3-(2-(2-(benzyloxy)ethoxy)ethoxy)-N-(3-methoxybenzyl)aniline; N-((1H-benzo[d]imidazol-6-yl)methyl)-N-(3-methoxybenzyl)-3-(2-(2-morpholinoethoxy)ethoxy)aniline; N-((1H-benzo[d]imidazol-6-yl)methyl)-3-(2-(3-(dimethylamino)phenoxy)ethoxy)-N-(3-methoxybenzyl)aniline; N-((1H-benzo[d]imidazol-6-yl)methyl)-N-(3-methoxybenzyl)-3-(2-morpholinoethoxy)aniline; N,N-bis(3-methoxybenzyl)-2-(2-(2-(3-methoxyphenoxy)ethoxy)ethoxy)pyridin-4-amine; 2-(2-(2-(benzyloxy)ethoxy)ethoxy)-N,N-bis(3-methoxybenzyl)pyridin-4-amine; N,N-bis(3-methoxybenzyl)-2-(2-(2-morpholinoethoxy)ethoxy)pyridin-4-amine; 2-(2-(3-(dimethylamino)phenoxy)ethoxy)-N,N-bis(3-methoxybenzyl)pyridin-4-amine; N,N-bis(3-methoxybenzyl)-2-(2-morpholinoethoxy)pyridin-4-amine; N-(3-(dimethylamino)benzyl)-N-(3-methoxybenzyl)-2-(2-(2-(3-methoxyphenoxy)ethoxy)ethoxy)pyridin-4-amine; 2-(2-(2-(benzyloxy)ethoxy)ethoxy)-N-(3-(dimethylamino)benzyl)-N-(3-methoxybenzyl)pyridin-4-amine; N-(3-(dimethylamino)benzyl)-N-(3-methoxybenzyl)-2-(2-(2-morpholinoethoxy)ethoxy)pyridin-4-amine; N-(3-(dimethylamino)benzyl)-2-(2-(3-(dimethylamino)phenoxy)ethoxy)-N-(3-methoxybenzyl)pyridin-4-amine; N-(3-(dimethylamino)benzyl)-N-(3-methoxybenzyl)-2-(2-morpholinoethoxy)pyridin-4-amine; N-(3-methoxybenzyl)-2-(2-(2-(3-methoxyphenoxy)ethoxy)ethoxy)-N-(3-(pyrrolidin-1-yl)benzyl)pyridin-4-amine; 2-(2-(2-(benzyloxy)ethoxy)ethoxy)-N-(3-methoxybenzyl)-N-(3-(pyrrolidin-1-yl)benzyl)pyridin-4-amine; N-(3-methoxybenzyl)-2-(2-(2-morpholinoethoxy)ethoxy)-N-(3-(pyrrolidin-1-yl)benzyl)pyridin-4-amine; 2-(2-(3-(dimethylamino)phenoxy)ethoxy)-N-(3-methoxybenzyl)-N-(3-(pyrrolidin-1-yl)benzyl)pyridin-4-amine; N-(3-methoxybenzyl)-2-(2-morpholinoethoxy)-N-(3-(pyrrolidin-1-yl)benzyl)pyridin-4-amine; N-(3-(1H-imidazol-1-yl)benzyl)-N-(3-methoxybenzyl)-2-(2-(2-(3-methoxyphenoxy)ethoxy)ethoxy)pyridin-4-amine; N-(3-(1H-imidazol-1-yl)benzyl)-2-(2-(2-(benzyloxy)ethoxy)ethoxy)-N-(3-methoxybenzyl)pyridin-4-amine; N-(3-(1H-imidazol-1-yl)benzyl)-N-(3-methoxybenzyl)-2-(2-(2-morpholinoethoxy)ethoxy)pyridin-4-amine; N-(3-(1H-imidazol-1-yl)benzyl)-2-(2-(3-(dimethylamino)phenoxy)ethoxy)-N-(3-methoxybenzyl)pyridin-4-amine; N-(3-(1H-imidazol-1-yl)benzyl)-N-(3-methoxybenzyl)-2-(2-morpholinoethoxy)pyridin-4-amine; N-(3-methoxybenzyl)-2-(2-(2-(3-methoxyphenoxy)ethoxy)ethoxy)-N-(4-morpholinobenzyl)pyridin-4-amine; 2-(2-(2-(benzyloxy)ethoxy)ethoxy)-N-(3-methoxybenzyl)-N-(4-morpholinobenzyl)pyridin-4-amine; N-(3-methoxybenzyl)-N-(4-morpholinobenzyl)-2-(2-(2-morpholinoethoxy)ethoxy)pyridin-4-amine; 2-(2-(3-(dimethylamino)phenoxy)ethoxy)-N-(3-methoxybenzyl)-N-(4-morpholinobenzyl)pyridin-4-amine; N-(3-methoxybenzyl)-N-(4-morpholinobenzyl)-2-(2-morpholinoethoxy)pyridin-4-amine; N-(4-(1H-pyrazol-1-yl)benzyl)-N-(3-methoxybenzyl)-2-(2-(2-(3-methoxyphenoxy)ethoxy)ethoxy)pyridin-4-amine; N-(4-(1H-pyrazol-1-yl)benzyl)-2-(2-(2-(benzyloxy)ethoxy)ethoxy)-N-(3-methoxybenzyl)pyridin-4-amine; N-(4-(1H-pyrazol-1-yl)benzyl)-N-(3-methoxybenzyl)-2-(2-(2-morpholinoethoxy)ethoxy)pyridin-4-amine; N-(4-(1H-pyrazol-1-yl)benzyl)-2-(2-(3-(dimethylamino)phenoxy)ethoxy)-N-(3-methoxybenzyl)pyridin-4-amine; N-(4-(1H-pyrazol-1-yl)benzyl)-N-(3-methoxybenzyl)-2-(2-morpholinoethoxy)pyridin-4-amine; N-((1H-benzo[d]imidazol-6-yl)methyl)-N-(3-methoxybenzyl)-2-(2-(2-(3-methoxyphenoxy)ethoxy)ethoxy)pyridin-4-amine; N-((1H-benzo[d]imidazol-6-yl)methyl)-2-(2-(2-(benzyloxy)ethoxy)ethoxy)-N-(3-methoxybenzyl)pyridin-4-amine; N-((1H-benzo[d]imidazol-6-yl)methyl)-N-(3-methoxybenzyl)-2-(2-(2-morpholinoethoxy)ethoxy)pyridin-4-amine; N-((1H-benzo[d]imidazol-6-yl)methyl)-2-(2-(3-(dimethylamino)phenoxy)ethoxy)-N-(3-methoxybenzyl)pyridin-4-amine; N-((1H-benzo[d]imidazol-6-yl)methyl)-N-(3-methoxybenzyl)-2-(2-morpholinoethoxy)pyridin-4-amine; N,N-bis(3-methoxybenzyl)-5-(2-(2-(3-methoxyphenoxy)ethoxy)ethoxy)pyridin-2-amine; 5-(2-(2-(benzyloxy)ethoxy)ethoxy)-N,N-bis(3-methoxybenzyl)pyridin-2-amine; N,N-bis(3-methoxybenzyl)-5-(2-(2-morpholinoethoxy)ethoxy)pyridin-2-amine; 5-(2-(3-(dimethylamino)phenoxy)ethoxy)-N,N-bis(3-methoxybenzyl)pyridin-2-amine; N,N-bis(3-methoxybenzyl)-5-(2-morpholinoethoxy)pyridin-2-amine; N-(3-(dimethylamino)benzyl)-N-(3-methoxybenzyl)-5-(2-(2-(3-methoxyphenoxy)ethoxy)ethoxy)pyridin-2-amine; 5-(2-(2-(benzyloxy)ethoxy)ethoxy)-N-(3-(dimethylamino)benzyl)-N-(3-methoxybenzyl)pyridin-2-amine; N-(3-(dimethylamino)benzyl)-N-(3-methoxybenzyl)-5-(2-(2-morpholinoethoxy)ethoxy)pyridin-2-amine; N-(3-(dimethylamino)benzyl)-5-(2-(3-(dimethylamino)phenoxy)ethoxy)-N-(3-methoxybenzyl)pyridin-2-amine; N-(3-(dimethylamino)benzyl)-N-(3-methoxybenzyl)-5-(2-morpholinoethoxy)pyridin-2-amine; N-(3-methoxybenzyl)-5-(2-(2-(3-methoxyphenoxy)ethoxy)ethoxy)-N-(3-(pyrrolidin-1-yl)benzyl)pyridin-2-amine; 5-(2-(2-(benzyloxy)ethoxy)ethoxy)-N-(3-methoxybenzyl)-N-(3-(pyrrolidin-1-yl)benzyl)pyridin-2-amine; N-(3-methoxybenzyl)-5-(2-(2-morpholinoethoxy)ethoxy)-N-(3-(pyrrolidin-1-yl)benzyl)pyridin-2-amine; 5-(2-(3-(dimethylamino)phenoxy)ethoxy)-N-(3-methoxybenzyl)-N-(3-(pyrrolidin-1-yl)benzyl)pyridin-2-amine; N-(3-methoxybenzyl)-5-(2-morpholinoethoxy)-N-(3-(pyrrolidin-1-yl)benzyl)pyridin-2-amine; N-(3-(1H-imidazol-1-yl)benzyl)-N-(3-methoxybenzyl)-5-(2-(2-(3-methoxyphenoxy)ethoxy)ethoxy)pyridin-2-amine; N-(3-(1H-imidazol-1-yl)benzyl)-5-(2-(2-(benzyloxy)ethoxy)ethoxy)-N-(3-methoxybenzyl)pyridin-2-amine; N-(3-(1H-imidazol-1-yl)benzyl)-N-(3-methoxybenzyl)-5-(2-(2-morpholinoethoxy)ethoxy)pyridin-2-amine; N-(3-(1H-imidazol-1-yl)benzyl)-5-(2-(3-(dimethylamino)phenoxy)ethoxy)-N-(3-methoxybenzyl)pyridin-2-amine; N-(3-(1H-imidazol-1-yl)benzyl)-N-(3-methoxybenzyl)-5-(2-morpholinoethoxy)pyridin-2-amine; N-(3-methoxybenzyl)-5-(2-(2-(3-methoxyphenoxy)ethoxy)ethoxy)-N-(4-morpholinobenzyl)pyridin-2-amine; 5-(2-(2-(benzyloxy)ethoxy)ethoxy)-N-(3-methoxybenzyl)-N-(4-morpholinobenzyl)pyridin-2-amine; N-(3-methoxybenzyl)-N-(4-morpholinobenzyl)-5-(2-(2-morpholinoethoxy)ethoxy)pyridin-2-amine; 5-(2-(3-(dimethylamino)phenoxy)ethoxy)-N-(3-methoxybenzyl)-N-(4-morpholinobenzyl)pyridin-2-amine; N-(3-methoxybenzyl)-N-(4-morpholinobenzyl)-5-(2-morpholinoethoxy)pyridin-2-amine; N-(4-(1H-pyrazol-1-yl)benzyl)-N-(3-methoxybenzyl)-5-(2-(2-(3-methoxyphenoxy)ethoxy)ethoxy)pyridin-2-amine; N-(4-(1H-pyrazol-1-yl)benzyl)-5-(2-(2-(benzyloxy)ethoxy)ethoxy)-N-(3-methoxybenzyl)pyridin-2-amine; N-(4-(1H-pyrazol-1-yl)benzyl)-N-(3-methoxybenzyl)-5-(2-(2-morpholinoethoxy)ethoxy)pyridin-2-amine; N-(4-(1H-pyrazol-1-yl)benzyl)-5-(2-(3-(dimethylamino)phenoxy)ethoxy)-N-(3-methoxybenzyl)pyridin-2-amine; N-(4-(1H-pyrazol-1-yl)benzyl)-N-(3-methoxybenzyl)-5-(2-morpholinoethoxy)pyridin-2-amine; N-((1H-benzo[d]imidazol-6-yl)methyl)-N-(3-methoxybenzyl)-5-(2-(2-(3-methoxyphenoxy)ethoxy)ethoxy)pyridin-2-amine; N-((1H-benzo[d]imidazol-6-yl)methyl)-5-(2-(2-(benzyloxy)ethoxy)ethoxy)-N-(3-methoxybenzyl)pyridin-2-amine; N-((1H-benzo[d]imidazol-6-yl)methyl)-N-(3-methoxybenzyl)-5-(2-(2-morpholinoethoxy)ethoxy)pyridin-2-amine; N-((1H-benzo[d]imidazol-6-yl)methyl)-5-(2-(3-(dimethylamino)phenoxy)ethoxy)-N-(3-methoxybenzyl)pyridin-2-amine; and N-((1H-benzo[d]imidazol-6-yl)methyl)-N-(3-methoxybenzyl)-5-(2-morpholinoethoxy)pyridin-2-amine.

### DETAILED DESCRIPTION

### Definitions

In addition to having their customary and usual meaning, the following definitions apply where the context permits in the specification and claims:

“Pharmaceutical composition” refers to a mixture of one or more chemicals, or pharmaceutically acceptable salts thereof, with a suitable carrier, for administration to a mammal as a medicine.

“Therapeutically effective amount” refers to that amount of the compound being administered that will relieve at least to some extent one or more of the symptoms of the disorder being treated. For example, an amount of the compound effective to prevent, alleviate or ameliorate symptoms of disease or prolong the survival of the subject being treated.

With respect to a disease or disorder, the term “treatment” refers to preventing, deterring the occurrence of the disease or disorder, arresting, regressing, or providing relief from symptoms or side effects of the disease or disorder and/or prolonging the survival of the subject being treated.

The term “alkyl” as used herein alone or in combination refers to C₁-C₁₂ straight or branched, substituted or unsubstituted saturated chain radicals derived from saturated hydrocarbons by the removal of one hydrogen atom. Representative examples of alkyl groups include methyl, ethyl, n-propyl, iso-propyl, n-butyl, sec-butyl, iso-butyl, and tert-butyl among others.

The term “alkenyl”, alone or in combination, refers to a substituted or unsubstituted straight-chain or substituted or unsubstituted branched-chain alkenyl radical containing from 2 to 10 carbon atoms. Examples of such radicals include, but are not limited to, ethenyl, E- and Z-pentenyl, decenyl and the like.

The term “lower” modifying “alkyl”, or “alkoxy” refers to a C₁-C₆ unit for a particular functionality. For example, lower alkyl means C₁-C₆ alkyl.

The term “cycloalkyl” as used herein alone or in combination refers to a substituted or unsubstituted aliphatic ring system having 3 to 10 carbon atoms and 1 to 3 rings, including, but not limited to cyclopropyl, cyclopentyl, cyclohexyl, norbornyl, and adamantyl among others. Cycloalkyl groups can be unsubstituted or substituted with one, two or three substituents independently selected from lower alkyl, haloalkyl, alkoxy, thioalkoxy, amino, alkylamino, dialkylamino, hydroxy, halo, mercapto, nitro, carboxaldehyde, carboxy, alkoxycarbonyl and carboxamide. This term is meant to encompass cycloalkenylgroups. “Cycloalkyl” includes cis or trans forms. Furthermore, the substituents may either be in endo or exo positions in the bridged bicyclic systems.

The term “cycloalkenyl” as used herein alone or in combination refers to a cyclic carbocycle containing from 4 to 8 carbon atoms and one or more double bonds. Examples of such cycloalkenyl radicals include, but are not limited to, cyclopentenyl, cyclohexenyl, cyclopentadienyl and the like.

The term “cycloalkylalkyl” as used herein refers to a cycloalkyl group appended to a lower alkyl radical, including, but not limited to cyclohexyl methyl.

The term “halo” or “halogen” as used herein refers to I, Br, Cl or F.

The term “haloalkyl” as used herein refers to a lower alkyl radical, to which is appended at least one halogen substituent, for example chloromethyl, fluoroethyl, trifluoromethyl and pentafluoroethyl among others.

The term “alkoxy”, alone or in combination, refers to a radical of the formula alkyl-O—, wherein the term “alkyl” is as defined above. Examples of suitable alkyl ether radicals include, but are not limited to, methoxy, ethoxy, n-propoxy, iso-propoxy, n-butoxy, iso-butoxy, sec-butoxy, tert-butoxy and the like.

The term “carboxyl” as used herein refers to —CO₂ H.

The term “thioalkoxy”, refers to a thioether radical of formula alkyl-S—, wherein “alkyl” is as defined above.

The term “carboxaldehyde” as used herein refers to —C(O)R wherein R is hydrogen.

The term “carboxamide” as used herein refers to —C(O)NR₂ wherein R is hydrogen, alkyl or any other suitable substituent.

The term “alkoxyalkoxy” as used herein refers to R_{b} O—R_{c} O— wherein R_{b} is lower alkyl as defined above and R_{c} is alkylene wherein alkylene is —(CH₂)_{n′}— wherein n′ is an integer from 1 to 6. Representative examples of alkoxyalkoxy groups include methoxymethoxy, ethoxymethoxy, and t-butoxymethoxy among others.

The term “alkylamino” as used herein refers to R_{d} NH— wherein R_{d} is a lower alkyl group, for example, ethylamino, butylamino, among others.

The term “dialkylamino” as used herein refers to Rₑ R_{f} N— wherein Rₑ and R_{f} are independently selected from lower alkyl, for example diethylamino, and methyl propylamino, among others.

The term “amino” as used herein refers to H₂N—.

The term “alkoxycarbonyl” as used herein refers to an alkoxyl group as previously defined appended to the parent molecular moiety through a carbonyl group. Examples of alkoxycarbonyl include methoxycarbonyl, ethoxycarbonyl, and isopropoxycarbonyl among others.

The term “aryl” or “aromatic” as used herein alone or in combination refers to a substituted or unsubstituted carbocyclic aromatic group having about 6 to 12 carbon atoms such as phenyl, naphthyl, indenyl, indanyl, azulenyl, fluorenyl and anthracenyl; or a heterocyclic aromatic group selected from the group consisting essentially of furyl, thienyl, pyridyl, pyrrolyl, oxazolyl, thiazolyl, imidazolyl, pyrazolyl, 2-isoxazolyl, isothiazolyl, 1,2,3-oxadiazolyl, 1,2,3-triazolyl, 1,3,4-thiadiazolyl, pyridazinyl, pyrimidinyl, pyrazinyl, 1,3,5-triazinyl, indolizinyl, indolyl, isoindolyl, 3H-indolyl, indolinyl, benzo[b]furanyl, 2,3-dihydrobenzofuranyl, benzo[b]thiophenyl, 1H-indazolyl, benzimidazolyl, benzthiazolyl, purinyl, 4H-quinolizinyl, isoquinolinyl, quinolinyl, cinnolinyl, phthalazinyl, quinazolinyl, quinoxalinyl, 1,8-naphthyridinyl, pteridinyl, carbazolyl, acridinyl, phenazinyl, phenothiazinyl, phenoxazinyl, pyrazolo[1,5-c]triazinyl and the like. “Arylalkyl” and “alkylaryl” employ the term “alkyl” as defined above. Rings may be multiply substituted. Aromatic rings may be fused with other aromatic or non-aromatic rings to form multicyclic rings, and are also encompassed by the term “aromatic,” as used herein.

The term “aralkyl”, alone or in combination, refers to an aryl substituted alkyl radical, wherein the terms “alkyl” and “aryl” are as defined above. Examples of suitable aralkyl radicals include, but are not limited to, phenylmethyl, phenethyl, phenylhexyl, diphenylmethyl, pyridylmethyl, tetrazolyl methyl, furylmethyl, imidazolyl methyl, indolylmethyl, thienylpropyl and the like.

The term “aralkenyl”, alone or in combination, refers to an aryl substituted alkenyl radical, wherein the terms “aryl” and “alkenyl” are as defined above.

The term “arylamino”, alone or in combination, refers to a radical of formula aryl-NRg-, wherein “aryl” is as defined above. Rg may be selected from the group consisting of H, lower alkyl, aryl and aralkyl among others. Examples of arylamino radicals include, but are not limited to, phenylamino (anilido), naphthlamino, 2-, 3-, and 4-pyridylamino and the like.

The term “biaryl”, alone or in combination, refers to a radical of formula aryl-aryl, wherein the term “aryl” is as defined above.

The term “aroyl” or “aromatic acyl”, alone or in combination, refers to a radical of formula aryl-CO—, wherein the term “aryl” is as defined above. Examples of suitable aromatic acyl radicals include, but are not limited to, benzoyl, 4-halobenzoyl, 4-carboxybenzoyl, naphthoyl, pyridylcarbonyl and the like.

The term “aliphatic acyl”, alone or in combination, refers to a radical of formula alkyl-CO—, wherein the term “alkyl” is as defined above. Examples of suitable alkyl acyl radicals include, but are not limited to, acetyl, propionyl, isobutyryl, and the like.

The term “heterocyclyl”, alone or in combination, refers to a non-aromatic 3- to 10-membered ring containing at least one endocyclic N, O, or S atom. The heterocycle may be optionally aryl-fused. The heterocycle may also optionally be substituted with at least one substituent which is independently selected from the group consisting of hydrogen, halogen, hydroxyl, amino, nitro, trifluoromethyl, trifluoromethoxy, alkyl, aralkyl, aryl, cyano, carboxyl, alkoxycarbonyl, carboxyalkyl, oxo, arylsulfonyl and aralkylaminocarbonyl among others.

The term “alkylheterocyclyl” as used herein refers to an alkyl group as previously defined appended to the parent molecular moiety through a heterocyclyl group.

The term “heterocyclylalkyl” as used herein refers to a heterocyclyl group as previously defined appended to the parent molecular moiety through an alkyl group.

The terms “electron-withdrawing” or “electron-donating” refer to the ability of a substituent to withdraw or donate electrons relative to that of hydrogen if hydrogen occupied the same position in the molecule. These terms are well-understood by one skilled in the art and are discussed in ADVANCED ORGANIC CHEMISTRY by J. March, 1985, pp. 16-18, incorporated herein by reference. Electron withdrawing groups include halo, nitro, carboxyl, carboxaldehyde, carboxyamido, aryl, quaternary ammonium, trifluoromethyl, and aryl lower alkanoyl among others. Electron donating groups include such groups as hydroxy, lower alkyl, amino, lower alkylamino, di(lower alkyl)amino, aryloxy, mercapto, lower alkylthio, lower alkylmercapto, and disulfide among others. One skilled in the art will appreciate that the aforesaid substituents may have electron donating or electron withdrawing properties under different chemical conditions. Moreover, the present invention contemplates any combination of substituents selected from the above-identified groups.

The most preferred electron donating or electron withdrawing substituents are halo, nitro, alkanoyl, carboxaldehyde, arylalkanoyl, aryloxy, carboxyl, carboxamide, cyano, sulfonyl, sulfoxide, heterocyclyl, guanidine, quaternary ammonium, sulfonium salts, hydroxy, lower alkoxy, lower alkyl, amino, lower alkylamino, di(lower alkyl)amino, amine lower alkyl mercapto, mercaptoalkyl, alkylthio and alkyldithio.

Use of the above terms is meant to encompass substituted and unsubstituted moieties. Substitution may be by one or more groups such as alcohols, ethers, esters, amides, sulfones, sulfides, hydroxyl, nitro, cyano, carboxy, amines, heteroatoms, lower alkyl, lower alkoxy, lower alkoxycarbonyl, alkoxyalkoxy, acyloxy, halogens, trifluoromethoxy, trifluoromethyl, alkyl, aralkyl, aryl, cyano, carboxy, carboalkoxy, carboxyalkyl, cycloalkyl, cycloalkylalkyl, heterocyclyl, alkylheterocyclyl, heterocyclylalkyl, oxo, arylsulfonyl and aralkylaminocarbonyl or any of the substituents of the preceding paragraphs or any of those substituents either attached directly or by suitable linkers. The linkers are typically short chains of 1-3 atoms containing any combination of —C—, —C(O)—, —NH—, —S—, —S(O)—, —O—, —C(O)O— or —S(O)O—. Rings may be substituted multiple times.

The term “mammals” includes humans and other animals.

The term “heteroatom” as used herein encompasses nitrogen, sulfur and oxygen.

The term “alpha” as used herein indicates the position immediately adjacent to the position described.

The term “one or a plurality”, “at least one” and “one or more” mean one item or multiple items (two or more).

As used herein, the term “about,” when used in conjunction with a percentage or other numerical amount, means plus or minus 10% of that percentage or other numerical amount. For example, the term “about 80%,” would encompass 80% plus or minus 8%. References cited herein are incorporated in their entirety by such reference.

### Abbreviations

The following abbreviations are used herein:
aq aqueous
Boc tert-butyloxycarbonyl
c concentrated
DCM dichloromethane (methlyene chloride)
DEAD diethyl azodicarboxylate
DIEA N,N-diisopropylethylamine
DMED N,N′-dimethylethylene diamine
DMSO dimethylsulfoxide
DMSO-d₆ dimethylsulfoxide-d6
DMF N,N-dimethylformamide
EtOAc ethyl acetate
dppf 1,1′-Bis(diphenylphosphino)ferrocene
HBTU O-(Benzotriazol-1-yl)-N,N,N′,N′-tetramethyluronium hexafluorophosphate
HPLC high performance liquid chromatography
LAH lithium aluminum hydride
MS liquid chromatography-mass spectrometry
Log P Log of the octanol water partition co-efficient
M molar concentration (mol/L)
MeCN acetonitrile
MeOH methanol
m/z mass/charge ratio
NMR nuclear magnetic resonance
OAc acetoxy
TEA triethylamine
THF tetrahydrofuran
TLC thin-layer chromatography

In some embodiments the small molecules disclosed herein, may enhance integrin-mediated adhesion which may be beneficial as therapeutic agents, and that some of those compounds may prove useful in many diseases or conditions that are amenable to cell-based therapy. Non-limiting examples of such diseases or conditions are myocardial infarction, heart failure, peripheral arterial disease, diabetes, renal failure, systemic lupus erythematosus, multiple sclerosis, pulmonary fibrosis, pulmonary hypertension, acute respiratory distress syndrome, Alzheimer's disease, Huntington's disease, Parkinson's disease, spinal cord injury, infertility, and bone marrow transplant. Accordingly, a group of chemical compounds have been synthesized which enhance the integrin-mediated binding of cells to their respective ligands. Integrins targeted by these compounds include, but are not limited to, α4β1, α4β7, α5β1, and αLβ2. Corresponding ligands include, but are not limited to, VCAM-1, fibronectin, MAdCAM-1, ICAM-1, and ICAM-2.

Agonist compounds, the ability of representative compounds to enhance binding of integrin-expressing cells, and therapeutic applications of agonist-treated cells are further disclosed herein.

While not being bound to any particular theory of action, the present integrin agonists are in some embodiments believed to bind to certain integrins and to affect a change in the integrins from their inactive state to their active state. The change may involve a change in conformation of the integrins from a closed configuration to an open configuration. The present integrin agonists, while activating the integrins, are displaced from the integrins when the integrins bind to their natural ligands involved in cell to cell adhesion. As opposed to integrin activating compounds that activate integrins by augmenting interior cell mechanisms or up regulate integrin gene expression or permanently damage or chemically alter the integrins, the present integrin activating compounds do not permanently damage or chemically alter their target integrins. This lack of permanent damage or chemical alteration of the integrins is critical for stem cell, immune cell, other cells used in cell based therapies as the present integrin activating compounds only facilitate activating, priming, homing, trafficking, infiltrating, targeting, and/or other cell movements of cells through the body of a patient, wherein the natural functioning of the cell is not permanently damaged or chemically altered, only enhanced.

### Agonist Pre-Treated Cells

One or more integrin-expressing cells are first treated (pre-treated) with an agonist compound having the general formula I, as described herein, to form agonist-bound integrin molecules on the cell's surface. The integrin-expressing cells may be embryonic stem cells, adult stem/progenitor cells, neutrophils, eosinophils, basophils, mast cells, monocytes, macrophages, dendritic cells, natural killer cells, and lymphocytes, immune cells or induced pluripotent stem cells, or a combination thereof, for example. In some cases, the cells express one or more of the integrins α4β1, α5β1, α4β7, and αLβ2. The treatment of the cells generally includes contacting the integrin-expressing cells in vitro with the agonist. In most applications the agonist compound in present in the treatment media at a concentration in the range of about 100 nM to about 30 μM. In some cases, the agonist concentration is in the range of about 1 μM to about 10 μM. After exposure to the agonist, the resulting agonist-treated cells have an enhanced ability to bind to a cognate ligand. The integrin is expressed on the surface of the cells, and may be either naturally occurring or transgenically expressed by a cell that has been transformed to express an exogenous integrin gene. The protein or other cognate ligand to which the integrin binds is expressed either on a cell surface or is part of the extracellular matrix.

### Enhanced Binding of Pre-Treated Cells to Integrin-Binding Ligands

The agonist, as described herein, dissolved in a pharmaceutically acceptable diluent, is added to cell culture media or cell suspension and mixed. The resulting agonist-treated cells are introduced to an integrin-binding ligand or binding site, whereupon the treated cells bind, attach or adhere to the cognate ligands in solution, or on a surface or target tissue. In some cases, an integrin binding protein is vascular cell adhesion molecule-1 (VCAM 1), fibronectin, mucosal addressin cell adhesion molecule-1 (MAdCAM-1), intercellular adhesion molecule-1 (ICAM-1), or intercellular adhesion molecule-2 (ICAM-2) or a combination thereof. As a result of the agonist treatment, the binding of the agonist-treated cells to the ligand is enhanced or increased compared to binding of integrin-expressing cells not treated with the agonist. In some cases, at least 3 fold more agonist-treated cells are bound to a ligand-coated surface than untreated integrin-expressing cells. In some cases, up to 3-fold more agonist-treated cells than untreated cells are bound to an integrin binding protein.

### Enhanced Retention of Pre-Treated Cells to Tissues Expressing Integrin-Binding Ligands

Regardless of the cell type, mechanism of action, or how they are delivered, for many applications it is critical that the cells home to, and are retained in, a relevant injured tissue. Low levels of cell retention observed in animal models and clinical trials are considered one of the major impediments to the progress of cell-based therapies. Even when cells are injected locally, less than 10% of injected cells are typically retained after one hour and this number decreases over time in conventional cell-based therapies. The retention rates are even lower when delivered systemically. By comparison, many embodiments of the presently disclosed methods increase the rate of retention of exogenously delivered cells and will potentially greatly further efforts in regenerative medicine.

A method of enhancing retention of exogenously-introduced cells at an in vivo target site in a mammal generally includes (a) treating integrin-expressing cells in vitro with an agonist of integrin, wherein the agonist is a compound having the general formula I, as described herein; (b) introducing at least a portion of the agonist-treated cells to an in vivo target site in the mammal; and (c) causing a greater number of said introduced agonist-treated cells to remain at said target site relative to the number of cells retained if integrin-expressing cells not treated with said agonist were introduced to said target site. The target site includes an integrin binding protein such as vascular cell adhesion molecule-1 (VCAM 1), fibronectin, mucosal addressin cellular adhesion molecule-1 (MAdCAM-1), inter-cellular adhesion molecule-1 (ICAM-1), or inter-cellular adhesion molecule-2 (ICAM-2), or combinations thereof, for example.

### Therapeutic Treatment of Damaged or Diseased Vascular Tissue

Agonist-treated cells prepared as described above are administered to a damaged or diseased vascular site in a vessel of a mammal in proximity of a damaged or diseased vascular site. The cells are injected directly into, or around a site of damaged or diseased vascular tissue, as often occurs in tissue due to ischemia following a heart attack or in peripheral arterial disease. Alternatively, in some cases the agonist-treated cells are injected intravenously for homing to a damaged or diseased site where treatment is desired. The damaged or diseased tissue contains cells (e.g., endothelial cells) that express VCAM-1, and in which VCAM-1 exists on the cell surface. Expression of VCAM-1 is induced in many cases by inflammatory cytokines such as tumor necrosis factor-α, interleukin-4 and interleukin-1β. In some instances, cells or extracellular matrix at or adjacent to a treatment site express and bear on their surface one or more other integrin-binding proteins such as fibronectin, mucosal addressin cellular adhesion molecule-1 (MAdCAM-1), intercellular adhesion molecule-1 (ICAM-1), or intercellular adhesion molecule-2 (ICAM-2). In those instances, the injected agonist-treated cells adhere to the cognate ligands at the damaged or diseased tissue site, causing a greater number of the administered agonist-treated cells to remain at the treatment site compared to the number of untreated integrin-expressing cells that would be retained if administered instead. The agonist-treated cells retained at the treatment site are allowed to grow and/or release paracrine factors, to regenerate vascular tissue at the damaged or diseased site, e.g., damage due to ischemia, autoimmune reactions, or mechanical injury. Paracrine factors are substances released from a cell that have effects on a neighboring cell, such as growth factors or cytokines.

### Cell-Based Therapies for Treatment of Other Diseases and Conditions

Use of the above-described agonist-treated cells for treatment of a number of diseases or conditions that are amenable to cell-based therapy is disclosed in various embodiments. For example, myocardial infarction, peripheral artery disease, diabetes, renal failure, systemic lupus erythematosus, multiple sclerosis, pulmonary fibrosis, pulmonary hypertension, acute respiratory distress syndrome, Alzheimer's disease, Huntington's disease, Parkinson's disease, spinal cord injury, infertility, bone marrow transplant, cord blood transplant, and cancer immunotherapies, including CAR-T cell therapy, are treated in some embodiments by injecting an above-described cell suspension intravenously, intraarterially, or directly in or around the injured area. New tissue is generated either by proliferation and differentiation of the injected cells and/or release of paracrine factors by the injected cells which induce proliferation and differentiation of neighboring host cells.

### Agonist Delivered Independent of Cells

Agonist can also be delivered independent of the cells for the cell-based therapies described above. In these cases, agonist would be delivered one or more times prior to and/or post cell treatment to promote cell homing, adhesion, and engraftment. Agonist could also be used to augment treatments unrelated to cell-based therapies, including as an adjuvant for vaccines, and as a treatment of cancer as a monotherapy or in combination with other therapies, including checkpoint blockade antibodies, radiation, or other small molecule anti-cancer drugs.

The compounds and processes described herein will be better understood in connection with the following synthetic schemes which illustrate the methods by which the disclosed compounds may be prepared. A detailed description of the preparation of representative agonist compounds is set forth in the Examples. It should be understood that the same or similar synthetic methods may also be used to synthesize other agonist compounds disclosed herein. These Examples are presented to describe preferred embodiments and uses of the compounds and agonist-treated cells, and are not meant to limit the invention unless otherwise stated in the claims appended hereto.

### Integrin Agonist Treating Compositions

Embodiments of this disclosure include compositions including one or more integrin agonist compounds of Formula (I) for treating cancer, especially treating solid tumor, wherein the one or more integrin agonist compounds of Formula (I) enhance homing, infiltration, engrafting, and invasion of natural T cell and/or treated and/or untreated effector cells and are present in a patient's blood and/or at a site of treatment in effective concentration of between about 1 fM and about 300 μM.

### Integrin Agonist and Stem/Progenitor Cell Treating Compositions

Embodiments of this disclosure include compositions including one or more integrin agonist compounds of Formula (I) and one or more stem/progenitor cells for stem cell therapies, especially treating solid tumor, wherein the one or more integrin agonist compounds of Formula (I) enhance homing, infiltration, engrafting, and invasion of natural T cell and/or treated and/or untreated effector cells and are present in a patient's blood and/or at a site of treatment in effective concentration of between about 1 fM and about 300 μM and wherein the one or more integrin agonist compounds of Formula (I) and one or more stem/progenitor cells may be administered separately, collectively, and/or concurrently. In certain embodiments, the one or more integrin agonist compounds of Formula (I) may be administered before, during and after the administration of the one or more stem/progenitor cells. In certain embodiments, the one or more integrin agonist compounds of Formula (I) may be administered in an administration schedule of separate doses before administration of the one or more stem/progenitor cells, concurrent with the administration of the one or more stem/progenitor cells, and/or in an administration schedule of separate doses after administration of the one or more stem/progenitor cells, wherein the administration schedules may be the same or different. In other embodiments, the administration schedules may comprise dosing several days before the administration of the one or more stem/progenitor cells and for several day after the administration of the one or more stem/progenitor cells.

### Integrin Agonist and Therapeutic Antibody Treating Compositions

Embodiments of this disclosure include compositions including one or more integrin agonist compounds of Formula (I) and one or more therapeutic antibodies for treating cancer, especially treating solid tumor, wherein the one or more integrin agonist compounds of Formula (I) enhance homing, infiltration, engrafting, and invasion of natural T cell and/or treated and/or untreated effector cells and are present in a patient's blood and/or at a site of treatment in effective concentration of between about 1 fM and about 300 μM and wherein the one or more integrin agonist compounds of Formula (I) and one or more therapeutic antibodies may be administered separately, collectively, and/or concurrently and methods for administering the compositions. In certain embodiments, the one or more integrin agonist compounds of Formula (I) may be administered before, during and after the administration of the one or more therapeutic antibodies. In certain embodiments, the one or more integrin agonist compounds of Formula (I) may be administered in an administration schedule of separate doses before administration of the one or more therapeutic antibodies, concurrent with the administration of the one or more therapeutic antibodies, and/or in an administration schedule of separate doses after administration of the one or more therapeutic antibodies, wherein the administration schedules may be the same or different. In other embodiments, the administration schedules may comprise dosing several days before the administration of the one or more therapeutic antibodies and for several day after the administration of the one or more therapeutic antibodies.

### Integrin Agonist and Immunomodulator Treating Compositions

Embodiments of this disclosure include compositions including one or more integrin agonist compounds of Formula (I) and one or more immunomodulators for treating cancer, especially treating solid tumor, wherein the one or more integrin agonist compounds of Formula (I) enhance homing, infiltration, engrafting, and invasion of natural T cell and/or treated and/or untreated effector cells and are present in a patient's blood and/or at a site of treatment in effective concentration of between about 1 fM and about 300 μM and wherein the one or more integrin agonist compounds of Formula (I) and one or more immunomodulators may be administered separately, collectively, and/or concurrently and methods for administering the compositions. In certain embodiments, the one or more integrin agonist compounds of Formula (I) may be administered before, during and after the administration of the one or more immunomodulators. In certain embodiments, the one or more integrin agonist compounds of Formula (I) may be administered in an administration schedule of separate doses before administration of the one or more immunomodulators, concurrent with the administration of the one or more immunomodulators, and/or in an administration schedule of separate doses after administration of the one or more immunomodulators, wherein the administration schedules may be the same or different. In other embodiments, the administration schedules may comprise dosing several days before the administration of the one or more immunomodulators and for several day after the administration of the one or more immunomodulators.

### Integrin Agonist and Antigen Treating Compositions

Embodiments of this disclosure include compositions including one or more integrin agonist compounds of Formula (I) and one or more antigens for treating cancer, especially treating solid tumor, wherein the one or more integrin agonist compounds of Formula (I) enhance homing, infiltration, engrafting, and invasion of natural T cell and/or treated and/or untreated effector cells and are present in a patient's blood and/or at a site of treatment in effective concentration of between about 1 fM and about 300 μM and wherein the one or more integrin agonist compounds of Formula (I) and one or more antigens may be administered separately, collectively, and/or concurrently and methods for administering the compositions. In certain embodiments, the one or more integrin agonist compounds of Formula (I) may be administered before, during and after the administration of the one or more antigens. In certain embodiments, the one or more integrin agonist compounds of Formula (I) may be administered in an administration schedule of separate doses before administration of the one or more antigens, concurrent with the administration of the one or more antigens, and/or in an administration schedule of separate doses after administration of the one or more antigens, wherein the administration schedules may be the same or different. In other embodiments, the administration schedules may comprise dosing several days before the administration of the one or more antigens and for several day after the administration of the one or more antigens. In one or more embodiments, a composition comprising an integrin agonist of the type disclosed herein is administered to a subject in need thereof. In one or more embodiments, a composition comprising an integrin agonist and an immodulator, both of the type disclosed herein, is administered to a subject in need thereof. The term “subject,” as used herein, comprises any and all organisms and includes the term “patient.” A subject to be treated according to the methods described herein may be one who has been diagnosed by a medical practitioner as having a disease, disorder, or dysfunction amenable to treatment with the methodologies disclosed herein. Herein “treating” refers to utilizing the disclosed methodologies and compositions for prophylactic and/or therapeutic purposes. Thus, in the claims and aspects described herein, treating refers to a subject undergoing, either for therapeutic or prophylactic purposes, the methodologies disclosed herein.

### Integrin Agonist and Vaccine Treating Compositions

Embodiments of this disclosure include compositions including one or more integrin agonist compounds of Formula (I) and one or more vaccines for treating cancer, especially treating solid tumor, wherein the one or more integrin agonist compounds of Formula (I) enhance homing, infiltration, engrafting, and invasion of natural T cell and/or treated and/or untreated effector cells and are present in a patient's blood and/or at a site of treatment in effective concentration of between about 1 fM and about 300 μM and wherein the one or more integrin agonist compounds of Formula (I) and one or more vaccines may be administered separately, collectively, and/or concurrently and methods for administering the compositions. In certain embodiments, the one or more integrin agonist compounds of Formula (I) may be administered before, during and after the administration of the one or more vaccines. In certain embodiments, the one or more integrin agonist compounds of Formula (I) may be administered in an administration schedule of separate doses before administration of the one or more vaccines, concurrent with the administration of the one or more vaccines, and/or in an administration schedule of separate doses after administration of the one or more vaccines, wherein the administration schedules may be the same or different. In other embodiments, the administration schedules may comprise dosing several days before the administration of the one or more vaccines and for several day after the administration of the one or more vaccines.

### Oral Gavage

In some embodiments, compounds were subjected to an initial determination of oral availability by oral gavage of 18-20 g mice using 4 mg/mL of compound and dosing up to 30 mg/Kg. Most compounds tested were seen at elevated levels over historical compounds such as THI0349.

Immunomodulators target CTLA-4, PD-1, PDL-1, 4-1BB, TIM-1, LAG-3, IDO-1, TIGIT, STING, interleukins, interferons, ionizing radiation, Toll Like Receptors, and other receptors.

Exemplary commercial examples of PD-1 inhibitors include, without limitation, Pembrolizumab (Keytruda), Nivolumab (Opdivo), Cemiplimab (Libtayo), any other PD-1 inhibitors, and mixtures or combinations thereof.

Exemplary commercial examples of PD-L1 inhibitors include, without limitation, Atezolizumab (Tecentriq), Avelumab (Bavencio), Durvalumab (Imfinzi), any other PD-L1 inhibitors, and mixtures or combinations thereof.

Exemplary commercial examples of CTLA-4 inhibitor include, without limitation, Ipilimumab (Yervoy), any other CTLA-4 inhibitors, and mixtures or combinations thereof.

Exemplary commercial examples of Interferons include, without limitation, interferon alpha (Roferon-A, Intron A, Alferon), any other interferon, and mixtures or combinations thereof.

Exemplary examples of Interleukins include, without limitation, interleukin-2 (IL-2) or aldesleukin (Proleukin), and mixtures or combinations thereof.

Exemplary examples of Oncolytic viruses include, without limitation, Talimogene laherparepve (Imlygic), any other Oncolytic virus, and mixtures or combinations thereof.

No exemplary examples of immunomodulators for TIM-1, LAG-3, IDO-1, STING, and TIGIT were available at the time of filing.

Exemplary examples of therapeutic antibodies include, without limitation, trastuzumab, cetuximab, ipilimumab, nivolumab rituximab, alemtuzumab, atumumab, tositumomab, any other similar therapeutic antibody, and mixtures or combinations thereof.

Exemplary examples of vaccines include, without limitation, Covid19 vaccines, Anti-Cancer vaccines, Adenovirus vaccines; Anthrax vaccines such as AVA (BioThrax); Cholera vaccines such as Vaxchora; Diphtheria vaccines such as DTaP (Daptacel, Infanrix, Td (Tenivac, generic, DT (-generic-, Tdap (Adacel, Boostrix, DTaP-IPV (Kinrix, Quadracel, DTaP-HepB-IPV (Pediarix, DTaP-IPV/Hib (Pentacel); Hepatitis A vaccines such as HepA (Havrix, Vaqta, HepA-HepB (Twinrix); Hepatitis B vaccines such as HepB (Engerix-B, Recombivax HB, Heplisav-B, DTaP-HepB-IPV (Pediarix, HepA-HepB (Twinrix); Hepatitis C vaccines, Haemophilus influenzae type b (Hib) vaccines such as Hib (ActHIB, PedvaxHIB, Hiberix, DTaP-IPV/Hib (Pentacel); Human Papillomavirus (HPV) vaccines such as HPV9 (Gardasil 9) (For scientific papers, the preferred abbreviation is 9vHPV); seasonal influenza (Flu) only vaccines such as IIV* (Afluria, Fluad, Flublok, Flucelvax, FluLaval, Fluarix, Fluvirin, Fluzone, Fluzone High-Dose, Fluzone Intradermal), LAIV (FluMist), and inactivated flu vaccines such as IIV3, IIV4, RIV3, RIV4 and ccIIV4; Japanese Encephalitis vaccines such as JE (Ixiaro); measles vaccines such as MMR (M-M-R II, MMRV (ProQuad); Meningococcal vaccines such as MenACWY (Menactra, Menveo, MenB (Bexsero, Trumenba); Mumps vaccines such as MMR (M-M-R II, MMRV (ProQuad); Pertussis vaccines such as DTaP (Daptacel, Infanrix, Tdap (Adacel, Boostrix, DTaP-IPV (Kinrix, Quadracel, DTaP-HepB-IPV (Pediarix, DTaP-IPV/Hib (Pentacel); Pneumococcal vaccines such as PCV13 (Prevnar13, PPSV23 (Pneumovax 23); Polio vaccines such as Polio (Ipol, DTaP-IPV (Kinrix, Quadracel, DTaP-HepB-IPV (Pediarix, DTaP-IPV/Hib (Pentacel); Rabies vaccines such as Rabies (Imovax Rabies, RabAvert); Rotavirus vaccines such as RV1 (Rotarix, RV5 (RotaTeq); Rubella vaccines such as MMR (M-M-R II, MMRV (ProQuad); Shingles vaccines such as RZV (Shingrix); Smallpox vaccines such as Vaccinia (ACAM2000); Tetanus vaccines such as DTaP (Daptacel, Infanrix, Td (Tenivac, generic, DT (-generic-, Tdap (Adacel, Boostrix, DTaP-IPV (Kinrix, Quadracel, DTaP-HepB-IPV (Pediarix, DTaP-IPV/Hib (Pentacel); Tuberculosis vaccines, Typhoid Fever vaccines such as Typhoid Oral (Vivotif, Typhoid Polysaccharide (Typhim Vi); Varicella vaccines such as VAR (Varivax, MMRV (ProQuad); and Yellow Fever vaccines such as YF (YF-Vax).

### ADDITIONAL DISCLOSURE

The following are non-limiting, specific aspects in accordance with the present disclosure:

A first aspect which is a compound comprising a VLA-4 integrin agonist of the Formula (I):

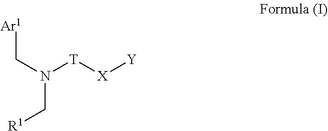

wherein, R¹ is hydrogen, lower alkyl, or aryl; Ar¹ is aryl, provided that when Ar¹ and R¹ are both phenyl, the 2 and 6 positions of each ring are substituted with hydrogen; T is aryl; X is a 1-20 atom linking chain containing any combination of —CH₂—, —C(O)—, —C(O)NR²—, —NR²—, —C(O)O— and —O—, each of which may independently be absent or present one or more times; provided that when X contains —C(O)N(R²)—, and —C(O)N(R²)— directly attaches to T, then R² is hydrogen; Y is selected from a group consisting essentially of hydrogen, (C₁-C₄)alkyl, aryl, cycloalkyl, N,N-dialkylamino, heterocyclyl, N,N-di(aralkyl)aminoaryl, or (N-aralkyl)(N-alkyl)aminoaryl; Ar¹ and R¹ may each be independently substituted or multiply substituted and selected at each occurrence from the group consisting essentially of lower alkyl, alkoxy, hydroxyalkyl, —OH, alkoxyalkyl, (C₁-C₃ alkyl)₂amino, alkoxyalkoxy, cycloalkyl, cycloalkylalkyl, aryl, heterocyclyl, alkylaryl, aralkyl, alkylheterocyclyl, and heterocyclylalkyl, groups; Y may be unsubstituted or substituted with one or more substituents selected from the group consisting essentially of halogen, lower alkyl, alkoxy, hydroxyalkyl, aliphatic acyl, —CF₃, oxo, —CN, —OH, alkoxyalkyl, (C₁-C₃ alkyl)₂amino, alkoxyalkoxy, cycloalkyl, cycloalkylalkyl, aryl, heterocyclyl, alkylaryl, aralkyl, alkylheterocyclyl, heterocyclylalkyl, and aryloxyalkyl groups; T may be unsubstituted or substituted with one or more substituents selected from the group consisting essentially of lower alkyl, alkoxy, hydroxyalkyl, aliphatic acyl, —CN, —OH, halogen, heterocycloyl, alkoxyalkyl, (C₁-C₃ alkyl)₂amino, alkoxyalkoxy, cycloalkyl, cycloalkylalkyl, heterocyclyl, alkylheterocyclyl, heterocyclylalkyl and a combination thereof, provided that when T is thiazolyl group and X and Y taken together form a C₁-C₈ alkyl group, then the 5-position of said thiazole ring is unsubstituted; substituents of T may be taken together with up to two atoms of X to form a ring; and R² when present, may be selected independently and at each occurrence from the groups consisting essentially of hydrogen, lower alkyl, acetyl and methanesulfonyl.

A second aspect which is the compound of the first aspect wherein (Ar¹CH₂)(R¹CH₂)N-T is selected from a group comprising:

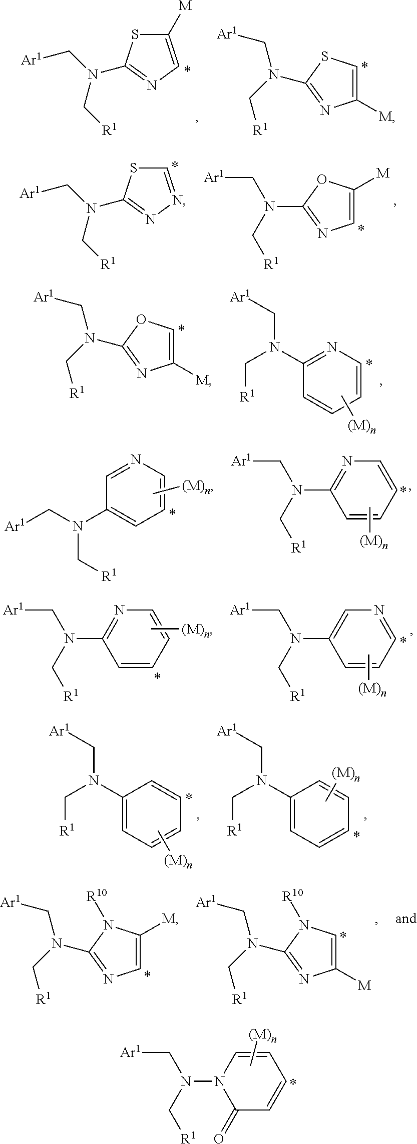

wherein the asterisks of each of the (Ar¹CH₂)(R¹CH₂)N-T groups indicate the attachment point for X; M, when present, may be selected, independently and at each occurrence, from a group comprising: lower alkyl, alkoxy, hydroxyalkyl, aliphatic acyl, —CF₃, —CN, —OH, halogen, heterocycloyl, alkoxyalkyl, (C₁-C₃ alkyl)₂amino, alkoxyalkoxy, cycloalkyl, cycloalkylalkyl, heterocyclyl, alkylheterocyclyl, and heterocyclylalkyl; n is an integer from 0-3; R¹⁰ when present is hydrogen or a C₁-C₄ lower alkyl; X is further selected from a group of radicals comprising: —(CH₂)_{z}—, —(CH₂)_{z}O—, —(CH₂)_{z}O(CH₂)_{z}—, —(CH₂CH₂O)ₚ—, —(CH₂)_{z}O(CH₂)_{z}, —(CH₂)_{z}O(CH₂)_{z}O(CH₂)_{z}—, —(CH₂)_{z}O(CH₂CH₂O)ₚ—, —(CH₂)_{z}O(CH₂CH₂O)ₚ—(CH₂)_{z}—, —(CH₂)_{z}NR²—, —(CH₂)_{z}NR²(CH₂)_{z}—, —(CH₂)_{z}NR²(CH₂CH₂O)ₚ—, —(CH₂CH₂O)ₚ(CH₂)_{z}NR²—, —C(O)N(R²)—, —C(O)N(R²)(CH₂)_{z}—, —(CH₂)_{z}C(O)N(R²)(CH₂)_{z}—, —(CH₂)_{z}C(O)N(R²)—, —O(CH₂)_{z}—, —O(CH₂)_{z}O(CH₂)_{z}—, —O(CH₂CH₂O)ₚ—, —O(CH₂)_{z}NR²(CH₂)_{z}—, —O(CH₂)_{z}NR²(CH₂CH₂O)ₚ—, —O(CH₂CH₂O)ₚ(CH₂)_{z}NR²—, —O(CH₂)_{z}C(O)N(R²)(CH₂)_{z}—, and —O(CH₂)_{z}C(O)N(R²)—; z is independently at each occurrence an integer of 1-20, and p is an integer from 0-6, provided that in X for any combination of groups, the integers p and z do not create a chain of greater than 20 atoms; and a radical of M and 1 or 2 atoms of X may be taken together to form a 5 or 6 membered ring.

A third aspect which is the compound of the second aspect wherein Ar¹ is selected from the group consisting essentially of substituted phenyl, substituted or unsubstituted or unsubstituted heteroaromatic groups thienyl, oxazolyl, isoxazolyl, pyrrolyl or pyridyl; and R¹ is hydrogen, C₁-C₄ alkyl, substituted phenyl, substituted or unsubstituted heteroaromatic groups selected from: thienyl, unsubstituted thienyl, oxazolyl, isoxazolyl, pyrrolyl and pyridinyl.

A fourth aspect which is the compound of the second aspect wherein (Ar¹CH₂)(R¹CH₂)N-T is selected from a group consisting essentially of

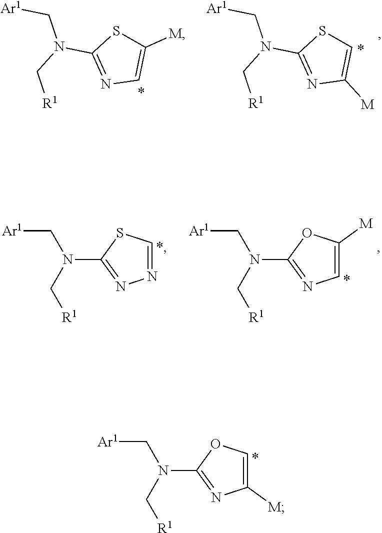

provided that in X for any combination of groups, the integers p and z do not create a chain of greater than 10 atoms; and M is hydrogen or a C₁-C₄ alkyl.

A fifth aspect which is the compound of the first aspect wherein Y is selected from a group consisting essentially of 4-morpholinyl, 1-piperidinyl, 1-piperazinyl, piperazin-2-one-1-yl, piperazine-2,5-dione-1-yl, 4-piperazin-2-one, 1-pyrrolidinyl, 2-oxa-5-azabicyclo[2.2.1]heptane, 3-pyrrolidinol, 4-hydroxypyrrolidine-2-one, 3-hydroxypyrrolidin-2-one, morpholin-3-one, 6-oxa-1-azaspiro[3.3]heptane, 6-oxa-3-azabicyclo[3.1.1]heptane, hexahydro-2H-furo[3,2-b]pyrole, 2-oxa-5-azabicyclo[4.1.0]heptane, phenyl, di(aralkyl)aminoaryl and (N-aralkyl)(N-alkyl)aminoaryl; including stereo-isomers thereof, and Y may be unsubstituted or substituted with one or more substituents selected from the group consisting essentially of halogen, lower alkyl, alkoxy, —CF₃, —CN, —OH, alkoxyalkyl, (C₁-C₃ alkyl)₂amino, alkoxyalkoxy, heterocyclyl, alkylheterocyclyl, and heterocyclylalkyl.

A sixth aspect which is the compound of the first aspect wherein the VLA-4 integrin agonist of the formula (I) is selected from the group consisting essentially of ethyl 2-(bis(thiophen-2-ylmethyl)amino)oxazole-4-carboxylate; 2-(bis(thiophen-2-ylmethyl)amino)-N-(3-methoxybenzyl)oxazole-4-carboxamide; 2-(bis(3-methoxybenzyl)amino)-N-(3-methoxybenzyl)oxazole-4-carboxamide; 2-(bis(3-methoxybenzyl)amino)-N-(4-methoxyphenethyl)oxazole-4-carboxamide; 2-(bis(3-methoxybenzyl)amino)-N-(3-(4-methoxyphenyl)propyl)oxazole-4-carboxamide; 2-(bis(3-methoxybenzyl)amino)-N-(3-(4-methoxyphenyl)propyl)oxazole-5-carboxamide; 2-(bis(3-methoxybenzyl)amino)-N-(4-methoxyphenethyl)thiazole-4-carboxamide; 2-(bis(3-methoxybenzyl)amino)-N-(3-(4-methoxyphenyl)propyl)thiazole-4-carboxamide; 4-((3-(4-methoxyphenyl)propoxy)methyl)-N,N-bis(thiophen-2-ylmethyl)oxazol-2-amine; ethyl 2-(bis(4-methoxybenzyl)amino)oxazole-4-carboxylate; (2-(bis(thiophen-2-ylmethyl)amino)oxazol-4-yl)methanol; ethyl 2-(bis(3-methoxybenzyl)amino)oxazole-4-carboxylate; ethyl 2-(bis(3-methoxybenzyl)amino)oxazole-4-carboxylate; (2-(bis(4-methoxybenzyl)amino)oxazol-4-yl)methanol; N,N-bis(3-methoxybenzyl)-2-(2-phenoxyethyl)oxazol-5-amine; ethyl 2-(bis(3-(2-methoxyethoxy)benzyl)amino)oxazole-4-carboxylate; 2-(2-((2-(bis(4-methoxybenzyl)amino)thiazol-4-yl)methoxy)ethoxy)ethanol; 4,4′-(2,2′-oxybis(ethane-2,1-diyl)bis(oxy))bis(methylene)bis(N,N-bis(4-methoxybenzyl)thiazol-2-amine); 2-((2-(bis(3-methoxybenzyl)amino)thiazol-4-yl)methoxy)ethanol; N,N-bis(3-methoxybenzyl)-4-((2-(2-(3-methoxyphenoxy)ethoxy)ethoxy)methyl)thiazol-2-amine; 4-((2-(2-(3-(dimethylamino)phenoxy)ethoxy)ethoxy)methyl)-N,N-bis(3-methoxybenzyl)thiazol-2-amine; 4-((2-(2-(benzyloxy)ethoxy)ethoxy)methyl)-N,N-bis(3-methoxybenzyl)thiazol-2-amine; N,N-bis(3-methoxybenzyl)-4-(((3-methoxybenzyl)oxy)methyl)thiazol-2-amine; N,N-bis(3-methoxybenzyl)-4-((3-(4-methoxyphenyl)propoxy)methyl)thiazol-2-amine; N,N-bis(3-methoxybenzyl)-4-((2-(3-methoxyphenoxy)ethoxy)methyl)thiazol-2-amine; N,N-bis(3-methoxybenzyl)-4-(((3-methoxybenzyl)oxy)methyl)thiazol-2-amine; N,N-bis(3-methoxybenzyl)-4-((3-(4-methoxyphenyl)propoxy)methyl)thiazol-2-amine; N,N-bis(3-methoxybenzyl)-4-((2-(3-methoxyphenoxy)ethoxy)methyl)thiazol-2-amine; 4-((2-(3-(dimethylamino)phenoxy)ethoxy)methyl)-N,N-bis(3-methoxybenzyl)thiazol-2-amine; 4-((2-(benzyloxy)ethoxy)methyl)-N,N-bis(3-methoxybenzyl)thiazol-2-amine; 4-((2-((2-chlorobenzyl)oxy)ethoxy)methyl)-N,N-bis(3-methoxybenzyl)thiazol-2-amine; N,N-bis(3-methoxybenzyl)-4-((2-(2-((2-methylbenzyl)oxy)ethoxy)ethoxy)methyl)thiazol-2-amine; N,N-bis(3-methoxybenzyl)-4-((4-methoxyphenethoxy)methyl)thiazol-2-amine; N,N-bis(3-methoxybenzyl)-4-((2-((2-methylbenzyl)oxy)ethoxy)methyl)thiazol-2-amine; N,N-bis(3-methoxybenzyl)-4-((2-(2-((3-methoxybenzyl)oxy)ethoxy)ethoxy)methyl)thiazol-2-amine; N,N-bis(3-methoxybenzyl)-4-((2-((3-methoxybenzyl)oxy)ethoxy)methyl)thiazol-2-amine; N,N-bis(3-methoxybenzyl)-4-((2-(2-phenoxyethoxy)ethoxy)methyl)thiazol-2-amine; 4-((2-(2-(4-chlorophenoxy)ethoxy)ethoxy)methyl)-N,N-bis(3-methoxybenzyl)thiazol-2-amine; 3-(2-(2-((2-(bis(3-methoxybenzyl)amino)thiazol-4-yl)methoxy)ethoxy)ethoxy)benzonitrile; 4-((2-(4-chlorophenoxy)ethoxy)methyl)-N,N-bis(3-methoxybenzyl)thiazol-2-amine; 4-((2-(2-(4-(dimethylamino)phenoxy)ethoxy)ethoxy)methyl)-N,N-bis(3-methoxybenzyl)thiazol-2-amine; 3-(2-((2-(bis(3-methoxybenzyl)amino)thiazol-4-yl)methoxy)ethoxy)benzonitrile; 4-((2-(4-(dimethylamino)phenoxy)ethoxy)methyl)-N,N-bis(3-methoxybenzyl)thiazol-2-amine; N,N-bis(3-methoxybenzyl)-4-(morpholinomethyl)thiazol-2-amine; 4-((2-(2-((2-chlorobenzyl)oxy)ethoxy)ethoxy)methyl)-N,N-bis(3-methoxybenzyl)thiazol-2-amine; N,N-bis(3-methoxybenzyl)-4-((2-phenoxyethoxy)methyl)thiazol-2-amine; N,N-bis(3-methoxybenzyl)-4-methylthiazol-2-amine; N,N-bis(3-(2-methoxyethoxy)benzyl)-4-methylthiazol-2-amine; N,N-bis(3-methoxybenzyl)-4-(piperidin-1-ylmethyl)thiazol-2-amine; N,N-bis(3-methoxybenzyl)-4-((2-(2-morpholinoethoxy)ethoxy)methyl)thiazol-2-amine; N,N-bis(3-methoxybenzyl)-4-((4-methylpiperazin-1-yl)methyl)thiazol-2-amine; N,N-bis(3-methoxybenzyl)-4-((2-morpholinoethoxy)methyl)thiazol-2-amine; N,N-bis(3-methoxybenzyl)-4-((2-(piperidin-1-yl)ethoxy)methyl)thiazol-2-amine; N,N-bis(3-methoxybenzyl)-4-((2-(4-methylpiperazin-1-yl)ethoxy)methyl)thiazol-2-amine; N,N-bis(3-methoxybenzyl)-4-((2-(2-(piperidin-1-yl)ethoxy)ethoxy)methyl)thiazol-2-amine; and N,N-bis(3-methoxybenzyl)-4-((2-(2-(4-methylpiperazin-1-yl)ethoxy)ethoxy)methyl)thiazol-2-amine.

A seventh aspect which is the compound of the first aspect wherein the VLA-4 integrin agonist of the formula (I) is selected from the group consisting essentially of 4-((2-(2-(dimethylamino)ethoxy)ethoxy)methyl)-N,N-bis(3-methoxybenzyl)thiazol-2-amine; 4-((2-(dimethylamino)ethoxy)methyl)-N,N-bis(3-methoxybenzyl)thiazol-2-amine; N,N-bis(3-methoxybenzyl)-4-(2-methyl-5,8,11-trioxa-2-azadodecan-12-yl)thizazol-2-amine; N,N-bis(3-methoxybenzyl)-4-((2-(2-(2-morpholinoethoxy)ethoxy)ethoxy)methyl)thiazol-2-amine; 4-((2-(2-(indolin-1-yl)ethoxy)ethoxy)methyl)-N,N-bis(3-methoxybenzyl)thiazol-2-amine; 4-((2-(2-(3,4-dihydroquinolin-1(2H)-yl)ethoxy)ethoxy)methyl)-N,N-bis(3-methoxybenzyl)thiazol-2-amine; 4-((2-(3,4-dihydroquinolin-1(2H)-yl)ethoxy)methyl)-N,N-bis(3-methoxybenzyl)thiazol-2-amine; 4-((2-(indolin-1-yl)ethoxy)methyl)-N,N-bis(3-methoxybenzyl)thiazol-2-amine; 1-((2-(bis(3-methoxybenzyl)amino)thiazol-4-yl)methyl)piperazin-2-one; 1-((2-(bis(3-methoxybenzyl)amino)thiazol-4-yl)methyl)piperazine-2,5-dione; 4-((2-(bis(3-methoxybenzyl)amino)thiazol-4-yl)methyl)piperazin-2-one; N,N-bis(3-methoxybenzyl)-4-methyloxazol-2-amine; N-(3-(dimethylamino)benzyl)-N-(3-methoxybenzyl)-4-methyloxazol-2-amine; N-(3-methoxybenzyl)-4-methyl-N-(4-(pyrrolidin-1-yl)benzyl)oxazol-2-amine; N-(3-methoxybenzyl)-4-methyl-N-(3-morpholinobenzyl)oxazol-2-amine; N-(3-methoxybenzyl)-4-methyl-N-(3-(pyrrolidin-1-yl)benzyl)oxazol-2-amine; N-(3-(1H-imidazol-1-yl)benzyl)-N-(3-methoxybenzyl)-4-methyloxazol-2-amine; N-(3-(1H-pyrazol-1-yl)benzyl)-N-(3-methoxybenzyl)-4-methyloxazol-2-amine; N-((1H-indazol-5-yl)methyl)-N-(3-methoxybenzyl)-4-methyloxazol-2-amine; N-((1H-benzo[d]imidazol-5-yl)methyl)-N-(3-methoxybenzyl)-4-methyloxazol-2-amine; N-(3-methoxybenzyl)-4-methyl-N-(quinolin-6-ylmethyl)oxazol-2-amine; N-(3-methoxybenzyl)-4-((2-(3-methoxybenzyloxy)ethoxy)methyl)-N-(quinolin-6-ylmethyl)oxazol-2-amine; 4-((2-(3-(dimethylamino)phenoxy)ethoxy)methyl)-N-(3-methoxybenzyl)-N-(quinolin-6-ylmethyl)oxazol-2-amine; N-(3-(dimethylamino)benzyl)-N-(3-methoxybenzyl)-4-((2-(3-methoxybenzyloxy)ethoxy)methyl)oxazol-2-amine; N-(3-methoxybenzyl)-4-((2-(3-methoxybenzyloxy)ethoxy)methyl)-N-(3-(pyrrolidin-1-yl)benzyl)oxazol-2-amine; N-(3-(1H-imidazol-1-yl)benzyl)-N-(3-methoxybenzyl)-4-((2-(3-methoxybenzyloxy)ethoxy)methyl)oxazol-2-amine; N-(3-(1H-pyrazol-1-yl)benzyl)-N-(3-methoxybenzyl)-4-((2-(3-methoxybenzyloxy)ethoxy)methyl)oxazol-2-amine; N-(3-methoxybenzyl)-4-((2-(3-methoxybenzyloxy)ethoxy)methyl)-N-(3-morpholinobenzyl)oxazol-2-amine; N-(3-methoxybenzyl)-4-((2-(3-methoxybenzyloxy)ethoxy)methyl)-N-(3-(4-methylpiperazin-1-yl)benzyl)oxazol-2-amine; N-((1H-benzo[d]imidazol-5-yl)methyl)-N-(3-methoxybenzyl)-4-((2-(3-methoxybenzyloxy)ethoxy)methyl)oxazol-2-amine; N-((1H-indazol-5-yl)methyl)-N-(3-methoxybenzyl)-4-((2-(3-methoxybenzyloxy)ethoxy)methyl)oxazol-2-amine; N-(3-methoxybenzyl)-4-((2-(3-methoxybenzyloxy)ethoxy)methyl)-N-(quinolin-6-ylmethyl)thiazol-2-amine; N-(3-methoxybenzyl)-4-((2-morpholinoethoxy)methyl)-N-(quinolin-6-ylmethyl)oxazol-2-amine; N-(4-(1H-imidazol-1-yl)benzyl)-N-(3-methoxybenzyl)-4-((2-morpholinoethoxy)methyl)oxazol-2-amine; N-(4-(1H-pyrazol-1-yl)benzyl)-N-(3-methoxybenzyl)-4-((2-morpholinoethoxy)methyl)oxazol-2-amine; N-(3-methoxybenzyl)-N-(4-morpholinobenzyl)-4-((2-morpholinoethoxy)methyl)oxazol-2-amine; N-(3-methoxybenzyl)-4-((2-morpholinoethoxy)methyl)-N-(4-(pyrrolidin-1-yl)benzyl)oxazol-2-amine; N-(3-methoxybenzyl)-N-(4-(4-methylpiperazin-1-yl)benzyl)-4-((2-morpholinoethoxy)methyl)oxazol-2-amine; N-((1H-benzo[d]imidazol-6-yl)methyl)-N-(3-methoxybenzyl)-4-((2-morpholinoethoxy)methyl)oxazol-2-amine; N-((1H-indazol-6-yl)methyl)-N-(3-methoxybenzyl)-4-((2-morpholinoethoxy)methyl)oxazol-2-amine; N-(3-(dimethylamino)benzyl)-N-(3-methoxybenzyl)-4-methylthiazol-2-amine; N-(3-methoxybenzyl)-4-methyl-N-(3-morpholinobenzyl)thiazol-2-amine; N-(3-methoxybenzyl)-4-methyl-N-(3-morpholinobenzyl)oxazol-2-amine; N-(3-(1H-imidazol-1-yl)benzyl)-N-(3-methoxybenzyl)-4-methyloxazol-2-amine; N-(3-(1H-pyrazol-1-yl)benzyl)-N-(3-methoxybenzyl)-4-methyloxazol-2-amine; N-(3-(1H-imidazol-1-yl)benzyl)-N-(3-methoxybenzyl)-4-methylthiazol-2-amine; N-(3-(1H-pyrazol-1-yl)benzyl)-N-(3-methoxybenzyl)-4-methylthiazol-2-amine; N-(3-methoxybenzyl)-4-methyl-N-(4-(pyrrolidin-1-yl)benzyl)thiazol-2-amine; N-(4-(1H-imidazol-1-yl)benzyl)-N-(3-methoxybenzyl)-4-methylthiazol-2-amine; N-(4-(1H-pyrazol-1-yl)benzyl)-N-(3-methoxybenzyl)-4-methylthiazol-2-amine; N-(4-(1H-imidazol-1-yl)benzyl)-N-(3-methoxybenzyl)-4-methyloxazol-2-amine; N-(3-methoxybenzyl)-4-methyl-N-(4-(4-methylpiperazin-1-yl)benzyl)thiazol-2-amine; N-(3-methoxybenzyl)-4-methyl-N-(4-morpholinobenzyl)thiazol-2-amine; N-((1H-benzo[d]imidazol-6-yl)methyl)-N-(3-methoxybenzyl)-4-methylthiazol-2-amine; N-((1H-indazol-6-yl)methyl)-N-(3-methoxybenzyl)-4-methylthiazol-2-amine; N-(3-methoxybenzyl)-4-methyl-N-(quinolin-6-ylmethyl)thiazol-2-amine; N,N-bis(3-methoxybenzyl)-4-((2-(2-(3-methoxyphenoxy)ethoxy)ethoxy)methyl)oxazol-2-amine; 4-((2-(2-(benzyloxy)ethoxy)ethoxy)methyl)-N,N-bis(3-methoxybenzyl)oxazol-2-amine; 4-((2-(3-(dimethylamino)phenoxy)ethoxy)methyl)-N,N-bis(3-methoxybenzyl)oxazol-2-amine; N,N-bis(3-methoxybenzyl)-4-((2-(2-(3-methoxybenzyloxy)ethoxy)ethoxy)methyl)oxazol-2-amine; N,N-bis(3-methoxybenzyl)-4-((2-(3-methoxybenzyloxy)ethoxy)methyl)oxazol-2-amine; N,N-bis(3-methoxybenzyl)-4-(morpholinomethyl)oxazol-2-amine; N,N-bis(3-(2-methoxyethoxy)benzyl)-4-methyloxazol-2-amine; N,N-bis(3-methoxybenzyl)-4-((2-(2-morpholinoethoxy)ethoxy)methyl)oxazol-2-amine; N,N-bis(3-methoxybenzyl)-4-((2-morpholinoethoxy)methyl)oxazol-2-amine; N-(3-methoxybenzyl)-4-((2-(3-methoxybenzyloxy)ethoxy)methyl)-N-(4-(pyrrolidin-1-yl)benzyl)oxazol-2-amine; N-(4-(1H-imidazol-1-yl)benzyl)-N-(3-methoxybenzyl)-4-((2-(3-methoxybenzyloxy)ethoxy)methyl)oxazol-2-amine; N-(4-(1H-pyrazol-1-yl)benzyl)-N-(3-methoxybenzyl)-4-((2-(3-methoxybenzyloxy)ethoxy)methyl)oxazol-2-amine; N-(3-methoxybenzyl)-4-((2-(3-methoxybenzyloxy)ethoxy)methyl)-N-(4-morpholinobenzyl)oxazol-2-amine; N-(3-methoxybenzyl)-4-((2-(3-methoxybenzyloxy)ethoxy)methyl)-N-(4-(4-methylpiperazin-1-yl)benzyl)oxazol-2-amine; N-(3-methoxybenzyl)-4-((2-(3-methoxybenzyloxy)ethoxy)methyl)-N-(quinolin-6-ylmethyl)thiazol-2-amine; N-(3-methoxybenzyl)-4-((2-(3-methoxybenzyloxy)ethoxy)methyl)-N-(3-(pyrrolidin-1-yl)benzyl)thiazol-2-amine; N-(3-(dimethylamino)benzyl)-N-(3-methoxybenzyl)-4-((2-(3-methoxybenzyloxy)ethoxy)methyl)thiazol-2-amine; N-(3-(1H-imidazol-1-yl)benzyl)-N-(3-methoxybenzyl)-4-((2-(3-methoxybenzyloxy)ethoxy)methyl)thiazol-2-amine; N-(3-(1H-pyrazol-1-yl)benzyl)-N-(3-methoxybenzyl)-4-((2-(3-methoxybenzyloxy)ethoxy)methyl)thiazol-2-amine; N-(3-methoxybenzyl)-4-((2-(3-methoxybenzyloxy)ethoxy)methyl)-N-(3-morpholinobenzyl)thiazol-2-amine; N-(3-methoxybenzyl)-4-((2-(3-methoxybenzyloxy)ethoxy)methyl)-N-(3-(4-methylpiperazin-1-yl)benzyl)thiazol-2-amine; N-((1H-benzo[d]imidazol-5-yl)methyl)-N-(3-methoxybenzyl)-4-((2-(3-methoxybenzyloxy)ethoxy)methyl)thiazol-2-amine; N-((1H-indazol-5-yl)methyl)-N-(3-methoxybenzyl)-4-((2-(3-methoxybenzyloxy)ethoxy)methyl)thiazol-2-amine; N-(3-methoxybenzyl)-4-((2-(3-methoxybenzyloxy)ethoxy)methyl)-N-(4-(pyrrolidin-1-yl)benzyl)thiazol-2-amine; N-(4-(1H-imidazol-1-yl)benzyl)-N-(3-methoxybenzyl)-4-((2-(3-methoxybenzyloxy)ethoxy)methyl)thiazol-2-amine; N-(4-(1H-pyrazol-1-yl)benzyl)-N-(3-methoxybenzyl)-4-((2-(3-methoxybenzyloxy)ethoxy)methyl)thiazol-2-amine; N-(3-methoxybenzyl)-4-((2-(3-methoxybenzyloxy)ethoxy)methyl)-N-(4-morpholinobenzyl)thiazol-2-amine; N-(3-methoxybenzyl)-4-((2-(3-methoxybenzyloxy)ethoxy)methyl)-N-(4-(4-methylpiperazin-1-yl)benzyl)thiazol-2-amine; N-(3-methoxybenzyl)-N-(3-(4-methylpiperazin-1-yl)benzyl)-4-((2-morpholinoethoxy)methyl)oxazol-2-amine; N-(3-methoxybenzyl)-N-(3-morpholinobenzyl)-4-((2-morpholinoethoxy)methyl)oxazol-2-amine; N-(3-methoxybenzyl)-4-((2-morpholinoethoxy)methyl)-N-(3-(pyrrolidin-1-yl)benzyl)oxazol-2-amine; N-(3-(1H-imidazol-1-yl)benzyl)-N-(3-methoxybenzyl)-4-((2-morpholinoethoxy)methyl)oxazol-2-amine; N-(3-(1H-pyrazol-1-yl)benzyl)-N-(3-methoxybenzyl)-4-((2-morpholinoethoxy)methyl)oxazol-2-amine; N-(3-(dimethylamino)benzyl)-N-(3-methoxybenzyl)-4-((2-morpholinoethoxy)methyl)oxazol-2-amine; N-(3-(dimethylamino)benzyl)-N-(3-methoxybenzyl)-4-((2-morpholinoethoxy)methyl)thiazol-2-amine; N-(3-methoxybenzyl)-4-((2-morpholinoethoxy)methyl)-N-(3-(pyrrolidin-1-yl)benzyl)thiazol-2-amine; N-(3-(1H-pyrazol-1-yl)benzyl)-N-(3-methoxybenzyl)-4-((2-morpholinoethoxy)methyl)thiazol-2-amine; N-(3-(1H-imidazol-1-yl)benzyl)-N-(3-methoxybenzyl)-4-((2-morpholinoethoxy)methyl)thiazol-2-amine; N-(3-methoxybenzyl)-4-((2-morpholinoethoxy)methyl)-N-(4-(pyrrolidin-1-yl)benzyl)thiazol-2-amine; N-(3-methoxybenzyl)-N-(4-morpholinobenzyl)-4-((2-morpholinoethoxy)methyl)thiazol-2-amine; N-(3-methoxybenzyl)-N-(4-(4-methylpiperazin-1-yl)benzyl)-4-((2-morpholinoethoxy)methyl)thiazol-2-amine; N-(3-methoxybenzyl)-N-(3-(4-methylpiperazin-1-yl)benzyl)-4-((2-morpholinoethoxy)methyl)thiazol-2-amine; N-(3-methoxybenzyl)-N-(3-morpholinobenzyl)-4-((2-morpholinoethoxy)methyl)thiazol-2-amine; N-(4-(1H-pyrazol-1-yl)benzyl)-N-(3-methoxybenzyl)-4-((2-morpholinoethoxy)methyl)thiazol-2-amine; N-(4-(1H-imidazol-1-yl)benzyl)-N-(3-methoxybenzyl)-4-((2-morpholinoethoxy)methyl)thiazol-2-amine; N-((1H-benzo[d]imidazol-6-yl)methyl)-N-(3-methoxybenzyl)-4-((2-morpholinoethoxy)methyl)thiazol-2-amine; N-((1H-indazol-5-yl)methyl)-N-(3-methoxybenzyl)-4-((2-morpholinoethoxy)methyl)thiazol-2-amine; N-(3-methoxybenzyl)-4-((2-morpholinoethoxy)methyl)-N-(quinolin-6-ylmethyl)thiazol-2-amine; 4-((2-(2-(dimethylamino)ethoxy)ethoxy)methyl)-N,N-bis(3-methoxybenzyl)oxazol-2-amine; 4-((2-(dimethylamino)ethoxy)methyl)-N,N-bis(3-methoxybenzyl)oxazol-2-amine; N,N-bis(3-methoxybenzyl)-4-(2-methyl-5,8,11-trioxa-2-azadodecan-12-yl)oxazol-2-amine; N,N-bis(3-methoxybenzyl)-4-((2-(2-(2-morpholinoethoxy)ethoxy)ethoxy)methyl)thiazol-2-amine; 4-((2-(2-(indolin-1-yl)ethoxy)ethoxy)methyl)-N,N-bis(3-methoxybenzyl)oxazol-2-amine; 4-((2-(2-(3,4-dihydroquinolin-1(2H)-yl)ethoxy)ethoxy)methyl)-N,N-bis(3-methoxybenzyl)oxazol-2-amine; 4-((2-(3,4-dihydroquinolin-1(2H)-yl)ethoxy)methyl)-N,N-bis(3-methoxybenzyl)oxazol-2-amine; 4-((2-(indolin-1-yl)ethoxy)methyl)-N,N-bis(3-methoxybenzyl)oxazol-2-amine; 1-((2-(bis(3-methoxybenzyl)amino)oxazol-4-yl)methyl)piperazin-2-one; 1-((2-(bis(3-methoxybenzyl)amino)oxazol-4-yl)methyl)piperazine-2,5-dione; 4-((2-(bis(3-methoxybenzyl)amino)oxazol-4-yl)methyl)piperazin-2-one; N-(4-(1H-imidazol-1-yl)benzyl)-4-((2-(3-(dimethylamino)phenoxy)ethoxy)methyl)-N-(3-methoxybenzyl)oxazol-2-amine; N-(4-(1H-pyrazol-1-yl)benzyl)-4-((2-(3-(dimethylamino)phenoxy)ethoxy)methyl)-N-(3-methoxybenzyl)oxazol-2-amine; 4-((2-(3-(dimethylamino)phenoxy)ethoxy)methyl)-N-(3-methoxybenzyl)-N-(4-morpholinobenzyl)oxazol-2-amine; 4-((2-(3-(dimethylamino)phenoxy)ethoxy)methyl)-N-(3-methoxybenzyl)-N-(4-(pyrrolidin-1-yl)benzyl)oxazol-2-amine; 4-((2-(3-(dimethylamino)phenoxy)ethoxy)methyl)-N-(3-methoxybenzyl)-N-(4-(4-methylpiperazin-1-yl)benzyl)oxazol-2-amine; N-(3-(dimethylamino)benzyl)-4-((2-(3-(dimethylamino)phenoxy)ethoxy)methyl)-N-(3-methoxybenzyl)oxazol-2-amine; 4-((2-(3-(dimethylamino)phenoxy)ethoxy)methyl)-N-(3-methoxybenzyl)-N-(3-(pyrrolidin-1-yl)benzyl)oxazol-2-amine; N-(3-(1H-imidazol-1-yl)benzyl)-4-((2-(3-(dimethylamino)phenoxy)ethoxy)methyl)-N-(3-methoxybenzyl)oxazol-2-amine; N-(3-(1H-pyrazol-1-yl)benzyl)-4-((2-(3-(dimethylamino)phenoxy)ethoxy)methyl)-N-(3-methoxybenzyl)oxazol-2-amine; 4-((2-(3-(dimethylamino)phenoxy)ethoxy)methyl)-N-(3-methoxybenzyl)-N-(3-morpholinobenzyl)oxazol-2-amine; 4-((2-(3-(dimethylamino)phenoxy)ethoxy)methyl)-N-(3-methoxybenzyl)-N-(3-(4-methylpiperazin-1-yl)benzyl)oxazol-2-amine; N-((1H-benzo[d]imidazol-6-yl)methyl)-4-((2-(3-(dimethylamino)phenoxy)ethoxy)methyl)-N-(3-methoxybenzyl)oxazol-2-amine; N-((1H-indazol-5-yl)methyl)-4-((2-(3-(dimethylamino)phenoxy)ethoxy)methyl)-N-(3-methoxybenzyl)oxazol-2-amine; 4-((2-(3-(dimethylamino)phenoxy)ethoxy)methyl)-N-(3-methoxybenzyl)-N-(quinolin-6-ylmethyl)oxazol-2-amine; N-(4-(1H-imidazol-1-yl)benzyl)-4-((2-(3-(dimethylamino)phenoxy)ethoxy)methyl)-N-(3-methoxybenzyl)thiazol-2-amine; N-(4-(1H-pyrazol-1-yl)benzyl)-4-((2-(3-(dimethylamino)phenoxy)ethoxy)methyl)-N-(3-methoxybenzyl)thiazol-2-amine; 4-((2-(3-(dimethylamino)phenoxy)ethoxy)methyl)-N-(3-methoxybenzyl)-N-(4-morpholinobenzyl)thiazol-2-amine; 4-((2-(3-(dimethylamino)phenoxy)ethoxy)methyl)-N-(3-methoxybenzyl)-N-(4-(pyrrolidin-1-yl)benzyl)thiazol-2-amine; 4-((2-(3-(dimethylamino)phenoxy)ethoxy)methyl)-N-(3-methoxybenzyl)-N-(4-(4-methylpiperazin-1-yl)benzyl)thiazol-2-amine; N-(3-(dimethylamino)benzyl)-4-((2-(3-(dimethylamino)phenoxy)ethoxy)methyl)-N-(3-methoxybenzyl)thiazol-2-amine; 4-((2-(3-(dimethylamino)phenoxy)ethoxy)methyl)-N-(3-methoxybenzyl)-N-(3-(pyrrolidin-1-yl)benzyl)thiazol-2-amine; N-(3-(1H-imidazol-1-yl)benzyl)-4-((2-(3-(dimethylamino)phenoxy)ethoxy)methyl)-N-(3-methoxybenzyl)thiazol-2-amine; N-(3-(1H-pyrazol-1-yl)benzyl)-4-((2-(3-(dimethylamino)phenoxy)ethoxy)methyl)-N-(3-methoxybenzyl)thiazol-2-amine; 4-((2-(3-(dimethylamino)phenoxy)ethoxy)methyl)-N-(3-methoxybenzyl)-N-(3-morpholinobenzyl)thiazol-2-amine; 4-((2-(3-(dimethylamino)phenoxy)ethoxy)methyl)-N-(3-methoxybenzyl)-N-(3-(4-methylpiperazin-1-yl)benzyl)thiazol-2-amine; N-((1H-benzo[d]imidazol-5-yl)methyl)-4-((2-(3-(dimethylamino)phenoxy)ethoxy)methyl)-N-(3-methoxybenzyl)thiazol-2-amine; N-((1H-indazol-5-yl)methyl)-4-((2-(3-(dimethylamino)phenoxy)ethoxy)methyl)-N-(3-methoxybenzyl)thiazol-2-amine; 4-((2-(3-(dimethylamino)phenoxy)ethoxy)methyl)-N-(3-methoxybenzyl)-N-(quinolin-6-ylmethyl)thiazol-2-amine; N-(3-methoxybenzyl)-4-(morpholinomethyl)-N-(3-(pyrrolidin-1-yl)benzyl)oxazol-2-amine; N-(3-(1H-pyrazol-1-yl)benzyl)-N-(3-methoxybenzyl)-4-(morpholinomethyl)oxazol-2-amine; N-(3-(1H-imidazol-1-yl)benzyl)-N-(3-methoxybenzyl)-4-(morpholinomethyl)oxazol-2-amine; N-(3-methoxybenzyl)-N-(3-morpholinobenzyl)-4-(morpholinomethyl)oxazol-2-amine; N-(3-methoxybenzyl)-N-(3-(4-methylpiperazin-1-yl)benzyl)-4-(morpholinomethyl)oxazol-2-amine; N-(3-methoxybenzyl)-4-(morpholinomethyl)-N-(4-(pyrrolidin-1-yl)benzyl)oxazol-2-amine; N-(3-methoxybenzyl)-N-(4-morpholinobenzyl)-4-(morpholinomethyl)oxazol-2-amine; N-(3-methoxybenzyl)-N-(4-(4-methylpiperazin-1-yl)benzyl)-4-(morpholinomethyl)oxazol-2-amine; N-(4-(1H-pyrazol-1-yl)benzyl)-N-(3-methoxybenzyl)-4-(morpholinomethyl)oxazol-2-amine; N-(4-(1H-imidazol-1-yl)benzyl)-N-(3-methoxybenzyl)-4-(morpholinomethyl)oxazol-2-amine; N-((1H-benzo[d]imidazol-5-yl)methyl)-N-(3-methoxybenzyl)-4-(morpholinomethyl)oxazol-2-amine; N-((1H-indazol-6-yl)methyl)-N-(3-methoxybenzyl)-4-(morpholinomethyl)oxazol-2-amine; N-(3-methoxybenzyl)-4-(morpholinomethyl)-N-(quinolin-6-ylmethyl)oxazol-2-amine; N-(3-(dimethylamino)benzyl)-N-(3-methoxybenzyl)-4-(morpholinomethyl)oxazol-2-amine; N-(3-methoxybenzyl)-4-(morpholinomethyl)-N-(3-(pyrrolidin-1-yl)benzyl)thiazol-2-amine; N-(3-(1H-pyrazol-1-yl)benzyl)-N-(3-methoxybenzyl)-4-(morpholinomethyl)thiazol-2-amine; N-(3-(1H-imidazol-1-yl)benzyl)-N-(3-methoxybenzyl)-4-(morpholinomethyl)thiazol-2-amine; N-(3-methoxybenzyl)-4-(morpholinomethyl)-N-(4-(pyrrolidin-1-yl)benzyl)thiazol-2-amine; N-(3-methoxybenzyl)-N-(4-morpholinobenzyl)-4-(morpholinomethyl)thiazol-2-amine; N-(3-methoxybenzyl)-N-(4-(4-methylpiperazin-1-yl)benzyl)-4-(morpholinomethyl)thiazol-2-amine; N-(4-(1H-pyrazol-1-yl)benzyl)-N-(3-methoxybenzyl)-4-(morpholinomethyl)thiazol-2-amine; N-(4-(1H-imidazol-1-yl)benzyl)-N-(3-methoxybenzyl)-4-(morpholinomethyl)thiazol-2-amine; N-(3-methoxybenzyl)-N-(3-morpholinobenzyl)-4-(morpholinomethyl)thiazol-2-amine; N-(3-methoxybenzyl)-N-(3-(4-methylpiperazin-1-yl)benzyl)-4-(morpholinomethyl)thiazol-2-amine; N-((1H-benzo[d]imidazol-6-yl)methyl)-N-(3-methoxybenzyl)-4-(morpholinomethyl)thiazol-2-amine; N-((1H-indazol-6-yl)methyl)-N-(3-methoxybenzyl)-4-(morpholinomethyl)thiazol-2-amine; N-(3-methoxybenzyl)-4-(morpholinomethyl)-N-(quinolin-6-ylmethyl)thiazol-2-amine; N-(3-(dimethylamino)benzyl)-N-(3-methoxybenzyl)-4-(morpholinomethyl)thiazol-2-amine; N-(3-methoxybenzyl)-4-(piperidin-1-ylmethyl)-N-(3-(pyrrolidin-1-yl)benzyl)oxazol-2-amine; N-(3-(1H-pyrazol-1-yl)benzyl)-N-(3-methoxybenzyl)-4-(piperidin-1-ylmethyl)oxazol-2-amine; N-(3-(1H-imidazol-1-yl)benzyl)-N-(3-methoxybenzyl)-4-(piperidin-1-ylmethyl)oxazol-2-amine; N-(3-methoxybenzyl)-N-(3-morpholinobenzyl)-4-(piperidin-1-ylmethyl)oxazol-2-amine; N-(3-methoxybenzyl)-N-(3-(4-methylpiperazin-1-yl)benzyl)-4-(piperidin-1-ylmethyl)oxazol-2-amine; N-(3-methoxybenzyl)-4-(piperidin-1-ylmethyl)-N-(4-(pyrrolidin-1-yl)benzyl)oxazol-2-amine; N-(3-methoxybenzyl)-N-(4-morpholinobenzyl)-4-(piperidin-1-ylmethyl)oxazol-2-amine; N-(3-methoxybenzyl)-N-(4-(4-methylpiperazin-1-yl)benzyl)-4-(piperidin-1-ylmethyl)oxazol-2-amine; N-(4-(1H-pyrazol-1-yl)benzyl)-N-(3-methoxybenzyl)-4-(piperidin-1-ylmethyl)oxazol-2-amine; N-(4-(1H-imidazol-1-yl)benzyl)-N-(3-methoxybenzyl)-4-(piperidin-1-ylmethyl)oxazol-2-amine; N-((1H-benzo[d]imidazol-5-yl)methyl)-N-(3-methoxybenzyl)-4-(piperidin-1-ylmethyl)oxazol-2-amine; N-((1H-indazol-6-yl)methyl)-N-(3-methoxybenzyl)-4-(piperidin-1-ylmethyl)oxazol-2-amine; N-(3-methoxybenzyl)-4-(piperidin-1-ylmethyl)-N-(quinolin-6-ylmethyl)oxazol-2-amine; N-(3-(dimethylamino)benzyl)-N-(3-methoxybenzyl)-4-(piperidin-1-ylmethyl)oxazol-2-amine; N-(3-methoxybenzyl)-4-(piperidin-1-ylmethyl)-N-(3-(pyrrolidin-1-yl)benzyl)thiazol-2-amine; N-(3-(1H-pyrazol-1-yl)benzyl)-N-(3-methoxybenzyl)-4-(piperidin-1-ylmethyl)thiazol-2-amine; N-(3-(1H-imidazol-1-yl)benzyl)-N-(3-methoxybenzyl)-4-(piperidin-1-ylmethyl)thiazol-2-amine; N-(3-methoxybenzyl)-4-(piperidin-1-ylmethyl)-N-(4-(pyrrolidin-1-yl)benzyl)thiazol-2-amine; N-(3-methoxybenzyl)-N-(4-morpholinobenzyl)-4-(piperidin-1-ylmethyl)thiazol-2-amine; N-(3-methoxybenzyl)-N-(4-(4-methylpiperazin-1-yl)benzyl)-4-(piperidin-1-ylmethyl)thiazol-2-amine; N-(4-(1H-pyrazol-1-yl)benzyl)-N-(3-methoxybenzyl)-4-(piperidin-1-ylmethyl)thiazol-2-amine; N-(4-(1H-imidazol-1-yl)benzyl)-N-(3-methoxybenzyl)-4-(piperidin-1-ylmethyl)thiazol-2-amine; N-(3-methoxybenzyl)-N-(3-morpholinobenzyl)-4-(piperidin-1-ylmethyl)thiazol-2-amine; N-(3-methoxybenzyl)-N-(3-(4-methylpiperazin-1-yl)benzyl)-4-(piperidin-1-ylmethyl)thiazol-2-amine; N-((1H-benzo[d]imidazol-6-yl)methyl)-N-(3-methoxybenzyl)-4-(piperidin-1-ylmethyl)thiazol-2-amine; N-((1H-indazol-6-yl)methyl)-N-(3-methoxybenzyl)-4-(piperidin-1-ylmethyl)thiazol-2-amine; N-(3-methoxybenzyl)-4-(piperidin-1-ylmethyl)-N-(quinolin-6-ylmethyl)thiazol-2-amine; N-(3-(dimethylamino)benzyl)-N-(3-methoxybenzyl)-4-(piperidin-1-ylmethyl)thiazol-2-amine; N-(3-methoxybenzyl)-4-((4-methylpiperazin-1-yl)methyl)-N-(3-(pyrrolidin-1-yl)benzyl)oxazol-2-amine; N-(3-(1H-pyrazol-1-yl)benzyl)-N-(3-methoxybenzyl)-4-((4-methylpiperazin-1-yl)methyl)oxazol-2-amine; N-(3-(1H-imidazol-1-yl)benzyl)-N-(3-methoxybenzyl)-4-((4-methylpiperazin-1-yl)methyl)oxazol-2-amine; N-(3-methoxybenzyl)-4-((4-methylpiperazin-1-yl)methyl)-N-(3-morpholinobenzyl)oxazol-2-amine; N-(3-methoxybenzyl)-N-(3-(4-methylpiperazin-1-yl)benzyl)-4-(piperidin-1-ylmethyl)oxazol-2-amine; N-(3-methoxybenzyl)-4-((4-methylpiperazin-1-yl)methyl)-N-(4-(pyrrolidin-1-yl)benzyl)oxazol-2-amine; N-(3-methoxybenzyl)-4-((4-methylpiperazin-1-yl)methyl)-N-(4-morpholinobenzyl)oxazol-2-amine; N-(3-methoxybenzyl)-N-(4-(4-methylpiperazin-1-yl)benzyl)-4-((4-methylpiperazin-1-yl)methyl)oxazol-2-amine; N-(4-(1H-pyrazol-1-yl)benzyl)-N-(3-methoxybenzyl)-4-((4-methylpiperazin-1-yl)methyl)oxazol-2-amine; N-(4-(1H-imidazol-1-yl)benzyl)-N-(3-methoxybenzyl)-4-((4-methylpiperazin-1-yl)methyl)oxazol-2-amine; N-((1H-benzo[d]imidazol-5-yl)methyl)-N-(3-methoxybenzyl)-4-((4-methylpiperazin-1-yl)methyl)oxazol-2-amine; N-((1H-indazol-6-yl)methyl)-N-(3-methoxybenzyl)-4-((4-methylpiperazin-1-yl)methyl)oxazol-2-amine; N-(3-methoxybenzyl)-4-((4-methylpiperazin-1-yl)methyl)-N-(quinolin-6-ylmethyl)oxazol-2-amine; N-(3-(dimethylamino)benzyl)-N-(3-methoxybenzyl)-4-((4-methylpiperazin-1-yl)methyl)oxazol-2-amine; N-(3-methoxybenzyl)-4-((4-methylpiperazin-1-yl)methyl)-N-(3-(pyrrolidin-1-yl)benzyl)thiazol-2-amine; N-(3-(1H-pyrazol-1-yl)benzyl)-N-(3-methoxybenzyl)-4-((4-methylpiperazin-1-yl)methyl)thiazol-2-amine; N-(3-(1H-imidazol-1-yl)benzyl)-N-(3-methoxybenzyl)-4-((4-methylpiperazin-1-yl)methyl)thiazol-2-amine; N-(3-methoxybenzyl)-4-((4-methylpiperazin-1-yl)methyl)-N-(4-morpholinobenzyl)thiazol-2-amine; N-(3-methoxybenzyl)-N-(4-(4-methylpiperazin-1-yl)benzyl)-4-((4-methylpiperazin-1-yl)methyl)thiazol-2-amine N-(4-(1H-pyrazol-1-yl)benzyl)-N-(3-methoxybenzyl)-4-((4-methylpiperazin-1-yl)methyl)thiazol-2-amine; N-(4-(1H-imidazol-1-yl)benzyl)-N-(3-methoxybenzyl)-4-((4-methylpiperazin-1-yl)methyl)thiazol-2-amine; N-(3-methoxybenzyl)-4-((4-methylpiperazin-1-yl)methyl)-N-(3-morpholinobenzyl)thiazol-2-amine; N-(3-methoxybenzyl)-N-(3-(4-methylpiperazin-1-yl)benzyl)-4-((4-methylpiperazin-1-yl)methyl)thiazol-2-amine; N-((1H-benzo[d]imidazol-6-yl)methyl)-N-(3-methoxybenzyl)-4-((4-methylpiperazin-1-yl)methyl)thiazol-2-amine; N-((1H-indazol-6-yl)methyl)-N-(3-methoxybenzyl)-4-((4-methylpiperazin-1-yl)methyl)thiazol-2-amine; N-(3-methoxybenzyl)-4-((4-methylpiperazin-1-yl)methyl)-N-(quinolin-6-ylmethyl)thiazol-2-amine; N-(3-(dimethylamino)benzyl)-N-(3-methoxybenzyl)-4-((4-methylpiperazin-1-yl)methyl)thiazol-2-amine; 4-((dimethylamino)methyl)-N-(3-methoxybenzyl)-N-(3-(pyrrolidin-1-yl)benzyl)oxazol-2-amine; N-(3-(1H-pyrazol-1-yl)benzyl)-4-((dimethylamino)methyl)-N-(3-methoxybenzyl)oxazol-2-amine; N-(3-(1H-imidazol-1-yl)benzyl)-4-((dimethylamino)methyl)-N-(3-methoxybenzyl)oxazol-2-amine; 4-((dimethylamino)methyl)-N-(3-methoxybenzyl)-N-(3-morpholinobenzyl)oxazol-2-amine 4-((dimethylamino)methyl)-N-(3-methoxybenzyl)-N-(3-(4-methylpiperazin-1-yl)benzyl)oxazol-2-amine; 4-((dimethylamino)methyl)-N-(3-methoxybenzyl)-N-(4-(pyrrolidin-1-yl)benzyl)oxazol-2-amine; 4-((dimethylamino)methyl)-N-(3-methoxybenzyl)-N-(4-morpholinobenzyl)oxazol-2-amine; 4-((dimethylamino)methyl)-N-(3-methoxybenzyl)-N-(4-(4-methylpiperazin-1-yl)benzyl)oxazol-2-amine; N-(4-(1H-pyrazol-1-yl)benzyl)-4-((dimethylamino)methyl)-N-(3-methoxybenzyl)oxazol-2-amine; N-(4-(1H-imidazol-1-yl)benzyl)-4-((dimethylamino)methyl)-N-(3-methoxybenzyl)oxazol-2-amine; N-((1H-indazol-6-yl)methyl)-4-((dimethylamino)methyl)-N-(3-methoxybenzyl)oxazol-2-amine; N-((1H-benzo[d]imidazol-6-yl)methyl)-4-((dimethylamino)methyl)-N-(3-methoxybenzyl)oxazol-2-amine; 4-((dimethylamino)methyl)-N-(3-methoxybenzyl)-N-(quinolin-6-ylmethyl)oxazol-2-amine; N-(3-(dimethylamino)benzyl)-4-((dimethylamino)methyl)-N-(3-methoxybenzyl)oxazol-2-amine; 4-((dimethylamino)methyl)-N-(3-methoxybenzyl)-N-(3-(pyrrolidin-1-yl)benzyl)thiazol-2-amine; N-(3-(1H-pyrazol-1-yl)benzyl)-4-((dimethylamino)methyl)-N-(3-methoxybenzyl)thiazol-2-amine; N-(3-(1H-imidazol-1-yl)benzyl)-4-((dimethylamino)methyl)-N-(3-methoxybenzyl)thiazol-2-amine 4-((dimethylamino)methyl)-N-(3-methoxybenzyl)-N-(4-(pyrrolidin-1-yl)benzyl)thiazol-2-amine; 4-((dimethylamino)methyl)-N-(3-methoxybenzyl)-N-(4-(4-methylpiperazin-1-yl)benzyl)thiazol-2-amine; N-(4-(1H-pyrazol-1-yl)benzyl)-4-((dimethylamino)methyl)-N-(3-methoxybenzyl)thiazol-2-amine; N-(4-(1H-imidazol-1-yl)benzyl)-4-((dimethylamino)methyl)-N-(3-methoxybenzyl)thiazol-2-amine; 4-((dimethylamino)methyl)-N-(3-methoxybenzyl)-N-(3-morpholinobenzyl)thiazol-2-amine; 4-((dimethylamino)methyl)-N-(3-methoxybenzyl)-N-(3-(4-methylpiperazin-1-yl)benzyl)thiazol-2-amine; N-((1H-indazol-6-yl)methyl)-4-((dimethylamino)methyl)-N-(3-methoxybenzyl)thiazol-2-amine; 4-((dimethylamino)methyl)-N-(3-methoxybenzyl)-N-(quinolin-6-ylmethyl)thiazol-2-amine; N-(3-(dimethylamino)benzyl)-4-((dimethylamino)methyl)-N-(3-methoxybenzyl)thiazol-2-amine; 4-((dimethylamino)methyl)-N,N-bis(3-methoxybenzyl)oxazol-2-amine; 4-((dimethylamino)methyl)-N,N-bis(3-methoxybenzyl)thiazol-2-amine; N,N-bis(3-methoxybenzyl)-4-((2-(2-(3-methoxybenzyloxy)ethoxy)ethoxy)methyl)aniline; 4-((2-(2-(benzyloxy)ethoxy)ethoxy)methyl)-N,N-bis(3-methoxybenzyl)aniline; N,N-bis(3-methoxybenzyl)-4-((2-(2-(3-methoxyphenoxy)ethoxy)ethoxy)methyl)aniline; N,N-bis(3-methoxybenzyl)-4-((2-(2-morpholinoethoxy)ethoxy)methyl)aniline; N,N-bis(3-methoxybenzyl)-4-((2-(3-methoxybenzyloxy)ethoxy)methyl)aniline; 3-(2-(4-(bis(3-methoxybenzyl)amino)benzyloxy)ethoxy)-N,N-dimethylaniline; N,N-bis(3-methoxybenzyl)-4-((2-morpholinoethoxy)methyl)aniline; N,N-bis(3-methoxybenzyl)-4-(morpholinomethyl)aniline; N,N-bis(3-methoxybenzyl)-4-(piperidin-1-ylmethyl)aniline; N,N-bis(3-methoxybenzyl)-4-((4-methylpiperazin-1-yl)methyl)aniline; 4-((dimethylamino)methyl)-N,N-bis(3-methoxybenzyl)aniline; 1-(4-(bis(3-methoxybenzyl)amino)benzyl)piperazin-2-one; 4-(4-(bis(3-methoxybenzyl)amino)benzyl)piperazin-2-one; 1-(4-(bis(3-methoxybenzyl)amino)benzyl)piperazine-2,5-dione; 3-(((3-methoxybenzyl)(4-((2-(2-(3-methoxybenzyloxy)ethoxy)ethoxy)methyl)phenyl)amino)methyl)-N,N-dimethylaniline; 3-(((4-((2-(2-(benzyloxy)ethoxy)ethoxy)methyl)phenyl)(3-methoxybenzyl)amino)methyl)-N,N-dimethylaniline 3-(((3-methoxybenzyl)(4-((2-(2-(3-methoxyphenoxy)ethoxy)ethoxy)methyl)phenyl)amino)methyl)-N,N-dimethylaniline; 3-(((3-methoxybenzyl)(4-((2-(2-morpholinoethoxy)ethoxy)methyl)phenyl)amino)methyl)-N,N-dimethylaniline; 3-(((3-methoxybenzyl)(4-((2-(3-methoxybenzyloxy)ethoxy)methyl)phenyl)amino)methyl)-N,N-dimethylaniline; 3-(2-(4-((3-(dimethylamino)benzyl)(3-methoxybenzyl)amino)benzyloxy)ethoxy)-N,N-dimethylaniline; 3-(((3-methoxybenzyl)(4-((2-morpholinoethoxy)methyl)phenyl)amino)methyl)-N,N-dimethylaniline; 3-(((3-methoxybenzyl)(4-(morpholinomethyl)phenyl)amino)methyl)-N,N-dimethylaniline; 3-(((3-methoxybenzyl)(4-(piperidin-1-ylmethyl)phenyl)amino)methyl)-N,N-dimethylaniline; 3-(((3-methoxybenzyl)(4-((4-methylpiperazin-1-yl)methyl)phenyl)amino)methyl)-N,N-dimethylaniline; 1-(4-((3-(dimethylamino)benzyl)(3-methoxybenzyl)amino)benzyl)piperazin-2-one; 1-(4-((3-(dimethylamino)benzyl)(3-methoxybenzyl)amino)benzyl)piperazine-2,5-dione; 4-(4-((3-(dimethylamino)benzyl)(3-methoxybenzyl)amino)benzyl)piperazin-2-one; 3-(((4-((dimethylamino)methyl)phenyl)(3-methoxybenzyl)amino)methyl)-N,N-dimethylaniline; N-(3-methoxybenzyl)-4-((2-(2-morpholinoethoxy)ethoxy)methyl)-N-(3-(pyrrolidin-1-yl)benzyl)aniline; N-(3-methoxybenzyl)-4-((2-(3-methoxybenzyloxy)ethoxy)methyl)-N-(3-(pyrrolidin-1-yl)benzyl)aniline; 3-(2-(4-((3-methoxybenzyl)(3-(pyrrolidin-1-yl)benzyl)amino)benzyloxy)ethoxy)-N,N-dimethylaniline; N-(3-methoxybenzyl)-4-((2-morpholinoethoxy)methyl)-N-(3-(pyrrolidin-1-yl)benzyl)aniline; N-(3-methoxybenzyl)-4-(morpholinomethyl)-N-(3-(pyrrolidin-1-yl)benzyl)aniline; N-(3-methoxybenzyl)-4-(piperidin-1-ylmethyl)-N-(3-(pyrrolidin-1-yl)benzyl)aniline; N-(3-methoxybenzyl)-4-((4-methylpiperazin-1-yl)methyl)-N-(3-(pyrrolidin-1-yl)benzyl)aniline; 1-(4-((3-methoxybenzyl)(3-(pyrrolidin-1-yl)benzyl)amino)benzyl)piperazin-2-one; 1-(4-((3-methoxybenzyl)(3-(pyrrolidin-1-yl)benzyl)amino)benzyl)piperazine-2,5-dione; 4-(4-((3-methoxybenzyl)(3-(pyrrolidin-1-yl)benzyl)amino)benzyl)piperazin-2-one; 4-((dimethylamino)methyl)-N-(3-methoxybenzyl)-N-(3-(pyrrolidin-1-yl)benzyl)aniline; N-(3-(1H-pyrazol-1-yl)benzyl)-N-(3-methoxybenzyl)-4-((2-(2-morpholinoethoxy)ethoxy)methyl)aniline; N-(3-(1H-pyrazol-1-yl)benzyl)-N-(3-methoxybenzyl)-4-((2-(3 methoxybenzyloxy)ethoxy)methyl)aniline; 3-(2-(4-((3-(1H-pyrazol-1-yl)benzyl)(3-methoxybenzyl)amino)benzyloxy)ethoxy)-N,N-dimethylaniline; N-(3-(1H-pyrazol-1-yl)benzyl)-N-(3-methoxybenzyl)-4-((2-morpholinoethoxy)methyl)aniline; N-(3-(1H-pyrazol-1-yl)benzyl)-N-(3-methoxybenzyl)-4-(morpholinomethyl)aniline; N-(3-(1H-pyrazol-1-yl)benzyl)-N-(3-methoxybenzyl)-4-(piperidin-1-ylmethyl)aniline; N-(3-(1H-pyrazol-1-yl)benzyl)-N-(3-methoxybenzyl)-4-((4-methylpiperazin-1-yl)methyl)aniline; 1-(4-((3-(1H-pyrazol-1-yl)benzyl)(3-methoxybenzyl)amino)benzyl)piperazin-2-one; 1-(4-((3-(1H-pyrazol-1-yl)benzyl)(3-methoxybenzyl)amino)benzyl)piperazine-2,5-dione; 4-(4-((3-(1H-pyrazol-1-yl)benzyl)(3-methoxybenzyl)amino)benzyl)piperazin-2-one; N-(3-(1H-pyrazol-1-yl)benzyl)-4-((dimethylamino)methyl)-N-(3-methoxybenzyl)aniline N-(3-(1H-imidazol-1-yl)benzyl)-N-(3-methoxybenzyl)-4-((2-(2-morpholinoethoxy)ethoxy)methyl)aniline; N-(3-(1H-imidazol-1-yl)benzyl)-N-(3-methoxybenzyl)-4-((2-(3-methoxybenzyloxy)ethoxy)methyl)aniline; 3-(2-(4-((3-(1H-imidazol-1-yl)benzyl)(3-methoxybenzyl)amino)benzyloxy)ethoxy)-N,N-dimethylaniline; N-(3-(1H-imidazol-1-yl)benzyl)-N-(3-methoxybenzyl)-4-((2-morpholinoethoxy)methyl)aniline; N-(3-(1H-imidazol-1-yl)benzyl)-N-(3-methoxybenzyl)-4-(morpholinomethyl)aniline; N-(3-(1H-imidazol-1-yl)benzyl)-N-(3-methoxybenzyl)-4-(piperidin-1-ylmethyl)aniline; N-(3-(1H-imidazol-1-yl)benzyl)-N-(3-methoxybenzyl)-4-((4-methylpiperazin-1-yl)methyl)aniline; 1-(4-((3-(1H-imidazol-1-yl)benzyl)(3-methoxybenzyl)amino)benzyl)piperazin-2-one; 1-(4-((3-(1H-imidazol-1-yl)benzyl)(3-methoxybenzyl)amino)benzyl)piperazine-2,5-dione; 4-(4-((3-(1H-imidazol-1-yl)benzyl)(3-methoxybenzyl)amino)benzyl)piperazin-2-one; N-(3-(1H-imidazol-1-yl)benzyl)-4-((dimethylamino)methyl)-N-(3-methoxybenzyl)aniline N-(3-methoxybenzyl)-4-((2-(2-morpholinoethoxy)ethoxy)methyl)-N-(4-(pyrrolidin-1-yl)benzyl)aniline; N-(3-methoxybenzyl)-4-((2-(3-methoxybenzyloxy)ethoxy)methyl)-N-(4-(pyrrolidin-1-yl)benzyl)aniline; 3-(2-(4-((3-methoxybenzyl)(4-(pyrrolidin-1-yl)benzyl)amino)benzyloxy)ethoxy)-N,N-dimethylaniline; N-(3-methoxybenzyl)-4-((2-morpholinoethoxy)methyl)-N-(4-(pyrrolidin-1-yl)benzyl)aniline; N-(3-methoxybenzyl)-4-(morpholinomethyl)-N-(4-(pyrrolidin-1-yl)benzyl)aniline; N-(3-methoxybenzyl)-4-(piperidin-1-ylmethyl)-N-(4-(pyrrolidin-1-yl)benzyl)aniline; N-(3-methoxybenzyl)-4-((4-methylpiperazin-1-yl)methyl)-N-(4-(pyrrolidin-1-yl)benzyl)aniline; 1-(4-((3-methoxybenzyl)(4-(pyrrolidin-1-yl)benzyl)amino)benzyl)piperazin-2-one; 1-(4-((3-methoxybenzyl)(4-(pyrrolidin-1-yl)benzyl)amino)benzyl)piperazine-2,5-dione; 4-(4-((3-methoxybenzyl)(4-(pyrrolidin-1-yl)benzyl)amino)benzyl)piperazin-2-one; 4-((dimethylamino)methyl)-N-(3-methoxybenzyl)-N-(4-(pyrrolidin-1-yl)benzyl)aniline; N-(3-methoxybenzyl)-N-(4-morpholinobenzyl)-4-((2-(2-morpholinoethoxy)ethoxy)methyl)aniline; N-(3-methoxybenzyl)-4-((2-(3-methoxybenzyloxy)ethoxy)methyl)-N-(4-morpholinobenzyl)aniline; 3-(2-(4-((3-methoxybenzyl)(4-morpholinobenzyl)amino)benzyloxy)ethoxy)-N,N-dimethylaniline; N-(3-methoxybenzyl)-N-(4-morpholinobenzyl)-4-((2-morpholinoethoxy)methyl)aniline; N-(3-methoxybenzyl)-N-(4-morpholinobenzyl)-4-(morpholinomethyl)aniline; N-(3-methoxybenzyl)-N-(4-morpholinobenzyl)-4-(piperidin-1-ylmethyl)aniline; N-(3-methoxybenzyl)-4-((4-methylpiperazin-1-yl)methyl)-N-(4-morpholinobenzyl)aniline; 1-(4-((3-methoxybenzyl)(4-morpholinobenzyl)amino)benzyl)piperazin-2-one; 1-(4-((3-methoxybenzyl)(4-morpholinobenzyl)amino)benzyl)piperazine-2,5-dione; 4-(4-((3-methoxybenzyl)(4-morpholinobenzyl)amino)benzyl)piperazin-2-one; 4-((dimethylamino)methyl)-N-(3-methoxybenzyl)-N-(4-morpholinobenzyl)aniline; N-(3-methoxybenzyl)-N-(4-(4-methylpiperazin-1-yl)benzyl)-4-((2-(2-morpholinoethoxy)ethoxy)methyl)aniline; N-(3-methoxybenzyl)-4-((2-(3-methoxybenzyloxy)ethoxy)methyl)-N-(4-(4-methylpiperazin-1-yl)benzyl)aniline; 3-(2-(4-((3-methoxybenzyl)(4-(4-methylpiperazin-1-yl)benzyl)amino)benzyloxy)ethoxy)-N,N-dimethylaniline N-(3-methoxybenzyl)-N-(4-(4-methylpiperazin-1-yl)benzyl)-4-((2-morpholinoethoxy)methyl)aniline; N-(3-methoxybenzyl)-N-(4-(4-methylpiperazin-1-yl)benzyl)-4-(morpholinomethyl)aniline; N-(3-methoxybenzyl)-N-(4-(4-methylpiperazin-1-yl)benzyl)-4-(piperidin-1-ylmethyl)aniline; N-(3-methoxybenzyl)-N-(4-(4-methylpiperazin-1-yl)benzyl)-4-((4-methylpiperazin-1-yl)methyl)aniline; 1-(4-((3-methoxybenzyl)(4-(4-methylpiperazin-1-yl)benzyl)amino)benzyl)piperazin-2-one; 1-(4-((3-methoxybenzyl)(4-(4-methylpiperazin-1-yl)benzyl)amino)benzyl)piperazine-2,5-dione; 4-(4-((3-methoxybenzyl)(4-(4-methylpiperazin-1-yl)benzyl)amino)benzyl)piperazin-2-one; 4-((dimethylamino)methyl)-N-(3-methoxybenzyl)-N-(4-(4-methylpiperazin-1-yl)benzyl)aniline; N-(3-methoxybenzyl)-N-(3-morpholinobenzyl)-4-((2-(2-morpholinoethoxy)ethoxy)methyl)aniline; N-(3-methoxybenzyl)-4-((2-(3-methoxybenzyloxy)ethoxy)methyl)-N-(3-morpholinobenzyl)aniline; 3-(2-(4-((3-methoxybenzyl)(3-morpholinobenzyl)amino)benzyloxy)ethoxy)-N,N-dimethylaniline; N-(3-methoxybenzyl)-N-(3-morpholinobenzyl)-4-((2-morpholinoethoxy)methyl)aniline; N-(3-methoxybenzyl)-N-(3-morpholinobenzyl)-4-(morpholinomethyl)aniline; N-(3-methoxybenzyl)-N-(3-morpholinobenzyl)-4-(piperidin-1-ylmethyl)aniline; N-(3-methoxybenzyl)-4-((4-methylpiperazin-1-yl)methyl)-N-(3-morpholinobenzyl)aniline; 1-(4-((3-methoxybenzyl)(3-morpholinobenzyl)amino)benzyl)piperazin-2-one; 1-(4-((3-methoxybenzyl)(3-morpholinobenzyl)amino)benzyl)piperazine-2,5-dione; 4-(4-((3-methoxybenzyl)(3-morpholinobenzyl)amino)benzyl)piperazin-2-one; 4-((dimethylamino)methyl)-N-(3-methoxybenzyl)-N-(3-morpholinobenzyl)aniline; N-(3-methoxybenzyl)-N-(3-(4-methylpiperazin-1-yl)benzyl)-4-((2-(2-morpholinoethoxy)ethoxy)methyl)aniline; N-(3-methoxybenzyl)-4-((2-(3-methoxybenzyloxy)ethoxy)methyl)-N-(3-(4-methylpiperazin-1-yl)benzyl)aniline; 3-(2-(4-((3-methoxybenzyl)(3-(4-methylpiperazin-1-yl)benzyl)amino)benzyloxy)ethoxy)-N,N-dimethylaniline; N-(3-methoxybenzyl)-N-(3-(4-methylpiperazin-1-yl)benzyl)-4-((2-morpholinoethoxy)methyl)aniline; N-(3-methoxybenzyl)-N-(3-(4-methylpiperazin-1-yl)benzyl)-4-(morpholinomethyl)aniline; N-(3-methoxybenzyl)-N-(3-(4-methylpiperazin-1-yl)benzyl)-4-(piperidin-1-ylmethyl)aniline; N-(3-methoxybenzyl)-N-(3-(4-methylpiperazin-1-yl)benzyl)-4-((4-methylpiperazin-1-yl)methyl)aniline; 1-(4-((3-methoxybenzyl)(3-(4-methylpiperazin-1-yl)benzyl)amino)benzyl)piperazin-2-one; 1-(4-((3-methoxybenzyl)(3-(4-methylpiperazin-1-yl)benzyl)amino)benzyl)piperazine-2,5-dione; 4-(4-((3-methoxybenzyl)(3-(4-methylpiperazin-1-yl)benzyl)amino)benzyl)piperazin-2-one; 4-((dimethylamino)methyl)-N-(3-methoxybenzyl)-N-(3-(4-methylpiperazin-1-yl)benzyl)aniline; N-(4-(1H-pyrazol-1-yl)benzyl)-N-(3-methoxybenzyl)-4-((2-(2-morpholinoethoxy)ethoxy)methyl)aniline; N-(4-(1H-pyrazol-1-yl)benzyl)-N-(3-methoxybenzyl)-4-((2-(3-methoxybenzyloxy)ethoxy)methyl)aniline; 3-(2-(4-((4-(1H-pyrazol-1-yl)benzyl)(3-methoxybenzyl)amino)benzyloxy)ethoxy)-N,N-dimethylaniline; N-(4-(1H-pyrazol-1-yl)benzyl)-N-(3-methoxybenzyl)-4-((2-morpholinoethoxy)methyl)aniline; N-(4-(1H-pyrazol-1-yl)benzyl)-N-(3-methoxybenzyl)-4-(morpholinomethyl)aniline; N-(4-(1H-pyrazol-1-yl)benzyl)-N-(3-methoxybenzyl)-4-(piperidin-1-ylmethyl)aniline; N-(4-(1H-pyrazol-1-yl)benzyl)-N-(3-methoxybenzyl)-4-((4-methylpiperazin-1-yl)methyl)aniline; 1-(4-((4-(1H-pyrazol-1-yl)benzyl)(3-methoxybenzyl)amino)benzyl)piperazin-2-one; 1-(4-((4-(1H-pyrazol-1-yl)benzyl)(3-methoxybenzyl)amino)benzyl)piperazine-2,5-dione; 4-(4-((4-(1H-pyrazol-1-yl)benzyl)(3-methoxybenzyl)amino)benzyl)piperazin-2-one; N-(4-(1H-pyrazol-1-yl)benzyl)-4-((dimethylamino)methyl)-N-(3-methoxybenzyl)aniline; N-(4-(1H-imidazol-1-yl)benzyl)-N-(3-methoxybenzyl)-4-((2-(2-morpholinoethoxy)ethoxy)methyl)aniline; N-(4-(1H-imidazol-1-yl)benzyl)-N-(3-methoxybenzyl)-4-((2-(3-methoxybenzyloxy)ethoxy)methyl)aniline; 3-(2-(4-((4-(1H-imidazol-1-yl)benzyl)(3-methoxybenzyl)amino)benzyloxy)ethoxy)-N,N-dimethylaniline; N-(4-(1H-imidazol-1-yl)benzyl)-N-(3-methoxybenzyl)-4-((2-morpholinoethoxy)methyl)aniline; N-(4-(1H-imidazol-1-yl)benzyl)-N-(3-methoxybenzyl)-4-(morpholinomethyl)aniline; N-(4-(1H-imidazol-1-yl)benzyl)-N-(3-methoxybenzyl)-4-(piperidin-1-ylmethyl)aniline; N-(4-(1H-imidazol-1-yl)benzyl)-N-(3-methoxybenzyl)-4-((4-methylpiperazin-1-yl)methyl)aniline; 1-(4-((4-(1H-imidazol-1-yl)benzyl)(3-methoxybenzyl)amino)benzyl)piperazin-2-one; 1-(4-((4-(1H-imidazol-1-yl)benzyl)(3-methoxybenzyl)amino)benzyl)piperazine-2,5-dione; 4-(4-((4-(1H-imidazol-1-yl)benzyl)(3-methoxybenzyl)amino)benzyl)piperazin-2-one; N-(4-(1H-imidazol-1-yl)benzyl)-4-((dimethylamino)methyl)-N-(3-methoxybenzyl)aniline; N-((1H-benzo[d]imidazol-6-yl)methyl)-N-(3-methoxybenzyl)-4-((2-(2-morpholinoethoxy)ethoxy)methyl)aniline; N-((1H-benzo[d]imidazol-6-yl)methyl)-N-(3-methoxybenzyl)-4-((2-(3-methoxybenzyloxy)ethoxy)methyl)aniline; 3-(2-(4-(((1H-benzo[d]imidazol-6-yl)methyl)(3-methoxybenzyl)amino)benzyloxy)ethoxy)-N,N-dimethylaniline; N-((1H-benzo[d]imidazol-6-yl)methyl)-N-(3-methoxybenzyl)-4-((2-morpholinoethoxy)methyl)aniline; N-((1H-benzo[d]imidazol-6-yl)methyl)-N-(3-methoxybenzyl)-4-(morpholinomethyl)aniline; N-((1H-benzo[d]imidazol-6-yl)methyl)-N-(3-methoxybenzyl)-4-(piperidin-1-ylmethyl)aniline; N-((1H-benzo[d]imidazol-6-yl)methyl)-N-(3-methoxybenzyl)-4-((4-methylpiperazin-1-yl)methyl)aniline; 1-(4-(((1H-benzo[d]imidazol-6-yl)methyl)(3-methoxybenzyl)amino)benzyl)piperazin-2-one; 1-(4-(((1H-benzo[d]imidazol-6-yl)methyl)(3-methoxybenzyl)amino)benzyl)piperazine-2,5-dione; 4-(4-(((1H-benzo[d]imidazol-6-yl)methyl)(3-methoxybenzyl)amino)benzyl)piperazin-2-one; N-((1H-benzo[d]imidazol-6-yl)methyl)-4-((dimethylamino)methyl)-N-(3-methoxybenzyl)aniline; N-((1H-indazol-6-yl)methyl)-N-(3-methoxybenzyl)-4-((2-(2-morpholinoethoxy)ethoxy)methyl)aniline; N-((1H-indazol-6-yl)methyl)-N-(3-methoxybenzyl)-4-((2-(3-methoxybenzyloxy)ethoxy)methyl)aniline; 3-(2-(4-(((1H-indazol-6-yl)methyl)(3-methoxybenzyl)amino)benzyloxy)ethoxy)-N,N-dimethylaniline; N-((1H-indazol-6-yl)methyl)-N-(3-methoxybenzyl)-4-((2-morpholinoethoxy)methyl)aniline; N-((1H-indazol-6-yl)methyl)-N-(3-methoxybenzyl)-4-(morpholinomethyl)aniline; N-((1H-indazol-6-yl)methyl)-N-(3-methoxybenzyl)-4-(piperidin-1-ylmethyl)aniline; N-((1H-indazol-6-yl)methyl)-N-(3-methoxybenzyl)-4-((4-methylpiperazin-1-yl)methyl)aniline; 1-(4-(((1H-indazol-6-yl)methyl)(3-methoxybenzyl)amino)benzyl)piperazin-2-one; 1-(4-(((1H-indazol-6-yl)methyl)(3-methoxybenzyl)amino)benzyl)piperazine-2,5-dione; 4-(4-(((1H-indazol-6-yl)methyl)(3-methoxybenzyl)amino)benzyl)piperazin-2-one; N-((1H-indazol-6-yl)methyl)-4-((dimethylamino)methyl)-N-(3-methoxybenzyl)aniline; N-(3-methoxybenzyl)-4-((2-(2-morpholinoethoxy)ethoxy)methyl)-N-(quinolin-7-ylmethyl)aniline; N-(3-methoxybenzyl)-4-((2-(3-methoxybenzyloxy)ethoxy)methyl)-N-(quinolin-7-ylmethyl)aniline; 3-(2-(4-((3-methoxybenzyl)(quinolin-7-ylmethyl)amino)benzyloxy)ethoxy)-N,N-dimethylaniline; N-(3-methoxybenzyl)-4-((2-morpholinoethoxy)methyl)-N-(quinolin-7-ylmethyl)aniline; N-(3-methoxybenzyl)-4-(morpholinomethyl)-N-(quinolin-7-ylmethyl)aniline; N-(3-methoxybenzyl)-4-((4-methylpiperazin-1-yl)methyl)-N-(quinolin-7-ylmethyl)aniline; 1-(4-((3-methoxybenzyl)(quinolin-7-ylmethyl)amino)benzyl)piperazin-2-one; 1-(4-((3-methoxybenzyl)(quinolin-7-ylmethyl)amino)benzyl)piperazine-2,5-dione; 4-(4-((3-methoxybenzyl)(quinolin-7-ylmethyl)amino)benzyl)piperazin-2-one; 4-((dimethylamino)methyl)-N-(3-methoxybenzyl)-N-(quinolin-7-ylmethyl)aniline; N,N-bis(3-methoxybenzyl)-3-((2-(2-(3-methoxybenzyloxy)ethoxy)ethoxy)methyl)aniline; 3-((2-(2-(benzyloxy)ethoxy)ethoxy)methyl)-N,N-bis(3-methoxybenzyl)aniline; N,N-bis(3-methoxybenzyl)-3-((2-(2-(3-methoxyphenoxy)ethoxy)ethoxy)methyl)aniline; N,N-bis(3-methoxybenzyl)-3-((2-(2-morpholinoethoxy)ethoxy)methyl)aniline; N,N-bis(3-methoxybenzyl)-3-((2-(3-methoxybenzyloxy)ethoxy)methyl)aniline; 3-(2-(3-(bis(3-methoxybenzyl)amino)benzyloxy)ethoxy)-N,N-dimethylaniline; N,N-bis(3-methoxybenzyl)-3-((2-morpholinoethoxy)methyl)aniline; N,N-bis(3-methoxybenzyl)-3-(morpholinomethyl)aniline; N,N-bis(3-methoxybenzyl)-3-(piperidin-1-ylmethyl)aniline; N,N-bis(3-methoxybenzyl)-3-((4-methylpiperazin-1-yl)methyl)aniline; 1-(3-(bis(3-methoxybenzyl)amino)benzyl)piperazin-2-one; 1-(3-(bis(3-methoxybenzyl)amino)benzyl)piperazine-2,5-dione; 4-(3-(bis(3-methoxybenzyl)amino)benzyl)piperazin-2-one; 3-((dimethylamino)methyl)-N,N-bis(3-methoxybenzyl)aniline; N-(3-(dimethylamino)benzyl)-N-(3-methoxybenzyl)-3-((2-(2-(3-methoxybenzyloxy)ethoxy)ethoxy)methyl)aniline; 3-((2-(2-(benzyloxy)ethoxy)ethoxy)methyl)-N-(3-(dimethylamino)benzyl)-N-(3-methoxybenzyl)aniline; N-(3-(dimethylamino)benzyl)-N-(3-methoxybenzyl)-3-((2-(2-(3-methoxyphenoxy)ethoxy)ethoxy)methyl)aniline; N-(3-(dimethylamino)benzyl)-N-(3-methoxybenzyl)-3-((2-(2-morpholinoethoxy)ethoxy)methyl)aniline; N-(3-(dimethylamino)benzyl)-N-(3-methoxybenzyl)-3-((2-(3-methoxybenzyloxy)ethoxy)methyl)aniline; N-(3-(dimethylamino)benzyl)-3-((2-(3-(dimethylamino)phenoxy)ethoxy)methyl)-N-(3-methoxybenzyl)aniline; N-(3-(dimethylamino)benzyl)-N-(3-methoxybenzyl)-3-((2-morpholinoethoxy)methyl)aniline; N-(3-(dimethylamino)benzyl)-N-(3-methoxybenzyl)-3-(morpholinomethyl)aniline; N-(3-(dimethylamino)benzyl)-N-(3-methoxybenzyl)-3-(piperidin-1-ylmethyl)aniline; N-(3-(dimethylamino)benzyl)-N-(3-methoxybenzyl)-3-((4-methylpiperazin-1-yl)methyl)aniline; 1-(3-((3-(dimethylamino)benzyl)(3-methoxybenzyl)amino)benzyl)piperazin-2-one; 1-(3-((3-(dimethylamino)benzyl)(3-methoxybenzyl)amino)benzyl)piperazine-2,5-dione; 4-(3-((3-(dimethylamino)benzyl)(3-methoxybenzyl)amino)benzyl)piperazin-2-one; N-(3-(dimethylamino)benzyl)-3-((dimethylamino)methyl)-N-(3-methoxybenzyl)aniline; N-(3-methoxybenzyl)-3-((2-(2-morpholinoethoxy)ethoxy)methyl)-N-(3-(pyrrolidin-1-yl)benzyl)aniline; N-(3-methoxybenzyl)-3-((2-(3-methoxybenzyloxy)ethoxy)methyl)-N-(3-(pyrrolidin-1-yl)benzyl)aniline; 3-((2-(3-(dimethylamino)phenoxy)ethoxy)methyl)-N-(3-methoxybenzyl)-N-(3-(pyrrolidin-1-yl)benzyl)aniline; N-(3-methoxybenzyl)-3-((2-morpholinoethoxy)methyl)-N-(3-(pyrrolidin-1-yl)benzyl)aniline; N-(3-methoxybenzyl)-3-(morpholinomethyl)-N-(3-(pyrrolidin-1-yl)benzyl)aniline; N-(3-methoxybenzyl)-3-(piperidin-1-ylmethyl)-N-(3-(pyrrolidin-1-yl)benzyl)aniline; N-(3-methoxybenzyl)-3-((4-methylpiperazin-1-yl)methyl)-N-(3-(pyrrolidin-1-yl)benzyl)aniline 1-(3-((3-methoxybenzyl)(3-(pyrrolidin-1-yl)benzyl)amino)benzyl)piperazin-2-one; 1-(3-((3-methoxybenzyl)(3-(pyrrolidin-1-yl)benzyl)amino)benzyl)piperazine-2,5-dione; 4-(3-((3-methoxybenzyl)(3-(pyrrolidin-1-yl)benzyl)amino)benzyl)piperazin-2-one; 3-((dimethylamino)methyl)-N-(3-methoxybenzyl)-N-(3-(pyrrolidin-1-yl)benzyl)aniline; N-(3-(1H-pyrazol-1-yl)benzyl)-N-(3-methoxybenzyl)-3-((2-(2-morpholinoethoxy)ethoxy)methyl)aniline; N-(3-(1H-pyrazol-1-yl)benzyl)-N-(3-methoxybenzyl)-3-((2-(3 methoxybenzyloxy)ethoxy)methyl)aniline; N-(3-(1H-pyrazol-1-yl)benzyl)-3-((2-(3-(dimethylamino)phenoxy)ethoxy)methyl)-N-(3-methoxybenzyl)aniline; N-(3-(1H-pyrazol-1-yl)benzyl)-N-(3-methoxybenzyl)-3-((2-morpholinoethoxy)methyl)aniline; N-(3-(1H-pyrazol-1-yl)benzyl)-N-(3-methoxybenzyl)-3-(morpholinomethyl)aniline; N-(3-(1H-pyrazol-1-yl)benzyl)-N-(3-methoxybenzyl)-3-(piperidin-1-ylmethyl)aniline; N-(3-(1H-pyrazol-1-yl)benzyl)-N-(3-methoxybenzyl)-3-((4-methylpiperazin-1-yl)methyl)aniline; 1-(3-((3-(1H-pyrazol-1-yl)benzyl)(3-methoxybenzyl)amino)benzyl)piperazin-2-one; 1-(3-((3-(1H-pyrazol-1-yl)benzyl)(3-methoxybenzyl)amino)benzyl)piperazine-2,5-dione; 4-(3-((3-(1H-pyrazol-1-yl)benzyl)(3-methoxybenzyl)amino)benzyl)piperazin-2-one; N-(3-(1H-pyrazol-1-yl)benzyl)-3-((dimethylamino)methyl)-N-(3-methoxybenzyl)aniline; N-(3-(1H-imidazol-1-yl)benzyl)-N-(3-methoxybenzyl)-3-((2-(2-morpholinoethoxy)ethoxy)methyl)aniline; N-(3-(1H-imidazol-1-yl)benzyl)-N-(3-methoxybenzyl)-3-((2-(3-methoxybenzyloxy)ethoxy)methyl)aniline; N-(3-(1H-imidazol-1-yl)benzyl)-3-((2-(3-(dimethylamino)phenoxy)ethoxy)methyl)-N-(3-methoxybenzyl)aniline; N-(3-(1H-imidazol-1-yl)benzyl)-N-(3-methoxybenzyl)-3-((2-morpholinoethoxy)methyl)aniline; N-(3-(1H-imidazol-1-yl)benzyl)-N-(3-methoxybenzyl)-3-(morpholinomethyl)aniline; N-(3-(1H-imidazol-1-yl)benzyl)-N-(3-methoxybenzyl)-3-(piperidin-1-ylmethyl)aniline; N-(3-(1H-imidazol-1-yl)benzyl)-N-(3-methoxybenzyl)-3-((4-methylpiperazin-1-yl)methyl)aniline; 1-(3-((3-(1H-imidazol-1-yl)benzyl)(3-methoxybenzyl)amino)benzyl)piperazin-2-one; 1-(3-((3-(1H-imidazol-1-yl)benzyl)(3-methoxybenzyl)amino)benzyl)piperazine-2,5-dione; 4-(3-((3-(1H-imidazol-1-yl)benzyl)(3-methoxybenzyl)amino)benzyl)piperazin-2-one; N-(3-(1H-imidazol-1-yl)benzyl)-3-((dimethylamino)methyl)-N-(3-methoxybenzyl)aniline; 3-((2-(3-(dimethylamino)phenoxy)ethoxy)methyl)-N-(3-methoxybenzyl)-N-(4-(pyrrolidin-1-yl)benzyl)aniline; N-(3-methoxybenzyl)-3-((2-(2-morpholinoethoxy)ethoxy)methyl)-N-(4-(pyrrolidin-1-yl)benzyl)aniline; N-(3-methoxybenzyl)-3-((2-(3-methoxybenzyloxy)ethoxy)methyl)-N-(4-(pyrrolidin-1-yl)benzyl)aniline; N-(3-methoxybenzyl)-3-((2-morpholinoethoxy)methyl)-N-(4-(pyrrolidin-1-yl)benzyl)aniline; N-(3-methoxybenzyl)-3-(morpholinomethyl)-N-(4-(pyrrolidin-1-yl)benzyl)aniline; N-(3-methoxybenzyl)-3-(piperidin-1-ylmethyl)-N-(4-(pyrrolidin-1-yl)benzyl)aniline; N-(3-methoxybenzyl)-3-((4-methylpiperazin-1-yl)methyl)-N-(4-(pyrrolidin-1-yl)benzyl)aniline; 1-(3-((3-methoxybenzyl)(4-(pyrrolidin-1-yl)benzyl)amino)benzyl)piperazin-2-one; 1-(3-((3-methoxybenzyl)(4-(pyrrolidin-1-yl)benzyl)amino)benzyl)piperazine-2,5-dione; 4-(3-((3-methoxybenzyl)(4-(pyrrolidin-1-yl)benzyl)amino)benzyl)piperazin-2-one; 3-((dimethylamino)methyl)-N-(3-methoxybenzyl)-N-(4-(pyrrolidin-1-yl)benzyl)aniline; N-(3-methoxybenzyl)-N-(4-morpholinobenzyl)-3-((2-(2-morpholinoethoxy)ethoxy)methyl)aniline; N-(3-methoxybenzyl)-3-((2-(3-methoxybenzyloxy)ethoxy)methyl)-N-(4-morpholinobenzyl)aniline; 3-((2-(3-(dimethylamino)phenoxy)ethoxy)methyl)-N-(3-methoxybenzyl)-N-(4-morpholinobenzyl)aniline; N-(3-methoxybenzyl)-N-(4-morpholinobenzyl)-3-((2-morpholinoethoxy)methyl)aniline; N-(3-methoxybenzyl)-N-(4-morpholinobenzyl)-3-(morpholinomethyl)aniline; N-(3-methoxybenzyl)-N-(4-morpholinobenzyl)-3-(piperidin-1-ylmethyl)aniline; N-(3-methoxybenzyl)-3-((4-methylpiperazin-1-yl)methyl)-N-(4-morpholinobenzyl)aniline; 1-(3-((3-methoxybenzyl)(4-morpholinobenzyl)amino)benzyl)piperazin-2-one; 1-(3-((3-methoxybenzyl)(4-morpholinobenzyl)amino)benzyl)piperazine-2,5-dione; 4-(3-((3-methoxybenzyl)(4-morpholinobenzyl)amino)benzyl)piperazin-2-one; 3-((dimethylamino)methyl)-N-(3-methoxybenzyl)-N-(4-morpholinobenzyl)aniline; N-(3-methoxybenzyl)-N-(4-(4-methylpiperazin-1-yl)benzyl)-3-((2-(2-morpholinoethoxy)ethoxy)methyl)aniline; N-(3-methoxybenzyl)-3-((2-(3-methoxybenzyloxy)ethoxy)methyl)-N-(4-(4-methylpiperazin-1-yl)benzyl)aniline; 3-((2-(3-(dimethylamino)phenoxy)ethoxy)methyl)-N-(3-methoxybenzyl)-N-(4-(4-methylpiperazin-1-yl)benzyl)aniline; N-(3-methoxybenzyl)-N-(4-(4-methylpiperazin-1-yl)benzyl)-3-((2-morpholinoethoxy)methyl)aniline; N-(3-methoxybenzyl)-N-(4-(4-methylpiperazin-1-yl)benzyl)-3-(morpholinomethyl)aniline; N-(3-methoxybenzyl)-N-(4-(4-methylpiperazin-1-yl)benzyl)-3-(piperidin-1-ylmethyl)aniline; N-(3-methoxybenzyl)-N-(4-(4-methylpiperazin-1-yl)benzyl)-3-((4-methylpiperazin-1-yl)methyl)aniline; 1-(3-((3-methoxybenzyl)(4-(4-methylpiperazin-1-yl)benzyl)amino)benzyl)piperazin-2-one; 1-(3-((3-methoxybenzyl)(4-(4-methylpiperazin-1-yl)benzyl)amino)benzyl)piperazine-2,5-dione; 3-((dimethylamino)methyl)-N-(3-methoxybenzyl)-N-(4-(4-methylpiperazin-1-yl)benzyl)aniline; N-(3-methoxybenzyl)-N-(3-morpholinobenzyl)-3-((2-(2-morpholinoethoxy)ethoxy)methyl)aniline; N-(3-methoxybenzyl)-3-((2-(3-methoxybenzyloxy)ethoxy)methyl)-N-(3-morpholinobenzyl)aniline; 3-((2-(3-(dimethylamino)phenoxy)ethoxy)methyl)-N-(3-methoxybenzyl)-N-(3-morpholinobenzyl)aniline; N-(3-methoxybenzyl)-N-(3-morpholinobenzyl)-3-((2-morpholinoethoxy)methyl)aniline; N-(3-methoxybenzyl)-N-(3-morpholinobenzyl)-3-(morpholinomethyl)aniline; N-(3-methoxybenzyl)-N-(3-morpholinobenzyl)-3-(piperidin-1-ylmethyl)aniline; N-(3-methoxybenzyl)-3-((4-methylpiperazin-1-yl)methyl)-N-(3-morpholinobenzyl)aniline; 1-(3-((3-methoxybenzyl)(3-morpholinobenzyl)amino)benzyl)piperazin-2-one; 1-(3-((3-methoxybenzyl)(3-morpholinobenzyl)amino)benzyl)piperazine-2,5-dione; 4-(3-((3-methoxybenzyl)(3-morpholinobenzyl)amino)benzyl)piperazin-2-one; 3-((dimethylamino)methyl)-N-(3-methoxybenzyl)-N-(3-morpholinobenzyl)aniline; N-(3-methoxybenzyl)-N-(3-(4-methylpiperazin-1-yl)benzyl)-3-((2-(2-morpholinoethoxy)ethoxy)methyl)aniline; N-(3-methoxybenzyl)-3-((2-(3-methoxybenzyloxy)ethoxy)methyl)-N-(3-(4-methylpiperazin-1-yl)benzyl)aniline; 3-((2-(3-(dimethylamino)phenoxy)ethoxy)methyl)-N-(3-methoxybenzyl)-N-(3-(4-methylpiperazin-1-yl)benzyl)aniline; N-(3-methoxybenzyl)-N-(3-(4-methylpiperazin-1-yl)benzyl)-3-((2-morpholinoethoxy)methyl)aniline; N-(3-methoxybenzyl)-N-(3-(4-methylpiperazin-1-yl)benzyl)-3-(morpholinomethyl)aniline; N-(3-methoxybenzyl)-N-(3-(4-methylpiperazin-1-yl)benzyl)-3-(piperidin-1-ylmethyl)aniline; N-(3-methoxybenzyl)-N-(3-(4-methylpiperazin-1-yl)benzyl)-3-((4-methylpiperazin-1-yl)methyl)aniline; 1-(3-((3-methoxybenzyl)(3-(4-methylpiperazin-1-yl)benzyl)amino)benzyl)piperazin-2-one; 1-(3-((3-methoxybenzyl)(3-(4-methylpiperazin-1-yl)benzyl)amino)benzyl)piperazine-2,5-dione; 4-(3-((3-methoxybenzyl)(3-(4-methylpiperazin-1-yl)benzyl)amino)benzyl)piperazin-2-one; 3-((dimethylamino)methyl)-N-(3-methoxybenzyl)-N-(3-(4-methylpiperazin-1-yl)benzyl)aniline; N-(4-(1H-pyrazol-1-yl)benzyl)-N-(3-methoxybenzyl)-3-((2-(2-morpholinoethoxy)ethoxy)methyl)aniline; N-(4-(1H-pyrazol-1-yl)benzyl)-N-(3-methoxybenzyl)-3-((2-(3-methoxybenzyloxy)ethoxy)methyl)aniline; N-(4-(1H-pyrazol-1-yl)benzyl)-3-((2-(3-(dimethylamino)phenoxy)ethoxy)methyl)-N-(3-methoxybenzyl)aniline; N-(4-(1H-pyrazol-1-yl)benzyl)-N-(3-methoxybenzyl)-3-((2-morpholinoethoxy)methyl)aniline; N-(4-(1H-pyrazol-1-yl)benzyl)-N-(3-methoxybenzyl)-3-(morpholinomethyl)aniline; N-(4-(1H-pyrazol-1-yl)benzyl)-N-(3-methoxybenzyl)-3-(piperidin-1-ylmethyl)aniline N-(4-(1H-pyrazol-1-yl)benzyl)-N-(3-methoxybenzyl)-3-((4-methylpiperazin-1-yl)methyl)aniline; 1-(3-((4-(1H-pyrazol-1-yl)benzyl)(3-methoxybenzyl)amino)benzyl)piperazin-2-one; 1-(3-((4-(1H-pyrazol-1-yl)benzyl)(3-methoxybenzyl)amino)benzyl)piperazine-2,5-dione; 4-(3-((4-(1H-pyrazol-1-yl)benzyl)(3-methoxybenzyl)amino)benzyl)piperazin-2-one; N-(4-(1H-pyrazol-1-yl)benzyl)-3-((dimethylamino)methyl)-N-(3-methoxybenzyl)aniline; N-(4-(1H-imidazol-1-yl)benzyl)-N-(3-methoxybenzyl)-3-((2-(2-morpholinoethoxy)ethoxy)methyl)aniline; N-(4-(1H-imidazol-1-yl)benzyl)-N-(3-methoxybenzyl)-3-((2-(3-methoxybenzyloxy)ethoxy)methyl)aniline; N-(4-(1H-imidazol-1-yl)benzyl)-3-((2-(3-(dimethylamino)phenoxy)ethoxy)methyl)-N-(3-methoxybenzyl)aniline; N-(4-(1H-imidazol-1-yl)benzyl)-N-(3-methoxybenzyl)-3-((2-morpholinoethoxy)methyl)aniline; N-(4-(1H-imidazol-1-yl)benzyl)-N-(3-methoxybenzyl)-3-(morpholinomethyl)aniline; N-(4-(1H-imidazol-1-yl)benzyl)-N-(3-methoxybenzyl)-3-(piperidin-1-ylmethyl)aniline; N-(4-(1H-imidazol-1-yl)benzyl)-N-(3-methoxybenzyl)-3-((4-methylpiperazin-1-yl)methyl)aniline; 1-(3-((4-(1H-imidazol-1-yl)benzyl)(3-methoxybenzyl)amino)benzyl)piperazin-2-one; 1-(3-((4-(1H-imidazol-1-yl)benzyl)(3-methoxybenzyl)amino)benzyl)piperazine-2,5-dione; 4-(3-((4-(1H-imidazol-1-yl)benzyl)(3-methoxybenzyl)amino)benzyl)piperazin-2-one; N-(4-(1H-imidazol-1-yl)benzyl)-3-((dimethylamino)methyl)-N-(3-methoxybenzyl)aniline; N-((1H-benzo[d]imidazol-6-yl)methyl)-N-(3-methoxybenzyl)-3-((2-(2-morpholinoethoxy)ethoxy)methyl)aniline; N-((1H-benzo[d]imidazol-6-yl)methyl)-N-(3-methoxybenzyl)-3-((2-(3-methoxybenzyloxy)ethoxy)methyl)aniline; N-((1H-benzo[d]imidazol-6-yl)methyl)-3-((2-(3-(dimethylamino)phenoxy)ethoxy)methyl)-N-(3-methoxybenzyl)aniline; N-((1H-benzo[d]imidazol-6-yl)methyl)-N-(3-methoxybenzyl)-3-((2-morpholinoethoxy)methyl)aniline; N-((1H-benzo[d]imidazol-6-yl)methyl)-N-(3-methoxybenzyl)-3-(morpholinomethyl)aniline; N-((1H-benzo[d]imidazol-6-yl)methyl)-N-(3-methoxybenzyl)-3-(piperidin-1-ylmethyl)aniline; N-((1H-benzo[d]imidazol-6-yl)methyl)-N-(3-methoxybenzyl)-3-((4-methylpiperazin-1-yl)methyl)aniline; 1-(3-(((1H-benzo[d]imidazol-6-yl)methyl)(3-methoxybenzyl)amino)benzyl)piperazin-2-one; 1-(3-(((1H-benzo[d]imidazol-6-yl)methyl)(3-methoxybenzyl)amino)benzyl)piperazine-2,5-dione; 4-(3-(((1H-benzo[d]imidazol-6-yl)methyl)(3-methoxybenzyl)amino)benzyl)piperazin-2-one; N-((1H-benzo[d]imidazol-6-yl)methyl)-3-((dimethylamino)methyl)-N-(3-methoxybenzyl)aniline; N-((1H-indazol-6-yl)methyl)-N-(3-methoxybenzyl)-3-((2-(2-morpholinoethoxy)ethoxy)methyl)aniline; N-((1H-indazol-6-yl)methyl)-N-(3-methoxybenzyl)-3-((2-(3-methoxybenzyloxy)ethoxy)methyl)aniline; N-((1H-indazol-6-yl)methyl)-3-((2-(3-(dimethylamino)phenoxy)ethoxy)methyl)-N-(3-methoxybenzyl)aniline; N-((1H-indazol-6-yl)methyl)-N-(3-methoxybenzyl)-3-((2-morpholinoethoxy)methyl)aniline; N-((1H-indazol-6-yl)methyl)-N-(3-methoxybenzyl)-3-(morpholinomethyl)aniline; N-((1H-indazol-6-yl)methyl)-N-(3-methoxybenzyl)-3-(piperidin-1-ylmethyl)aniline; N-((1H-indazol-6-yl)methyl)-N-(3-methoxybenzyl)-3-((4-methylpiperazin-1-yl)methyl)aniline; 1-(3-(((1H-indazol-6-yl)methyl)(3-methoxybenzyl)amino)benzyl)piperazin-2-one; 1-(3-(((1H-indazol-6-yl)methyl)(3-methoxybenzyl)amino)benzyl)piperazine-2,5-dione; 4-(3-(((1H-indazol-6-yl)methyl)(3-methoxybenzyl)amino)benzyl)piperazin-2-one; N-((1H-indazol-6-yl)methyl)-3-((dimethylamino)methyl)-N-(3-methoxybenzyl)aniline; N-(3-methoxybenzyl)-3-((2-(2-morpholinoethoxy)ethoxy)methyl)-N-(quinolin-7-ylmethyl)aniline; N-(3-methoxybenzyl)-3-((2-(3-methoxybenzyloxy)ethoxy)methyl)-N-(quinolin-7-ylmethyl)aniline; 3-((2-(3-(dimethylamino)phenoxy)ethoxy)methyl)-N-(3-methoxybenzyl)-N-(quinolin-7-ylmethyl)aniline; N-(3-methoxybenzyl)-3-((2-morpholinoethoxy)methyl)-N-(quinolin-7-ylmethyl)aniline N-(3-methoxybenzyl)-3-(morpholinomethyl)-N-(quinolin-7-ylmethyl)aniline; N-(3-methoxybenzyl)-3-(piperidin-1-ylmethyl)-N-(quinolin-7-ylmethyl)aniline; N-(3-methoxybenzyl)-3-((4-methylpiperazin-1-yl)methyl)-N-(quinolin-7-ylmethyl)aniline; 1-(3-((3-methoxybenzyl)(quinolin-7-ylmethyl)amino)benzyl)piperazin-2-one; 1-(3-((3-methoxybenzyl)(quinolin-7-ylmethyl)amino)benzyl)piperazine-2,5-dione; 4-(3-((3-methoxybenzyl)(quinolin-7-ylmethyl)amino)benzyl)piperazin-2-one; 3-((dimethylamino)methyl)-N-(3-methoxybenzyl)-N-(quinolin-7-ylmethyl)aniline; N,N-bis(3-methoxybenzyl)-2-((2-(2-(3-methoxybenzyloxy)ethoxy)ethoxy)methyl)pyridin-4-amine; 2-((2-(2-(benzyloxy)ethoxy)ethoxy)methyl)-N,N-bis(3-methoxybenzyl)pyridin-4-amine; N,N-bis(3-methoxybenzyl)-2-((2-(2-(3-methoxyphenoxy)ethoxy)ethoxy)methyl)pyridin-4-amine; N,N-bis(3-methoxybenzyl)-2-((2-(2-morpholinoethoxy)ethoxy)methyl)pyridin-4-amine; N,N-bis(3-methoxybenzyl)-2-((2-(3-methoxybenzyloxy)ethoxy)methyl)pyridin-4-amine; 2-((2-(3-(dimethylamino)phenoxy)ethoxy)methyl)-N,N-bis(3-methoxybenzyl)pyridin-4-amine; N,N-bis(3-methoxybenzyl)-2-((2-morpholinoethoxy)methyl)pyridin-4-amine; N,N-bis(3-methoxybenzyl)-2-(morpholinomethyl)pyridin-4-amine; N,N-bis(3-methoxybenzyl)-2-(piperidin-1-ylmethyl)pyridin-4-amine; N,N-bis(3-methoxybenzyl)-2-((4-methylpiperazin-1-yl)methyl)pyridin-4-amine; 1-((4-(bis(3-methoxybenzyl)amino)pyridin-2-yl)methyl)piperazin-2-one; 1-((4-(bis(3-methoxybenzyl)amino)pyridin-2-yl)methyl)piperazine-2,5-dione; 4-((4-(bis(3-methoxybenzyl)amino)pyridin-2-yl)methyl)piperazin-2-one; 2-((dimethylamino)methyl)-N,N-bis(3-methoxybenzyl)pyridin-4-amine; N-(3-(dimethylamino)benzyl)-N-(3-methoxybenzyl)-2-((2-(2-(3-methoxybenzyloxy)ethoxy)ethoxy)methyl)pyridin-4-amine; 2-((2-(2-(benzyloxy)ethoxy)ethoxy)methyl)-N-(3-(dimethylamino)benzyl)-N-(3-methoxybenzyl)pyridin-4-amine; N-(3-(dimethylamino)benzyl)-N-(3-methoxybenzyl)-2-((2-(2-(3-methoxyphenoxy)ethoxy)ethoxy)methyl)pyridin-4-amine; N-(3-(dimethylamino)benzyl)-N-(3-methoxybenzyl)-2-((2-(2-morpholinoethoxy)ethoxy)methyl)pyridin-4-amine; N-(3-(dimethylamino)benzyl)-N-(3-methoxybenzyl)-2-((2-(3-methoxybenzyloxy)ethoxy)methyl)pyridin-4-amine; N-(3-(dimethylamino)benzyl)-2-((2-(3-(dimethylamino)phenoxy)ethoxy)methyl)-N-(3-methoxybenzyl)pyridin-4-amine; N-(3-(dimethylamino)benzyl)-N-(3-methoxybenzyl)-2-((2-morpholinoethoxy)methyl)pyridin-4-amine; N-(3-(dimethylamino)benzyl)-N-(3-methoxybenzyl)-3-(morpholinomethyl)aniline; N-(3-(dimethylamino)benzyl)-N-(3-methoxybenzyl)-2-(piperidin-1-ylmethyl)pyridin-4-amine; N-(3-(dimethylamino)benzyl)-N-(3-methoxybenzyl)-2-((4-methylpiperazin-1-yl)methyl)pyridin-4-amine; 1-((4-((3-(dimethylamino)benzyl)(3-methoxybenzyl)amino)pyridin-2-yl)methyl)piperazin-2-one; 1-((4-((3-(dimethylamino)benzyl)(3-methoxybenzyl)amino)pyridin-2-yl)methyl)piperazine-2,5-dione; 4-((4-((3-(dimethylamino)benzyl)(3-methoxybenzyl)amino)pyridin-2-yl)methyl)piperazin-2-one; N-(3-(dimethylamino)benzyl)-2-((dimethylamino)methyl)-N-(3-methoxybenzyl)pyridin-4-amine; N-(3-methoxybenzyl)-2-((2-(2-morpholinoethoxy)ethoxy)methyl)-N-(3-(pyrrolidin-1-yl)benzyl)pyridin-4-amine; N-(3-methoxybenzyl)-2-((2-(3-methoxybenzyloxy)ethoxy)methyl)-N-(3-(pyrrolidin-1-yl)benzyl)pyridin-4-amine; 2-((2-(3-(dimethylamino)phenoxy)ethoxy)methyl)-N-(3-methoxybenzyl)-N-(3-(pyrrolidin-1-yl)benzyl)pyridin-4-amine; N-(3-methoxybenzyl)-2-((2-morpholinoethoxy)methyl)-N-(3-(pyrrolidin-1-yl)benzyl)pyridin-4-amine; N-(3-methoxybenzyl)-2-(morpholinomethyl)-N-(3-(pyrrolidin-1-yl)benzyl)pyridin-4-amine; N-(3-methoxybenzyl)-2-(piperidin-1-ylmethyl)-N-(3-(pyrrolidin-1-yl)benzyl)pyridin-4-amine; N-(3-methoxybenzyl)-2-((4-methylpiperazin-1-yl)methyl)-N-(3-(pyrrolidin-1-yl)benzyl)pyridin-4-amine; 1-((4-((3-methoxybenzyl)(3-(pyrrolidin-1-yl)benzyl)amino)pyridin-2-yl)methyl)piperazin-2-one; 1-((4-((3-methoxybenzyl)(3-(pyrrolidin-1-yl)benzyl)amino)pyridin-2-yl)methyl)piperazine-2,5-dione; 4-((4-((3-methoxybenzyl)(3-(pyrrolidin-1-yl)benzyl)amino)pyridin-2-yl)methyl)piperazin-2-one; 2-((dimethylamino)methyl)-N-(3-methoxybenzyl)-N-(3-(pyrrolidin-1-yl)benzyl)pyridin-4-amine; N-(3-(1H-pyrazol-1-yl)benzyl)-N-(3-methoxybenzyl)-2-((2-(2-morpholinoethoxy)ethoxy)methyl)pyridin-4-amine; N-(3-(1H-pyrazol-1-yl)benzyl)-N-(3-methoxybenzyl)-2-((2-(3-methoxybenzyloxy)ethoxy)methyl)pyridin-4-amine; N-(3-(1H-pyrazol-1-yl)benzyl)-2-((2-(3-(dimethylamino)phenoxy)ethoxy)methyl)-N-(3-methoxybenzyl)pyridin-4-amine; N-(3-(1H-pyrazol-1-yl)benzyl)-N-(3-methoxybenzyl)-2-((2-morpholinoethoxy)methyl)pyridin-4-amine; N-(3-(1H-pyrazol-1-yl)benzyl)-N-(3-methoxybenzyl)-2-(morpholinomethyl)pyridin-4-amine; N-(3-(1H-pyrazol-1-yl)benzyl)-N-(3-methoxybenzyl)-2-(piperidin-1-ylmethyl)pyridin-4-amine; N-(3-(1H-pyrazol-1-yl)benzyl)-N-(3-methoxybenzyl)-2-((4-methylpiperazin-1-yl)methyl)pyridin-4-amine; 1-((4-((3-(1H-pyrazol-1-yl)benzyl)(3-methoxybenzyl)amino)pyridin-2-yl)methyl)piperazin-2-one; 1-((4-((3-(1H-pyrazol-1-yl)benzyl)(3-methoxybenzyl)amino)pyridin-2-yl)methyl)piperazine-2,5-dione; 4-((4-((3-(1H-pyrazol-1-yl)benzyl)(3-methoxybenzyl)amino)pyridin-2-yl)methyl)piperazin-2-one; N-(3-(1H-pyrazol-1-yl)benzyl)-2-((dimethylamino)methyl)-N-(3-methoxybenzyl)pyridin-4-amine; N-(3-(1H-imidazol-1-yl)benzyl)-N-(3-methoxybenzyl)-2-((2-(2-morpholinoethoxy)ethoxy)methyl)pyridin-4-amine; N-(3-(1H-imidazol-1-yl)benzyl)-N-(3-methoxybenzyl)-2-((2-(3 methoxybenzyloxy)ethoxy)methyl)pyridin-4-amine; N-(3-(1H-imidazol-1-yl)benzyl)-2-((2-(3-(dimethylamino)phenoxy)ethoxy)methyl)-N-(3-methoxybenzyl)pyridin-4-amine; N-(3-(1H-imidazol-1-yl)benzyl)-N-(3-methoxybenzyl)-2-((2-morpholinoethoxy)methyl)pyridin-4-amine; N-(3-(1H-imidazol-1-yl)benzyl)-N-(3-methoxybenzyl)-2-(morpholinomethyl)pyridin-4-amine; N-(3-(1H-imidazol-1-yl)benzyl)-N-(3-methoxybenzyl)-2-(piperidin-1-ylmethyl)pyridin-4-amine; N-(3-(1H-imidazol-1-yl)benzyl)-N-(3-methoxybenzyl)-2-((4-methylpiperazin-1-yl)methyl)pyridin-4-amine; 1-((4-((3-(1H-imidazol-1-yl)benzyl)(3-methoxybenzyl)amino)pyridin-2-yl)methyl)piperazin-2-one; 1-((4-((3-(1H-imidazol-1-yl)benzyl)(3-methoxybenzyl)amino)pyridin-2-yl)methyl)piperazine-2,5-dione; 4-((4-((3-(1H-imidazol-1-yl)benzyl)(3-methoxybenzyl)amino)pyridin-2-yl)methyl)piperazin-2-one; N-(3-(1H-imidazol-1-yl)benzyl)-2-((dimethylamino)methyl)-N-(3-methoxybenzyl)pyridin-4-amine; 2-((2-(3-(dimethylamino)phenoxy)ethoxy)methyl)-N-(3-methoxybenzyl)-N-(4-(pyrrolidin-1-yl)benzyl)pyridin-4-amine; N-(3-methoxybenzyl)-2-((2-(2-morpholinoethoxy)ethoxy)methyl)-N-(4-(pyrrolidin-1-yl)benzyl)pyridin-4-amine; N-(3-methoxybenzyl)-2-((2-(3-methoxybenzyloxy)ethoxy)methyl)-N-(4-(pyrrolidin-1-yl)benzyl)pyridin-4-amine; N-(3-methoxybenzyl)-2-((2-morpholinoethoxy)methyl)-N-(4-(pyrrolidin-1-yl)benzyl)pyridin-4-amine; N-(3-methoxybenzyl)-2-(morpholinomethyl)-N-(4-(pyrrolidin-1-yl)benzyl)pyridin-4-amine; N-(3-methoxybenzyl)-2-(piperidin-1-ylmethyl)-N-(4-(pyrrolidin-1-yl)benzyl)pyridin-4-amine; N-(3-methoxybenzyl)-2-((4-methylpiperazin-1-yl)methyl)-N-(4-(pyrrolidin-1-yl)benzyl)pyridin-4-amine; 1-((4-((3-methoxybenzyl)(4-(pyrrolidin-1-yl)benzyl)amino)pyridin-2-yl)methyl)piperazin-2-one; 1-((4-((3-methoxybenzyl)(4-(pyrrolidin-1-yl)benzyl)amino)pyridin-2-yl)methyl)piperazine-2,5-dione; 4-((4-((3-methoxybenzyl)(4-(pyrrolidin-1-yl)benzyl)amino)pyridin-2-yl)methyl)piperazin-2-one; 2-((dimethylamino)methyl)-N-(3-methoxybenzyl)-N-(4-(pyrrolidin-1-yl)benzyl)pyridin-4-amine; N-(3-methoxybenzyl)-N-(4-morpholinobenzyl)-2-((2-(2-morpholinoethoxy)ethoxy)methyl)pyridin-4-amine; N-(3-methoxybenzyl)-2-((2-(3-methoxybenzyloxy)ethoxy)methyl)-N-(4-morpholinobenzyl)pyridin-4-amine; 2-((2-(3-(dimethylamino)phenoxy)ethoxy)methyl)-N-(3-methoxybenzyl)-N-(4-morpholinobenzyl)pyridin-4-amine; N-(3-methoxybenzyl)-N-(4-morpholinobenzyl)-2-((2-morpholinoethoxy)methyl)pyridin-4-amine; N-(3-methoxybenzyl)-N-(4-morpholinobenzyl)-2-(morpholinomethyl)pyridin-4-amine; N-(3-methoxybenzyl)-N-(4-morpholinobenzyl)-2-(piperidin-1-ylmethyl)pyridin-4-amine; N-(3-methoxybenzyl)-2-((4-methylpiperazin-1-yl)methyl)-N-(4-morpholinobenzyl)pyridin-4-amine; 1-((4-((3-methoxybenzyl)(4-morpholinobenzyl)amino)pyridin-2-yl)methyl)piperazin-2-one; 1-((4-((3-methoxybenzyl)(4-morpholinobenzyl)amino)pyridin-2-yl)methyl)piperazine-2,5-dione; 4-((4-((3-methoxybenzyl)(4-morpholinobenzyl)amino)pyridin-2-yl)methyl)piperazin-2-one; 2-((dimethylamino)methyl)-N-(3-methoxybenzyl)-N-(4-morpholinobenzyl)pyridin-4-amine; N-(3-methoxybenzyl)-N-(4-(4-methylpiperazin-1-yl)benzyl)-2-((2-(2 morpholinoethoxy)ethoxy)methyl)pyridin-4-amine; N-(3-methoxybenzyl)-2-((2-(3-methoxybenzyloxy)ethoxy)methyl)-N-(4-(4-methylpiperazin-1-yl)benzyl)pyridin-4-amine; 2-((2-(3-(dimethylamino)phenoxy)ethoxy)methyl)-N-(3-methoxybenzyl)-N-(4-(4-methylpiperazin-1-yl)benzyl)pyridin-4-amine; N-(3-methoxybenzyl)-N-(4-(4-methylpiperazin-1-yl)benzyl)-2-((2-morpholinoethoxy)methyl)pyridin-4-amine; N-(3-methoxybenzyl)-N-(4-(4-methylpiperazin-1-yl)benzyl)-2-(morpholinomethyl)pyridin-4-amine; N-(3-methoxybenzyl)-N-(4-(4-methylpiperazin-1-yl)benzyl)-2-(piperidin-1-ylmethyl)pyridin-4-amine; N-(3-methoxybenzyl)-N-(4-(4-methylpiperazin-1-yl)benzyl)-2-((4-methylpiperazin-1-yl)methyl)pyridin-4-amine; 1-((4-((3-methoxybenzyl)(4-(4-methylpiperazin-1-yl)benzyl)amino)pyridin-2-yl)methyl)piperazin-2-one; 1-((4-((3-methoxybenzyl)(4-(4-methylpiperazin-1-yl)benzyl)amino)pyridin-2-yl)methyl)piperazine-2,5-dione; 4-((4-((3-methoxybenzyl)(4-(4-methylpiperazin-1-yl)benzyl)amino)pyridin-2-yl)methyl)piperazin-2-one; 2-((dimethylamino)methyl)-N-(3-methoxybenzyl)-N-(4-(4-methylpiperazin-1-yl)benzyl)pyridin-4-amine; N-(3-methoxybenzyl)-N-(3-morpholinobenzyl)-2-((2-(2-morpholinoethoxy)ethoxy)methyl)pyridin-4-amine; N-(3-methoxybenzyl)-2-((2-(3-methoxybenzyloxy)ethoxy)methyl)-N-(3-morpholinobenzyl)pyridin-4-amine; 2-((2-(3-(dimethylamino)phenoxy)ethoxy)methyl)-N-(3-methoxybenzyl)-N-(3-morpholinobenzyl)pyridin-4-amine; N-(3-methoxybenzyl)-N-(3-morpholinobenzyl)-2-((2-morpholinoethoxy)methyl)pyridin-4-amine; N-(3-methoxybenzyl)-N-(3-morpholinobenzyl)-2-(morpholinomethyl)pyridin-4-amine; N-(3-methoxybenzyl)-N-(3-morpholinobenzyl)-2-(piperidin-1-ylmethyl)pyridin-4-amine; N-(3-methoxybenzyl)-2-((4-methylpiperazin-1-yl)methyl)-N-(3-morpholinobenzyl)pyridin-4-amine; 1-((4-((3-methoxybenzyl)(3-morpholinobenzyl)amino)pyridin-2-yl)methyl)piperazin-2-one; 1-((4-((3-methoxybenzyl)(3-morpholinobenzyl)amino)pyridin-2-yl)methyl)piperazine-2,5-dione; 4-((4-((3-methoxybenzyl)(3-morpholinobenzyl)amino)pyridin-2-yl)methyl)piperazin-2-one; 2-((dimethylamino)methyl)-N-(3-methoxybenzyl)-N-(3-morpholinobenzyl)pyridin-4-amine; N-(3-methoxybenzyl)-N-(3-(4-methylpiperazin-1-yl)benzyl)-2-((2-(2-morpholinoethoxy)ethoxy)methyl)pyridin-4-amine; N-(3-methoxybenzyl)-2-((2-(3-methoxybenzyloxy)ethoxy)methyl)-N-(3-(4-methylpiperazin-1-yl)benzyl)pyridin-4-amine; 2-((2-(3-(dimethylamino)phenoxy)ethoxy)methyl)-N-(3-methoxybenzyl)-N-(3-(4-methylpiperazin-1-yl)benzyl)pyridin-4-amine; N-(3-methoxybenzyl)-N-(3-(4-methylpiperazin-1-yl)benzyl)-2-((2-morpholinoethoxy)methyl)pyridin-4-amine; N-(3-methoxybenzyl)-N-(3-(4-methylpiperazin-1-yl)benzyl)-2-(morpholinomethyl)pyridin-4-amine; N-(3-methoxybenzyl)-N-(3-(4-methylpiperazin-1-yl)benzyl)-2-(piperidin-1-ylmethyl)pyridin-4-amine; N-(3-methoxybenzyl)-N-(3-(4-methylpiperazin-1-yl)benzyl)-2-((4-methylpiperazin-1-yl)methyl)pyridin-4-amine; 1-((4-((3-methoxybenzyl)(3-(4-methylpiperazin-1-yl)benzyl)amino)pyridin-2-yl)methyl)piperazin-2-one; 1-((4-((3-methoxybenzyl)(3-(4-methylpiperazin-1-yl)benzyl)amino)pyridin-2-yl)methyl)piperazine-2,5-dione; 4-((4-((3-methoxybenzyl)(3-(4-methylpiperazin-1-yl)benzyl)amino)pyridin-2-yl)methyl)piperazin-2-one; 2-((dimethylamino)methyl)-N-(3-methoxybenzyl)-N-(3-(4-methylpiperazin-1-yl)benzyl)pyridin-4-amine; N-(4-(1H-pyrazol-1-yl)benzyl)-N-(3-methoxybenzyl)-2-((2-(2-morpholinoethoxy)ethoxy)methyl)pyridin-4-amine; N-(4-(1H-pyrazol-1-yl)benzyl)-N-(3-methoxybenzyl)-2-((2-(3-methoxybenzyloxy)ethoxy)methyl)pyridin-4-amine; N-(4-(1H-pyrazol-1-yl)benzyl)-2-((2-(3-(dimethylamino)phenoxy)ethoxy)methyl)-N-(3-methoxybenzyl)pyridin-4-amine; N-(4-(1H-pyrazol-1-yl)benzyl)-N-(3-methoxybenzyl)-2-((2-morpholinoethoxy)methyl)pyridin-4-amine; N-(4-(1H-pyrazol-1-yl)benzyl)-N-(3-methoxybenzyl)-2-(morpholinomethyl)pyridin-4-amine; N-(4-(1H-pyrazol-1-yl)benzyl)-N-(3-methoxybenzyl)-2-(piperidin-1-ylmethyl)pyridin-4-amine; 1-((4-((4-(1H-pyrazol-1-yl)benzyl)(3-methoxybenzyl)amino)pyridin-2-yl)methyl)piperazin-2-one; 1-((4-((4-(1H-pyrazol-1-yl)benzyl)(3-methoxybenzyl)amino)pyridin-2-yl)methyl)piperazine-2,5-dione; 4-((4-((4-(1H-pyrazol-1-yl)benzyl)(3-methoxybenzyl)amino)pyridin-2-yl)methyl)piperazin-2-one; N-(4-(1H-pyrazol-1-yl)benzyl)-2-((dimethylamino)methyl)-N-(3-methoxybenzyl)pyridin-4-amine; N-(4-(1H-imidazol-1-yl)benzyl)-N-(3-methoxybenzyl)-2-((2-(2-morpholinoethoxy)ethoxy)methyl)pyridin-4-amine; N-(4-(1H-imidazol-1-yl)benzyl)-N-(3-methoxybenzyl)-2-((2-(3-methoxybenzyloxy)ethoxy)methyl)pyridin-4-amine; N-(4-(1H-imidazol-1-yl)benzyl)-2-((2-(3-(dimethylamino)phenoxy)ethoxy)methyl)-N-(3-methoxybenzyl)pyridin-4-amine; N-(4-(1H-imidazol-1-yl)benzyl)-N-(3-methoxybenzyl)-2-((2-morpholinoethoxy)methyl)pyridin-4-amine; N-(4-(1H-imidazol-1-yl)benzyl)-N-(3-methoxybenzyl)-2-(morpholinomethyl)pyridin-4-amine; N-(4-(1H-imidazol-1-yl)benzyl)-N-(3-methoxybenzyl)-2-(piperidin-1-ylmethyl)pyridin-4-amine; N-(4-(1H-imidazol-1-yl)benzyl)-N-(3-methoxybenzyl)-2-((4-methylpiperazin-1-yl)methyl)pyridin-4-amine; 1-((4-((4-(1H-imidazol-1-yl)benzyl)(3-methoxybenzyl)amino)pyridin-2-yl)methyl)piperazin-2-one; 1-((4-((4-(1H-imidazol-1-yl)benzyl)(3-methoxybenzyl)amino)pyridin-2-yl)methyl)piperazine-2,5-dione; 4-((4-((4-(1H-imidazol-1-yl)benzyl)(3-methoxybenzyl)amino)pyridin-2-yl)methyl)piperazin-2-one; N-(4-(1H-imidazol-1-yl)benzyl)-2-((dimethylamino)methyl)-N-(3-methoxybenzyl)pyridin-4-amine; N-((1H-benzo[d]imidazol-6-yl)methyl)-N-(3-methoxybenzyl)-2-((2-(2-morpholinoethoxy)ethoxy)methyl)pyridin-4-amine; N-((1H-benzo[d]imidazol-6-yl)methyl)-N-(3-methoxybenzyl)-2-((2-(3-methoxybenzyloxy)ethoxy)methyl)pyridin-4-amine; N-((1H-benzo[d]imidazol-6-yl)methyl)-2-((2-(3-(dimethylamino)phenoxy)ethoxy)methyl)-N-(3-methoxybenzyl)pyridin-4-amine; N-((1H-benzo[d]imidazol-6-yl)methyl)-N-(3-methoxybenzyl)-2-((2-morpholinoethoxy)methyl)pyridin-4-amine; N-((1H-benzo[d]imidazol-6-yl)methyl)-N-(3-methoxybenzyl)-2-(morpholinomethyl)pyridin-4-amine; N-((1H-benzo[d]imidazol-6-yl)methyl)-N-(3-methoxybenzyl)-2-(piperidin-1-ylmethyl)pyridin-4-amine; N-((1H-benzo[d]imidazol-6-yl)methyl)-N-(3-methoxybenzyl)-2-((4-methylpiperazin-1-yl)methyl)pyridin-4-amine; 1-((4-(((1H-benzo[d]imidazol-6-yl)methyl)(3-methoxybenzyl)amino)pyridin-2-yl)methyl)piperazin-2-one; 1-((4-(((1H-benzo[d]imidazol-6-yl)methyl)(3-methoxybenzyl)amino)pyridin-2-yl)methyl)piperazine-2,5-dione; 4-((4-(((1H-benzo[d]imidazol-6-yl)methyl)(3-methoxybenzyl)amino)pyridin-2-yl)methyl)piperazin-2-one; N-((1H-benzo[d]imidazol-6-yl)methyl)-2-((dimethylamino)methyl)-N-(3-methoxybenzyl)pyridin-4-amine; N-((1H-indazol-6-yl)methyl)-N-(3-methoxybenzyl)-2-((2-(2-morpholinoethoxy)ethoxy)methyl)pyridin-4-amine; N-((1H-indazol-6-yl)methyl)-N-(3-methoxybenzyl)-2-((2-((3-methoxybenzyl)oxy)ethoxy)methyl)pyridin-4-amine; N-((1H-indazol-6-yl)methyl)-2-((2-(3-(dimethylamino)phenoxy)ethoxy)methyl)-N-(3-methoxybenzyl)pyridin-4-amine; N-((1H-indazol-6-yl)methyl)-N-(3-methoxybenzyl)-2-((2-morpholinoethoxy)methyl)pyridin-4-amine; N-((1H-indazol-6-yl)methyl)-N-(3-methoxybenzyl)-2-(morpholinomethyl)pyridin-4-amine; N-((1H-indazol-6-yl)methyl)-N-(3-methoxybenzyl)-2-(piperidin-1-ylmethyl)pyridin-4-amine; N-((1H-indazol-6-yl)methyl)-N-(3-methoxybenzyl)-2-((4-methylpiperazin-1-yl)methyl)pyridin-4-amine; 1-((4-(((1H-indazol-6-yl)methyl)(3-methoxybenzyl)amino)pyridin-2-yl)methyl)piperazin-2-one; 1-((4-(((1H-indazol-6-yl)methyl)(3-methoxybenzyl)amino)pyridin-2-yl)methyl)piperazine-2,5-dione; 4-((4-(((1H-indazol-6-yl)methyl)(3-methoxybenzyl)amino)pyridin-2-yl)methyl)piperazin-2-one; N-((1H-indazol-6-yl)methyl)-2-((dimethylamino)methyl)-N-(3-methoxybenzyl)pyridin-4-amine; N-(3-methoxybenzyl)-2-((2-(2-morpholinoethoxy)ethoxy)methyl)-N-(quinolin-7-ylmethyl)pyridin-4-amine; N-(3-methoxybenzyl)-2-((2-((3-methoxybenzyl)oxy)ethoxy)methyl)-N-(quinolin-7-ylmethyl)pyridin-4-amine; 2-((2-(3-(dimethylamino)phenoxy)ethoxy)methyl)-N-(3-methoxybenzyl)-N-(quinolin-7-ylmethyl)pyridin-4-amine; N-(3-methoxybenzyl)-2-((2-morpholinoethoxy)methyl)-N-(quinolin-7-ylmethyl)pyridin-4-amine; N-(3-methoxybenzyl)-2-(morpholinomethyl)-N-(quinolin-7-ylmethyl)pyridin-4-amine; N-(3-methoxybenzyl)-2-(piperidin-1-ylmethyl)-N-(quinolin-7-ylmethyl)pyridin-4-amine; N-(3-methoxybenzyl)-2-((4-methylpiperazin-1-yl)methyl)-N-(quinolin-7-ylmethyl)pyridin-4-amine; 1-((4-((3-methoxybenzyl)(quinolin-7-ylmethyl)amino)pyridin-2-yl)methyl)piperazin-2-one; 1-((4-((3-methoxybenzyl)(quinolin-7-ylmethyl)amino)pyridin-2-yl)methyl)piperazine-2,5-dione; 4-((4-((3-methoxybenzyl)(quinolin-7-ylmethyl)amino)pyridin-2-yl)methyl)piperazin-2-one; 2-((dimethylamino)methyl)-N-(3-methoxybenzyl)-N-(quinolin-7-ylmethyl)pyridin-4-amine; 4-((2-(2-((3-methoxybenzyl)oxy)ethoxy)ethoxy)methyl)-N,N-bis(3-(2-methoxyethoxy)benzyl)thiazol-2-amine; 4-((2-(2-(benzyloxy)ethoxy)ethoxy)methyl)-N,N-bis(3-(2-methoxyethoxy)benzyl)thiazol-2-amine; N,N-bis(3-(2-methoxyethoxy)benzyl)-4-((2-(2-(3-methoxyphenoxy)ethoxy)ethoxy)methyl)thiazol-2-amine; 4-((2-((3-methoxybenzyl)oxy)ethoxy)methyl)-N,N-bis(3-(2-methoxyethoxy)benzyl)thiazol-2-amine; 4-((2-(3-(dimethylamino)phenoxy)ethoxy)methyl)-N,N-bis(3-(2-methoxyethoxy)benzyl)thiazol-2-amine; N,N-bis(3-(2-methoxyethoxy)benzyl)-4-((2-(2-morpholinoethoxy)ethoxy)methyl)thiazol-2-amine; N,N-bis(3-(2-methoxyethoxy)benzyl)-4-((2-morpholinoethoxy)methyl)thiazol-2-amine; N,N-bis(3-(2-methoxyethoxy)benzyl)-4-(morpholinomethyl)thiazol-2-amine; 4-((2-(2-((3-methoxybenzyl)oxy)ethoxy)ethoxy)methyl)-N,N-bis(3-(2-methoxyethoxy)benzyl)oxazol-2-amine; 4-((2-(2-(benzyloxy)ethoxy)ethoxy)methyl)-N,N-bis(3-(2-methoxyethoxy)benzyl)oxazol-2-amine; N,N-bis(3-(2-methoxyethoxy)benzyl)-4-((2-(2-(3-methoxyphenoxy)ethoxy)ethoxy)methyl)oxazol-2-amine; 4-((2-((3-methoxybenzyl)oxy)ethoxy)methyl)-N,N-bis(3-(2-methoxyethoxy)benzyl)oxazol-2-amine; 4-((2-(3-(dimethylamino)phenoxy)ethoxy)methyl)-N,N-bis(3-(2-methoxyethoxy)benzyl)oxazol-2-amine; N,N-bis(3-(2-methoxyethoxy)benzyl)-4-((2-(2-morpholinoethoxy)ethoxy)methyl)oxazol-2-amine; N,N-bis(3-(2-methoxyethoxy)benzyl)-4-((2-morpholinoethoxy)methyl)oxazol-2-amine; N,N-bis(3-(2-methoxyethoxy)benzyl)-4-(morpholinomethyl)oxazol-2-amine; N-(3-methoxybenzyl)-4-((2-(2-((3-methoxybenzyl)oxy)ethoxy)ethoxy)methyl)-N-(3-(2-methoxyethoxy)benzyl)thiazol-2-amine; 4-((2-(2-(benzyloxy)ethoxy)ethoxy)methyl)-N-(3-methoxybenzyl)-N-(3-(2-methoxyethoxy)benzyl)thiazol-2-amine; N-(3-methoxybenzyl)-N-(3-(2-methoxyethoxy)benzyl)-4-((2-(2-(3-methoxyphenoxy)ethoxy)ethoxy)methyl)thiazol-2-amine; N-(3-methoxybenzyl)-4-((2-((3-methoxybenzyl)oxy)ethoxy)methyl)-N-(3-(2-methoxyethoxy)benzyl)thiazol-2-amine; 4-((2-(3-(dimethylamino)phenoxy)ethoxy)methyl)-N-(3-methoxybenzyl)-N-(3-(2-methoxyethoxy)benzyl)thiazol-2-amine; N-(3-methoxybenzyl)-N-(3-(2-methoxyethoxy)benzyl)-4-((2-(2-morpholinoethoxy)ethoxy)methyl)thiazol-2-amine; N-(3-methoxybenzyl)-N-(3-(2-methoxyethoxy)benzyl)-4-((2-morpholinoethoxy)methyl)thiazol-2-amine; N-(3-methoxybenzyl)-N-(3-(2-methoxyethoxy)benzyl)-4-(morpholinomethyl)thiazol-2-amine; N-(3-methoxybenzyl)-4-((2-(2-((3-methoxybenzyl)oxy)ethoxy)ethoxy)methyl)-N-(3-(2-methoxyethoxy)benzyl)oxazol-2-amine; 4-((2-(2-(benzyloxy)ethoxy)ethoxy)methyl)-N-(3-methoxybenzyl)-N-(3-(2-methoxyethoxy)benzyl)oxazol-2-amine; N-(3-methoxybenzyl)-N-(3-(2-methoxyethoxy)benzyl)-4-((2-(2-(3-methoxyphenoxy)ethoxy)ethoxy)methyl)oxazol-2-amine; 4-((2-(3-(dimethylamino)phenoxy)ethoxy)methyl)-N-(3-methoxybenzyl)-N-(3-(2-methoxyethoxy)benzyl)oxazol-2-amine; N-(3-methoxybenzyl)-N-(3-(2-methoxyethoxy)benzyl)-4-((2-(2-morpholinoethoxy)ethoxy)methyl)oxazol-2-amine; N-(3-methoxybenzyl)-N-(3-(2-methoxyethoxy)benzyl)-4-((2-morpholinoethoxy)methyl)oxazol-2-amine; N-(3-methoxybenzyl)-N-(3-(2-methoxyethoxy)benzyl)-4-(morpholinomethyl)oxazol-2-amine; N,N-bis(3-methoxybenzyl)-4-(2-morpholinoethyl)thiazol-2-amine; N,N-bis(3-methoxybenzyl)-4-methyl-5-(morpholinomethyl)thiazol-2-amine; N-(3-(dimethylamino)benzyl)-N-(3-methoxybenzyl)-4-(2-morpholinoethyl)thiazol-2-amine; N-(3-(dimethylamino)benzyl)-N-(3-methoxybenzyl)-4-methyl-5-(morpholinomethyl)thiazol-2-amine; N-(3-methoxybenzyl)-4-(2-morpholinoethyl)-N-(3-(pyrrolidin-1-yl)benzyl)thiazol-2-amine; N-(3-methoxybenzyl)-4-methyl-5-(morpholinomethyl)-N-(3-(pyrrolidin-1-yl)benzyl)thiazol-2-amine; N-(3-(1H-pyrazol-1-yl)benzyl)-N-(3-methoxybenzyl)-4-(2-morpholinoethyl)thiazol-2-amine; N-(3-(1H-pyrazol-1-yl)benzyl)-N-(3-methoxybenzyl)-4-methyl-5-(morpholinomethyl)thiazol-2-amine; N-(3-(1H-imidazol-1-yl)benzyl)-N-(3-methoxybenzyl)-4-(2-morpholinoethyl)thiazol-2-amine; N-(3-(1H-imidazol-1-yl)benzyl)-N-(3-methoxybenzyl)-4-methyl-5-(morpholinomethyl)thiazol-2-amine; N-(3-methoxybenzyl)-4-(2-morpholinoethyl)-N-(4-(pyrrolidin-1-yl)benzyl)thiazol-2-amine; N-(3-methoxybenzyl)-4-methyl-5-(morpholinomethyl)-N-(4-(pyrrolidin-1-yl)benzyl)thiazol-2-amine; N-(3-methoxybenzyl)-N-(4-morpholinobenzyl)-4-(2-morpholinoethyl)thiazol-2-amine; N-(3-methoxybenzyl)-4-methyl-N-(4-morpholinobenzyl)-5-(morpholinomethyl)thiazol-2-amine; N-(3-methoxybenzyl)-N-(4-(4-methylpiperazin-1-yl)benzyl)-4-(2-morpholinoethyl)thiazol-2-amine; N-(3-methoxybenzyl)-4-methyl-N-(4-(4-methylpiperazin-1-yl)benzyl)-5-(morpholinomethyl)thiazol-2-amine; N-(3-methoxybenzyl)-N-(3-morpholinobenzyl)-4-(2-morpholinoethyl)thiazol-2-amine; N-(3-methoxybenzyl)-4-methyl-N-(3-morpholinobenzyl)-5-(morpholinomethyl)thiazol-2-amine; N-(3-methoxybenzyl)-N-(3-(4-methylpiperazin-1-yl)benzyl)-4-(2-morpholinoethyl)thiazol-2-amine; N-(3-methoxybenzyl)-4-methyl-N-(3-(4-methylpiperazin-1-yl)benzyl)-5-(morpholinomethyl)thiazol-2-amine; N-(4-(1H-pyrazol-1-yl)benzyl)-N-(3-methoxybenzyl)-4-(2-morpholinoethyl)thiazol-2-amine; N-(4-(1H-pyrazol-1-yl)benzyl)-N-(3-methoxybenzyl)-4-methyl-5-(morpholinomethyl)thiazol-2-amine; N-(4-(1H-imidazol-1-yl)benzyl)-N-(3-methoxybenzyl)-4-(2-morpholinoethyl)thiazol-2-amine; N-(4-(1H-imidazol-1-yl)benzyl)-N-(3-methoxybenzyl)-4-methyl-5-(morpholinomethyl)thiazol-2-amine; N-((1H-benzo[d]imidazol-6-yl)methyl)-N-(3-methoxybenzyl)-4-(2-morpholinoethyl)thiazol-2-amine; N-((1H-benzo[d]imidazol-6-yl)methyl)-N-(3-methoxybenzyl)-4-methyl-5-(morpholinomethyl)thiazol-2-amine; N-((1H-indazol-6-yl)methyl)-N-(3-methoxybenzyl)-4-(2-morpholinoethyl)thiazol-2-amine; N-((1H-indazol-6-yl)methyl)-N-(3-methoxybenzyl)-4-methyl-5-(morpholinomethyl)thiazol-2-amine; N-(3-methoxybenzyl)-4-(2-morpholinoethyl)-N-(quinolin-7-ylmethyl)thiazol-2-amine; N-(3-methoxybenzyl)-4-methyl-5-(morpholinomethyl)-N-(quinolin-7-ylmethyl)thiazol-2-amine; N,N-bis(3-methoxybenzyl)-4-(2-morpholinoethyl)oxazol-2-amine; N,N-bis(3-methoxybenzyl)-4-methyl-5-(morpholinomethyl)oxazol-2-amine; N-(3-(dimethylamino)benzyl)-N-(3-methoxybenzyl)-4-(2-morpholinoethyl)oxazol-2-amine; N-(3-(dimethylamino)benzyl)-N-(3-methoxybenzyl)-4-methyl-5-(morpholinomethyl)oxazol-2-amine; N-(3-methoxybenzyl)-4-(2-morpholinoethyl)-N-(3-(pyrrolidin-1-yl)benzyl)oxazol-2-amine; N-(3-methoxybenzyl)-4-methyl-5-(morpholinomethyl)-N-(3-(pyrrolidin-1-yl)benzyl)oxazol-2-amine; N-(3-(1H-pyrazol-1-yl)benzyl)-N-(3-methoxybenzyl)-4-(2-morpholinoethyl)oxazol-2-amine; N-(3-(1H-pyrazol-1-yl)benzyl)-N-(3-methoxybenzyl)-4-methyl-5-(morpholinomethyl)oxazol-2-amine; N-(3-(1H-imidazol-1-yl)benzyl)-N-(3-methoxybenzyl)-4-(2-morpholinoethyl)oxazol-2-amine; N-(3-(1H-imidazol-1-yl)benzyl)-N-(3-methoxybenzyl)-4-methyl-5-(morpholinomethyl)oxazol-2-amine; N-(3-methoxybenzyl)-4-(2-morpholinoethyl)-N-(4-(pyrrolidin-1-yl)benzyl)oxazol-2-amine; N-(3-methoxybenzyl)-4-methyl-5-(morpholinomethyl)-N-(4-(pyrrolidin-1-yl)benzyl)oxazol-2-amine; N-(3-methoxybenzyl)-N-(4-morpholinobenzyl)-4-(2-morpholinoethyl)oxazol-2-amine; N-(3-methoxybenzyl)-4-methyl-N-(4-morpholinobenzyl)-5-(morpholinomethyl)oxazol-2-amine; N-(3-methoxybenzyl)-N-(4-(4-methylpiperazin-1-yl)benzyl)-4-(2-morpholinoethyl)oxazol-2-amine; N-(3-methoxybenzyl)-4-methyl-N-(4-(4-methylpiperazin-1-yl)benzyl)-5-(morpholinomethyl)oxazol-2-amine; N-(3-methoxybenzyl)-N-(3-morpholinobenzyl)-4-(2-morpholinoethyl)oxazol-2-amine; N-(3-methoxybenzyl)-4-methyl-N-(3-morpholinobenzyl)-5-(morpholinomethyl)oxazol-2-amine; N-(3-methoxybenzyl)-N-(3-(4-methylpiperazin-1-yl)benzyl)-4-(2-morpholinoethyl)oxazol-2-amine; N-(3-methoxybenzyl)-4-methyl-N-(3-(4-methylpiperazin-1-yl)benzyl)-5-(morpholinomethyl)oxazol-2-amine; N-(4-(1H-imidazol-1-yl)benzyl)-N-(3-methoxybenzyl)-4-(2-morpholinoethyl)oxazol-2-amine; N-(4-(1H-imidazol-1-yl)benzyl)-N-(3-methoxybenzyl)-4-methyl-5-(morpholinomethyl)oxazol-2-amine; N-(4-(1H-pyrazol-1-yl)benzyl)-N-(3-methoxybenzyl)-4-(2-morpholinoethyl)oxazol-2-amine; N-(4-(1H-pyrazol-1-yl)benzyl)-N-(3-methoxybenzyl)-4-methyl-5-(morpholinomethyl)oxazol-2-amine; N-((1H-benzo[d]imidazol-6-yl)methyl)-N-(3-methoxybenzyl)-4-(2-morpholinoethyl)oxazol-2-amine; N-((1H-benzo[d]imidazol-6-yl)methyl)-N-(3-methoxybenzyl)-4-methyl-5-(morpholinomethyl)oxazol-2-amine; N-((1H-indazol-6-yl)methyl)-N-(3-methoxybenzyl)-4-(2-morpholinoethyl)oxazol-2-amine; N-((1H-indazol-6-yl)methyl)-N-(3-methoxybenzyl)-4-methyl-5-(morpholinomethyl)oxazol-2-amine; N-(3-methoxybenzyl)-4-(2-morpholinoethyl)-N-(quinolin-7-ylmethyl)oxazol-2-amine; N-(3-methoxybenzyl)-4-methyl-5-(morpholinomethyl)-N-(quinolin-7-ylmethyl)oxazol-2-amine; 5-ethyl-N,N-bis(3-methoxybenzyl)-1,3,4-thiadiazol-2-amine; N,N-bis(3-methoxybenzyl)-5-((2-(2-(3-methoxyphenoxy)ethoxy)ethoxy)methyl)pyridin-2-amine; 5-((2-(2-(benzyloxy)ethoxy)ethoxy)methyl)-N,N-bis(3-methoxybenzyl)pyridin-2-amine; N,N-bis(3-methoxybenzyl)-5-((2-(2-((3-methoxybenzyl)oxy)ethoxy)ethoxy)methyl)pyridin-2-amine; 5-((2-(3-(dimethylamino)phenoxy)ethoxy)methyl)-N,N-bis(3-methoxybenzyl)pyridin-2-amine; N,N-bis(3-methoxybenzyl)-5-((2-((3-methoxybenzyl)oxy)ethoxy)methyl)pyridin-2-amine; N,N-bis(3-methoxybenzyl)-5-((2-(2-morpholinoethoxy)ethoxy)methyl)pyridin-2-amine; N,N-bis(3-methoxybenzyl)-5-((2-morpholinoethoxy)methyl)pyridin-2-amine; N,N-bis(3-methoxybenzyl)-5-(morpholinomethyl)pyridin-2-amine; N-(3-(dimethylamino)benzyl)-N-(3-methoxybenzyl)-5-((2-(2-(3-methoxyphenoxy)ethoxy)ethoxy)methyl)pyridin-2-amine; 5-((2-(2-(benzyloxy)ethoxy)ethoxy)methyl)-N-(3-(dimethylamino)benzyl)-N-(3-methoxybenzyl)pyridin-2-amine; N-(3-(dimethylamino)benzyl)-N-(3-methoxybenzyl)-5-((2-(2-((3-methoxybenzyl)oxy)ethoxy)ethoxy)methyl)pyridin-2-amine; N-(3-(dimethylamino)benzyl)-5-((2-(3-(dimethylamino)phenoxy)ethoxy)methyl)-N-(3-methoxybenzyl)pyridin-2-amine; N-(3-(dimethylamino)benzyl)-N-(3-methoxybenzyl)-5-((2-((3-methoxybenzyl)oxy)ethoxy)methyl)pyridin-2-amine; N-(3-(dimethylamino)benzyl)-N-(3-methoxybenzyl)-5-((2-(2-morpholinoethoxy)ethoxy)methyl)pyridin-2-amine; N-(3-(dimethylamino)benzyl)-N-(3-methoxybenzyl)-5-((2-morpholinoethoxy)methyl)pyridin-2-amine; N-(3-(dimethylamino)benzyl)-N-(3-methoxybenzyl)-5-(morpholinomethyl)pyridin-2-amine; N-(3-methoxybenzyl)-5-((2-(2-(3-methoxyphenoxy)ethoxy)ethoxy)methyl)-N-(4-morpholinobenzyl)pyridin-2-amine; 5-((2-(2-(benzyloxy)ethoxy)ethoxy)methyl)-N-(3-methoxybenzyl)-N-(4-morpholinobenzyl)pyridin-2-amine; N-(3-methoxybenzyl)-5-((2-(2-((3-methoxybenzyl)oxy)ethoxy)ethoxy)methyl)-N-(4-morpholinobenzyl)pyridin-2-amine; 5-((2-(3-(dimethylamino)phenoxy)ethoxy)methyl)-N-(3-methoxybenzyl)-N-(4-morpholinobenzyl)pyridin-2-amine; N-(3-methoxybenzyl)-5-((2-((3-methoxybenzyl)oxy)ethoxy)methyl)-N-(4-morpholinobenzyl)pyridin-2-amine; N-(3-methoxybenzyl)-N-(4-morpholinobenzyl)-5-((2-(2-morpholinoethoxy)ethoxy)methyl)pyridin-2-amine; N-(3-methoxybenzyl)-N-(4-morpholinobenzyl)-5-((2-morpholinoethoxy)methyl)pyridin-2-amine; N-(3-methoxybenzyl)-N-(4-morpholinobenzyl)-5-(morpholinomethyl)pyridin-2-amine; N-((1H-indazol-6-yl)methyl)-N-(3-methoxybenzyl)-5-((2-(2-(3-methoxyphenoxy)ethoxy)ethoxy)methyl)pyridin-2-amine; N-((1H-indazol-6-yl)methyl)-5-((2-(2-(benzyloxy)ethoxy)ethoxy)methyl)-N-(3-methoxybenzyl)pyridin-2-amine; N-((1H-indazol-6-yl)methyl)-N-(3-methoxybenzyl)-5-((2-(2-((3-methoxybenzyl)oxy)ethoxy)ethoxy)methyl)pyridin-2-amine; N-((1H-indazol-6-yl)methyl)-5-((2-(3-(dimethylamino)phenoxy)ethoxy)methyl)-N-(3-methoxybenzyl)pyridin-2-amine; N-((1H-indazol-6-yl)methyl)-N-(3-methoxybenzyl)-5-((2-((3-methoxybenzyl)oxy)ethoxy)methyl)pyridin-2-amine; N-((1H-indazol-6-yl)methyl)-N-(3-methoxybenzyl)-5-((2-(2 morpholinoethoxy)ethoxy)methyl)pyridin-2-amine; N-((1H-indazol-6-yl)methyl)-N-(3-methoxybenzyl)-5-((2-morpholinoethoxy)methyl)pyridin-2-amine; and N-((1H-indazol-6-yl)methyl)-N-(3-methoxybenzyl)-5-(morpholinomethyl)pyridin-2-amine. N,N-bis(3-methoxybenzyl)-4-(2-(2-(3-methoxyphenoxy)ethoxy)ethoxy)aniline; 4-(2-(2-(benzyloxy)ethoxy)ethoxy)-N,N-bis(3-methoxybenzyl)aniline; N,N-bis(3-methoxybenzyl)-4-(2-(2-morpholinoethoxy)ethoxy)aniline; 3-(2-(4-(bis(3-methoxybenzyl)amino)phenoxy)ethoxy)-N,N-dimethylaniline; N,N-bis(3-methoxybenzyl)-4-(2-morpholinoethoxy)aniline; 3-(((3-methoxybenzyl)(4-(2-(2-(3-methoxyphenoxy)ethoxy)ethoxy)phenyl)amino)methyl)-N,N-dimethylaniline; 3-(((4-(2-(2-(benzyloxy)ethoxy)ethoxy)phenyl)(3-methoxybenzyl)amino)methyl)-N,N-dimethylaniline; 3-(((3-methoxybenzyl)(4-(2-(2-morpholinoethoxy)ethoxy)phenyl)amino)methyl)-N,N-dimethylaniline; 3-(2-(4-((3-(dimethylamino)benzyl)(3-methoxybenzyl)amino)phenoxy)ethoxy)-N,N-dimethylaniline; 3-(((3-methoxybenzyl)(4-(2-morpholinoethoxy)phenyl)amino)methyl)-N,N-dimethylaniline; N-(3-methoxybenzyl)-4-(2-(2-(3-methoxyphenoxy)ethoxy)ethoxy)-N-(3-(pyrrolidin-1-yl)benzyl)aniline; 4-(2-(2-(benzyloxy)ethoxy)ethoxy)-N-(3-methoxybenzyl)-N-(3-(pyrrolidin-1-yl)benzyl)aniline; N-(3-methoxybenzyl)-4-(2-(2-morpholinoethoxy)ethoxy)-N-(3-(pyrrolidin-1-yl)benzyl)aniline; 3-(2-(4-((3-methoxybenzyl)(3-(pyrrolidin-1-yl)benzyl)amino)phenoxy)ethoxy)-N,N-dimethylaniline; N-(3-methoxybenzyl)-4-(2-morpholinoethoxy)-N-(3-(pyrrolidin-1-yl)benzyl)aniline; N-(3-(1H-imidazol-1-yl)benzyl)-N-(3-methoxybenzyl)-4-(2-(2-(3-methoxyphenoxy)ethoxy)ethoxy)aniline; N-(3-(1H-imidazol-1-yl)benzyl)-4-(2-(2-(benzyloxy)ethoxy)ethoxy)-N-(3-methoxybenzyl)aniline; N-(3-(1H-imidazol-1-yl)benzyl)-N-(3-methoxybenzyl)-4-(2-(2-morpholinoethoxy)ethoxy)aniline; 3-(2-(4-((3-(1H-imidazol-1-yl)benzyl)(3-methoxybenzyl)amino)phenoxy)ethoxy)-N,N-dimethylaniline; N-(3-(1H-imidazol-1-yl)benzyl)-N-(3-methoxybenzyl)-4-(2-morpholinoethoxy)aniline; N-(3-methoxybenzyl)-4-(2-(2-(3-methoxyphenoxy)ethoxy)ethoxy)-N-(4-morpholinobenzyl)aniline; 4-(2-(2-(benzyloxy)ethoxy)ethoxy)-N-(3-methoxybenzyl)-N-(4-morpholinobenzyl)aniline; N-(3-methoxybenzyl)-N-(4-morpholinobenzyl)-4-(2-(2-morpholinoethoxy)ethoxy)aniline; 3-(2-(4-((3-methoxybenzyl)(4-morpholinobenzyl)amino)phenoxy)ethoxy)-N,N-dimethylaniline; N-(3-methoxybenzyl)-N-(4-morpholinobenzyl)-4-(2-morpholinoethoxy)aniline; N-(4-(1H-pyrazol-1-yl)benzyl)-N-(3-methoxybenzyl)-4-(2-(2-(3-methoxyphenoxy)ethoxy)ethoxy)aniline; N-(4-(1H-pyrazol-1-yl)benzyl)-4-(2-(2-(benzyloxy)ethoxy)ethoxy)-N-(3-methoxybenzyl)aniline; N-(4-(1H-pyrazol-1-yl)benzyl)-N-(3-methoxybenzyl)-4-(2-(2-morpholinoethoxy)ethoxy)aniline; 3-(2-(4-((4-(1H-pyrazol-1-yl)benzyl)(3-methoxybenzyl)amino)phenoxy)ethoxy)-N,N-dimethylaniline; N-(4-(1H-pyrazol-1-yl)benzyl)-N-(3-methoxybenzyl)-4-(2-morpholinoethoxy)aniline; N-((1H-benzo[d]imidazol-6-yl)methyl)-N-(3-methoxybenzyl)-4-(2-(2-(3-methoxyphenoxy)ethoxy)ethoxy)aniline; N-((1H-benzo[d]imidazol-6-yl)methyl)-4-(2-(2-(benzyloxy)ethoxy)ethoxy)-N-(3-methoxybenzyl)aniline; N-((1H-benzo[d]imidazol-6-yl)methyl)-N-(3-methoxybenzyl)-4-(2-(2-morpholinoethoxy)ethoxy)aniline; 3-(2-(4-(((1H-benzo[d]imidazol-6-yl)methyl)(3-methoxybenzyl)amino)phenoxy)ethoxy)-N,N-dimethylaniline; N-((1H-benzo[d]imidazol-6-yl)methyl)-N-(3-methoxybenzyl)-4-(2-morpholinoethoxy)aniline; N,N-bis(3-methoxybenzyl)-3-(2-(2-(3-methoxyphenoxy)ethoxy)ethoxy)aniline; 3-(2-(2-(benzyloxy)ethoxy)ethoxy)-N,N-bis(3-methoxybenzyl)aniline; N,N-bis(3-methoxybenzyl)-3-(2-(2-morpholinoethoxy)ethoxy)aniline; 3-(2-(3-(bis(3-methoxybenzyl)amino)phenoxy)ethoxy)-N,N-dimethylaniline; N,N-bis(3-methoxybenzyl)-3-(2-morpholinoethoxy)aniline; N-(3-(dimethylamino)benzyl)-N-(3-methoxybenzyl)-3-(2-(2-(3-methoxyphenoxy)ethoxy)ethoxy)aniline; 3-(2-(2-(benzyloxy)ethoxy)ethoxy)-N-(3-(dimethylamino)benzyl)-N-(3-methoxybenzyl)aniline; N-(3-(dimethylamino)benzyl)-N-(3-methoxybenzyl)-3-(2-(2-morpholinoethoxy)ethoxy)aniline; N-(3-(dimethylamino)benzyl)-3-(2-(3-(dimethylamino)phenoxy)ethoxy)-N-(3-methoxybenzyl)aniline; N-(3-(dimethylamino)benzyl)-N-(3-methoxybenzyl)-3-(2-morpholinoethoxy)aniline; N-(3-methoxybenzyl)-3-(2-(2-(3-methoxyphenoxy)ethoxy)ethoxy)-N-(3-(pyrrolidin-1-yl)benzyl)aniline; 3-(2-(2-(benzyloxy)ethoxy)ethoxy)-N-(3-methoxybenzyl)-N-(3-(pyrrolidin-1-yl)benzyl)aniline; N-(3-methoxybenzyl)-3-(2-(2-morpholinoethoxy)ethoxy)-N-(3-(pyrrolidin-1-yl)benzyl)aniline; 3-(2-(3-(dimethylamino)phenoxy)ethoxy)-N-(3-methoxybenzyl)-N-(3-(pyrrolidin-1-yl)benzyl)aniline; N-(3-methoxybenzyl)-3-(2-morpholinoethoxy)-N-(3-(pyrrolidin-1-yl)benzyl)aniline; N-(3-(1H-imidazol-1-yl)benzyl)-N-(3-methoxybenzyl)-3-(2-(2-(3-methoxyphenoxy)ethoxy)ethoxy)aniline; N-(3-(1H-imidazol-1-yl)benzyl)-3-(2-(2-(benzyloxy)ethoxy)ethoxy)-N-(3-methoxybenzyl)aniline; N-(3-(1H-imidazol-1-yl)benzyl)-N-(3-methoxybenzyl)-3-(2-(2-morpholinoethoxy)ethoxy)aniline; N-(3-(1H-imidazol-1-yl)benzyl)-3-(2-(3-(dimethylamino)phenoxy)ethoxy)-N-(3-methoxybenzyl)aniline; N-(3-(1H-imidazol-1-yl)benzyl)-N-(3-methoxybenzyl)-3-(2-morpholinoethoxy)aniline; N-(3-methoxybenzyl)-3-(2-(2-(3-methoxyphenoxy)ethoxy)ethoxy)-N-(4-morpholinobenzyl)aniline; N-(3-methoxybenzyl)-3-(2-(2-(3-methoxyphenoxy)ethoxy)ethoxy)-N-(4-morpholinobenzyl)aniline; N-(3-methoxybenzyl)-N-(4-morpholinobenzyl)-3-(2-(2-morpholinoethoxy)ethoxy)aniline; 3-(2-(3-(dimethylamino)phenoxy)ethoxy)-N-(3-methoxybenzyl)-N-(4-morpholinobenzyl)aniline; N-(3-methoxybenzyl)-N-(4-morpholinobenzyl)-3-(2-morpholinoethoxy)aniline; N-(4-(1H-pyrazol-1-yl)benzyl)-N-(3-methoxybenzyl)-3-(2-(2-(3-methoxyphenoxy)ethoxy)ethoxy)aniline; N-(4-(1H-pyrazol-1-yl)benzyl)-3-(2-(2-(benzyloxy)ethoxy)ethoxy)-N-(3-methoxybenzyl)aniline; N-(4-(1H-pyrazol-1-yl)benzyl)-N-(3-methoxybenzyl)-3-(2-(2-morpholinoethoxy)ethoxy)aniline; N-(4-(1H-pyrazol-1-yl)benzyl)-3-(2-(3-(dimethylamino)phenoxy)ethoxy)-N-(3-methoxybenzyl)aniline; N-(4-(1H-pyrazol-1-yl)benzyl)-N-(3-methoxybenzyl)-3-(2-morpholinoethoxy)aniline; N-((1H-benzo[d]imidazol-6-yl)methyl)-N-(3-methoxybenzyl)-3-(2-(2-(3-methoxyphenoxy)ethoxy)ethoxy)aniline; N-((1H-benzo[d]imidazol-6-yl)methyl)-3-(2-(2-(benzyloxy)ethoxy)ethoxy)-N-(3-methoxybenzyl)aniline; N-((1H-benzo[d]imidazol-6-yl)methyl)-N-(3-methoxybenzyl)-3-(2-(2-morpholinoethoxy)ethoxy)aniline; N-((1H-benzo[d]imidazol-6-yl)methyl)-3-(2-(3-(dimethylamino)phenoxy)ethoxy)-N-(3-methoxybenzyl)aniline; N-((1H-benzo[d]imidazol-6-yl)methyl)-N-(3-methoxybenzyl)-3-(2-morpholinoethoxy)aniline; N,N-bis(3-methoxybenzyl)-2-(2-(2-(3-methoxyphenoxy)ethoxy)ethoxy)pyridin-4-amine; 2-(2-(2-(benzyloxy)ethoxy)ethoxy)-N,N-bis(3-methoxybenzyl)pyridin-4-amine; N,N-bis(3-methoxybenzyl)-2-(2-(2-morpholinoethoxy)ethoxy)pyridin-4-amine; 2-(2-(3-(dimethylamino)phenoxy)ethoxy)-N,N-bis(3-methoxybenzyl)pyridin-4-amine; N,N-bis(3-methoxybenzyl)-2-(2-morpholinoethoxy)pyridin-4-amine; N-(3-(dimethylamino)benzyl)-N-(3-methoxybenzyl)-2-(2-(2-(3-methoxyphenoxy)ethoxy)ethoxy)pyridin-4-amine; 2-(2-(2-(benzyloxy)ethoxy)ethoxy)-N-(3-(dimethylamino)benzyl)-N-(3-methoxybenzyl)pyridin-4-amine; N-(3-(dimethylamino)benzyl)-N-(3-methoxybenzyl)-2-(2-(2-morpholinoethoxy)ethoxy)pyridin-4-amine; N-(3-(dimethylamino)benzyl)-2-(2-(3-(dimethylamino)phenoxy)ethoxy)-N-(3-methoxybenzyl)pyridin-4-amine; N-(3-(dimethylamino)benzyl)-N-(3-methoxybenzyl)-2-(2-morpholinoethoxy)pyridin-4-amine; N-(3-methoxybenzyl)-2-(2-(2-(3-methoxyphenoxy)ethoxy)ethoxy)-N-(3-(pyrrolidin-1-yl)benzyl)pyridin-4-amine; 2-(2-(2-(benzyloxy)ethoxy)ethoxy)-N-(3-methoxybenzyl)-N-(3-(pyrrolidin-1-yl)benzyl)pyridin-4-amine; N-(3-methoxybenzyl)-2-(2-(2-morpholinoethoxy)ethoxy)-N-(3-(pyrrolidin-1-yl)benzyl)pyridin-4-amine; 2-(2-(3-(dimethylamino)phenoxy)ethoxy)-N-(3-methoxybenzyl)-N-(3-(pyrrolidin-1-yl)benzyl)pyridin-4-amine; N-(3-methoxybenzyl)-2-(2-morpholinoethoxy)-N-(3-(pyrrolidin-1-yl)benzyl)pyridin-4-amine; N-(3-(1H-imidazol-1-yl)benzyl)-N-(3-methoxybenzyl)-2-(2-(2-(3-methoxyphenoxy)ethoxy)ethoxy)pyridin-4-amine; N-(3-(1H-imidazol-1-yl)benzyl)-2-(2-(2-(benzyloxy)ethoxy)ethoxy)-N-(3-methoxybenzyl)pyridin-4-amine; N-(3-(1H-imidazol-1-yl)benzyl)-N-(3-methoxybenzyl)-2-(2-(2-morpholinoethoxy)ethoxy)pyridin-4-amine; N-(3-(1H-imidazol-1-yl)benzyl)-2-(2-(3-(dimethylamino)phenoxy)ethoxy)-N-(3-methoxybenzyl)pyridin-4-amine; N-(3-(1H-imidazol-1-yl)benzyl)-N-(3-methoxybenzyl)-2-(2-morpholinoethoxy)pyridin-4-amine; N-(3-methoxybenzyl)-2-(2-(2-(3-methoxyphenoxy)ethoxy)ethoxy)-N-(4-morpholinobenzyl)pyridin-4-amine; 2-(2-(2-(benzyloxy)ethoxy)ethoxy)-N-(3-methoxybenzyl)-N-(4-morpholinobenzyl)pyridin-4-amine; N-(3-methoxybenzyl)-N-(4-morpholinobenzyl)-2-(2-(2-morpholinoethoxy)ethoxy)pyridin-4-amine; 2-(2-(3-(dimethylamino)phenoxy)ethoxy)-N-(3-methoxybenzyl)-N-(4-morpholinobenzyl)pyridin-4-amine; N-(3-methoxybenzyl)-N-(4-morpholinobenzyl)-2-(2-morpholinoethoxy)pyridin-4-amine; N-(4-(1H-pyrazol-1-yl)benzyl)-N-(3-methoxybenzyl)-2-(2-(2-(3 methoxyphenoxy)ethoxy)ethoxy)pyridin-4-amine; N-(4-(1H-pyrazol-1-yl)benzyl)-2-(2-(2-(benzyloxy)ethoxy)ethoxy)-N-(3-methoxybenzyl)pyridin-4-amine; N-(4-(1H-pyrazol-1-yl)benzyl)-N-(3-methoxybenzyl)-2-(2-(2-morpholinoethoxy)ethoxy)pyridin-4-amine; N-(4-(1H-pyrazol-1-yl)benzyl)-2-(2-(3-(dimethylamino)phenoxy)ethoxy)-N-(3-methoxybenzyl)pyridin-4-amine; N-(4-(1H-pyrazol-1-yl)benzyl)-N-(3-methoxybenzyl)-2-(2-morpholinoethoxy)pyridin-4-amine; N-((1H-benzo[d]imidazol-6-yl)methyl)-N-(3-methoxybenzyl)-2-(2-(2-(3-methoxyphenoxy)ethoxy)ethoxy)pyridin-4-amine; N-((1H-benzo[d]imidazol-6-yl)methyl)-2-(2-(2-(benzyloxy)ethoxy)ethoxy)-N-(3-methoxybenzyl)pyridin-4-amine; N-((1H-benzo[d]imidazol-6-yl)methyl)-N-(3-methoxybenzyl)-2-(2-(2-morpholinoethoxy)ethoxy)pyridin-4-amine; N-((1H-benzo[d]imidazol-6-yl)methyl)-2-(2-(3-(dimethylamino)phenoxy)ethoxy)-N-(3-methoxybenzyl)pyridin-4-amine; N-((1H-benzo[d]imidazol-6-yl)methyl)-N-(3-methoxybenzyl)-2-(2-morpholinoethoxy)pyridin-4-amine; N,N-bis(3-methoxybenzyl)-5-(2-(2-(3-methoxyphenoxy)ethoxy)ethoxy)pyridin-2-amine; 5-(2-(2-(benzyloxy)ethoxy)ethoxy)-N,N-bis(3-methoxybenzyl)pyridin-2-amine; N,N-bis(3-methoxybenzyl)-5-(2-(2-morpholinoethoxy)ethoxy)pyridin-2-amine; 5-(2-(3-(dimethylamino)phenoxy)ethoxy)-N,N-bis(3-methoxybenzyl)pyridin-2-amine; N,N-bis(3-methoxybenzyl)-5-(2-morpholinoethoxy)pyridin-2-amine; N-(3-(dimethylamino)benzyl)-N-(3-methoxybenzyl)-5-(2-(2-(3-methoxyphenoxy)ethoxy)ethoxy)pyridin-2-amine; 5-(2-(2-(benzyloxy)ethoxy)ethoxy)-N-(3-(dimethylamino)benzyl)-N-(3-methoxybenzyl)pyridin-2-amine; N-(3-(dimethylamino)benzyl)-N-(3-methoxybenzyl)-5-(2-(2-morpholinoethoxy)ethoxy)pyridin-2-amine; N-(3-(dimethylamino)benzyl)-5-(2-(3-(dimethylamino)phenoxy)ethoxy)-N-(3-methoxybenzyl)pyridin-2-amine; N-(3-(dimethylamino)benzyl)-N-(3-methoxybenzyl)-5-(2-morpholinoethoxy)pyridin-2-amine; N-(3-methoxybenzyl)-5-(2-(2-(3-methoxyphenoxy)ethoxy)ethoxy)-N-(3-(pyrrolidin-1-yl)benzyl)pyridin-2-amine; 5-(2-(2-(benzyloxy)ethoxy)ethoxy)-N-(3-methoxybenzyl)-N-(3-(pyrrolidin-1-yl)benzyl)pyridin-2-amine; N-(3-methoxybenzyl)-5-(2-(2-morpholinoethoxy)ethoxy)-N-(3-(pyrrolidin-1-yl)benzyl)pyridin-2-amine; 5-(2-(3-(dimethylamino)phenoxy)ethoxy)-N-(3-methoxybenzyl)-N-(3-(pyrrolidin-1-yl)benzyl)pyridin-2-amine; N-(3-methoxybenzyl)-5-(2-morpholinoethoxy)-N-(3-(pyrrolidin-1-yl)benzyl)pyridin-2-amine; N-(3-(1H-imidazol-1-yl)benzyl)-N-(3-methoxybenzyl)-5-(2-(2-(3-methoxyphenoxy)ethoxy)ethoxy)pyridin-2-amine; N-(3-(1H-imidazol-1-yl)benzyl)-5-(2-(2-(benzyloxy)ethoxy)ethoxy)-N-(3-methoxybenzyl)pyridin-2-amine; N-(3-(1H-imidazol-1-yl)benzyl)-N-(3-methoxybenzyl)-5-(2-(2-morpholinoethoxy)ethoxy)pyridin-2-amine; N-(3-(1H-imidazol-1-yl)benzyl)-5-(2-(3-(dimethylamino)phenoxy)ethoxy)-N-(3-methoxybenzyl)pyridin-2-amine; N-(3-(1H-imidazol-1-yl)benzyl)-N-(3-methoxybenzyl)-5-(2-morpholinoethoxy)pyridin-2-amine; N-(3-methoxybenzyl)-5-(2-(2-(3-methoxyphenoxy)ethoxy)ethoxy)-N-(4-morpholinobenzyl)pyridin-2-amine; 5-(2-(2-(benzyloxy)ethoxy)ethoxy)-N-(3-methoxybenzyl)-N-(4-morpholinobenzyl)pyridin-2-amine; N-(3-methoxybenzyl)-N-(4-morpholinobenzyl)-5-(2-(2-morpholinoethoxy)ethoxy)pyridin-2-amine; 5-(2-(3-(dimethylamino)phenoxy)ethoxy)-N-(3-methoxybenzyl)-N-(4-morpholinobenzyl)pyridin-2-amine; N-(3-methoxybenzyl)-N-(4-morpholinobenzyl)-5-(2-morpholinoethoxy)pyridin-2-amine; N-(4-(1H-pyrazol-1-yl)benzyl)-N-(3-methoxybenzyl)-5-(2-(2-(3 methoxyphenoxy)ethoxy)ethoxy)pyridin-2-amine; N-(4-(1H-pyrazol-1-yl)benzyl)-5-(2-(2-(benzyloxy)ethoxy)ethoxy)-N-(3-methoxybenzyl)pyridin-2-amine; N-(4-(1H-pyrazol-1-yl)benzyl)-N-(3-methoxybenzyl)-5-(2-(2-morpholinoethoxy)ethoxy)pyridin-2-amine; N-(4-(1H-pyrazol-1-yl)benzyl)-5-(2-(3-(dimethylamino)phenoxy)ethoxy)-N-(3-methoxybenzyl)pyridin-2-amine; N-(4-(1H-pyrazol-1-yl)benzyl)-N-(3-methoxybenzyl)-5-(2-morpholinoethoxy)pyridin-2-amine; N-((1H-benzo[d]imidazol-6-yl)methyl)-N-(3-methoxybenzyl)-5-(2-(2-(3-methoxyphenoxy)ethoxy)ethoxy)pyridin-2-amine; N-((1H-benzo[d]imidazol-6-yl)methyl)-5-(2-(2-(benzyloxy)ethoxy)ethoxy)-N-(3-methoxybenzyl)pyridin-2-amine; N-((1H-benzo[d]imidazol-6-yl)methyl)-N-(3-methoxybenzyl)-5-(2-(2-morpholinoethoxy)ethoxy)pyridin-2-amine; N-((1H-benzo[d]imidazol-6-yl)methyl)-5-(2-(3-(dimethylamino)phenoxy)ethoxy)-N-(3-methoxybenzyl)pyridin-2-amine; and N-((1H-benzo[d]imidazol-6-yl)methyl)-N-(3-methoxybenzyl)-5-(2-morpholinoethoxy)pyridin-2-amine.

An eighth aspect which is the compound of the first aspect wherein the VLA-4 integrin agonist is selected from the group consisting essentially of N,N-bis(3-methoxybenzyl)-4-((2-(3-methoxyphenoxy)ethoxy)methyl)thiazol-2-amine; N,N-bis(3-methoxybenzyl)-4-((2-(2-morpholinoethoxy)ethoxy)methyl)thiazol-2-amine; N,N-bis(3-methoxybenzyl)-4-((2-(2-morpholinoethoxy)ethoxy)methyl)thiazol-2-amine; N,N-bis(3-methoxybenzyl)-4-((2-(2-(piperidin-1-yl)ethoxy)ethoxy)methyl)thiazol-2-amine; N,N-bis(3-methoxybenzyl)-4-(morpholinomethyl)thiazol-2-amine; N,N-bis(3-methoxybenzyl)-4-((2-morpholinoethoxy)methyl)thiazol-2-amine; N,N-bis(3-methoxybenzyl)-4-((2-(piperidin-1-yl)ethoxy)methyl)thiazol-2-amine; N-(3-methoxybenzyl)-N-(3-(2-methoxyethoxy)benzyl)-4-(morpholinomethyl)thiazol-2-amine; N-(4-(1H-pyrazol-1-yl)benzyl)-N-(3-methoxybenzyl)-4-(morpholinomethyl)thiazol-2-amine; N-(3-(1H-pyrazol-1-yl)benzyl)-N-(3-methoxybenzyl)-4-(morpholinomethyl)thiazol-2-amine; N,N-bis(3-methoxybenzyl)-4-(2-morpholinoethyl)thiazol-2-amine; N,N-bis(3-methoxybenzyl)-4-((2-morpholinoethoxy)methyl)oxazol-2-amine; N,N-bis(3-methoxybenzyl)-4-((tetrahydro-2H-pyran-4-ylamino)methyl)thiazol-2-amine, N,N-bis(3-methoxybenzyl)-4-((2-(tetrahydro-2H-pyran-4-yloxy)ethoxy)methyl)thiazol-2-amine, and N-(4-(1H-pyrazol-1-yl)benzyl)-N-(3-methoxybenzyl)-4-((3-methoxybenzyloxy)methyl)thiazol-2-amine.

A ninth aspect which is a pharmaceutical composition comprising (i) the compound of the first aspect, or a pharmaceutically acceptable salt thereof, and (ii) a pharmaceutically acceptable carrier.

A tenth aspect which is a pharmaceutical composition comprising (i) the compound of the fifth aspect, or a pharmaceutically acceptable salt thereof, and (ii) a pharmaceutically acceptable carrier.

An eleventh aspect which is a pharmaceutical composition comprising (i) the compound of the sixth aspect, or a pharmaceutically acceptable salt thereof, and (ii) a pharmaceutically acceptable carrier.

A twelfth aspect which is a pharmaceutical composition comprising (i) the compound of the seventh aspect, or a pharmaceutically acceptable salt thereof, and (ii) a pharmaceutically acceptable carrier.

A thirteenth aspect which is a method comprising contacting integrin expressing cells with a VLA-4 integrin agonist having formula (I):

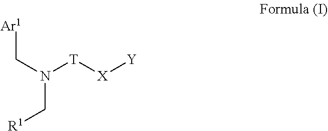

wherein, R¹ is hydrogen, lower alkyl, or aryl; Ar¹ is aryl, provided that when Ar¹ and R¹ are both phenyl, the 2 and 6 positions of each ring are substituted with hydrogen; T is aryl; X is a 1-20 atom linking chain containing any combination of —CH₂—, —C(O)—, —C(O)NR²—, —NR²—, —C(O)O— and —O—, each of which may independently be absent or present one or more times; provided that when X contains —C(O)N(R²)—, and —C(O)N(R²)— directly attaches to T, then R² is hydrogen; Y is selected from a group consisting essentially of hydrogen, (C₁-C₄)alkyl, aryl, cycloalkyl, N,N-dialkylamino, heterocyclyl, N,N-di(aralkyl)aminoaryl, (N-aralkyl)(N-alkyl)aminoaryl and a combination thereof, Ar¹ and R¹ may each be independently substituted or multiply substituted and selected at each occurrence from the group consisting essentially of lower alkyl, alkoxy, hydroxyalkyl, —OH, alkoxyalkyl, (C₁-C₃ alkyl)₂amino, alkoxyalkoxy, cycloalkyl, cycloalkylalkyl, aryl, heterocyclyl, alkylaryl, aralkyl, alkylheterocyclyl, and heterocyclylalkyl, groups; Y may be unsubstituted or substituted with one or more substituents selected from the group consisting essentially of halogen, lower alkyl, alkoxy, hydroxyalkyl, aliphatic acyl, —CF₃, oxo, —CN, —OH, alkoxyalkyl, (C₁-C₃ alkyl)₂amino, alkoxyalkoxy, cycloalkyl, cycloalkylalkyl, aryl, heterocyclyl, alkylaryl, aralkyl, alkylheterocyclyl, heterocyclylalkyl, and aryloxyalkyl groups; T may be unsubstituted or substituted with one or more substituents selected from the group consisting essentially of lower alkyl, alkoxy, hydroxyalkyl, aliphatic acyl, —CN, —OH, halogen, heterocycloyl, alkoxyalkyl, (C₁-C₃ alkyl)₂amino, alkoxyalkoxy, cycloalkyl, cycloalkylalkyl, heterocyclyl, alkylheterocyclyl, heterocyclylalkyl and a combination thereof, provided that when T is thiazolyl group and X and Y taken together form a C₁-C₈ alkyl group, then the 5-position of said thiazole ring is unsubstituted; substituents of T may be taken together with up to two atoms of X to form ring; and R² when present, may be selected independently and at each occurrence from the groups consisting essentially of hydrogen, lower alkyl, acetyl and methanesulfonyl.

A fourteenth aspect which is the method of the thirteenth aspect wherein the VLA-4 integrin agonist is selected from the group consisting essentially of N,N-bis(3-methoxybenzyl)-4-((2-(3-methoxyphenoxy)ethoxy)methyl)thiazol-2-amine; N,N-bis(3-methoxybenzyl)-4-((2-(2-morpholinoethoxy)ethoxy)methyl)thiazol-2-amine; N,N-bis(3-methoxybenzyl)-4-((2-(2-morpholinoethoxy)ethoxy)methyl)thiazol-2-amine; N,N-bis(3-methoxybenzyl)-4-((2-(2-(piperidin-1-yl)ethoxy)ethoxy)methyl)thiazol-2-amine; N,N-bis(3-methoxybenzyl)-4-(morpholinomethyl)thiazol-2-amine; N,N-bis(3-methoxybenzyl)-4-((2-morpholinoethoxy)methyl)thiazol-2-amine; N,N-bis(3-methoxybenzyl)-4-((2-(piperidin-1-yl)ethoxy)methyl)thiazol-2-amine; N-(3-methoxybenzyl)-N-(3-(2-methoxyethoxy)benzyl)-4-(morpholinomethyl)thiazol-2-amine; N-(4-(1H-pyrazol-1-yl)benzyl)-N-(3-methoxybenzyl)-4-(morpholinomethyl)thiazol-2-amine; N-(3-(1H-pyrazol-1-yl)benzyl)-N-(3-methoxybenzyl)-4-(morpholinomethyl)thiazol-2-amine; N,N-bis(3-methoxybenzyl)-4-(2-morpholinoethyl)thiazol-2-amine; N,N-bis(3-methoxybenzyl)-4-((2-morpholinoethoxy)methyl)oxazol-2-amine; N,N-bis(3-methoxybenzyl)-4-((tetrahydro-2H-pyran-4-ylamino)methyl)thiazol-2-amine, N,N-bis(3-methoxybenzyl)-4-((2-(tetrahydro-2H-pyran-4-yloxy)ethoxy)methyl)thiazol-2-amine, and N-(4-(1H-pyrazol-1-yl)benzyl)-N-(3-methoxybenzyl)-4-((3-methoxybenzyloxy)methyl)thiazol-2-amine.

A fifteenth aspect which is the method of any of the thirteenth through fifteenth aspects wherein the integrin expressing cells comprise neutrophils, eosinophils, basophils, mast cells, monocytes, macrophages, dendritic cells, natural killer cells, and lymphocytes, immune cells, embryonic stem cells, adult stem cells, progenitor cells, induced pluripotent stem cells, or a combination thereof.

A sixteenth aspect which is the method of any of the thirteenth through fifteenth aspects wherein the integrin expressing cells express a naturally occurring integrin.

A seventeenth aspect which is the method of any of the thirteenth through fifteenth aspects wherein the integrin expressing cells express a transgenic integrin.

An eighteenth aspect which is a complex formed between (i) an integrin expressing cell and a integrin agonist; and/or (ii) an integrin binding protein wherein the integrin agonist has general formula (I)

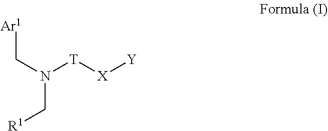

wherein, R¹ is hydrogen, lower alkyl, or aryl; Ar¹ is aryl, provided that when Ar¹ and R¹ are both phenyl, the 2 and 6 positions of each ring are substituted with hydrogen; T is aryl; X is a 1-20 atom linking chain containing any combination of —CH₂—, —C(O)—, —C(O)NR²—, —NR²—, —C(O)O— and —O—, each of which may independently be absent or present one or more times; provided that when X contains —C(O)N(R²)—, and —C(O)N(R²)— directly attaches to T, then R² is hydrogen; Y is selected from a group consisting essentially of hydrogen, (C₁-C₄)alkyl, aryl, cycloalkyl, N,N-dialkylamino, heterocyclyl, N,N-di(aralkyl)aminoaryl, (N-aralkyl)(N-alkyl)aminoaryl and a combination thereof, Ar¹ and R¹ may each be independently substituted or multiply substituted and selected at each occurrence from the group consisting essentially of lower alkyl, alkoxy, hydroxyalkyl, —OH, alkoxyalkyl, (C₁-C₃ alkyl)₂amino, alkoxyalkoxy, cycloalkyl, cycloalkylalkyl, aryl, heterocyclyl, alkylaryl, aralkyl, alkylheterocyclyl, and heterocyclylalkyl, groups; Y may be unsubstituted or substituted with one or more substituents selected from the group consisting essentially of halogen, lower alkyl, alkoxy, hydroxyalkyl, aliphatic acyl, —CF₃, oxo, —CN, —OH, alkoxyalkyl, (C₁-C₃ alkyl)₂amino, alkoxyalkoxy, cycloalkyl, cycloalkylalkyl, aryl, heterocyclyl, alkylaryl, aralkyl, alkylheterocyclyl, heterocyclylalkyl, and aryloxyalkyl groups; T may be unsubstituted or substituted with one or more substituents selected from the group consisting essentially of lower alkyl, alkoxy, hydroxyalkyl, aliphatic acyl, —CN, —OH, halogen, heterocycloyl, alkoxyalkyl, (C₁-C₃ alkyl)₂amino, alkoxyalkoxy, cycloalkyl, cycloalkylalkyl, heterocyclyl, alkylheterocyclyl, heterocyclylalkyl and a combination thereof, provided that when T is thiazolyl group and X and Y taken together form a C₁-C₈ alkyl group, then the 5-position of said thiazole ring is unsubstituted; substituents of T may be taken together with up to two atoms of X to form ring; and R² when present, may be selected independently and at each occurrence from the groups consisting essentially of hydrogen, lower alkyl, acetyl and methanesulfonyl.

A nineteenth aspect which is the complex of the eighteenth aspect wherein the VLA-4 integrin agonist is selected from the group consisting essentially of N,N-bis(3-methoxybenzyl)-4-((2-(3-methoxyphenoxy)ethoxy)methyl)thiazol-2-amine; N,N-bis(3-methoxybenzyl)-4-((2-(2-morpholinoethoxy)ethoxy)methyl)thiazol-2-amine; N,N-bis(3-methoxybenzyl)-4-((2-(2-morpholinoethoxy)ethoxy)methyl)thiazol-2-amine; N,N-bis(3-methoxybenzyl)-4-((2-(2-(piperidin-1-yl)ethoxy)ethoxy)methyl)thiazol-2-amine; N,N-bis(3-methoxybenzyl)-4-(morpholinomethyl)thiazol-2-amine; N,N-bis(3-methoxybenzyl)-4-((2-morpholinoethoxy)methyl)thiazol-2-amine; N,N-bis(3-methoxybenzyl)-4-((2-(piperidin-1-yl)ethoxy)methyl)thiazol-2-amine; N-(3-methoxybenzyl)-N-(3-(2-methoxyethoxy)benzyl)-4-(morpholinomethyl)thiazol-2-amine; N-(4-(1H-pyrazol-1-yl)benzyl)-N-(3-methoxybenzyl)-4-(morpholinomethyl)thiazol-2-amine; N-(3-(1H-pyrazol-1-yl)benzyl)-N-(3-methoxybenzyl)-4-(morpholinomethyl)thiazol-2-amine; N,N-bis(3-methoxybenzyl)-4-(2-morpholinoethyl)thiazol-2-amine; N,N-bis(3-methoxybenzyl)-4-((2-morpholinoethoxy)methyl)oxazol-2-amine; N,N-bis(3-methoxybenzyl)-4-((tetrahydro-2H-pyran-4-ylamino)methyl)thiazol-2-amine, N,N-bis(3-methoxybenzyl)-4-((2-(tetrahydro-2H-pyran-4-yloxy)ethoxy)methyl)thiazol-2-amine, and N-(4-(1H-pyrazol-1-yl)benzyl)-N-(3-methoxybenzyl)-4-((3-methoxybenzyloxy)methyl)thiazol-2-amine.

A twentieth aspect which is the compound of any of the eighteenth through nineteenth aspects wherein the integrin binding protein comprises vascular cell adhesion molecule-1 (VCAM 1), fibronectin, mucosal addressin cell adhesion molecule-1 (MAdCAM-1), intercellular adhesion molecule-1 (ICAM-1), intercellular adhesion molecule-2 (ICAM-2) or a combination thereof.

A twenty-first aspect which is a method of enhancing retention of exogenously-introduced cells at an in vivo target site in a mammal generally comprising: treating integrin-expressing cells in vitro with an agonist of integrin to produce agonist-treated cells; and introducing at least a portion the agonist-treated cells to an in vivo target site in a mammal; wherein a greater number of the agonist-treated cells remain at the in vivo target site when compared to the number of cells retained when the same integrin-expressing cells are not treated and introduced to the in vivo target site and wherein the agonist of integrin is a VLA-4 integrin agonist.

A twenty-second aspect which is the method of the twenty-first aspect wherein the integrin expressing cells comprise embryonic stem cells, adult stem cells, neutrophils, eosinophils, basophils, mast cells, monocytes, macrophages, dendritic cells, natural killer cells, and lymphocytes, immune cells progenitor cells, induced pluripotent stem cells, or a combination thereof.

A twenty-third aspect which is the method of any of the twenty-first through twenty-second aspects wherein the treated cells are injected directly or in proximity to a site of damaged vascular tissue, diseased vascular tissue or a combination thereof.

A twenty-fourth aspect which is the method of any of the twenty-first through twenty-third aspects wherein the target site comprises vascular cell adhesion molecule-1 (VCAM 1), fibronectin, mucosal address in cell adhesion molecule-1 (MAdCAM-1), intercellular adhesion molecule-1 (ICAM-1), or intercellular adhesion molecule-2 (ICAM-2).

A twenty-fifth aspect which is the method of any of the twenty-first through twenty-fourth aspects wherein the VLA-4 integrin agonist is selected from the group consisting essentially of N,N-bis(3-methoxybenzyl)-4-((2-(3-methoxyphenoxy)ethoxy)methyl)thiazol-2-amine; N,N-bis(3-methoxybenzyl)-4-((2-(2-morpholinoethoxy)ethoxy)methyl)thiazol-2-amine; N,N-bis(3-methoxybenzyl)-4-((2-(2-morpholinoethoxy)ethoxy)methyl)thiazol-2-amine; N,N-bis(3-methoxybenzyl)-4-((2-(2-(piperidin-1-yl)ethoxy)ethoxy)methyl)thiazol-2-amine; N,N-bis(3-methoxybenzyl)-4-(morpholinomethyl)thiazol-2-amine; N,N-bis(3-methoxybenzyl)-4-((2-morpholinoethoxy)methyl)thiazol-2-amine; N,N-bis(3-methoxybenzyl)-4-((2-(piperidin-1-yl)ethoxy)methyl)thiazol-2-amine; N-(3-methoxybenzyl)-N-(3-(2-methoxyethoxy)benzyl)-4-(morpholinomethyl)thiazol-2-amine; N-(4-(1H-pyrazol-1-yl)benzyl)-N-(3-methoxybenzyl)-4-(morpholinomethyl)thiazol-2-amine; N-(3-(1H-pyrazol-1-yl)benzyl)-N-(3-methoxybenzyl)-4-(morpholinomethyl)thiazol-2-amine; N,N-bis(3-methoxybenzyl)-4-(2-morpholinoethyl)thiazol-2-amine; N,N-bis(3-methoxybenzyl)-4-((2-morpholinoethoxy)methyl)oxazol-2-amine; N,N-bis(3-methoxybenzyl)-4-((tetrahydro-2H-pyran-4-ylamino)methyl)thiazol-2-amine; N,N-bis(3-methoxybenzyl)-4-((2-(tetrahydro-2H-pyran-4-yloxy)ethoxy)methyl)thiazol-2-amine; and N-(4-(1H-pyrazol-1-yl)benzyl)-N-(3-methoxybenzyl)-4-((3-methoxybenzyloxy)methyl)thiazol-2-amine.

A twenty-fifth aspect which is a medicament comprising (i) an integrin expressing cell and (ii) a VLA-4 integrin agonist wherein the VLA-4 integrin agonist is selected from the group consisting essentially of N,N-bis(3-methoxybenzyl)-4-((2-(3-methoxyphenoxy)ethoxy)methyl)thiazol-2-amine; N,N-bis(3-methoxybenzyl)-4-((2-(2-morpholinoethoxy)ethoxy)methyl)thiazol-2-amine; N,N-bis(3-methoxybenzyl)-4-((2-(2-morpholinoethoxy)ethoxy)methyl)thiazol-2-amine; N,N-bis(3-methoxybenzyl)-4-((2-(2-(piperidin-1-yl)ethoxy)ethoxy)methyl)thiazol-2-amine; N,N-bis(3-methoxybenzyl)-4-(morpholinomethyl)thiazol-2-amine; N,N-bis(3-methoxybenzyl)-4-((2-morpholinoethoxy)methyl)thiazol-2-amine; N,N-bis(3-methoxybenzyl)-4-((2-(piperidin-1-yl)ethoxy)methyl)thiazol-2-amine; N-(3-methoxybenzyl)-N-(3-(2-methoxyethoxy)benzyl)-4-(morpholinomethyl)thiazol-2-amine; N-(4-(1H-pyrazol-1-yl)benzyl)-N-(3-methoxybenzyl)-4-(morpholinomethyl)thiazol-2-amine; N-(3-(1H-pyrazol-1-yl)benzyl)-N-(3-methoxybenzyl)-4-(morpholinomethyl)thiazol-2-amine; N,N-bis(3-methoxybenzyl)-4-(2-morpholinoethyl)thiazol-2-amine; N,N-bis(3-methoxybenzyl)-4-((2-morpholinoethoxy)methyl)oxazol-2-amine; N,N-bis(3-methoxybenzyl)-4-((tetrahydro-2H-pyran-4-ylamino)methyl)thiazol-2-amine; N,N-bis(3-methoxybenzyl)-4-((2-(tetrahydro-2H-pyran-4-yloxy)ethoxy)methyl)thiazol-2-amine; and N-(4-(1H-pyrazol-1-yl)benzyl)-N-(3-methoxybenzyl)-4-((3-methoxybenzyloxy)methyl)thiazol-2-amine.

A twenty-sixth aspect which is the medicament of the twenty-fifth aspect, further comprising an immunomodulator.

A twenty-seventh aspect which is a method of treatment comprising administering to a subject in need thereof the medicament of the twenty-fifth aspect.

A twenty-eighth aspect which is a method of treatment comprising administering to a subject in need thereof the medicament of the twenty-sixth aspect.

### EXAMPLES

### Adhesion Assays

### Reagents and Cell Lines

For in vitro cell adhesion assays, compounds were dissolved in DMSO to make a series of stock solutions such that a 1:100 dilution in assay buffer would yield the desired final working concentrations in 1% DMSO (vehicle). Human VCAM-1 and ICAM-1 were purchased from R&D Systems (Minneapolis, MN). The Jurkat (VCAM-1 assays) and HSB (ICAM-1 assays) cell lines were obtained from American Type Culture Collection (Manassas, VA) and were maintained in recommended culture media.

### Static Cell Adhesion Assays

Assays were performed as previously described in Vanderslice et al., (2010), Biochem Biophys Res Commun 400, 619-624; and Vanderslice et al., (2013) J Biol Chem 288, 19414-19428. VCAM-1 or ICAM-1 ligand in 50 μL of 50 mM Tris-HCl (pH 7.4), 150 mM NaCl, (TBS) was added to wells of a 96-well plate and allowed to coat overnight at 4° C. To maximize the window to evaluate agonist activity, a sub-optimal coating concentration of ligand was used, typically 0.5 and 5 μg/mL in 50 μL TBS for VCAM-1 and ICAM-1 respectively. This ligand concentration corresponded approximately to that which would yield <5% adhesion as determined by dose-response curves. Briefly, 2×10⁶ cells were labeled for 30 minutes with calcein-AM (Molecular Probes), washed, resuspended in binding buffer, and added to triplicate wells of ligand-coated plates (2×10⁵ cells/well) that had been blocked with 2% BSA. After a 30-minute incubation at 37° C., the plates were washed 3 times with binding buffer, the adherent cells were lysed, and fluorescence was measured on a Tecan Safire² plate reader. The binding buffer was PBS with 1 mM MgCl₂, 50% FBS for α4β1/VCAM-1 assays and PBS with 2 mM MgCl₂, 5 mM EGTA, 50% FBS for αLβ2/ICAM-1 assays. EC₅₀ is defined as the concentration of compound required to achieve 50% of the maximal response.

### Example 1: Preparation of 1-(2-thienyl)-N-(2-thienylmethyl)methanamine hydrochloride

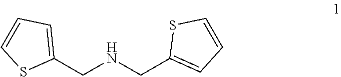 [SMILES: c1csc(CNCc2cccs2)c1]

Method A: To a solution of 2-thiophenemethylamine (3.87 g, 34.5 mmol) in toluene (69 mL), 2-thiophenecarboxaldehyde (3.90 g, 34.5 mmol) was added. The resulting solution was heated at vigorous reflux for 18 hours while removing the water formed by means of a Dean-Stark trap. The resulting mixture was cooled to room temperature and concentrated under reduced pressure. The residue was dissolved in anhydrous ethanol (69 mL), the mixture was cooled to 0° C., and sodium borohydride (2.71 g, 69 mmol) was added in portions over the course of 30 minutes. The mixture was allowed to warm to room temperature overnight, was poured over ice, and acidified slowly with aqueous hydrochloric acid (2N). The precipitate was filtered and dried under vacuum to give the hydrochloride salt as an off-white to tan solid (5.35 g). Compound 1 was generated from 1·HCl by extracting with three portions of ethyl acetate from aqueous sodium bicarbonate with methanol added for solubility. The organic layers were combined, dried, filtered and concentrated.

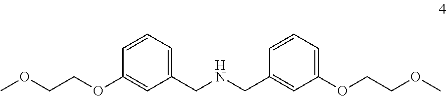

The procedure was also used to prepare bis(3-(2-methoxyethoxy)benzyl)amine (4) from 3-(2-methoxyethoxy)benzaldehyde and (3-(2-methoxyethoxy)phenyl)methanamine. Preparation of 3-(2-methoxyethoxy)benzaldehyde: To a solution of 3-hydroxybenzaldehyde (2.0 g, 16.37 mmol) in DMF (60 ml) potassium carbonate was added (9.0 g, 65.5 mmol) followed by bromoethyl methyl ether (3.41 g, 24.6 mmol) The mixture was stirred overnight. TLC indicated incomplete conversion of phenol by this time, and an additional 2 equivalents of the bromoethyl methyl ether was added and the mixture and stirred over 48 h following which time TLC indicated complete conversion of phenol. The mixture was concentrated, and the residue was dissolved in EtOAc and washed with water, brine, and dried over sodium sulfate giving 2.74 g of intermediate.

This procedure was also used to prepare 2-(3-(2-methoxyethoxy)phenyl)acetonitrile from 3-hydroxybenzonitrile.

Preparation of (3-(2-methoxyethoxy)phenyl)methanamine: To a solution of 2-(3-(2-methoxyethoxy)phenyl)acetonitrile in THF (40 ml) at 0° C. 16.1 ml (32.16 mmol) LAH (2M in THF, 16.1 ml, 32.2 mmol) was added dropwise. The solution which was allowed to warm to room temperature while stirring overnight. The mixture was again placed in an ice-water bath and 1.3 ml of water was added dropwise and the mixture was stirred for 10 minutes. Aqueous NaOH (20%) was added dropwise the mixture was stirred for another 10 minutes, and an additional 4.5 ml of water was added. The mixture was stirred 30 minutes, then was filtered through a pad of Celite, washing with diethyl ether. The filtrate was concentrated to give (3-(2-methoxyethoxy)phenyl)methanamine (2.7 g) as a colorless oil.

In a modification to this procedure, an ester was reduced to an alcohol. This modification was used to prepare (3-(bis(3-methoxybenzyl)amino)phenyl)methanol from ethyl 3-(bis(3-methoxybenzyl)amino)benzoate; and (4-(bis(3-methoxybenzyl)amino)phenyl)methanol from ethyl 4-(bis(3-methoxybenzyl)amino)benzoate.

### Example 2: Preparation of bis(3-methoxybenzyl)amine

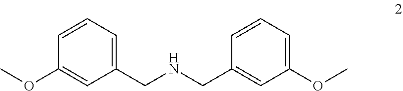

Method B: (Org. Biomol. Chem. 2012, pg. 7610). To a solution of 3-methoxybenzylamine (7.5 ml, 58.6 mmol) in absolute ethanol (30 ml), 3-methoxybenzyaldehyde was added (7.14 ml, 58.6 mmol) followed by anhydrous magnesium sulfate (1.5 g). The mixture was heated at reflux for 4 hours, then was allowed to cool to room temperature. Sodium borohydride (1.5 g, 39.7 mmol) was then added portion-wise to the mixture and stirred overnight. The flask was placed in an ice-water bath, and saturated ammonium chloride aqueous solution was added (50 ml) and stirred for 0.5 h. The ethanol was removed under vacuum, and remaining aqueous layer was extracted with DCM (3×25 ml). The combined extracts were washed with water and brine, and dried over sodium sulfate, filtered and concentrated. The residue was redissolved in EtOAc (150 ml) and 5.0 ml of c. HCl was added dropwise with stirring. The resulting precipitate was collected by vacuum filtration and washed with EtOAc, to give the dihydrochloride salt (14 g) as a white solid. Compound 2 was generated from 2·HCl salt by extracting with three portions of DCM from 2 M KOH. The organic layers were combined, dried, filtered and concentrated.

This procedure was used to prepare 3-((3-methoxybenzylamino)methyl)-N,N-dimethylaniline (3), ethyl 6-(3-methoxybenzylamino)nicotinate hydrochloride [97, MS (m/z): 287.2 (M+H)⁺] using ethyl 6-aminonicotinate and 3-methoxybenzaldehyde, N-((1H-indazol-5-yl)methyl)-1-(3-methoxyphenyl)methanamine, and N-(3-(1H-pyrazol-1-yl)benzyl)-1-(3-methoxyphenyl)methanamine.

Procedures from examples 1 and 2 may also be used to prepare: N-(3-methoxybenzyl)-1-(3-(pyrrolidin-1-yl)phenyl)methanamine; N-(3-(1H-imidazol-1-yl)benzyl)-1-(3-methoxyphenyl)methanamine; N-(3-methoxybenzyl)-1-(4-(pyrrolidin-1-yl)phenyl)methanamine; N-(3-methoxybenzyl)-1-(4-morpholinophenyl)methanamine; N-(3-methoxybenzyl)-1-(4-(4-methylpiperazin-1-yl)phenyl)methanamine; N-(3-methoxybenzyl)-1-(3-morpholinophenyl)methanamine; N-(3-methoxybenzyl)-1-(3-(4-methylpiperazin-1-yl)phenyl)methanamine; N-(4-(1H-pyrazol-1-yl)benzyl)-1-(3-methoxyphenyl)methanamine; N-(4-(1H-imidazol-1-yl)benzyl)-1-(3-methoxyphenyl)methanamine; N-((1H-benzo[d]imidazol-6-yl)methyl)-1-(3-methoxyphenyl)methanamine; N-((1H-indazol-6-yl)methyl)-1-(3-methoxyphenyl)methanamine; N-(3-methoxybenzyl)-1-(quinolin-6-yl)methanamine; and N-(3-methoxybenzyl)-1-(3-(2-methoxyethoxy)phenyl)methanamine.

### Example 3: General Preparation of 1,1-disubstituted ureas: 1,1-bis(3-methoxybenzyl)urea

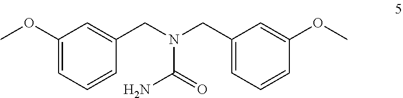

Method C: To a solution of bis(3-methoxybenzyl)amine (0.93 g, 3.6 mmol) in DCM (5 ml) under argon, trimethylsilyl isocyanate (0.73 ml, 5.4 mmol) was added. The mixture was stirred overnight and then diluted with DCM and washed with water (25 ml). The aqueous was extracted with DCM (2×20 ml) and the combined extracts washed with brine and dried with sodium sulfate, filtered and concentrated. The material was resubjected to the reaction conditions twice more, using the same amounts of solvent and reagents. A small amount of amine starting material remained, and the crude was then subjected to flash chromatography using a 0 to 20% acetone in DCM gradient to give 5, (0.8 g, 76% yield) as a pale yellow syrup.

This procedure was used to prepare 1,1-bis(3-(2-methoxyethoxy)benzyl)urea 6 from bis(3-(2-methoxyethoxy)benzyl)amine (4), 1,1-bis(thiophen-2-ylmethyl)urea (7) from 1-(2-thienyl)-N-(2-thienylmethyl)methanamine (1), 1,1-bis(4-methoxybenzyl)urea (8) from bis(4-methoxybenzyl)amine.

This same procedure of Example 3 may also be used to prepare the following urea intermediates: 1-(4-(dimethylamino)benzyl)-1-(4-methoxybenzyl)urea; 1-(4-methoxybenzyl)-1-(4-(pyrrolidin-1-yl)benzyl)urea; 1-(4-(1H-pyrazol-1-yl)benzyl)-1-(4-methoxybenzyl)urea; 1-(4-(1H-imidazol-1-yl)benzyl)-1-(4-methoxybenzyl)urea; 1-(4-methoxybenzyl)-1-(3-(pyrrolidin-1-yl)benzyl)urea; 1-(4-methoxybenzyl)-1-(3-morpholinobenzyl)urea; 1-(4-methoxybenzyl)-1-(3-(4-methylpiperazin-1-yl)benzyl)urea; 1-(4-methoxybenzyl)-1-(4-morpholinobenzyl)urea; 1-(4-methoxybenzyl)-1-(4-(4-methylpiperazin-1-yl)benzyl)urea; 1-(4-(1H-pyrazol-1-yl)benzyl)-1-(4-methoxybenzyl)urea; 1-(4-(1H-imidazol-1-yl)benzyl)-1-(4-methoxybenzyl)urea; 1-((1H-benzo[d]imidazol-6-yl)methyl)-1-(4-methoxybenzyl)urea; 1-((1H-indazol-6-yl)methyl)-1-(4-methoxybenzyl)urea; 1-(4-methoxybenzyl)-1-(quinolin-6-ylmethyl)urea; and 1-(4-methoxybenzyl)-1-(3-(2-methoxyethoxy)benzyl)urea.

### Example 4: Preparation of thioureas

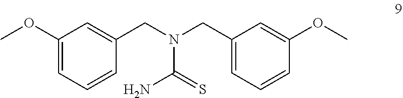

Method D: To a solution of 2-(bis(3-methoxybenzyl)amine, 2.2 g, 8.56 mmol) in DCM (50 mml) ethoxycarbonyl isothiocyanate (1.2 mL, 10.3 mmol) was added, and the solution stirred for 1.5 h., The mixture was washed with water (2×50 ml). The aqueous layers were extracted with DCM (50 ml). The combined organic layers were washed with brine, dried over sodium sulfate, filtered, concentrated (3.69 g), and was subjected to flash chromatography using a 10-20% gradient of EtOAc in hexanes to give 1,1-bis(3-methoxybenzyl)-3-(ethoxycarbonyl)thiourea (3.48 g). This material was dissolved in 140 ml of absolute ethanol, 85 ml (0.34 mol) of 4 M NaOH was added to the mixture and was allowed to reflux under argon for 48 hours at the end of which time TLC/HPLC indicated near complete conversion of starting material. The mixture was allowed to cool, and then ethanol was removed by rotary evaporation. Brine was added to the resulting suspension, which was then extracted with DCM until no more UV absorption was observed when spotting the extract on a TLC plate. The combined organics layers were washed with brine, dried over sodium sulfate, filtered, and concentrated. The residue was then subjected to flash chromatography using 17-34% gradient of EtOAc in hexanes to give 1,1-bis(3-methoxybenzyl)thiourea (1.9 g) (73% yield).

This procedure was used to prepare 1,1-bis(4-methoxybenzyl)thiourea 10 using (bis(4-methoxybenzyl)amine, 1-(3-(dimethylamino)benzyl)-1-(3-methoxybenzyl)thiourea 11 using 3-((3-methoxybenzylamino)methyl)-N,N-dimethylaniline 3, and 1,1-bis(3-(2-methoxyethoxy)benzyl)thiourea 12 using bis(3-(2-methoxyethoxy)benzyl)amine (4), 1-(4-(1H-imidazol-1-yl)benzyl)-1-(3-methoxybenzyl)thiourea from N-(4-(1H-imidazol-1-yl)benzyl)-1-(3-methoxyphenyl)methanamine; 1-(3-methoxybenzyl)-1-(quinolin-6-ylmethyl)thiourea from N-(3-methoxybenzyl)-1-(quinolin-6-yl)methanamine; 1-benzyl-1-(3-methoxybenzyl)thiourea from N— benzyl-1-(3-methoxyphenyl)methanamine; 1-((1H-indazol-6-yl)methyl)-1-(3-methoxybenzyl)thiourea from N-((1H-indazol-6-yl)methyl)-1-(3-methoxyphenyl)methanamine; 1-(3-methoxybenzyl)-1-(3-morpholinobenzyl)thiourea from N-(3-methoxybenzyl)-1-(3-morpholinophenyl)methanamine; 1-(3-(1H-imidazol-1-yl)benzyl)-1-(3-methoxybenzyl)thiourea from N-(3-(1H-imidazol-1-yl)benzyl)-1-(3-methoxyphenyl)methanamine; 1-(3-(1H-pyrazol-4-yl)benzyl)-1-(3-methoxybenzyl)thiourea from N-(3-(1H-pyrazol-4-yl)benzyl)-1-(3-methoxyphenyl)methanamine; 1-((1H-benzo[d]imidazol-6-yl)methyl)-1-(3-methoxybenzyl)thiourea from N-((1H-benzo[d]imidazol-6-yl)methyl)-1-(3-methoxyphenyl)methanamine; 1-(3-methoxybenzyl)-1-methylthiourea from N-methyl-3-methoxybenzylamine; 1-((1H-indazol-5-yl)methyl)-1-(3-methoxybenzyl)thiourea from N-((1H-indazol-5-yl)methyl)-1-(3-methoxyphenyl)methanamine; and 1-(3-(1H-pyrazol-1-yl)benzyl)-1-(3-methoxybenzyl)thiourea from N-(3-(1H-pyrazol-1-yl)benzyl)-1-(3-methoxyphenyl)methanamine.

The same procedure of Example 4 may also be used to prepare intermediates: 1-(4-(dimethylamino)benzyl)-1-(4-methoxybenzyl)thiourea; 1-(4-methoxybenzyl)-1-(4-(pyrrolidin-1-yl)benzyl)thiourea; 1-(4-(1H-pyrazol-1-yl)benzyl)-1-(4-methoxybenzyl)thiourea; 1-(4-(1H-imidazol-1-yl)benzyl)-1-(4-methoxybenzyl)thiourea; 1-(4-methoxybenzyl)-1-(3-(pyrrolidin-1-yl)benzyl)thiourea; 1-(4-methoxybenzyl)-1-(3-morpholinobenzyl)thiourea; 1-(4-methoxybenzyl)-1-(3-(4-methylpiperazin-1-yl)benzyl)thiourea; 1-(4-methoxybenzyl)-1-(4-morpholinobenzyl)thiourea; 1-(4-methoxybenzyl)-1-(4-(4-methylpiperazin-1-yl)benzyl)thiourea; 1-(4-(1H-pyrazol-1-yl)benzyl)-1-(4-methoxybenzyl)thiourea; 1-(4-(1H-imidazol-1-yl)benzyl)-1-(4-methoxybenzyl)thiourea; 1-((1H-benzo[d]imidazol-6-yl)methyl)-1-(4-methoxybenzyl)thiourea; 1-((1H-indazol-6-yl)methyl)-1-(4-methoxybenzyl)thiourea; 1-(4 methoxybenzyl)-1-(quinolin-6-ylmethyl)thiourea; and 1-(4-methoxybenzyl)-1-(3-(2-methoxyethoxy)benzyl)thiourea.

Preparation of 1-(3-methoxybenzyl)thiourea: To a solution of thiophosgene (3 mmol) in THF at 0° C. under argon, DIEA (6.6 mmol) was added followed by dropwise addition of 3-methoxybenzylamine (2 mmol). The mixture was allowed to gradually warm up to room temperature overnight then the mixture was diluted with saturated sodium bicarbonate and water. Ether (×3) was used to extract, and the combined extracts were washed with brine, dried over sodium sulfate, filtered, and concentrated. The residue was purified by automated silica gel chromatography to give 3-methoxybenzylisocyanate (0.5 mmol, 25% yield). This material was taken up in methanol and treated with ammonium hydroxide and the mixture was stirred overnight. The mixture was diluted with 2 M HCl, extracted with DCM. The DCM extracts were washed with brine, dried over sodium sulfate, filtered, and concentrated. The residue was purified by automated silica gel chromatography to give 1-(3-methoxybenzylthiourea (79% yield).

### Example 5—Preparation of ethyl 2-(bis(thiophen-2-ylmethyl)amino)oxazole-4-carboxylate

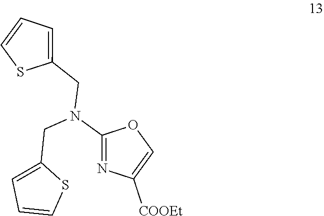 [SMILES: CCOC(=O)c1coc(N(Cc2cccs2)Cc2cccs2)n1]

(7, 0.54 g, 2.14 mmol) in absolute ethanol (10 ml) was added ethyl bromopyruvate (0.55 g, 2.78 mmol), and the mixture was refluxed. The reaction mixture was concentrated and the residue was redissolved in EtOAc (25 ml) and washed with water, brine, dried with magnesium sulfate, filtered, and concentrated. he residue was purified by flash chromatography using 10% EtOAc in hexanes to give ethyl 2-(bis(thiophen-2-ylmethyl)amino)oxazole-4-carboxylate (13, 0.38 g, 75% yield) as a clear oil (75% yield). ¹H NMR (DMSO-d₆, 300 MHz) δ 8.37 (s, 1H), 7.47 (dd, J=5.0 Hz, 1.2 Hz, 2H), 7.10-7.06 (m, 2H), 7.01-6.75 (m, 2H), 4.72 (s, 4H), 4.24 (q, J=7.2 Hz, 2H), 1.26 (t, J=6.8 Hz, 3H).

This procedure was also used to prepare ethyl 2-(bis(3-methoxybenzyl)amino)oxazole-4-carboxylate (14)¹H NMR (DMSO-d₆, 300 MHz) δ 8.28 (s, 1H), 7.26 (t, J=5.6 Hz, 2H), 6.88-6.78 (m, 6H), 4.57 (s, 4H), 4.21 (q, J=6.8 Hz, 2H), 3.71 (s, 6H), 1.17 (t, J=6.9 Hz, 3H) from 1,1-bis(3-methoxybenzyl)urea (5), ethyl 2-(bis(4-methoxybenzyl)amino)oxazole-4-carboxylate 15 LCMS m/z 397.3 (M+H)⁺ from 1,1-bis(4-methoxybenzyl)urea (8), and ethyl 2-(bis(3-(2-methoxyethoxy)benzyl)amino)oxazole-4-carboxylate 16 LCMS m/z 485.3 (M+H)⁺ from 1,1-bis(3-(2-methoxyethoxy)benzyl)urea (6).

This procedure was also used to prepare the following thiazoles from thioureas: methyl 2-(bis(3-methoxybenzyl)amino)-5-methylthiazole-4-carboxylate [101, MS (m/z): 413.3 (M+H)⁺] from 1,1-bis(3-methoxybenzyl)thiourea (9) and methyl 3-bromo-2-oxobutanoate; ethyl 2-(benzylamino)thiazole-4-carboxylate [103, MS (m/z): 263.0 (M+H)⁺] from 1-benzylthiourea; ethyl 2-(3-methoxybenzylamino)thiazole-4-carboxylate [105, MS m/z 293.3 (M+H)⁺] from 1-(3-methoxybenzyl)thiourea; ethyl 2-((3-methoxybenzyl)(methyl)amino)thiazole-4-carboxylate [106, MS (m/z): 307.1 (M+H)⁺) from 1-(3-methoxybenzyl)-1-methylthiourea; ethyl 2-((4-(1H-imidazol-1-yl)benzyl)(3-methoxybenzyl)amino)thiazole-4-carboxylate [113, MS (m/z): 449.2 (M+H)⁺] from 1-(4-(1H-imidazol-1-yl)benzyl)-1-(3-methoxybenzyl)thiourea; ethyl 2-((3-methoxybenzyl)(quinolin-6-ylmethyl)amino)thiazole-4-carboxylate [114, MS (m/z): 434.3 (M+H)⁺] from 1-(3-methoxybenzyl)-1-(quinolin-6-ylmethyl)thiourea; ethyl 2-(benzyl(3-methoxybenzyl)amino)thiazole-4-carboxylate [115, MS (m/z): 383.3 (M+H)⁺] from 1-benzyl-1-(3-methoxybenzyl)thiourea; ethyl 2-(((1H-indazol-5-yl)methyl)(3-methoxybenzyl)amino)thiazole-4-carboxylate [116, MS (m/z): 423.3 (M+H)⁺] from 1-((1H-indazol-6-yl)methyl)-1-(3-methoxybenzyl)thiourea; ethyl 2-((3-methoxybenzyl)(4-morpholinobenzyl)amino)thiazole-4-carboxylate [117, MS (m/z): 468.3 (M+H)⁺] from 1-(3-methoxybenzyl)-1-(3-morpholinobenzyl)thiourea; ethyl 2-115((3-(1H-imidazol-1-yl)benzyl)(3-methoxybenzyl)amino)thiazole-4-carboxylate [118, MS (m/z): 449.2 (M+H)⁺] from 1-(3-(1H-imidazol-1-yl)benzyl)-1-(3-methoxybenzyl)thiourea; ethyl 2-((3-(1H-pyrazol-1-yl)benzyl)(3-methoxybenzyl)amino)thiazole-4-carboxylate [119, MS (m/z): 449.2 (M+H)⁺] from 1-(3-(1H-pyrazol-4-yl)benzyl)-1-(3-methoxybenzyl)thiourea; and ethyl 2-(((1H-benzo[d]imidazol-6-yl)methyl)(3-methoxybenzyl)amino)thiazole-4-carboxylate [120, MS (m/z): 423.3 (M+H)⁺] from 1-((1H-benzo[d]imidazol-6-yl)methyl)-1-(3-methoxybenzyl)thiourea.

This procedure could also be performed using acetonitrile as the solvent instead of ethanol. This modification was used to prepare ethyl 2-(bis(3-methoxybenzyl)amino)thiazole-4-carboxylate [102, MS (m/z): 413.3 (M+H)⁺] using 1,1-bis(3-methoxybenzyl)thiourea (9).

The urea and thiourea intermediates of Examples 3 and 4 may also be used to cyclize to the respective oxazole or thiazole rings by way of Method E to give a corresponding 4-substituted-oxazole or 4-substituted-thiazole rings and for further conversion by way of Methods F through H to arrive at the analogous products for which one skilled in the art would readily recognize compounds of the present invention.

### Example 6—Preparation of 2-(bis(thiophen-2-ylmethyl)amino)-N-(3-methoxybenzyl)oxazole-4-carboxamide

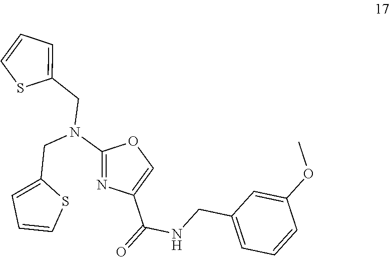 [SMILES: COc1cccc(CNC(=O)c2coc(N(Cc3cccs3)Cc3cccs3)n2)c1]

Method F: To a solution of 3-methoxybenzylamine (35 mg, 0.26 mmol) in toluene (1 ml) at 0° C., trioctylaluminum (1.17 mL, 25% by weight in hexanes, 0.56 mmol) was added dropwise. The mixture was stirred for ten minutes allowing it to warm to room temperature. A solution of ethyl 2-(bis(thiophen-2-ylmethyl)amino)oxazole-4-carboxylate 13 (70 mg, 0.2 mmol) was added dropwise and stirred for two hours, then refluxed overnight. The mixture was diluted with EtOAc and washed with 2 M HCl, water, brine. The organic layer was dried over sodium sulfate, filtered, and concentrated. The residue was purified by flash chromatography using 10% EtOAc-hexanes. 35 mg of product was recovered (40% yield) LCMS m/z 440.2 (M+H)⁺.

This method was used to generate 2-(bis(3-methoxybenzyl)amino)-N-(3-methoxybenzyl)oxazole-4-carboxamide 18 ¹H NMR (DMSO-d₆, 300 MHz), d 8.31 (s, 1H), 7.44-7.23 (m, 3H), 6.91-6.74 (m, 9H), 4.74 (m, 4H), 4.44-4.38 (m, 2H), 3.75 (s, 6H), 3.72 (s, 3H), from ethyl 2-(bis(3-methoxybenzyl)amino)oxazole-4-carboxylate (14) and 3-methoxybenzylamine, 2-(bis(3-methoxybenzyl)amino)-N-(4-methoxyphenethyl)oxazole-4-carboxamide (19) MS m/z 502.3 (M+H)⁺ from ethyl 2-(bis(3-methoxybenzyl)amino)oxazole-4-carboxylate (14) and 2-(4-methoxyphenyl)ethanamine, and 2-(bis(3-methoxybenzyl)amino)-N-(3-(4-methoxyphenyl)propyl)oxazole-4-carboxamide (20) LCMS m/z 516.3 (M+H)⁺, from ethyl 2-(bis(3-methoxybenzyl)amino)oxazole-4-carboxylate (14) and 3-(4-methoxyphenyl)propan-1-amine.

### Example 7—Preparation of 2-(bis(3-methoxybenzyl)amino)-N-(4-methoxyphenethyl)thiazole-4-carboxamide

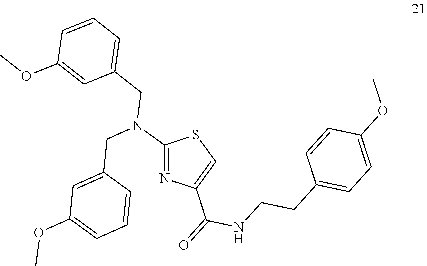 [SMILES: COc1ccc(CCNC(=O)c2csc(N(Cc3cccc(OC)c3)Cc3cccc(OC)c3)n2)cc1]

Method G: To a solution of bis(3-methoxybenzyl)amine 2 (206 mg, 0.8 mmol) and ethyl 2-bromothiazole-4-carboxylate (208 mg, 0.88 mmol) in toluene (3 ml) under argon, palladium acetate (42 mg), dppf (9 mg), and cesium carbonate (762 mg, 2.34 mmol) were added. The mixture was deoxygenated by toggling between vacuum and argon several times. The reaction mixture was heated to 100° C. in an oil bath overnight, cooled to room temperature, filtered through Celite and concentrated. The material was purified by flash chromatography using a 0-9% gradient of EtOAc in hexanes to afford ethyl 2-(bis(3-methoxybenzyl)amino)thiazole-4-carboxylate [102, MS (m/z): 413.3 (M+H)⁺].

This procedure was also used to prepare ethyl 2-(bis(3-methoxybenzyl)amino)oxazole-5-carboxylate from 1,1-bis(3-methoxybenzyl)amine (2) and ethyl 2-chlorooxazole-5-carboxylate.

To a solution of (102) (35 mg) in 3 ml MeOH 2 M NaOH (0.13 ml, 0.25 mmol) was added, and the mixture was stirred at room temperature for 30 minutes. The mixture diluted with EtOAc (10 ml) washed with 2 M HCl, brine, and the organic layer was dried over magnesium sulfate filtered and concentrated to give 2-(bis(3-methoxybenzyl)amino)thiazole-4-carboxylic acid (32 mg).

This procedure was also used to prepare 2-(bis(3-methoxybenzyl)amino)oxazole-5-carboxylic acid from ethyl 2-(bis(3-methoxybenzyl)amino)oxazole-5-carboxylate.

To a solution of 2-(bis(3-methoxybenzyl)amino)thiazole-4-carboxylic acid (32 mg) and 2-(4-methoxyphenyl)ethanamine (16 mg, 0.102 mg HBTU (27 mg) in 3 ml DMF was added DIEA (0.045 ml) and stirred at room temperature overnight. The DMF was removed under reduced pressure. The residue was resuspended in EtOAc, washed with brine, dried over magnesium sulfate, filtered, and concentrated. The resulting material was purified by flash chromatography using a 0-9% gradient of EtOAc in hexanes gradient to afford 27 mg MS m/z 518.3 (M+H)⁺ of the title compound.

This method was also used to synthesize 2-(bis(3-methoxybenzyl)amino)-N-(3-(4-methoxyphenyl)propyl)thiazole-4-carboxamide 23 [MS m/z 532.3 (M+H)⁺] from 2-(bis(3-methoxybenzyl)amino)thiazole-4-carboxylic acid and 3-(4-methoxyphenyl)propan-1-amine; and 2-(bis(3-methoxybenzyl)amino)-N-(3-(4-methoxyphenyl)propyl)oxazole-5-carboxamide [22, MS (m/z): 516.3 (M+H)⁺] from 2-(bis(3-methoxybenzyl)amino)oxazole-5-carboxylic acid and 3-(4-methoxyphenyl)propan-1-amine.

### Example 8—Preparation of (2-(bis(thiophen-2-ylmethyl)amino)oxazol-4-yl)methanol

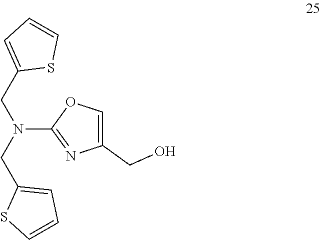 [SMILES: OCc1coc(N(Cc2cccs2)Cc2cccs2)n1]

Method H: To a solution of ethyl 2-(bis(thiophen-2-ylmethyl)amino)oxazole-4-carboxylate (13, 0.38 g, 1.1 mmol) in anhydrous THF (10 ml) under argon at 0° C., LiBH₄ was added (50 mg, 2.2 mmol) followed by MeOH (0.09 ml). The reaction was allowed to warm to room temperature with stirring overnight. The mixture was concentrated, redissolved in EtOAc, washed with water and brine, dried over magnesium sulfate, filtered, and concentrated. The residue was purified by flash chromatography to give (2-(bis(thiophen-2-ylmethyl)amino)oxazol-4-yl)methanol (25, 0.15 g, 47% yield). ¹H NMR (DMSO-d₆, 300 MHz) δ 7.46-7.43 (m, 2H), 7.09-7.02 (m, 2H), 7.01-6.94 (m, 2H), 5.04 (t, J 4.8 Hz, 1H), 4.67 (s, 4H), 4.25 (d, J=4.5 Hz, 2H).

This method was also used to prepare (2-(bis(4-methoxybenzyl)amino)oxazol-4-yl)methanol (26) MS m/z 355.3 (M+H)⁺ by the reduction of ethyl 2-(bis(3-methoxybenzyl)amino)oxazole-4-carboxylate (14).

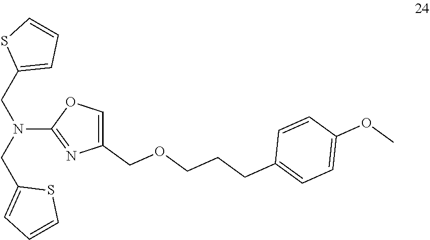 [SMILES: COc1ccc(CCCOCc2coc(N(Cc3cccs3)Cc3cccs3)n2)cc1]

### Preparation of 4-((3-(4-methoxyphenyl)propoxy)methyl)-N,N-bis(thiophen-2-ylmethyl)oxazol-2-amine

To a solution of (2-(bis(thiophen-2-ylmethyl)amino)oxazol-4-yl)methanol 25 (65 mg, 0.21 mmol) and 1-(3-bromopropyl)-4-methoxybenzene (63 mg, 0.28 mmol) in DMF (1 ml), NaH (60% dispersion in mineral oil, 30 mg, 0.32 mmol) was added. The mixture was stirred overnight at room temperature. The DMF was removed by distillation, and the residue dissolved in EtOAc, washed with water, brine, dried over sodium sulfate, filtered, and concentrated. The resulting material was purified using flash chromatography to afford 4-((3-(4-methoxyphenyl)propoxy)methyl)-N,N-bis(thiophen-2-ylmethyl)oxazol-2-amine [24, 70 mg, 73% yield, MS m/z 455.2 (M+H)⁺].

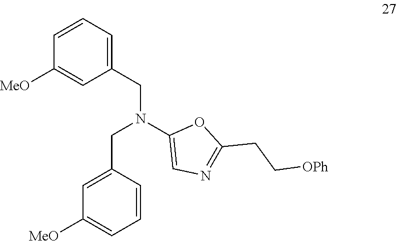 [SMILES: COc1cccc(CN(Cc2cccc(OC)c2)c2cnc(CCOc3ccccc3)o2)c1]

### Preparation of N,N-bis(3-methoxybenzyl)-2-(2-phenoxyethyl)oxazol-5-amine

To a solution of bis(3-methoxybenzyl)amine (2) (1.0 g, 3.4 mmol) and N-Boc-glycine (0.54 g, 3.1 mmol) in DMF (12 ml) at room temperature under argon, DIEA (1.3 ml, 7.44 mmol) and HBTU (1.53 g, 4.03 mmol) were added, and the mixture was allowed to stir overnight, diluted with 1:3 hexanes:EtOAc (50 ml), washed with 2 M HCl, water, saturated sodium bicarbonate (aq), water, brine, dried over sodium sulfate, filtered and concentrated. This material was purified by flash chromatography using a 10 to 20% EtOAc in hexanes gradient To give 1.27 g of tert-butyl 2-(bis(3-methoxybenzyl)amino)-2-oxoethylcarbamate was recovered (1.27 g, 99% yield). This material was dissolved in dioxane (5 mL), HCl in dioxane (4.0 M, 40 mmol) was added and the mixture was stirred overnight, then was concentrated. The residue was partitioned between EtOAc and saturated aqueous sodium bicarbonate, and the organic layer was washed with water and brine, dried over sodium sulfate, filtered and concentrated to give 2-amino-N,N-bis(3-methoxybenzyl)acetamide hydrochloride (0.90 g). 0.9 g of free amine (2-amino-N,N-bis(3-methoxybenzyl)acetamide) was recovered after removal of volatiles. To a solution of this material and 3-phenoxypropanoic acid (0.44 g, 2.58 mmol) in DMF (10 ml), HBTU (1.28 g), DIEA (0.85 ml, 6.2 mmol) were added. The mixture was stirred overnight and concentrated, and the residue was taken up in 1:1 EtOAc:hexanes and was washed with saturated aqueous sodium bicarbonate, water (×2) and brine. The organic layer was dried over sodium sulfate, filtered and concentrated. The residue was purified by and flash chromatography utilizing a 30-40% EtOAc in hexanes gradient to give N-(2-(bis(3-methoxybenzyl)amino)-2-oxoethyl)-3-phenoxypropanamide (955 mg, 80% yield) To a solution of this material (955 mg, 2.07 mmol) in DCM and TEA (0.91 mL), triphenylphosphine dibromide (1.08 g, 1.2 equivalents) was added. The mixture heated to reflux for 2 hours, then was diluted with hot hexanes (20 ml) and the resulting precipitate was separated by vacuum filtration. The residue was extracted with hot hexanes three times, and the combined hexanes extracts were concentrated. The residue was purified by flash chromatography using a 20-30% EtOAc in hexanes gradient to give N,N-bis(3-methoxybenzyl)-2-(2-phenoxyethyl)oxazol-5-amine [27, 180 mg, MS (m/z) 445.3 (M+H)⁺].

### Example 9—Preparation of 4-(chloromethyl)-N,N-bis(3-methoxybenzyl)thiazol-2-amine

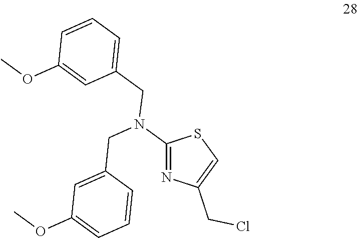 [SMILES: COc1cccc(CN(Cc2cccc(OC)c2)c2nc(CCl)cs2)c1]

Method I. A solution of 1,1-bis(3-methoxybenzyl)thiourea (9, 1.9 g, 6.0 mmol) and 1,3-dichloroacetone (0.76 g, 6.0 mmol) in MeCN (40 ml) was heated to 70° C. for 1.5 h then was allowed to cool to room temperature. and the mixture was diluted with DCM and washed with saturated sodium bicarbonate solution (aq). The combined organic layers were washed with brine, dried over sodium sulfate, filtered, and concentrated. The residue was purified by flash chromatography using a gradient of 5 to 10% EtOAc in hexanes, to give 4-(chloromethyl)-N,N-bis(3-methoxybenzyl)thiazol-2-amine (28, 1.58 g, 68% yield) of as a clear oil.

This method was used to prepare 4-(chloromethyl)-N,N-bis(4-methoxybenzyl)thiazol-2-amine from 1,1-bis(4-methoxybenzyl)thiourea (10) and 4-(chloromethyl)-N,N-bis(3-(2-methoxyethoxy)benzyl)thiazol-2-amine from 1,1-bis(3-(2-methoxyethoxy)benzyl)thiourea (12) N-(4-(1H-pyrazol-1-yl)benzyl)-4-(chloromethyl)-N-(3-methoxybenzyl)thiazol-2-amine from −1-(4-(1H-pyrazol-1-yl)benzyl)-1-(3-methoxybenzyl)thiourea; 4-(chloromethyl)-N-(3-(dimethylamino)benzyl)-N-(3-methoxybenzyl)thiazol-2-amine from 1-(3-(dimethylamino)benzyl)-1-(3-methoxybenzyl)thiourea; 4-(chloromethyl)-N-(3-methoxybenzyl)-N-methylthiazol-2-amine from 1-(3-methoxybenzyl)-1-methylthiourea; 4-(chloromethyl)-N-(3-methoxybenzyl)-N-(3-(2-methoxyethoxy)benzyl)thiazol-2-amine from 1-(3-methoxybenzyl)-1-(3-(2-methoxyethoxy)benzyl)thiourea; 4-(chloromethyl)-N-(3-methoxybenzyl)-N-(quinolin-6-ylmethyl)thiazol-2-amine from 1-(3-methoxybenzyl)-1-(quinolin-6-ylmethyl)thiourea; N-((1H-indazol-5-yl)methyl)-4-(chloromethyl)-N-(3-methoxybenzyl)thiazol-2-amine from 1-((1H-indazol-5-yl)methyl)-1-(3-methoxybenzyl)thiourea; 4-(chloromethyl)-N-(3-methoxybenzyl)-N-(3-morpholinobenzyl)thiazol-2-amine from 1-(3-methoxybenzyl)-1-(3-morpholinobenzyl)thiourea; N-(3-(1H-imidazol-1-yl)benzyl)-4-(chloromethyl)-N-(3-methoxybenzyl)thiazol-2-amine from 1-(3-(1H-imidazol-1-yl)benzyl)-1-(3-methoxybenzyl)thiourea; N-(3-(1H-pyrazol-1-yl)benzyl)-4-(chloromethyl)-N-(3-methoxybenzyl)thiazol-2-amine from 1-(3-(1H-pyrazol-1-yl)benzyl)-1-(3-methoxybenzyl)thiourea; N-benzyl-4-(chloromethyl)thiazol-2-amine; and 4-(chloromethyl)-N-(3-methoxybenzyl)thiazol-2-amine from 1-(3-methoxybenzyl)thiourea. A slight modification to the procedure used chloroacetone instead of 1,3-dichloroacetone. This modification was used to prepare: furnished N,N-bis(3-methoxybenzyl)-4-methylthiazol-2-amine [29, MS m/z 355.2 (M+H)⁺ from 1,1-bis(3-methoxybenzyl)thiourea (9); N,N-bis(3-(2-methoxyethoxy)benzyl)-4-methylthiazol-2-amine [30, MS (m/z): 443.3 (M+H)⁺] from 1,1-bis(3-(2-methoxyethoxy)benzyl)thiourea (12). N-(4-(1H-pyrazol-1-yl)benzyl)-N-(3-methoxybenzyl)-4-methylthiazol-2-amine [98, MS (m/z): 391.3 μM+H]+] from 1-(4-(1H-pyrazol-1-yl)benzyl)-1-(3-methoxybenzyl)thiourea; N-(3-methoxybenzyl)-4-methylthiazol-2-amine [99, MS (m/z): 235.2 (M+H)⁺] from 1-(3-methoxybenzyl)thiourea; and N-(3-methoxybenzyl)-N,4-dimethylthiazol-2-amine [108, MS (m/z): 249.2 (M+H)⁺] from 1-(3-methoxybenzyl)-1-methylthiourea.

This procedure was further modified by reacting 1-bromo-4-morpholinobutan-2-one hydrobromide salt (Djerassi, C.; Scholz, C. R. Brominations with pyridine hydrobromide perbromide, C₅H5N·HBr·Br₂. J. Am. Chem. Soc. 1948, 70, 417) with (9) to give N,N-bis(3-methoxybenzyl)-4-(2-morpholinoethyl)thiazol-2-amine [84, MS (m/z): 454.3 (M+H)⁺].

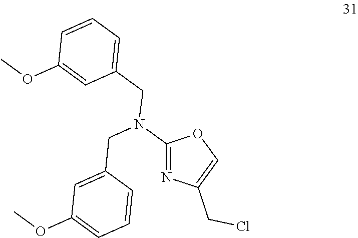 [SMILES: COc1cccc(CN(Cc2cccc(OC)c2)c2nc(CCl)co2)c1]

### Preparation of 4-(chloromethyl)-N,N-bis(3-methoxybenzyl)oxazol-2-amine

A flask containing 1,1-bis(3-methoxybenzyl)urea 5 (1.1 g, 3.7 mmol) and 1,3-dichloroacetone (0.47 g, 3.7 mmol) in DMF (15 ml) was placed in an oil bath pre-heated to 105° C. The mixture was stirred at temperature for 5 hours, cooled to room temperature, diluted with 50 ml of saturated sodium bicarbonate (aq), and extracted with diethyl ether (4×20 ml). The combined organic layers were washed with water (50 ml), brine, and dried over sodium sulfate, filtered and concentrated to give 1.28 g crude material. This was subjected to flash chromatography using a 5 to 20% gradient of EtOAc in hexanes, yielding 4-(chloromethyl)-N,N-bis(3-methoxybenzyl)oxazol-2-amine (452 mg, 33% isolated yield).

Alternatively, 1-chloro-3,3,3-trimethylacetone could be substituted for 1-3-dichloroacetone to produce 4-(tert-butyl)-N,N-bis(3-methoxybenzyl)oxazol-2-amine amine [MS (m/z): 397.3 (M+H)⁺].

### Example 10—Preparation of 2-(2-((2-(bis(3-methoxybenzyl)amino)thiazol-4-yl)methoxy)ethoxy)ethanol

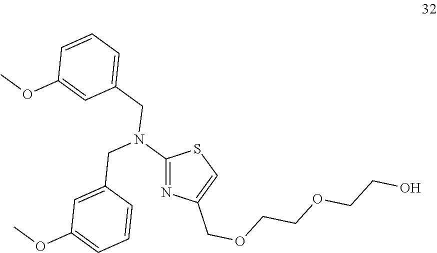 [SMILES: COc1cccc(CN(Cc2cccc(OC)c2)c2nc(COCCOCCO)cs2)c1]

Method J: To a flask containing potassium hydroxide (1.51 g 27 mmol), (pulverized to powder), diethylene glycol (20 ml) was added, and the mixture was heated at 90° C. for 0.5 h, resulting in a thick, homogenous mixture. This mixture was then added to a flask containing of 4-(chloromethyl)-N,N-bis(3-methoxybenzyl)thiazol-2-amine (28, 1.05 g, 2.7 mmol), followed by potassium iodide (90 mg, 0.54 mmol) to the mixture. The mixture was stirred for 48 hours, diluted with saturated 100 ml ammonium chloride (aq) and extracted with diethyl (7×25 ml). The combined organic extracts were washed with water (100 ml), brine, and dried over sodium sulfate, filtered and concentrated to give 2-(2-((2-(bis(3-methoxybenzyl)amino)thiazol-4-yl)methoxy)ethoxy)ethanol (32, 1.18 g, 95% yield).

The method was used to prepare 2-(2-((2-(bis(4-methoxybenzyl)amino)thiazol-4-yl)methoxy)ethoxy)ethanol [34, MS (m/z): 459.2 (M+H)⁺] from 4-(chloromethyl)-N,N-bis(4-methoxybenzyl)thiazol-2-amine and 2-(2-((2-(bis(3-methoxybenzyl)amino)oxaazol-4-yl)methoxy)ethoxy)ethanol from 4-(chloromethyl)-N,N-bis(3-methoxybenzyl)ozazol-2-amine (31).

This method could also be used to prepare 2-(2-(2-((2-(bis(3-methoxybenzyl)amino)thiazol-4-yl)methoxy)ethoxy)ethoxy)ethanol from (28) and triethylene glycol; and 1-(2-(bis(3-methoxybenzyl)amino)thiazol-4-yl)-2,5,8,11-tetraoxatridecan-13-ol from (28) and tetraethylene glycol.

A slight modification to this procedure was used to prepare 2-((2-(bis(3-methoxybenzyl)amino)thiazol-4-yl)methoxy)ethanol [33, MS (m/z) 415.3 (M+H)⁺]. Ethylene glycol was used instead of diethylene glycol, THF (20% v/v) was used as a co-solvent, and the mixture was heated to 70° C.

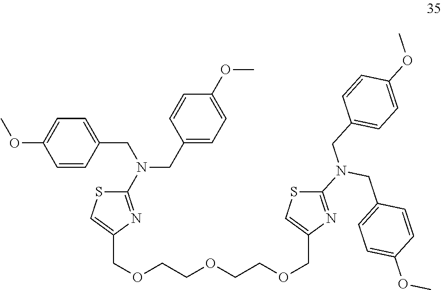

### Preparation of 4,4′-(2,2′-oxybis(ethane-2,1-diyl)bis(oxy))bis(methylene)bis(N,N-bis(4-methoxybenzyl)thiazol-2-amine)

To a flask containing sodium hydride (60% dispersion in mineral oil, 6.5 mg, 0.16 mmol), a solution of 2-(2-((2-(bis(4-methoxybenzyl)amino)thiazol-4-yl)methoxy)ethoxy)ethanol (34, 75 mg, 0.16 mmol) in THF (1.5 ml) was added. the mixture stirred for 30 minutes then a solution of 4-(chloromethyl)-N,N-bis(3-methoxybenzyl)thiazol-2-amine (28, 130 mg, 0.33 mmol) in THF (1.5 ml) was added followed by KI (5 mg, 0.032 mmol). The mixture was heated to 71° C. for 18 hours, cooled to room temperature, diluted with saturated aqueous ammonium chloride (10 ml), and extracted with DCM (2×10 ml). The combined organic layers were washed with water, brine, and dried over sodium sulfate, filtered, and concentrated. The residue was subjected to column chromatography using a 5 to 30% gradient of acetone in DCM to afford 4,4′-(2,2′-oxybis(ethane-2,1-diyl)bis(oxy))bis(methylene)bis(N,N-bis(4-methoxybenzyl)thiazol-2-amine) [35, 55 mg, 42% yield MS (m/z): 406.6 (M+2H)²⁺].

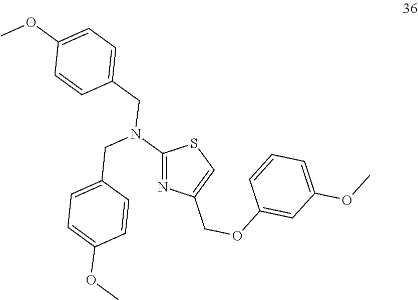

### Preparation of N,N-bis(4-methoxybenzyl)-4-((3-methoxyphenoxy)methyl)thiazol-2-amine

To a solution of 4-(chloromethyl)-N,N-bis(4-methoxybenzyl)thiazol-2-amine (64 mg, 0.16 mmol) and 3-methoxyphenol (25 mg, 0.2 mmol) in DMF (2 ml), potassium carbonate (46 mg, 0.33 mmol) and potassium iodide (25 mg, 0.16 mmol) were added. The mixture was heated to 60° C. for 18 h then diluted with water (20 ml) and extracted with EtOAc. The organic layers were washed with 2 M KOH, water (×2), brine, dried over sodium sulfate, filtered, and concentrated. The residue 190 mg was purified by flash chromatography using a 5-10% gradient of EtOAc in hexanes to obtain the N,N-bis(4-methoxybenzyl)-4-((3-methoxyphenoxy)methyl)thiazol-2-amine [36, 18 mg, 23% yield, MS (m/z): 477.2 (M+H)⁺].

The procedure was also used to prepare 4-((3-(dimethylamino)phenoxy)methyl)-N,N-bis(4-methoxybenzyl)thiazol-2-amine 37 LCMS m/z 490.2 (M+H) using 3-dimethylaminophenol in lieu of 3-methoxyphenol.

### Example 11—Preparation of N,N-bis(3-methoxybenzyl)-4-((3-methoxybenzyloxy)methyl)thiazol-2-amine

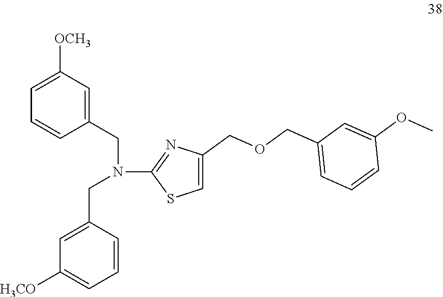 [SMILES: COc1cccc(COCc2csc(N(Cc3cccc(OC)c3)Cc3cccc(OC)c3)n2)c1]

Method K: 3-Methoxybenzyl alcohol (90 mg, 0.64 mmol) was dissolved in THF (1 ml). Sodium hydride (60% dispersion in mineral oil, 31 mg, 0.77 mmol) was placed in the flask followed by addition of tetraethylammonium iodide (66 mg, 0.257 mmol). The mixture was allowed to stir for 20 minutes and then a solution of 4-(chloromethyl)-N,N-bis(3-methoxybenzyl)thiazol-2-amine 28 (0.1 g) in THF (1 ml) was added. The mixture was allowed to stir for 18 h at 50° C. following which time the THF was removed by distillation, the crude dissolved in DCM and washed with water (×2), brine, dried over sodium sulfate and purified by chromatography to give 62 mg of the title compound (49% yield). LCMS m/z 491.2 (M+H)⁺.

The procedure was also used to prepare N,N-bis(3-methoxybenzyl)-4-((3-(4-methoxyphenyl)propoxy)methyl)thiazol-2-amine [39, MS (m/z): 519.3 (M+H)⁺] from 3-(4-methoxyphenyl)propan-1-ol; N,N-bis(3-methoxybenzyl)-4-((4-methoxyphenethoxy)methyl)thiazol-2-amine [40, MS (m/z): 505.3 (M+H)⁺] from 28 and 2-(4-methoxyphenyl)ethanol N,N-dimethylaminoethanol; N,N-bis(3-methoxybenzyl)-4-((2-morpholinoethoxy)methyl)oxazol-2-amine [87, MS (m/z): 468.3 (M+H)⁺] from 28 and 2-morpholinoethanol; 4-((2-(indolin-1-yl)ethoxy)methyl)-N,N-bis(3-methoxybenzyl)thiazol-2-amine [88, MS (m/z) 516.3 (M+H)⁺] from 28 and 2-(indolin-1-yl)ethanol; N-(4-(1H-pyrazol-1-yl)benzyl)-N-(3-methoxybenzyl)-4-((3-methoxybenzyloxy)methyl)thiazol-2-amine [104, MS (m/z): 527.3 (M+H)⁺] from N-(4-(1H-pyrazol-1-yl)benzyl)-4-(chloromethyl)-N-(3-methoxybenzyl)thiazol-2-amine and 3-methoxybenzyl alcohol; N-(3-methoxybenzyl)-4-((3-methoxybenzyloxy)methyl)-N-methylthiazol-2-amine [112, MS (m/z): 385.4 (M+H)⁺] from 4-(chloromethyl)-N-(3-methoxybenzyl)-N-methylthiazol-2-amine and 3-methoxybenzyl alcohol; N,N-bis(3-methoxybenzyl)-4-((tetrahydro-2H-pyran-4-yloxy)methyl)thiazol-2-amine [122, MS (m/z): 455.3 (M+H)⁺] from 28 and 4-hydroxytetrahydropyran; N,N-bis(3-methoxybenzyl)-4-((oxetan-3-yloxy)methyl)thiazol-2-amine [123, MS (m/z): 427.3 (M+H)⁺] from 28 and oxetan-3-ol; N,N-bis(3-methoxybenzyl)-4-(((tetrahydro-2H-pyran-4-yl)methoxy)methyl)thiazol-2-amine [124, MS (m/z): 469.3 (M+H)⁺] from (28) and (tetrahydro-2H-pyran-4-yl)methanol; N,N-bis(3-methoxybenzyl)-4-((oxetan-3-ylmethoxy)methyl)thiazol-2-amine from (28) and oxetan-3-ylmethanol; and N,N-bis(3-methoxybenzyl)-4-((2-(tetrahydro-2H-pyran-4-yloxy)ethoxy)methyl)thiazol-2-amine from (28) and 2-((tetrahydro-2H-pyran-4-yl)oxy)ethanol [131, MS (m/z): 499.2 (M+H)⁺].

### Example 12—Preparation of 4-((2-(2-(3-(dimethylamino)phenoxy)ethoxy)ethoxy)methyl)-N,N-bis(3-methoxybenzyl)thiazol-2-amine

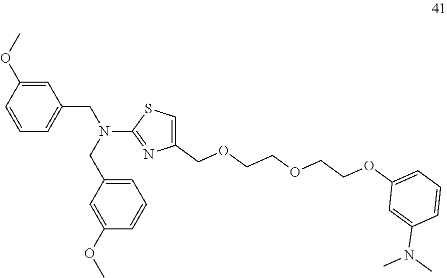 [SMILES: COc1cccc(CN(Cc2cccc(OC)c2)c2nc(COCCOCCOc3cccc(N(C)C)c3)cs2)c1]

Method L: To a solution of triphenylphosphine (129 mg, 0.49 mmol) in toluene (3 ml), DEAD (40% solution in toluene, 0.22 ml, 0.49 mmmol) was added. The mixture was stirred for ten minutes, then 3-dimethylaminophenol (45 mg, 0.033 mmol) was added. The mixture was stirred an additional fifteen minutes then a solution of 2-(2-((2-(bis(3-methoxybenzyl)amino)thiazol-4-yl)methoxy)ethoxy)ethanol (32, 150 mg, 0.33 mmol) in toluene (2 ml) was added. The mixture was stirred overnight. The next day toluene was removed by distillation. The residue was dissolved in DCM and washed with 2 M KOH. The combined organic extracts were washed with brine and dried over sodium sulfate, filtered, and concentrated. The crude was subjected to flash chromatography (20-40% EtOAc in hexanes gradient), and then reverse-phase silica gel to give 4-((2-(2-(3-(dimethylamino)phenoxy)ethoxy)ethoxy)methyl)-N,N-bis(3-methoxybenzyl)thiazol-2-amine (41, 41 mg, 22% yield) as a colorless oil. [MS (m/z): 289.7 (M+2H)²⁺].

This same procedure was used to prepare N,N-bis(3-methoxybenzyl)-4-((2-(2-(3-methoxyphenoxy)ethoxy)ethoxy)methyl)thiazol-2-amine [42, MS (m/z): 565.3 (M+H)⁺] from 32 and 3-methoxyphenol; N,N-bis(3-methoxybenzyl)-4-((2-(2-phenoxyethoxy)ethoxy)methyl)thiazol-2-amine [43, MS (m/z): 535.3 (M+H)⁺] from 32 and phenol; 4-((2-(2-(4-chlorophenoxy)ethoxy)ethoxy)methyl)-N,N-bis(3-methoxybenzyl)thiazol-2-amine amine [44, MS (m/z): 569.3 (M+H)⁺] from (32) and 4-chlorophenol; 3-(2-(2-((2-(bis(3-methoxybenzyl)amino)thiazol-4-yl)methoxy)ethoxy)ethoxy)benzonitrile [45, MS (m/z): 560.3 (M+H)⁺] from (32) and 3-cyanophenol; 4-((2-(2-(4-(dimethylamino)phenoxy)ethoxy)ethoxy)methyl)-N,N-bis(3-methoxybenzyl)thiazol-2-amine [45, MS (m/z): 560.3 (M+H)⁺] from (32) and 4-dimethylaminophenol.

N,N-bis(3-methoxybenzyl)-4-((2-(3-methoxyphenoxy)ethoxy)methyl)thiazol-2-amine [47, MS (m/z) 521.2 (M+H)⁺ from (33) and 3-methoxyphenol; 4-((2-(3-(dimethylamino)phenoxy)ethoxy)methyl)-N,N-bis(3-methoxybenzyl)thiazol-2-amine [48, MS (m/z): 267.8 (M+H)⁺] from (33) and 3-dimethylaminophenol; 4-((2-(4-chlorophenoxy)ethoxy)methyl)-N,N-bis(3-methoxybenzyl)thiazol-2-amine [49, MS (m/z): 525.2 (M+H)⁺] from (33) and 4-chlorophenol; 3-(2-((2-(bis(3-methoxybenzyl)amino)thiazol-4-yl)methoxy)ethoxy)benzonitrile [50, MS (m/z): 516.3 (M+H)⁺] from 33 and 3-cyanophenol; 4-((2-(4-(dimethylamino)phenoxy)ethoxy)methyl)-N,N-bis(3-methoxybenzyl)thiazol-2-amine [51, MS (m/z): 534.3 (M+H)⁺] from (33) and 4-dimethylaminophenol; N,N-bis(3-methoxybenzyl)-4-((2-phenoxyethoxy)methyl)thiazol-2-amine [52, MS (m/z): 491.2 (M+H)⁺] from (33) and phenol; N,N-bis(3-methoxybenzyl)-4-((2-(2-(3-methoxyphenoxy)ethoxy)ethoxy)methyl)oxazol-2-amine [72, MS (m/z): 549.4 (M+H)⁺] from 2-(2-((2-(bis(3-methoxybenzyl)amino)oxaazol-4-yl)methoxy)ethoxy)ethanol and 3-methoxyphenol.

### Example 13—Preparation of 4-((2-(2-(benzyloxy)ethoxy)ethoxy)methyl)-N,N-bis(3-methoxybenzyl)thiazol-2-amine

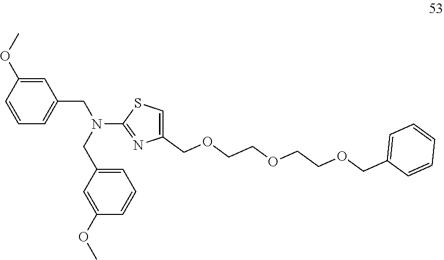 [SMILES: COc1cccc(CN(Cc2cccc(OC)c2)c2nc(COCCOCCOCc3ccccc3)cs2)c1]

Method M: To a suspension of sodium hydride (60% dispersion in mineral oil, 7.2 mg, 0.18 mmol) in THF (1 ml), a solution of 2-(2-((2-(bis(3-methoxybenzyl)amino)thiazol-4-yl)methoxy)ethoxy)ethanol (32, 84 mg, 0.18 mmol) in THF (2 ml) was added. The mixture was stirred for 30 minutes then benzyl chloride and potassium iodide were added. The mixture was heated to reflux overnight. The residue obtained after concentration of the reaction mixture was resuspended in DCM and washed with saturated ammonium chloride (aq). The organic layers were combined, washed with brine, dried over sodium sulfate, filtered and concentrated. The resulting material was purified by flash chromatography using a 20-40% gradient of EtOAc in hexanes to give 4-((2-(2-(benzyloxy)ethoxy)ethoxy)methyl)-N,N-bis(3-methoxybenzyl)thiazol-2-amine [53, 57 mg, 58% yield, MS (m/z): 549.3 (M+H)⁺].

The procedure was also used to prepare N,N-bis(3-methoxybenzyl)-4-((2-(2-((2-methylbenzyl)oxy)ethoxy)ethoxy)methyl)thiazol-2-amine [54, MS (m/z): 563.3 (M+H)⁺] from (32) and 2-methylbenzyl chloride; N,N-bis(3-methoxybenzyl)-4-((2-(2-((3-methoxybenzyl)oxy)ethoxy)ethoxy)methyl)thiazol-2-amine amine [55, MS (m/z): 579.3 (M+H)⁺ from (32) and 3-methoxybenzyl chloride; 4-((2-(2-((2-chlorobenzyl)oxy)ethoxy)ethoxy)methyl)-N,N-bis(3-methoxybenzyl)thiazol-2-amine [56, MS (m/z): 583.2 (M+H)⁺] from 32 and 2-chlorobenzyl bromide.

N,N-bis(3-methoxybenzyl)-4-((2-(3-methoxybenzyloxy)ethoxy)methyl)thiazol-2-amine [57, MS m/z 535.2 (M+H)⁺] from (33) and 3-methoxybenzyl chloride; 4-((2-(benzyloxy)ethoxy)methyl)-N,N-bis(3-methoxybenzyl)thiazol-2-amine [58, MS (m/z): 505.2 (M+H)⁺] from (33) and benzyl chloride; 4-((2-((2-chlorobenzyl)oxy)ethoxy)methyl)-N,N-bis(3-methoxybenzyl)thiazol-2-amine [59, MS (m/z): 539.2 (M+H)⁺] from (33) and 2-chlorobenzyl bromide; and N,N-bis(3-methoxybenzyl)-4-((2-((2-methylbenzyl)oxy)ethoxy)methyl)thiazol-2-amine amine [60, MS (m/z) 519.3 (M+H)⁺] from 33 and 2-methylbenzyl chloride.

### Example 14—Preparation of N,N-bis(3-methoxybenzyl)-4-((2-(2-morpholinoethoxy)ethoxy)methyl)thiazol-2-amine

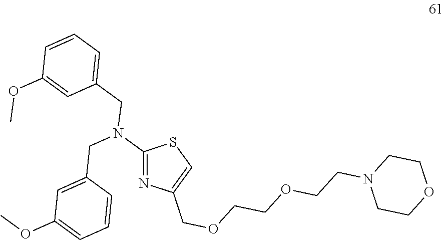 [SMILES: COc1cccc(CN(Cc2cccc(OC)c2)c2nc(COCCOCCN3CCOCC3)cs2)c1]

Method N: To a solution of 2-(2-((2-(bis(3-methoxybenzyl)amino)thiazol-4-yl)methoxy)ethoxy)ethanol 32 (229 mg, 0.5 mmol) in DCM (1 ml) anhydrous pyridine was added (0.16 ml, 2 mmol) and followed by tosyl chloride (191 mg, 1 mmol). The reaction was stirred under argon overnight. The mixture was diluted with DCM and washed with 2 M HCl. The aqueous layer was extracted again with DCM. The combined organic layers were washed with sodium bicarbonate (aq), brine, and dried over sodium sulfate, filtered and concentrated. The resulting crude was subjected to flash chromatography using a 25-50% gradient of EtOAc in hexanes to give 183 mg of 2-(2-((2-(bis(3-methoxybenzyl)amino)thiazol-4-yl)methoxy)ethoxy)ethyl 4-methylbenzenesulfonate (60% yield). To a flask containing 33 mg (0.054 mmol) of this material, 1 ml of morpholine was added. The mixture was heated to 85° C. for 1 h. The mixture was cooled to room temperature, diluted with 2 M KOH (10 ml) and extracted with DCM. The combined organic layers were washed with water (3×10 ml), brine, dried over sodium sulfate, filtered and concentrated to give N,N-bis(3-methoxybenzyl)-4-((2-(2-morpholinoethoxy)ethoxy)methyl)thiazol-2-amine (61, 25 mg, 89% yield, MS (m/z): 264.8 (M+2H)²⁺].

This two-step procedure was also used to prepare N,N-bis(3-methoxybenzyl)-4-((2-(2-(piperidin-1-yl)ethoxy)ethoxy)methyl)thiazol-2-amine [62, MS (m/z): 263.8 (M+2H)²⁺] from 32 and piperidine; and N,N-bis(3-methoxybenzyl)-4-((2-(2-(4-methylpiperazin-1-yl)ethoxy)ethoxy)methyl)thiazol-2-amine [63, MS (m/z): 271.3 (M+2H)⁺²] from 32 and N— methylpiperazine.

N,N-bis(3-methoxybenzyl)-4-((2-morpholinoethoxy)methyl)thiazol-2-amine [64, MS m/z 484.3 (M+H)⁺] from 33 and morpholine; N,N-bis(3-methoxybenzyl)-4-((2-(piperidin-1-yl)ethoxy)methyl)thiazol-2-amine [65, MS (m/z): 482.3 (M+H)⁺] from (33) and piperidine; and N,N-bis(3-methoxybenzyl)-4-((2-(4-methylpiperazin-1-yl)ethoxy)methyl)thiazol-2-amine [66, MS (m/z) 249.3 (M+2H)²⁺] from (33) and N-methylpiperazine.

### Example 15—Preparation of N,N-bis(3-methoxybenzyl)-4-(morpholinomethyl)thiazol-2-amine

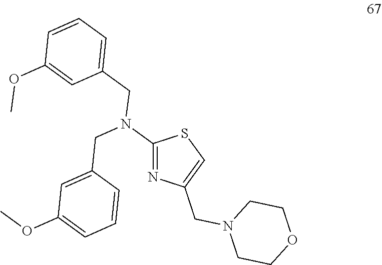 [SMILES: COc1cccc(CN(Cc2cccc(OC)c2)c2nc(CN3CCOCC3)cs2)c1]

Method O: A solution of 4-(chloromethyl)-N,N-bis(3-methoxybenzyl)thiazol-2-amine (28, 50 mg, 0.13 mmol) in morpholine (2 ml) was heated to 85° C. under argon for 2 hours. The mixture was allowed to cool to room temperature, and was partitioned between saturated sodium bicarbonate (aq) and DCM. The aqueous layer was extracted with DCM, and the combined organic layers were washed with water and brine, dried over sodium sulfate, filtered then concentrated. The residue was purified by flash chromatography using a 10-25% acetone in DCM gradient to give N,N-bis(3-methoxybenzyl)-4-(morpholinomethyl)thiazol-2-amine [67, 44 mg, 77% yield, MS (m/z) 440.2 (M+H)⁺].

This method was also used to prepare: N,N-bis(3-methoxybenzyl)-4-(piperidin-1-ylmethyl)thiazol-2-amine [68, MS (m/z): 438.3 (M+H)⁺] from 28 and piperidine; N,N-bis(3-methoxybenzyl)-4-((4-methylpiperazin-1-yl)methyl)thiazol-2-amine [69, MS (m/z): 453.3 (M+H)⁺] from (28) and N-methylpiperazine; N-(3-(dimethylamino)benzyl)-N-(3-methoxybenzyl)-4-(morpholinomethyl)thiazol-2-amine [73, MS (m/z): 453.3 (M+H)⁺ from 4-(chloromethyl)-N-(3-(dimethylamino)benzyl)-N-(3-methoxybenzyl)thiazol-2-amine and morpholine; N-(3-methoxybenzyl)-N-(3-(2-methoxyethoxy)benzyl)-4-(morpholinomethyl)thiazol-2-amine [74, MS (m/z) 484.3 (M+H)⁺] from 4-(chloromethyl)-N-(3-methoxybenzyl)-N-(3-(2-methoxyethoxy)benzyl)thiazol-2-amine and morpholine; N-(4-(1H-pyrazol-1-yl)benzyl)-N-(3-methoxybenzyl)-4-(morpholinomethyl)thiazol-2-amine [75, MS (m/z): 476.3 (M+H)⁺] from N-(4-(1H-pyrazol-1-yl)benzyl)-4-(chloromethyl)-N-(3-methoxybenzyl)thiazol-2-amine and morpholine; N-(3-methoxybenzyl)-4-(morpholinomethyl)-N-(quinolin-6-ylmethyl)thiazol-2-amine, chromatography was used to isolate compound, [76, MS (m/z) 461.3 (M+H)⁺] from 4-(chloromethyl)-N-(3-methoxybenzyl)-N-(quinolin-6-ylmethyl)thiazol-2-amine and morpholine; N-((1H-indazol-5-yl)methyl)-N-(3-methoxybenzyl)-4-(morpholinomethyl)thiazol-2-amine [77, MS (m/z): 450.3 (M+H)⁺] from N-((1H-indazol-5-yl)methyl)-4-(chloromethyl)-N-(3-methoxybenzyl)thiazol-2-amine and morpholine; N-(3-methoxybenzyl)-N-(3-morpholinobenzyl)-4-(morpholinomethyl)thiazol-2-amine, [78, MS (m/z) 495.3 (M+H]⁺ from 4-(chloromethyl)-N-(3-methoxybenzyl)-N-(3-morpholinobenzyl)thiazol-2-amine and morpholine; N-(3-(1H-imidazol-1-yl)benzyl)-N-(3-methoxybenzyl)-4-(morpholinomethyl)thiazol-2-amine [79, MS (m/z): 476.2 (M+H)⁺] from N-(3-(1H-imidazol-1-yl)benzyl)-4-(chloromethyl)-N-(3-methoxybenzyl)thiazol-2-amine and morpholine; N-(3-(1H-pyrazol-1-yl)benzyl)-N-(3-methoxybenzyl)-4-(morpholinomethyl)thiazol-2-amine [80, MS (m/): 476.3 (M+H)⁺] from N-(3-(1H-pyrazol-1-yl)benzyl)-4-(chloromethyl)-N-(3-methoxybenzyl)thiazol-2-amine and morpholine; N,N-bis(3-methoxybenzyl)-4-(morpholinomethyl)oxazol-2-amine [81, MS (m/z): 424.3 (M+H)⁺] from 4-(chloromethyl)-N,N-bis(3-methoxybenzyl)oxazol-2-amine and morpholine; N-benzyl-4-(morpholinomethyl)thiazol-2-amine [109, MS (m/z): 290.0 (M+H)⁺] from N-benzyl-4-(chloromethyl)thiazol-2-amine and morpholine; N-(3-methoxybenzyl)-4-(morpholinomethyl)thiazol-2-amine [110, MS (m/z): 320.2 (M+H)⁺] from 4-(chloromethyl)-N-(3-methoxybenzyl)thiazol-2-amine and morpholine; N-(3-methoxybenzyl)-N-methyl-4-(morpholinomethyl)thiazol-2-amine [111, MS (m/z): 334.2 (M+H)⁺] from 4-(chloromethyl)-N-(3-methoxybenzyl)-N-methylthiazol-2-amine and morpholine; N,N-bis(3-methoxybenzyl)-4-((methyl(tetrahydro-2H-pyran-4-yl)amino)methyl)thiazol-2-amine [125, MS (m/z): 468.2 (M+H)⁺] from (28) and N-methyltetrahydro-2H-pyran-4-amine; N,N-bis(3-methoxybenzyl)-4-((1-methylpiperidin-4-ylamino)methyl)thiazol-2-amine [126, MS (m/z) 467.2 (M+H)⁺] from (28) and 1-methylpiperidin-4-amine, N,N-bis(3-methoxybenzyl)-4-((tetrahydro-2H-pyran-4-ylamino)methyl)thiazol-2-amine from (28) and tetrahydro-2H-pyran-4-amine; and N,N-bis(3-methoxybenzyl)-4-(((1-methylpiperidin-4-yl)methylamino)methyl)thiazol-2-amine from (28) and (1-methylpiperidin-4-yl)methanamine.

In some cases, chromatography was not necessary. When purification was required, chromatography was sometimes done using amine functionalized silica gel.

### Example 16—Preparation of tert-butyl 2-(bis(3-methoxybenzyl)amino)-6,7-dihydrothiazolo[5,4-c]pyridine-5(4H)-carboxylate

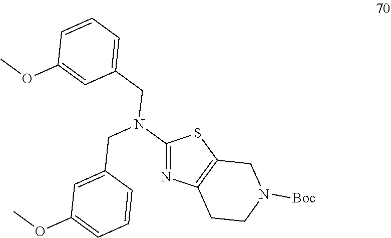 [SMILES: COc1cccc(CN(Cc2cccc(OC)c2)c2nc3c(s2)CN(C(=O)OC(C)(C)C)CC3)c1]

Method P:—To a solution of 1,1-bis(3-methoxybenzyl)thiourea (9, 125 mg, 0.4 mmol) in THF (3 ml) and TEA (0.1 ml, 0.71 mmol) at 0° C., tert-butyl 3-bromo-4-oxopiperidine-1-carboxylate (165 mg, 0.6 mmol) was added to the mixture, was removed from the ice bath and heated to 90° C. for 8 hrs, then was concentrated. The residue was dissolved in DCM (20 ml) which was then washed with water and brine, dried with sodium sulfate, filtered and concentrated. The resulting material (243 mg) was then purified by chromatography followed by trituration with hot hexanes to give tert-butyl 2-(bis(3-methoxybenzyl)amino)-6,7-dihydrothiazolo[5,4-c]pyridine-5(4H)-carboxylate [70, 110 mg, 56% yield, MS (m/z): 496.2 (M+H)⁺].

This method was also used to prepare N,N-bis(3-methoxybenzyl)-4-(4-methoxyphenethyl)thiazol-2-amine [71, MS (m/z) 475.3 (M+H)⁺] from (9) and 1-bromo-4-(4-methoxyphenyl)butan-2-one.

The method may also be used to prepare: N,N-bis(3-methoxybenzyl)-4-methyl-5-(morpholinomethyl)thiazol-2-amine using 3-bromo-4-morpholinobutan-2-one as the bromo-ketone starting material.

### Example 17: Preparation of 1-(bis(3-methoxybenzyl)amino)-4-((3-methoxybenzyl)oxy)-6-methylpyridin-2(1H)-one (127)

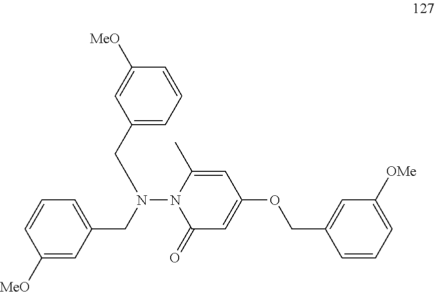 [SMILES: COc1cccc(COc2cc(C)n(N(Cc3cccc(OC)c3)Cc3cccc(OC)c3)c(=O)c2)c1]

To a solution of 1-amino-4-hydroxy-6-methylpyridin-2(1H)-one (102 mg, 0.73 mmol) and 3-methoxybenzyl bromide (0.36 mL, 2.56 mmol) in DMF (3.6 mL) under argon, sodium hydride (60: dispersion in mineral oil, 102 mg, 2.56 mmol) was added. The mixture was stirred at room temperature overnight, diluted with 1:1 hexanes:ethyl acetate, washed with water (3×) and brine. The organic layer was dried over MgSO₄, filtered and concentrated, and the residue was purified by automated silica gel chromatography to give 1-(bis(3-methoxybenzyl)amino)-4-((3-methoxybenzyl)oxy)-6-methylpyridin-2(1H)-one [127, 63 mg, MS (m/z): 501.3 (M+H)⁺] and 1-((3-methoxybenzyl)amino)-4-((3-methoxybenzyl)oxy)-6-methylpyridin-2(1H)-one [127 mg, MS (m/z): 381.4 (M+H)⁺].

This procedure was also used to prepare: N,N-bis(3-methoxybenzyl)-4-((3-methoxybenzyl)oxy)aniline [91, MS (m/z): 470.3 (M+H)⁺] from 4-aminophenol; and N,N-bis(3-methoxybenzyl)-3-((3-methoxybenzyl)oxy)aniline [92, MS (m/z): 370.3 (M+H)⁺] from 3-aminophenol.

In a modification to this procedure, only an amino group was bis-alkylated, and catalytic potassium iodide was added. This modification was used to prepare: 5-ethyl-N,N-bis(3-methoxybenzyl)-1,3,4-thiadiazol-2-amine [94, MS (m/z): 370.3 (M+H)⁺] and N-(5-ethyl-3-(3-methoxybenzyl)-1,3,4-thiadiazol-2(3H)-ylidene)-1-(3-methoxyphenyl)methanamine [93, MS (m/z): (M+H)⁺] from 5-ethyl-1,3,4-thiadiazol-2-amine; and ethyl 4-(bis(3-methoxybenzyl)amino)benzoate [85, MS (m/z): 406.3 (M+H)⁺] from ethyl 4-aminobenzoate; ethyl 3-(bis(3-methoxybenzyl)amino)benzoate [85, MS (m/z): 406.3 (M+H)⁺] from ethyl 3-aminobenzoate; ethyl 6-(bis(3-methoxybenzyl)amino)nicotinate [95, MS (m/z): 407.3 (M+H)⁺] from ethyl 6-aminonicotinate; and ethyl 2-(bis(3-methoxybenzyl)amino)pyrimidine-5-carboxylate [96, MS (m/z): 408.3 (M+H)⁺] from ethyl 2-aminopyrimidine-5-carboxylate. This modification could be further altered to alkylate a mono-substituted amino group. This altered modification was used to prepare ethyl 2-(isobutyl(3-methoxybenzyl)amino)thiazole-4-carboxylate [121, MS (m/z): 349.3 (M+H)⁺] from N-(3-methoxybenzyl)-4-(morpholinomethyl)thiazol-2-amine and isobutylbromide. In another modification to this procedure, only an alcohol was benzylated, again in using catalytic potassium iodide. This modification was used to prepare N,N-bis(3-methoxybenzyl)-3-(((3-methoxybenzyl)oxy)methyl)aniline [90, MS (m/z): 484.3 (M+H)⁺] from (3-(bis(3-methoxybenzyl)amino)phenyl)methanol; and N,N-bis(3-methoxybenzyl)-4-((3-methoxybenzyloxy)methyl)aniline [89, MS (m/z): 484.3 (M+H)⁺] from (4-(bis(3-methoxybenzyl)amino)phenyl)methanol.

The thiourea intermediates of Example 4 may also be used to cyclize to the corresponding thiazole ring by way of Example 7-16 (Methods G-P) to give a correspondingly substituted thiazole rings to arrive at the analogous products for which one skilled in the art would readily recognize as the desired products.

The methods presented here are not intended to limit the invention to the specific examples but to inform the person of ordinary skill. The inventions contemplates the different aryl rings may be used in place of those presented here.

Lipinski's rule of five (Lipinski, A., Drug Discovery Today: Technologies, Volume 1, Issue 4, 2004, Pages 337-341) discusses the advantages of molecular weights less than 500 and Log Ps less than five in order to effect greater possibility of oral availability and optimal water solubility. Other published compounds acting as VLA-4 integrin agonists have had less than desirable properties in this regard. One such example is THI0019.

### Structures of THI0019 and THI0349:

| | | | |
|---|---|---|---|
| THI0019 (33uM against VCAM) | 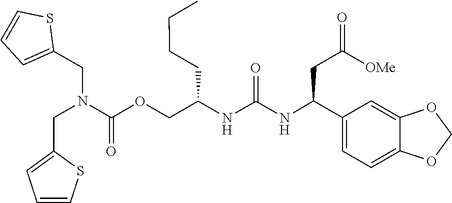 [SMILES: CCCC[C@@H](COC(=O)N(Cc1cccs1)Cc1cccs1)NC(=O)N[C@@H](CC(=O)OC)c1ccc2c(c1)OCO2] | LogP 5.42 | MW 601.73 |
| THI0349 | 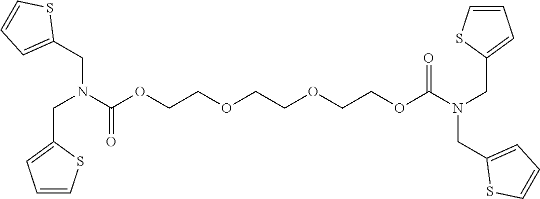 [SMILES: O=C(OCCOCCOCCOC(=O)N(Cc1cccs1)Cc1cccs1)N(Cc1cccs1)Cc1cccs1] | LogP 6.5 CLogP 6.4248 | MW 620.82 |

THI0019 (Vanderslice et al., (2013) J Biol Chem 288, 19414-19428) has reasonable Log P, but has significantly higher molecular weight than dictated by Lipinski's rules. THI0349, currently in clinical development, likewise has a molecular weight in excess of 600 and Log P of 6.5 or greater. THI0019 and THI0349 reported values were obtained under the assay conditions reported herein and ran higher in the presence of serum than those values presented previously. The following compounds illustrate the class of compounds disclosed herein having properties more closely aligned with those suggested by Lipinski's rule of five without sacrificing the range of potency relative to previously disclosed classes of compounds. Moreover, one skilled in the art would recognize that the log P values will not reflect the increased water solubility where an ionizable group is also present. The chart that follows illustrates calculated log P values as well as activities relative to THI0349 (average 11 uM) as a control, activities are shown by Groups relative to VCAM. Group I maintains at least the same potency or better up to more than twice that of the EC50 of THI0349 ran on the same plate; Group II is more than twice that of THI0349 but less than five times THI0349's EC50; and Group III more than five times the EC50 of THI0349:

| | | | | | LAD |
|---|---|---|---|---|---|
| | | | | | Raw Fluorescence |
| | | | | | Units against |
| | | | | | LFA-1; |
| | | | | | Dose 3mM; |
| | | | | Group | Ratio of raw |
| Comp. | | | | against | counts Compound/ |
| No. | Compound Structure | Log P | MW | VCAM | THI0349 |
| 13 | 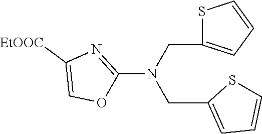 [SMILES: CCOC(=O)c1coc(N(Cc2cccs2)Cc2cccs2)n1] | 4.44 | 348.44 | I | 0.1 |
| 18 | 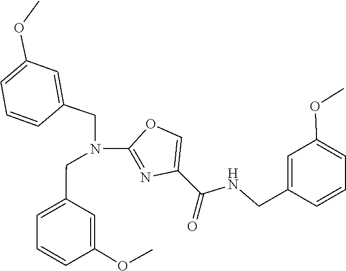 [SMILES: COc1cccc(CNC(=O)c2coc(N(Cc3cccc(OC)c3)Cc3cccc(OC)c3)n2)c1] | 4.81 | 487.55 | II | 0.17 |
| 19 | 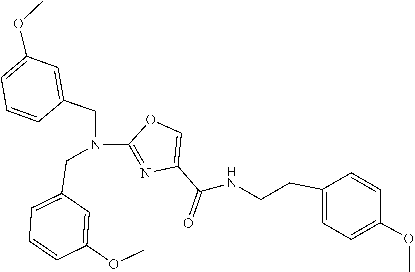 [SMILES: COc1ccc(CCNC(=O)c2coc(N(Cc3cccc(OC)c3)Cc3cccc(OC)c3)n2)cc1] | 5.09 | 501.57 | II | 0.12 |
| 20 | 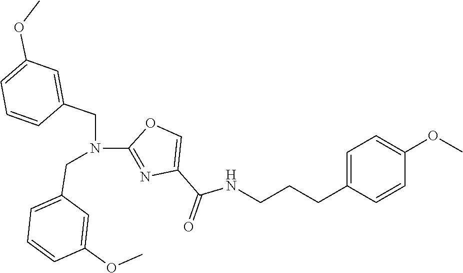 [SMILES: COc1ccc(CCCNC(=O)c2coc(N(Cc3cccc(OC)c3)Cc3cccc(OC)c3)n2)cc1] | 5.51 | 515.60 | II | 0.19 |
| 21 | 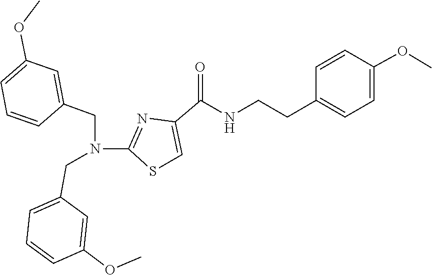 [SMILES: COc1ccc(CCNC(=O)c2csc(N(Cc3cccc(OC)c3)Cc3cccc(OC)c3)n2)cc1] | 6.45 | 517.64 | I | NR |
| 22 | 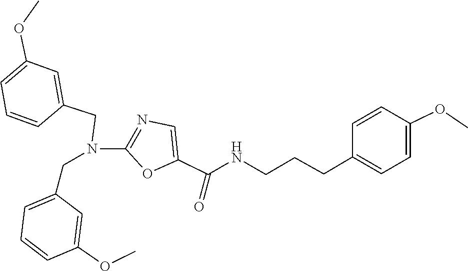 [SMILES: COc1ccc(CCCNC(=O)c2cnc(N(Cc3cccc(OC)c3)Cc3cccc(OC)c3)o2)cc1] | 5.14 | 515.60 | I | 0.69 |
| 23 | 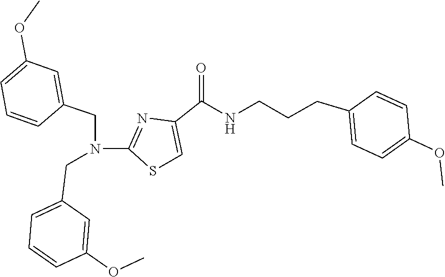 [SMILES: COc1ccc(CCCNC(=O)c2csc(N(Cc3cccc(OC)c3)Cc3cccc(OC)c3)n2)cc1] | 6.87 | 531.67 | I | 0.85 |
| 24 | 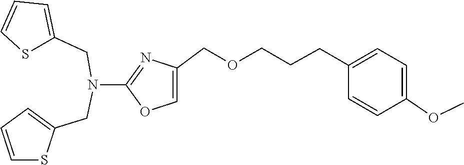 [SMILES: COc1ccc(CCCOCc2coc(N(Cc3cccs3)Cc3cccs3)n2)cc1] | 6.37 | 454.60 | II | 0.85 |
| 15 | 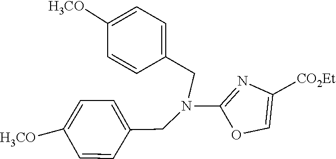 [SMILES: CCOC(=O)c1coc(N(Cc2ccc(OC)cc2)Cc2ccc(OC)cc2)n1] | 4.22 | 396.44 | III | 0.52 |
| 25 | 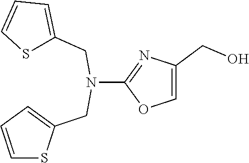 [SMILES: OCc1coc(N(Cc2cccs2)Cc2cccs2)n1] | 3.7 | 306.40 | II | 0.74 |
| 14 | 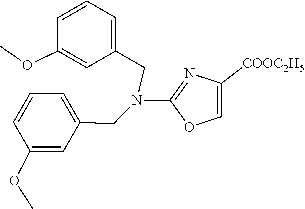 | 4.22 | 396.44 | I | 1.0 |
| 26 | 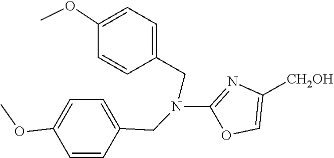 [SMILES: COc1ccc(CN(Cc2ccc(OC)cc2)c2nc(CO)co2)cc1] | 3.49 | 354.4 | I | 0.79 |
| 27 | 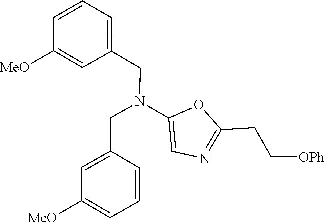 [SMILES: COc1cccc(CN(Cc2cccc(OC)c2)c2cnc(CCOc3ccccc3)o2)c1] | 5.38 | 442.52 | III | 0.28 |
| 16 | 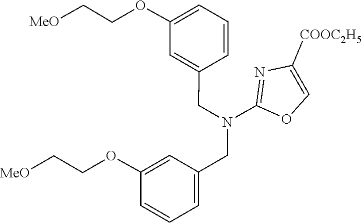 | 3.91 | 484.54 | II | 0.49 |
| 34 | 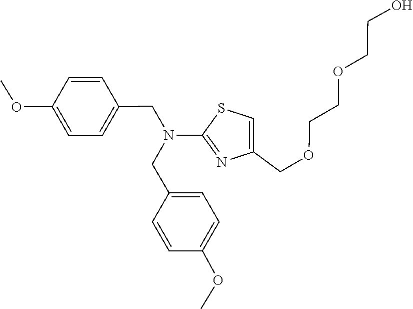 [SMILES: COc1ccc(CN(Cc2ccc(OC)cc2)c2nc(COCCOCCO)cs2)cc1] | 4.54 | 458.6 | III | 0.34 |
| 35 | 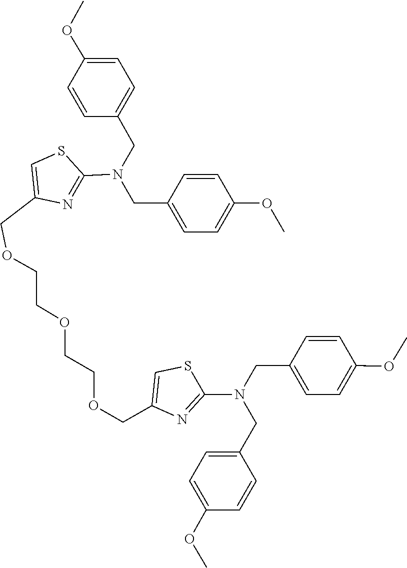 [SMILES: COc1ccc(CN(Cc2ccc(OC)cc2)c2nc(COCCOCCOCc3csc(N(Cc4ccc(OC)cc4)Cc4ccc(OC)cc4)n3)cs2)cc1] | 4.7 | 811.0 | II | 0.98 |
| 33 | 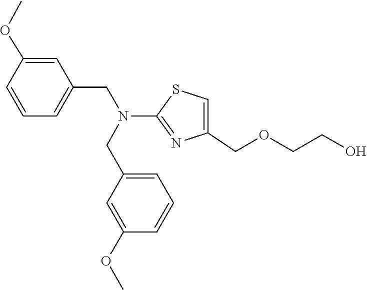 [SMILES: COc1cccc(CN(Cc2cccc(OC)c2)c2nc(COCCO)cs2)c1] | 4.7 | 414.52 | III | 0.31 |
| 42 | 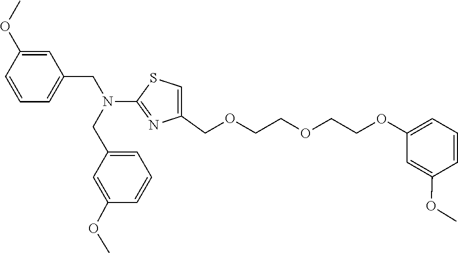 [SMILES: COc1cccc(CN(Cc2cccc(OC)c2)c2nc(COCCOCCOc3cccc(OC)c3)cs2)c1] | 6.6 | 564.69 | I | 1.6 |
| 41 | 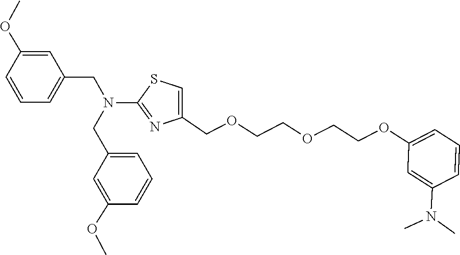 [SMILES: COc1cccc(CN(Cc2cccc(OC)c2)c2nc(COCCOCCOc3cccc(N(C)C)c3)cs2)c1] | 7.01 | 577.73 | I | 1.3 |
| 53 | 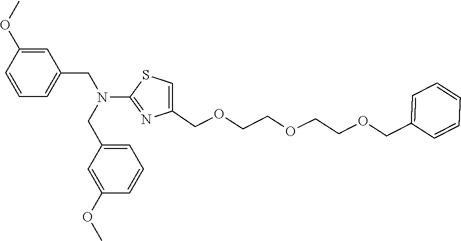 [SMILES: COc1cccc(CN(Cc2cccc(OC)c2)c2nc(COCCOCCOCc3ccccc3)cs2)c1] | 6.64 | 548.69 | I | 1.06 |
| 38 | 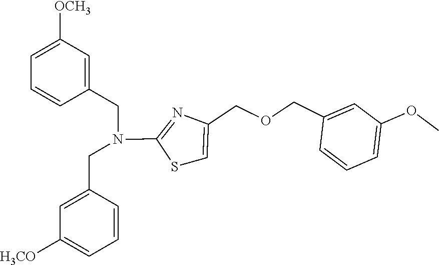 [SMILES: COc1cccc(COCc2csc(N(Cc3cccc(OC)c3)Cc3cccc(OC)c3)n2)c1] | 6.82 | 490.61 | III | 0.16 |
| 39 | 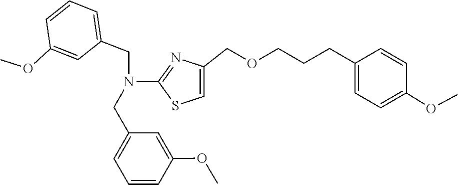 | 7.52 | 518.67 | III | 0.23 |
| 47 | 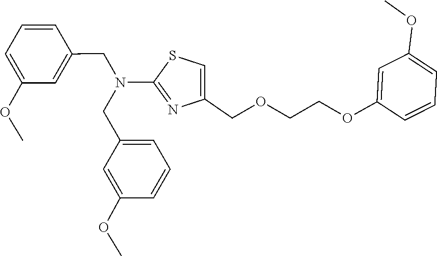 [SMILES: COc1cccc(CN(Cc2cccc(OC)c2)c2nc(COCCOc3cccc(OC)c3)cs2)c1] | 6.75 | 520.64 | I | 0.7 |
| 48 | 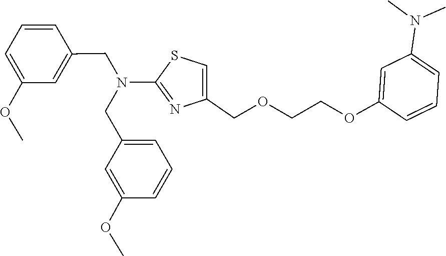 [SMILES: COc1cccc(CN(Cc2cccc(OC)c2)c2nc(COCCOc3cccc(N(C)C)c3)cs2)c1] | 7.16 | 533.68 | I | 0.43 |
| 58 | 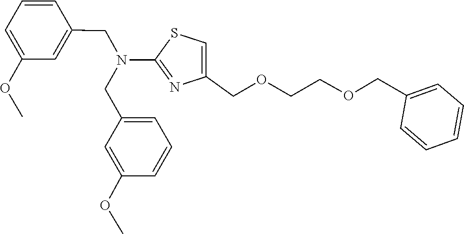 [SMILES: COc1cccc(CN(Cc2cccc(OC)c2)c2nc(COCCOCc3ccccc3)cs2)c1] | 6.79 | 504.64 | I | 0.48 |
| 59 | 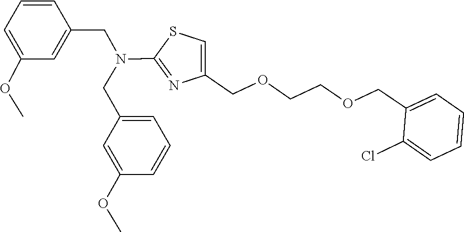 [SMILES: COc1cccc(CN(Cc2cccc(OC)c2)c2nc(COCCOCc3ccccc3Cl)cs2)c1] | 7.35 | 539.09 | II | 1.0 |
| 54 | 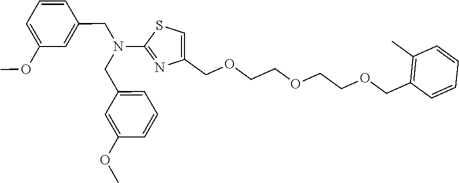 [SMILES: COc1cccc(CN(Cc2cccc(OC)c2)c2nc(COCCOCCOCc3ccccc3C)cs2)c1] | 7.12 | 562.72 | I | 1.8 |
| 40 | 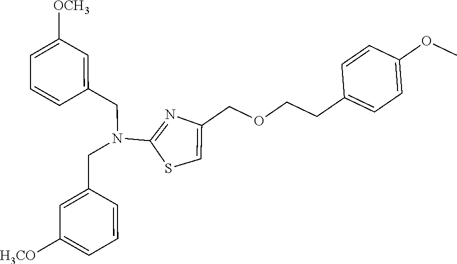 [SMILES: COc1ccc(CCOCc2csc(N(Cc3cccc(OC)c3)Cc3cccc(OC)c3)n2)cc1] | 7.1 | 504.64 | III | 0.4 |
| 60 | 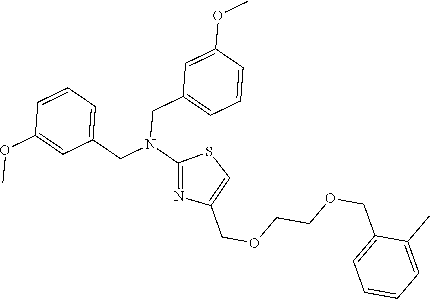 | 7.28 | 518.67 | I | 0.92 |
| 55 | 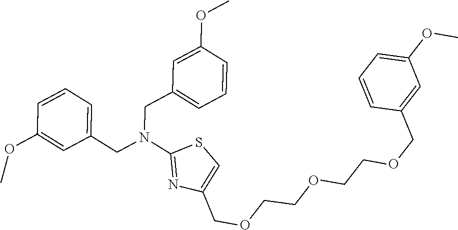 | 6.51 | 578.72 | I | 1.1 |
| 57 | 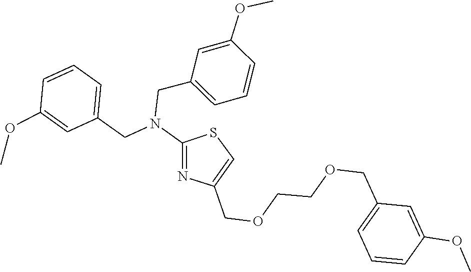 | 6.66 | 534.67 | I | 0.25 |
| 43 | 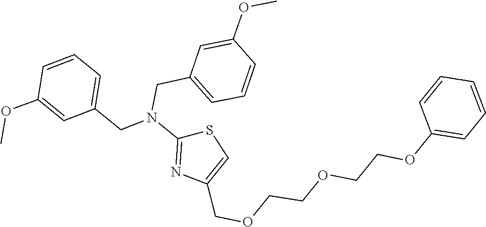 [SMILES: COc1cccc(CN(Cc2cccc(OC)c2)c2nc(COCCOCCOc3ccccc3)cs2)c1] | 6.72 | 534.67 | I | 0.80 |
| 44 | 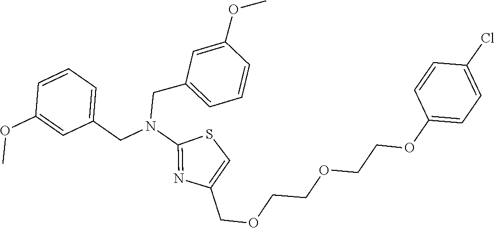 | 7.28 | 569.11 | I | 0.77 |
| 45 | 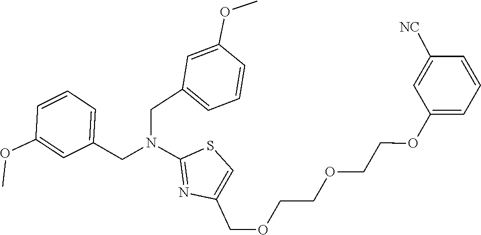 | 6.76 | 559.68 | I | 0.76 |
| 49 | 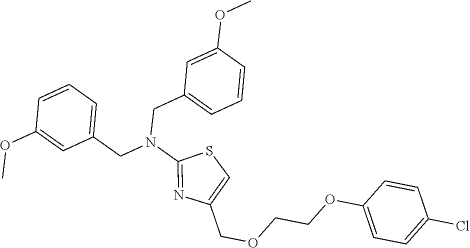 [SMILES: COc1cccc(CN(Cc2cccc(OC)c2)c2nc(COCCOc3ccc(Cl)cc3)cs2)c1] | 7.44 | 525.06 | II | 0.63 |
| 46 | 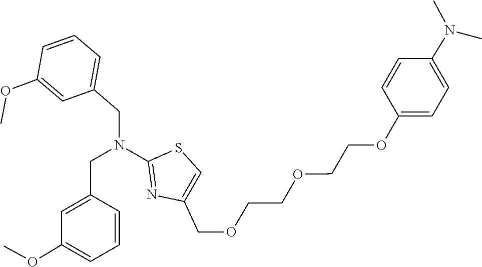 | 7.01 | 577.73 | I | 1.5 |
| 50 | 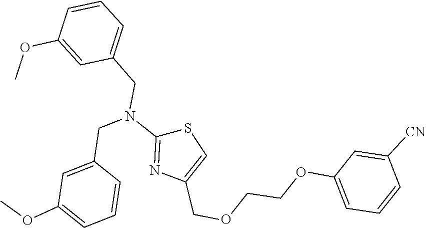 [SMILES: COc1cccc(CN(Cc2cccc(OC)c2)c2nc(COCCOc3cccc(C#N)c3)cs2)c1] | 6.91 | 515.62 | III | 1.2 |
| 51 | 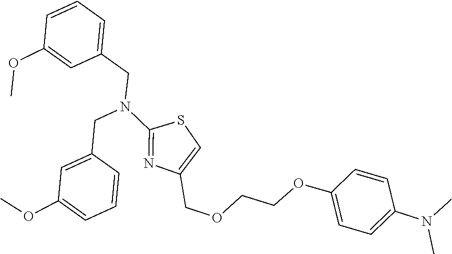 | 7.16 | 533.68 | III | 1.3 |
| 67 | 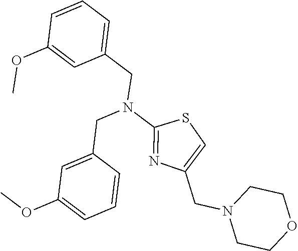 [SMILES: COc1cccc(CN(Cc2cccc(OC)c2)c2nc(CN3CCOCC3)cs2)c1] | 4.97 | 439.57 | I | 1.2 |
| 56 | 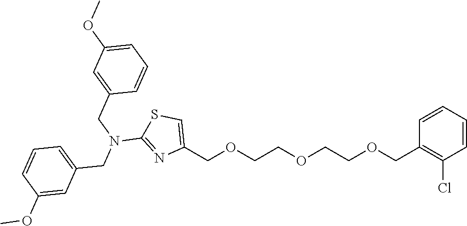 | 7.19 | 583.14 | III | 1.2 |
| 52 | 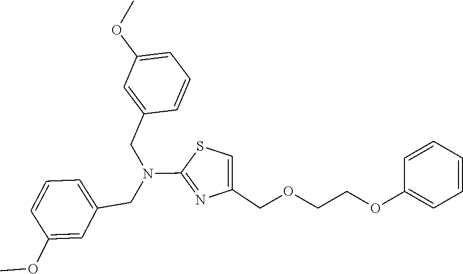 | 6.88 | 490.61 | II | 1.2 |
| 29 | 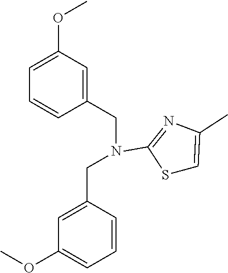 | 5.71 | 354.47 | III | 0.88 |
| 30 | 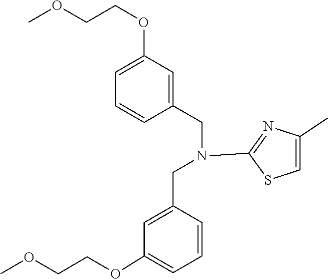 | 5.4 | 442.57 | III | 1.2 |
| 68 | 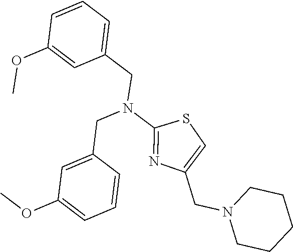 [SMILES: COc1cccc(CN(Cc2cccc(OC)c2)c2nc(CN3CCCCC3)cs2)c1] | 6.1 | 437.60 | II | 1.6 |
| 61 | 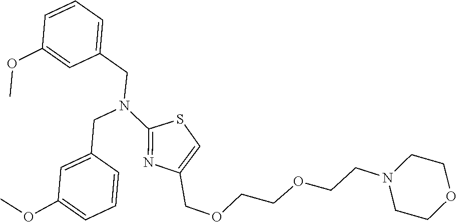 [SMILES: COc1cccc(CN(Cc2cccc(OC)c2)c2nc(COCCOCCN3CCOCC3)cs2)c1] | 4.66 | 527.68 | I | 1.9 |
| 69 | 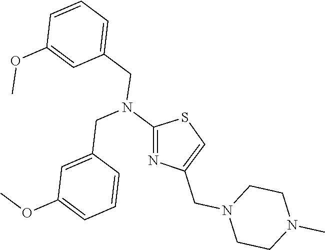 | 5.12 | 452.61 | II | 0.33 |
| 64 | 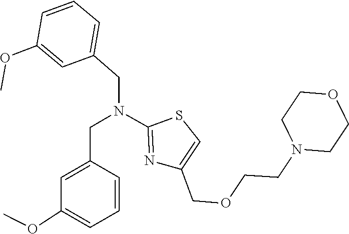 [SMILES: COc1cccc(CN(Cc2cccc(OC)c2)c2nc(COCCN3CCOCC3)cs2)c1] | 4.81 | 483.62 | I | 0.31 |
| 65 | 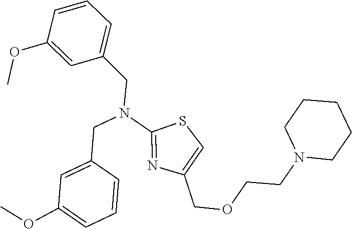 [SMILES: COc1cccc(CN(Cc2cccc(OC)c2)c2nc(COCCN3CCCCC3)cs2)c1] | 5.95 | 481.65 | I | 0.29 |
| 66 | 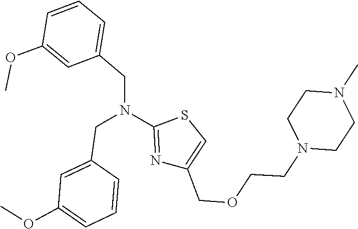 | 4.97 | 496.665.79 | II | 0.27 |
| 62 | 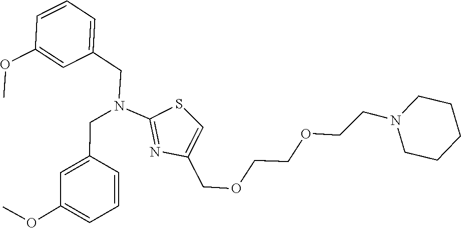 | 5.79 | 525.70 | I | 0.37 |
| 63 | 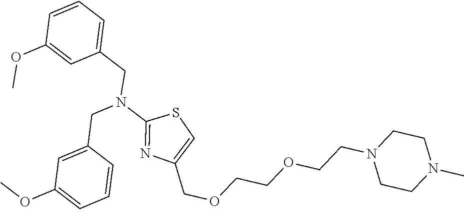 | 4.81 | 540.72 | III | 0.5 |
| 70 | 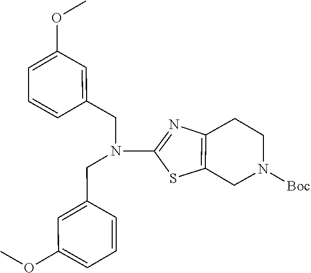 [SMILES: COc1cccc(CN(Cc2cccc(OC)c2)c2nc3c(s2)CN(C(=O)OC(C)(C)C)CC3)c1] | 5.96 | 495.63 | III | 0.14 |
| 71 | 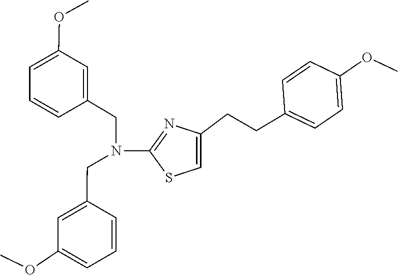 | 7.67 | 474.61 | III | 0.1 |
| 72 | 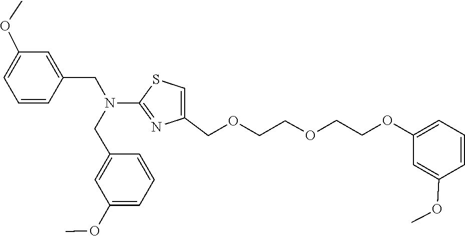 [SMILES: COc1cccc(CN(Cc2cccc(OC)c2)c2nc(COCCOCCOc3cccc(OC)c3)cs2)c1] | 6.6 | 564.69 | I | 0.85 |
| 73 | 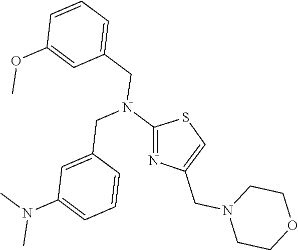 [SMILES: COc1cccc(CN(Cc2cccc(N(C)C)c2)c2nc(CN3CCOCC3)cs2)c1] | 5.38 | 452.61 | II | 0.12 |
| 74 | 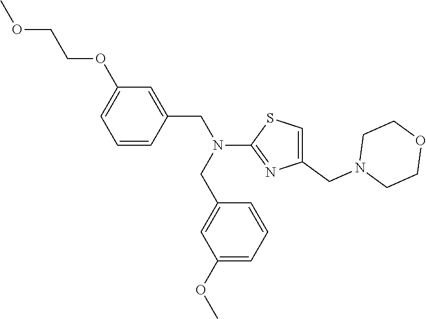 [SMILES: COCCOc1cccc(CN(Cc2cccc(OC)c2)c2nc(CN3CCOCC3)cs2)c1] | 4.81 | 483.62 | I | 0.3 |
| 75 | 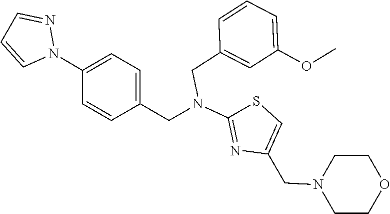 [SMILES: COc1cccc(CN(Cc2ccc(-n3cccn3)cc2)c2nc(CN3CCOCC3)cs2)c1] | 5.1 | 475.61 | I | 0.59 |
| 76 | 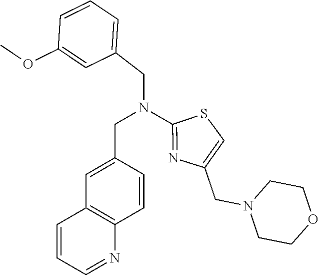 [SMILES: COc1cccc(CN(Cc2ccc3ncccc3c2)c2nc(CN3CCOCC3)cs2)c1] | 5.18 | 460.59 | II | 0.31 |
| 77 | 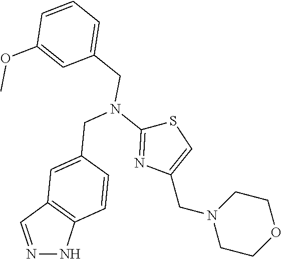 [SMILES: COc1cccc(CN(Cc2ccc3[nH]ncc3c2)c2nc(CN3CCOCC3)cs2)c1] | 4.25 | 449.57 | II | 0.34 |
| 78 | 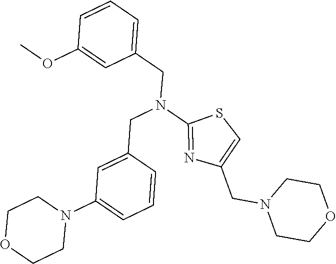 [SMILES: COc1cccc(CN(Cc2cccc(N3CCOCC3)c2)c2nc(CN3CCOCC3)cs2)c1] | 4.98 | 494.65 | II | 0.4 |
| 79 | 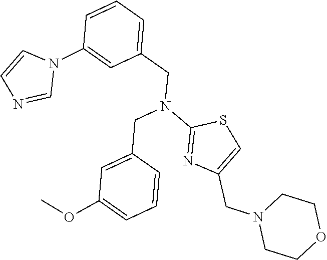 [SMILES: COc1cccc(CN(Cc2cccc(-n3ccnc3)c2)c2nc(CN3CCOCC3)cs2)c1] | 4.3 | 475.61 | II | 0.32 |
| 80 | 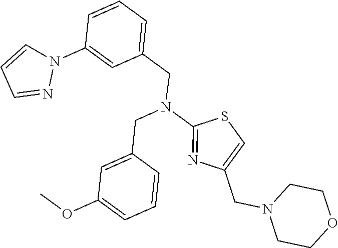 [SMILES: COc1cccc(CN(Cc2cccc(-n3cccn3)c2)c2nc(CN3CCOCC3)cs2)c1] | 5.1 | 475.61 | I | 0.53 |
| 81 | 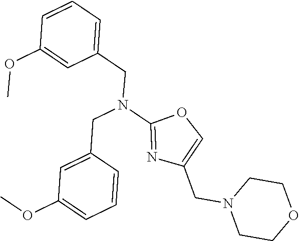 [SMILES: COc1cccc(CN(Cc2cccc(OC)c2)c2nc(CN3CCOCC3)co2)c1] | 3.6 | 423.50 | II | 0.36 |
| 82 | 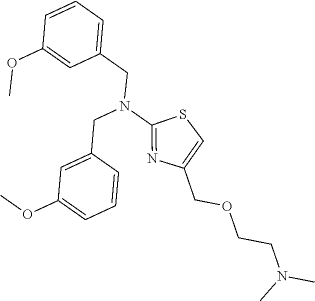 | 5,21 | 441.59 | II | 0.05 |
| 84 | 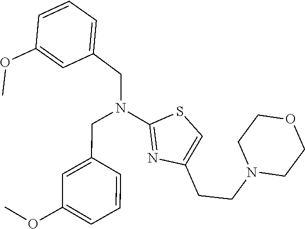 [SMILES: COc1cccc(CN(Cc2cccc(OC)c2)c2nc(CCN3CCOCC3)cs2)c1] | 5.08 | 453.60 | I | 0.14 |
| 85 | 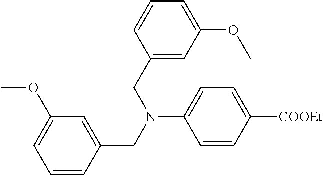 [SMILES: CCOC(=O)c1ccc(N(Cc2cccc(OC)c2)Cc2cccc(OC)c2)cc1] | 5.44 | 405.49 | III | 0.03 |
| 87 | 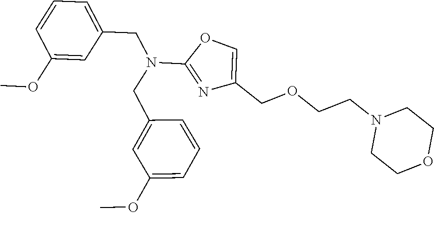 | 3.45 | 467.56 | I | 0.2 |
| 88 | 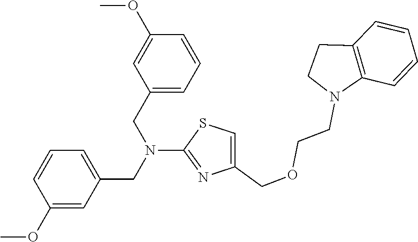 [SMILES: COc1cccc(CN(Cc2cccc(OC)c2)c2nc(COCCN3CCc4ccccc43)cs2)c1] | 7.27 | 515.67 | II | 0.18 |
| 89 | 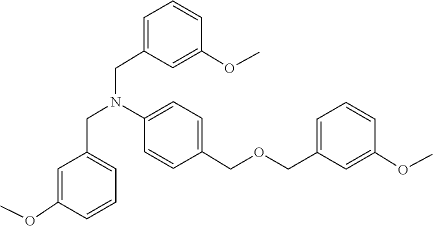 | 6.7 | 483.60 | III | 0.34 |
| 90 | 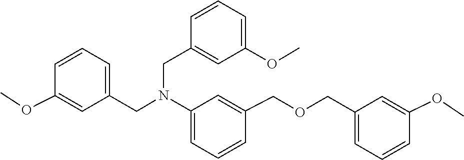 | 6.7 | 483.60 | III | 0.09 |
| 91 | 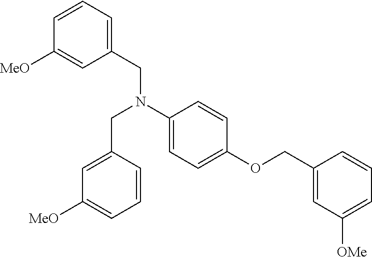 [SMILES: COc1cccc(COc2ccc(N(Cc3cccc(OC)c3)Cc3cccc(OC)c3)cc2)c1] | 6.8 | 469.57 | III | 0.09 |
| 92 | 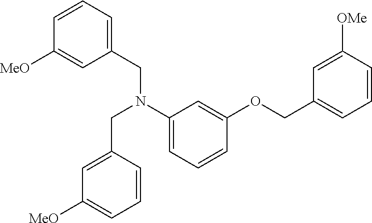 | 6.8 | 469.57 | III | 0.6 |
| 94 | 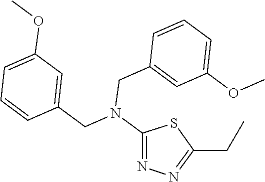 | 6.0 | 369.48 | III | 0.29 |
| 95 | 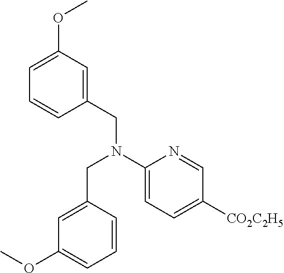 [SMILES: CCOC(=O)c1ccc(N(Cc2cccc(OC)c2)Cc2cccc(OC)c2)nc1] | 4.8 | 406.47 | III | 0.09 |
| 96 | 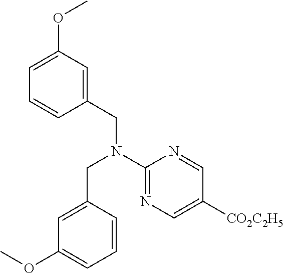 [SMILES: CCOC(=O)c1cnc(N(Cc2cccc(OC)c2)Cc2cccc(OC)c2)nc1] | 4.0 | 407.46 | III | 0.08 |
| 98 | 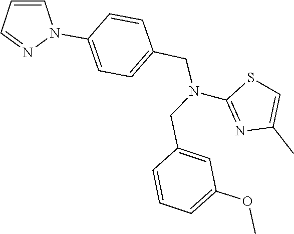 [SMILES: COc1cccc(CN(Cc2ccc(-n3cccn3)cc2)c2nc(C)cs2)c1] | 5.8 | 390.50 | III | 0.03 |
| 100 | 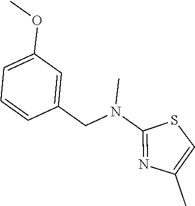 [SMILES: COc1cccc(CN(C)c2nc(C)cs2)c1] | 4.4 | 248.34 | III | 0.05 |
| 101 | 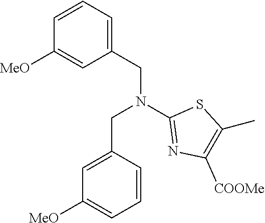 [SMILES: COC(=O)c1nc(N(Cc2cccc(OC)c2)Cc2cccc(OC)c2)sc1C] | 5.58 | 412.50 | II | 0.03 |
| 102 | 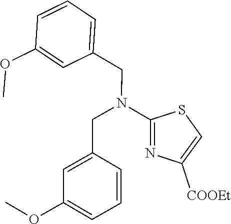 [SMILES: CCOC(=O)c1csc(N(Cc2cccc(OC)c2)Cc2cccc(OC)c2)n1] | 5.58 | 412.50 | II | 0.5 |
| 104 | 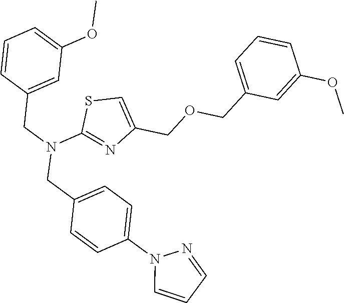 [SMILES: COc1cccc(COCc2csc(N(Cc3ccc(-n4cccn4)cc3)Cc3cccc(OC)c3)n2)c1] | 6.95 | 526.65 | I | 0.28 |
| 106 | 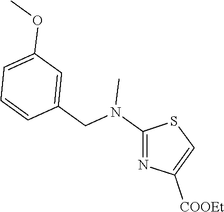 [SMILES: CCOC(=O)c1csc(N(C)Cc2cccc(OC)c2)n1] | 4.2 | 306.38 | III | 0.05 |
| 111 | 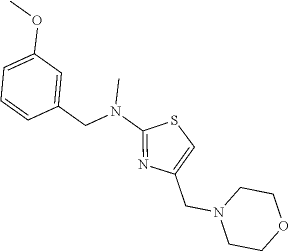 [SMILES: COc1cccc(CN(C)c2nc(CN3CCOCC3)cs2)c1] | 3.6 | 333.45 | III | 0.06 |
| 112 | 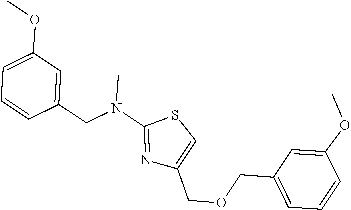 [SMILES: COc1cccc(COCc2csc(N(C)Cc3cccc(OC)c3)n2)c1] | 5.5 | 384.49 | III | 0.04 |
| 113 | 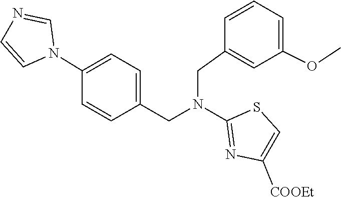 [SMILES: CCOC(=O)c1csc(N(Cc2ccc(-n3ccnc3)cc2)Cc2cccc(OC)c2)n1] | 4.91 | 448.54 | II | 0.64 |
| 114 | 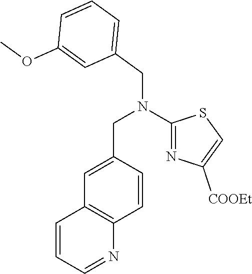 [SMILES: CCOC(=O)c1csc(N(Cc2cccc(OC)c2)Cc2ccc3ncccc3c2)n1] | 5.8 | 433.52 | II | 0.39 |
| 116 | 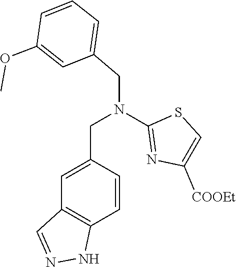 [SMILES: CCOC(=O)c1csc(N(Cc2cccc(OC)c2)Cc2ccc3[nH]ncc3c2)n1] | 4.86 | 422.50 | III | 0.09 |
| 117 | 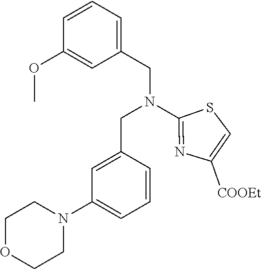 [SMILES: CCOC(=O)c1csc(N(Cc2cccc(OC)c2)Cc2cccc(N3CCOCC3)c2)n1] | 5.6 | 467.58 | II | 0.79 |
| 118 | 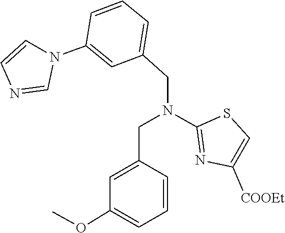 [SMILES: CCOC(=O)c1csc(N(Cc2cccc(OC)c2)Cc2cccc(-n3ccnc3)c2)n1] | 4.91 | 448.54 | II | 0.15 |
| 119 | 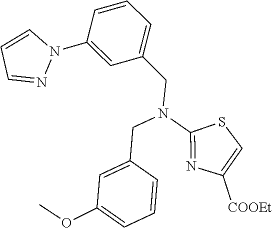 [SMILES: CCOC(=O)c1csc(N(Cc2cccc(OC)c2)Cc2cccc(-n3cccn3)c2)n1] | 5.71 | 448.54 | II | 0.2 |
| 120 | 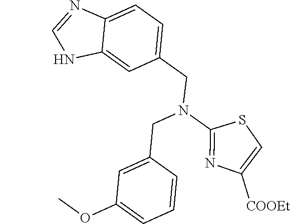 | 4.49 | 422.50 | III | nd |
| 121 | 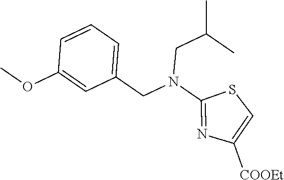 [SMILES: CCOC(=O)c1csc(N(Cc2cccc(OC)c2)CC(C)C)n1] | 5.45 | 348.46 | III | 0.18 |
| 122 | 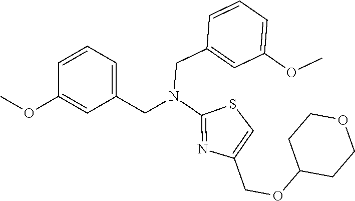 [SMILES: COc1cccc(CN(Cc2cccc(OC)c2)c2nc(COC3CCOCC3)cs2)c1] | 5.01 | 454.58 | II | nd |
| 123 | 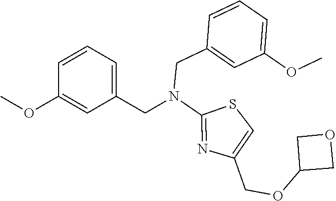 [SMILES: COc1cccc(CN(Cc2cccc(OC)c2)c2nc(COC3COC3)cs2)c1] | 4.79 | 426.53 | II | nd |
| 124 | 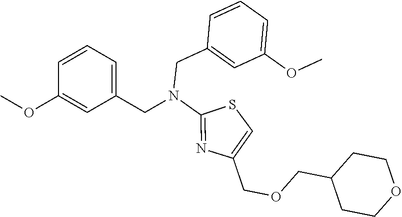 [SMILES: COc1cccc(CN(Cc2cccc(OC)c2)c2nc(COCC3CCOCC3)cs2)c1] | 5.51 | 468.61 | II | nd |
| 125 | 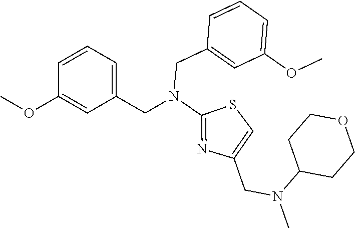 [SMILES: COc1cccc(CN(Cc2cccc(OC)c2)c2nc(CN(C)C3CCOCC3)cs2)c1] | 5.16 | 467.62 | II | nd |
| 126 | 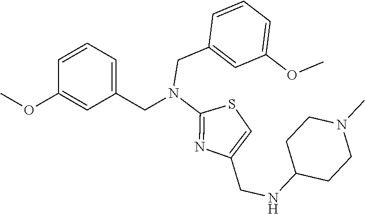 | 4.94 | 466.64 | III | nd |
| 127 | 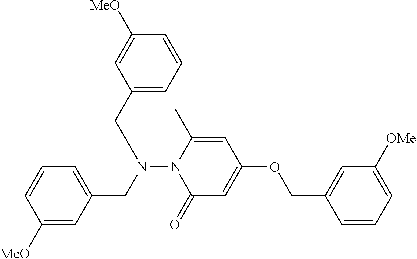 [SMILES: COc1cccc(COc2cc(C)n(N(Cc3cccc(OC)c3)Cc3cccc(OC)c3)c(=O)c2)c1] | 5.2 | 500.59 | II | 0.75 |
| 128 | 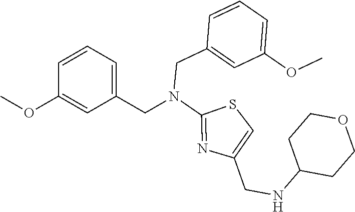 [SMILES: COc1cccc(CN(Cc2cccc(OC)c2)c2nc(CNC3CCOCC3)cs2)c1] | 4.78 | 453.60 | I | nd |
| 129 | 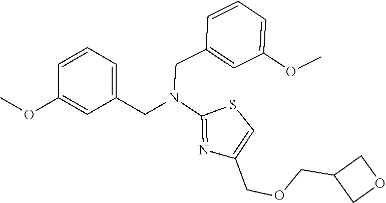 [SMILES: COc1cccc(CN(Cc2cccc(OC)c2)c2nc(COCC3COC3)cs2)c1] | 4.95 | 440.56 | II | nd |
| 130 | 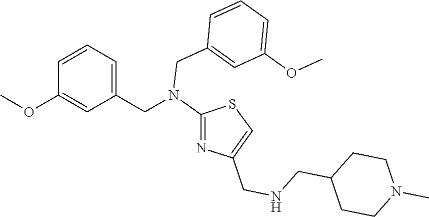 | 5.44 | 480.67 | III | nd |
| 131 | 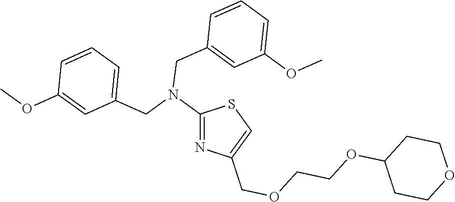 [SMILES: COc1cccc(CN(Cc2cccc(OC)c2)c2nc(COCCOC3CCOCC3)cs2)c1] | 4.85 | 498.63 | I | nd |

The examples presented herein demonstrates drug-like compounds possessing lower molecular weights generally around 500 or less and improved log Ps. Of the compounds listed herein, 733 possess calculated Log P of less than 6. Of those, 499 compounds have a log P of less than 5. In relation to LAD (Leukocyte Adhesion Deficiency), LFA-1 ratios are determined by dividing the relative fluorescence units (RFU) of each compound at a concentration of 3 μM over that of the reference standard of THI0349, also at 3 μM, (RFU Compound/RFU of THI0349). Log Ps were calculated by either Chemdraw® version 12 (CambridgeSoft) or ACD/Chemsketch® (ACD/Labs).

While many embodiments of the disclosed agonist compounds are synthetic compounds of Formula I, in some embodiments an agonist compound is formed by in vivo conversion of a precursor compound to a disclosed compound. For example, a disclosed compound may exist as a stereoisomer wherein asymmetric or chiral centers are present. These stereoisomers are “R” or “S” depending on the configuration of substituents around the chiral carbon atom. The present invention contemplates various stereoisomers and mixtures thereof. Stereoisomers include enantiomers and diastereomers, and mixtures of enantiomers or diastereomers. Individual stereoisomers of some agonist compounds may be prepared synthetically from commercially available starting materials which contain asymmetric or chiral centers or by preparation of racemic mixtures followed by resolution well-known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary or (2) direct separation of the mixture of optical enantiomers on chiral chromatographic columns.

Various embodiments of the disclosed agonist compounds may exist in unsolvated or solvated forms, including hydrated forms, such as hemi-hydrates. In general, the solvated forms, with pharmaceutically acceptable solvents such as water and ethanol among others are equivalent to the unsolvated forms for the purposes of this disclosure. Pharmaceutical compositions containing the disclosed agonist compounds are described below.

### Pharmaceutical Compositions

The compounds described herein may be used in the form of pharmaceutically acceptable salts derived from inorganic or organic acids. The phrase “pharmaceutically acceptable salt” means those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, S. M. Berge et al. describe pharmaceutically acceptable salts in detail in J. Pharmaceutical Sciences, 1977, 66: 1 et seq. The salts may be prepared in situ during the final isolation and purification of the compounds or separately by reacting a free base function with a suitable inorganic or organic acid. Representative acid addition salts include, but are not limited to acetate, adipate, alginate, citrate, aspartate, benzoate, benzenesulfonate, bisulfate, butyrate, camphorate, camphor sulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methane sulfonate, nicotinate, 2-naphthalene sulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

In some embodiments, basic addition salts are prepared in situ during the final isolation and purification of a disclosed compound by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylammonium, dimethylammonium, trimethylammonium, triethylammonium, diethylammonium, and ethylammonium among others. Other representative organic amines useful for the formation of base addition salts include ethylenediamine, ethanolamine, diethanolamine, piperidine, piperazine and the like.

Dosage forms for topical administration of a disclosed agonist compound include powders, sprays, ointments and inhalants. The active compound is mixed under sterile conditions with a pharmaceutically acceptable carrier and any needed preservatives, buffers or propellants which can be required. Ophthalmic formulations, eye ointments, powders and solutions are also contemplated in some embodiments.

Actual dosage levels of active ingredients in the pharmaceutical compositions may be varied so as to obtain an amount of the active compound(s) which is effective to achieve the desired therapeutic response for a particular patient, compositions and mode of administration. The selected dosage level will depend upon the activity of the particular compound, the route of administration, the severity of the condition being treated and the condition and prior medical history of the patient being treated. However, it is within the skill of the art to start doses of the compound at levels lower than required to achieve the desired therapeutic effect and to gradually increase the dosage until the desired effect is achieved.

In certain embodiments, the integrin agonists of Formula (I) and other components, when present, may be administered separately, collectively or concurrently, via any parenteral or non-parenteral administration procedure, wherein the other components include, without limitation, therapeutic antibodies, check point inhibitors, treated and/or untreated effector cells, antigens, adjuvants, excipients, stem cells, progenitor cells, other integrin expressing cells, or any combination thereof.

In other embodiments, the integrin agonists of Formula (I) and the other components are non-parenterally administered.

In other embodiments, the integrin agonists of Formula (I) and the other components are parenterally administered.

In other embodiments, the integrin agonists of Formula (I) non-parenterally administered before, during, and/or after administration of other components, wherein the administration of the other components may be via any acceptable administration procedure such as, without limitation, systemic administration, oral administration, IV administration, arterial administration, direct into tissue administration, any other administration procedure or any combination thereof.

When used for various therapeutic treatments, a therapeutically effective amount of one or more of the disclosed compounds be employed in pure form or, where such forms exist, in pharmaceutically acceptable salt, ester or pro-drug form. In some cases, the compound is administered as a pharmaceutical composition containing the compound of interest in combination with one or more pharmaceutically acceptable excipients. The phrase “therapeutically effective amount” of a disclosed agonist compound means a sufficient amount of the compound to treat disorders, at a reasonable benefit/risk ratio applicable to any medical treatment. It will be understood, however, that the total daily usage of the disclosed compounds and compositions will be decided by the attending physician within the scope of sound medical judgment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than required to achieve the desired therapeutic effect and to gradually increase the dosage until the desired effect is achieved.

The total daily dose of the disclosed compounds administered to a human or lower animal may range from about 0.0001 to about 1000 mg/kg/day. For purposes of oral administration, in some embodiments, doses are in the range of from about 0.001 to about 25 mg/kg/day. If desired, the effective daily dose can be divided into multiple doses for purposes of administration; consequently, single dose compositions may contain such amounts or submultiples thereof to make up the daily dose.

In other embodiments, a dose of the integrin agonists of Formula (I) are adapted to produce an effective concentration measure in molarity units of the integrin agonists in a patient's blood or at a site of action of a patient such as a tumor stroma, a bone marrow stroma, or a site of any other treatable disease or malady. The effective concentration is generally between about 1 fM and about 300 μM, between about 1 nM and about 300 μM, between about 10 nM and about 300 μM, or between about 25 nM and 300 μM.

In some embodiments, a pharmaceutical composition comprises one or more of the disclosed compounds formulated together with one or more non-toxic pharmaceutically acceptable carriers. The pharmaceutical compositions may be specially formulated for oral administration in solid or liquid form, for parenteral injection or for rectal administration.

Disclosed pharmaceutical compositions may be administered to humans and other mammals orally, rectally, parenterally, intracisternally, intravaginally, intraperitoneally, topically (as by powders, ointments or drops), bucally or as an oral or nasal spray. The term “parenterally,” as used herein, refers to modes of administration which include intravenous, intramuscular, intraperitoneal, intrasternal, subcutaneous and intraarticular injection and infusion. In some implementations, a pharmaceutical composition comprises a disclosed compound and a physiologically tolerable or acceptable diluent, carrier, adjuvant or vehicle, which are collectively referred to herein as diluents, for parenteral injection, for intranasal delivery, for oral administration in solid or liquid form, for rectal or topical administration, or the like.

In some embodiments, a composition is delivered through a catheter for local delivery at a target site, via an intracoronary stent (a tubular device composed of a fine wire mesh), or via a biodegradable polymer. In some embodiments, an agonist compound is complexed to a ligand such as an antibody, for targeted delivery.

Compositions suitable for parenteral injection may comprise physiologically acceptable, sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), vegetable oils (such as olive oil), injectable organic esters such as ethyl oleate, and suitable mixtures thereof. These compositions can also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride and the like. Prolonged absorption of the injectable pharmaceutical form can be brought about by the use of agents delaying absorption, for example, aluminum monostearate and gelatin.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like. Proper fluidity can be maintained, for example, by the use of coating materials such as lecithin, by the maintenance of the required particle size in the case of dispersions and by the use of surfactants.

In some embodiments, in order to prolong the effect of the drug, it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline foam. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders and granules. In such solid dosage forms, the active compound may be mixed with at least one inert, pharmaceutically acceptable excipient or carrier, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes. The active compounds can also be in micro-encapsulated form, if appropriate, with one or more of the above-mentioned excipients. Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

Besides inert diluents, the oral compositions may also include adjuvants such as wetting agents, emulsifying and suspending agents, sweetening, flavoring and perfuming agents. Compositions for rectal or vaginal administration are preferably suppositories which can be prepared by mixing one or more of the disclosed compounds with suitable non-irritating excipients or carriers such as cocoa butter, polyethylene glycol or a suppository wax which are solid at room temperature but liquid at body temperature and therefore melt in the rectum or vaginal cavity and release the active compound.

For some applications, a disclosed compound is administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. In some embodiments, a composition in liposome form contains, in addition to a disclosed agonist compound, stabilizers, preservatives, excipients and the like. The preferred lipids are natural and synthetic phospholipids and phosphatidyl cholines (lecithins) used separately or together. Methods of forming liposomes are known in the art. See, for example, Prescott, Ed., METHODS IN CELL BIOLOGY, Volume XIV, Academic Press, New York, N.Y. (1976), p. 33 et seq.

The term “pharmaceutically acceptable pro-drugs” as used herein represents those pro-drugs of the disclosed compounds which are, within the scope of sound medical judgment, suitable for use in contact with the tissues of humans and lower animals without undue toxicity, irritation, allergic response, and the like, commensurate with a reasonable benefit/risk ratio, and effective for their intended use, as well as the zwitterionic forms, where possible, of the disclosed compounds. Pro-drugs according to certain embodiments may be rapidly transformed in vivo to the parent compound of the above formula, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., BIOREVERSIBLE CARRIERS IN DRUG DESIGN, American Pharmaceutical Association and Pergamon Press (1987), hereby incorporated by reference.

While the preferred embodiments have been shown and described, modifications thereof can be made by one skilled in the art without departing from the spirit and teachings of the invention. The embodiments described herein are exemplary and representative and are not intended to be limiting. Many variations and modifications of the invention disclosed herein are possible and are within the scope of the invention. Accordingly, the scope of protection is not limited by the description set out above, but is only limited by the claims which follow, that scope including all equivalents of the subject matter of the claims. All patents, patent applications and publications cited herein are hereby incorporated herein by reference to the extent that they provide materials, methods and explanatory details supplementary to those set forth herein.

1. A compound comprising a VLA-4 integrin agonist of the Formula (I): 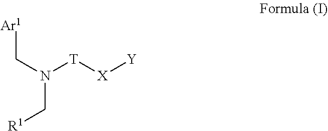
wherein, R¹ is hydrogen, lower alkyl, or aryl; Ar¹ is aryl, provided that when Ar and R¹ are both phenyl, the 2 and 6 positions of each ring are substituted with hydrogen; T is aryl; X is a 1-20 atom linking chain containing any combination of —CH₂—, —C(O)—, —C(O)NR²—, —NR²—, —C(O)O— and —O—, each of which may independently be absent or present one or more times; provided that when X contains —C(O)N(R²)—, and —C(O)N(R²)— directly attaches to T, then R² is hydrogen; Y is selected from a group consisting essentially of hydrogen, (C₁-C₄)alkyl, aryl, cycloalkyl, N,N-dialkylamino, heterocyclyl, N,N-di(aralkyl)aminoaryl, or (N-aralkyl)(N-alkyl)aminoaryl; Ar¹ and R¹ may each be independently substituted or multiply substituted and selected at each occurrence from the group consisting essentially of lower alkyl, alkoxy, hydroxyalkyl, —OH, alkoxyalkyl, (C₁-C₃ alkyl)₂amino, alkoxyalkoxy, cycloalkyl, cycloalkylalkyl, aryl, heterocyclyl, alkylaryl, aralkyl, alkylheterocyclyl, and heterocyclylalkyl, groups; Y may be unsubstituted or substituted with one or more substituents selected from the group consisting essentially of halogen, lower alkyl, alkoxy, hydroxyalkyl, aliphatic acyl, —CF₃, oxo, —CN, —OH, alkoxyalkyl, (C₁-C₃ alkyl)₂amino, alkoxyalkoxy, cycloalkyl, cycloalkylalkyl, aryl, heterocyclyl, alkylaryl, aralkyl, alkylheterocyclyl, heterocyclylalkyl, and aryloxyalkyl groups; T may be unsubstituted or substituted with one or more substituents selected from the group consisting essentially of lower alkyl, alkoxy, hydroxyalkyl, aliphatic acyl, —CN, —OH, halogen, heterocycloyl, alkoxyalkyl, (C₁-C₃ alkyl)₂amino, alkoxyalkoxy, cycloalkyl, cycloalkylalkyl, heterocyclyl, alkylheterocyclyl, heterocyclylalkyl and a combination thereof, provided that when T is thiazolyl group and X and Y taken together form a C₁-C₈ alkyl group, then the 5-position of said thiazole ring is unsubstituted; substituents of T may be taken together with up to two atoms of X to form a ring; and R² when present, may be selected independently and at each occurrence from the groups consisting essentially of hydrogen, lower alkyl, acetyl and methanesulfonyl.

2. The compound of claim 1, wherein (Ar¹CH₂)(R¹CH₂)N-T is selected from a group comprising: 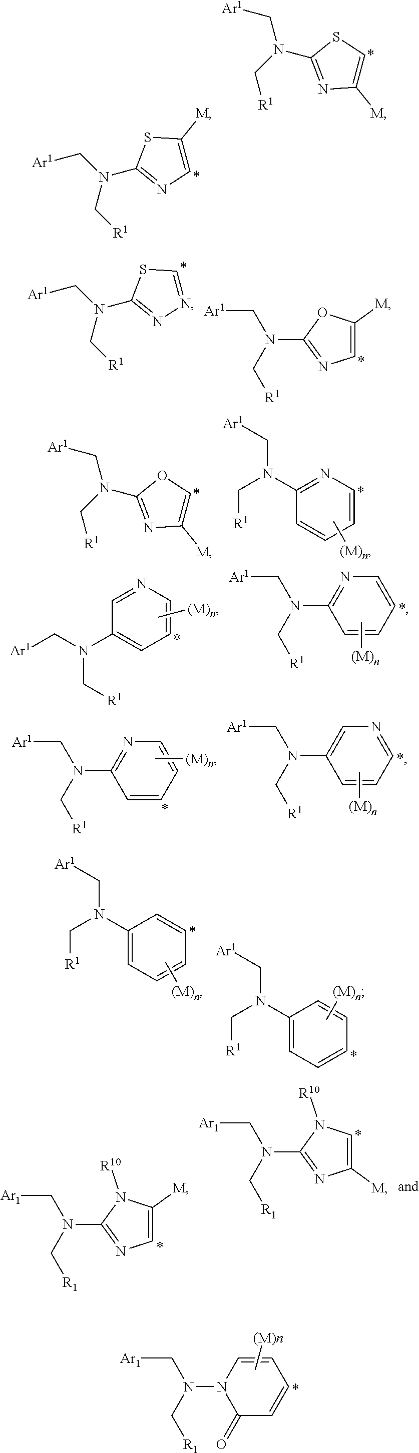
wherein the asterisks of each of the (Ar¹CH₂)(R¹CH₂)N-T groups indicate the attachment point for X; M, when present, may be selected, independently and at each occurrence, from a group comprising: lower alkyl, alkoxy, hydroxyalkyl, aliphatic acyl, —CF₃, —CN, —OH, halogen, heterocycloyl, alkoxyalkyl, (C₁-C₃ alkyl)₂amino, alkoxyalkoxy, cycloalkyl, cycloalkylalkyl, heterocyclyl, alkylheterocyclyl, and heterocyclylalkyl;
n is an integer from 0-3;
R¹⁰ when present is Hydrogen or C₁-C₄ lower alkyl;
X is further selected from a group of radicals comprising:
—(CH₂)_{z}—, —(CH₂)_{z}O—, —(CH₂)_{z}O(CH₂)_{z}—, —(CH₂CH₂O)ₚ—, —(CH₂)_{z}O(CH₂)_{z}, —(CH₂)_{z}O(CH₂)_{z}O(CH₂)_{z}—, —(CH₂)_{z}O(CH₂CH₂O)ₚ—, —(CH₂)_{z}O(CH₂CH₂O)ₚ—(CH₂)_{z}—, —(CH₂)_{z}NR²—, —(CH₂)_{z}NR²(CH₂)_{z}—, —(CH₂)_{z}NR²(CH₂CH₂O)ₚ—, —(CH₂CH₂O)ₚ(CH₂)_{z}NR²—, —C(O)N(R²)—, —C(O)N(R²)(CH₂)_{z}—, —(CH₂)_{z}C(O)N(R²)(CH₂)_{z}—, —(CH₂)_{z}C(O)N(R²)—, —O(CH₂)_{z}—, —O(CH₂)_{z}O(CH₂)_{z}—, —O(CH₂CH₂O)ₚ—, —O(CH₂)_{z}NR²(CH₂)_{z}—, —O(CH₂)_{z}NR²(CH₂CH₂O)ₚ—, —O(CH₂CH₂O)ₚ(CH₂)_{z}NR²—, —O(CH₂)_{z}C(O)N(R²)(CH₂)_{z}—, and —O(CH₂)_{z}C(O)N(R²)—;
z is independently at each occurrence an integer of 1-20, and p is an integer from 0-6,
provided that in X for any combination of groups, the integers p and z do not create a chain of greater than 20 atoms; and a radical of M and 1 or 2 atoms of X may be taken together to form a 5 or 6 membered ring.

3. The compound of claim 2, wherein Ar¹ is selected from the group consisting essentially of substituted phenyl, substituted or unsubstituted or unsubstituted heteroaromatic groups thienyl, oxazolyl, isoxazolyl, pyrrolyl or pyridyl; and R¹ is hydrogen, C₁-C₄ alkyl, substituted phenyl, substituted or unsubstituted heteroaromatic groups selected from: thienyl, unsubstituted thienyl, oxazolyl, isoxazolyl, pyrrolyl and pyridinyl.

4. The compound of claim 2, wherein (Ar¹CH₂)(R¹CH₂)N-T is selected from a group consisting essentially of 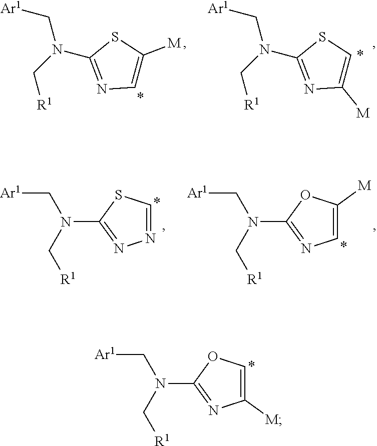
provided that in X for any combination of groups, the integers p and z do not create a chain of greater than 10 atoms; and M is hydrogen or C₁-C₄ alkyl.

5. The compound of claim 1, wherein Y is selected from a group consisting essentially of: 4-morpholinyl, 1-piperidinyl, 1-piperazinyl, piperazin-2-one-1-yl, piperazine-2,5-dione-1-yl, piperazin-2-one-4-yl, 1-pyrrolidinyl, 2-oxa-5-azabicyclo[2.2.1]heptan-5-yl, 3-hydroxypyrrolidin-1-yl, 4-hydroxypyrrolidine-2-one-1-yl, 3-hydroxypyrrolidin-2-one-1-yl, morpholin-3-one-4-yl, 6-oxa-1-azaspiro[3.3]heptan-1-yl, 6-oxa-3-azabicyclo[3.1.1]heptan-3-yl, hexahydro-2H-furo[3,2-b]pyrrol-4(5H)-yl, 2-oxa-5-azabicyclo[4.1.0]heptan-5-yl, phenyl, tetrahydro-2H-pyran-4-yl, oxetan-3-yl, tetrahydrofuran-3-yl, imidazol-1-yl, di(aralkyl)aminoaryl and (N-aralkyl)(N-alkyl)aminoaryl; including stereo-isomers thereof, and Y may be unsubstituted or substituted with one or more substituents selected from the group consisting essentially of halogen, lower alkyl, alkoxy, —CF₃, —CN, —OH, alkoxyalkyl, (C₁-C₃ alkyl)₂amino, alkoxyalkoxy, heterocyclyl, alkylheterocyclyl, and heterocyclylalkyl.

6. The compound of claim 1, wherein the VLA-4 integrin agonist of the formula (I) is selected from the group consisting essentially of: ethyl 2-(bis(thiophen-2-ylmethyl)amino)oxazole-4-carboxylate; 2-(bis(thiophen-2-ylmethyl)amino)-N-(3-methoxybenzyl)oxazole-4-carboxamide; 2-(bis(3-methoxybenzyl)amino)-N-(3-methoxybenzyl)oxazole-4-carboxamide; 2-(bis(3-methoxybenzyl)amino)-N-(4-methoxyphenethyl)oxazole-4-carboxamide; 2-(bis(3-methoxybenzyl)amino)-N-(3-(4-methoxyphenyl)propyl)oxazole-4-carboxamide; 2-(bis(3-methoxybenzyl)amino)-N-(3-(4-methoxyphenyl)propyl)oxazole-5-carboxamide; 2-(bis(3-methoxybenzyl)amino)-N-(4-methoxyphenethyl)thiazole-4-carboxamide; 2-(bis(3-methoxybenzyl)amino)-N-(3-(4-methoxyphenyl)propyl)thiazole-4-carboxamide; 4-((3-(4-methoxyphenyl)propoxy)methyl)-N,N-bis(thiophen-2-ylmethyl)oxazol-2-amine; ethyl 2-(bis(4-methoxybenzyl)amino)oxazole-4-carboxylate; (2-(bis(thiophen-2-ylmethyl)amino)oxazol-4-yl)methanol; ethyl 2-(bis(3-methoxybenzyl)amino)oxazole-4-carboxylate; ethyl 2-(bis(3-methoxybenzyl)amino)oxazole-4-carboxylate; (2-(bis(4-methoxybenzyl)amino)oxazol-4-yl)methanol; N,N-bis(3-methoxybenzyl)-2-(2-phenoxyethyl)oxazol-5-amine; ethyl 2-(bis(3-(2-methoxyethoxy)benzyl)amino)oxazole-4-carboxylate; 2-(2-((2-(bis(4-methoxybenzyl)amino)thiazol-4-yl)methoxy)ethoxy)ethanol; 4,4′-(2,2′-oxybis(ethane-2,1-diyl)bis(oxy))bis(methylene)bis(N,N-bis(4-methoxybenzyl)thiazol-2-amine); 2-((2-(bis(3-methoxybenzyl)amino)thiazol-4-yl)methoxy)ethanol; N,N-bis(3-methoxybenzyl)-4-((2-(2-(3-methoxyphenoxy)ethoxy)ethoxy)methyl)thiazol-2-amine; 4-((2-(2-(3-(dimethylamino)phenoxy)ethoxy)ethoxy)methyl)-N,N-bis(3-methoxybenzyl)thiazol-2-amine; 4-((2-(2-(benzyloxy)ethoxy)ethoxy)methyl)-N,N-bis(3-methoxybenzyl)thiazol-2-amine; N,N-bis(3-methoxybenzyl)-4-(((3-methoxybenzyl)oxy)methyl)thiazol-2-amine; N,N-bis(3-methoxybenzyl)-4-((3-(4-methoxyphenyl)propoxy)methyl)thiazol-2-amine; N,N-bis(3-methoxybenzyl)-4-((2-(3-methoxyphenoxy)ethoxy)methyl)thiazol-2-amine; N,N-bis(3-methoxybenzyl)-4-(((3-methoxybenzyl)oxy)methyl)thiazol-2-amine; N,N-bis(3-methoxybenzyl)-4-((3-(4-methoxyphenyl)propoxy)methyl)thiazol-2-amine; N,N-bis(3-methoxybenzyl)-4-((2-(3-methoxyphenoxy)ethoxy)methyl)thiazol-2-amine; 4-((2-(3-(dimethylamino)phenoxy)ethoxy)methyl)-N,N-bis(3-methoxybenzyl)thiazol-2-amine; 4-((2-(benzyloxy)ethoxy)methyl)-N,N-bis(3-methoxybenzyl)thiazol-2-amine; 4-((2-((2-chlorobenzyl)oxy)ethoxy)methyl)-N,N-bis(3-methoxybenzyl)thiazol-2-amine; N,N-bis(3-methoxybenzyl)-4-((2-(2-((2-methylbenzyl)oxy)ethoxy)ethoxy)methyl)thiazol-2-amine; N,N-bis(3-methoxybenzyl)-4-((4-methoxyphenethoxy)methyl)thiazol-2-amine; N,N-bis(3-methoxybenzyl)-4-((2-((2-methylbenzyl)oxy)ethoxy)methyl)thiazol-2-amine; N,N-bis(3-methoxybenzyl)-4-((2-(2-((3-methoxybenzyl)oxy)ethoxy)ethoxy)methyl)thiazol-2-amine; N,N-bis(3-methoxybenzyl)-4-((2-((3-methoxybenzyl)oxy)ethoxy)methyl)thiazol-2-amine; N,N-bis(3-methoxybenzyl)-4-((2-(2-phenoxyethoxy)ethoxy)methyl)thiazol-2-amine; 4-((2-(2-(4-chlorophenoxy)ethoxy)ethoxy)methyl)-N,N-bis(3-methoxybenzyl)thiazol-2-amine; 3-(2-(2-((2-(bis(3-methoxybenzyl)amino)thiazol-4-yl)methoxy)ethoxy)ethoxy)benzonitrile; 4-((2-(4-chlorophenoxy)ethoxy)methyl)-N,N-bis(3-methoxybenzyl)thiazol-2-amine; 4-((2-(2-(4-(dimethylamino)phenoxy)ethoxy)ethoxy)methyl)-N,N-bis(3-methoxybenzyl)thiazol-2-amine; 3-(2-((2-(bis(3-methoxybenzyl)amino)thiazol-4-yl)methoxy)ethoxy)benzonitrile; 4-((2-(4-(dimethylamino)phenoxy)ethoxy)methyl)-N,N-bis(3-methoxybenzyl)thiazol-2-amine; N,N-bis(3-methoxybenzyl)-4-(morpholinomethyl)thiazol-2-amine; 4-((2-(2-((2-chlorobenzyl)oxy)ethoxy)ethoxy)methyl)-N,N-bis(3-methoxybenzyl)thiazol-2-amine; N,N-bis(3-methoxybenzyl)-4-((2-phenoxyethoxy)methyl)thiazol-2-amine; N,N-bis(3-methoxybenzyl)-4-methylthiazol-2-amine; N,N-bis(3-(2-methoxyethoxy)benzyl)-4-methylthiazol-2-amine; N,N-bis(3-methoxybenzyl)-4-(piperidin-1-ylmethyl)thiazol-2-amine; N,N-bis(3-methoxybenzyl)-4-((2-(2-morpholinoethoxy)ethoxy)methyl)thiazol-2-amine; N,N-bis(3-methoxybenzyl)-4-((4-methylpiperazin-1-yl)methyl)thiazol-2-amine; N,N-bis(3-methoxybenzyl)-4-((2-morpholinoethoxy)methyl)thiazol-2-amine; N,N-bis(3-methoxybenzyl)-4-((2-(piperidin-1-yl)ethoxy)methyl)thiazol-2-amine; N,N-bis(3-methoxybenzyl)-4-((2-(4-methylpiperazin-1-yl)ethoxy)methyl)thiazol-2-amine; N,N-bis(3-methoxybenzyl)-4-((2-(2-(piperidin-1-yl)ethoxy)ethoxy)methyl)thiazol-2-amine; and N,N-bis(3-methoxybenzyl)-4-((2-(2-(4-methylpiperazin-1-yl)ethoxy)ethoxy)methyl)thiazol-2-amine.

7. The compound of claim 1, wherein the VLA-4 integrin agonist of the formula (I) is selected from the group consisting essentially of 4-((2-(2-(dimethylamino)ethoxy)ethoxy)methyl)-N,N-bis(3-methoxybenzyl)thiazol-2-amine; 4-((2-(dimethylamino)ethoxy)methyl)-N,N-bis(3-methoxybenzyl)thiazol-2-amine; N,N-bis(3-methoxybenzyl)-4-(2-methyl-5,8,11-trioxa-2-azadodecan-12-yl)thizazol-2-amine; N,N-bis(3-methoxybenzyl)-4-((2-(2-(2-morpholinoethoxy)ethoxy)ethoxy)methyl)thiazol-2-amine; 4-((2-(2-(indolin-1-yl)ethoxy)ethoxy)methyl)-N,N-bis(3-methoxybenzyl)thiazol-2-amine; 4-((2-(2-(3,4-dihydroquinolin-1(2H)-yl)ethoxy)ethoxy)methyl)-N,N-bis(3-methoxybenzyl)thiazol-2-amine; 4-((2-(3,4-dihydroquinolin-1(2H)-yl)ethoxy)methyl)-N,N-bis(3-methoxybenzyl)thiazol-2-amine; 4-((2-(indolin-1-yl)ethoxy)methyl)-N,N-bis(3-methoxybenzyl)thiazol-2-amine; 1-((2-(bis(3-methoxybenzyl)amino)thiazol-4-yl)methyl)piperazin-2-one; 1-((2-(bis(3-methoxybenzyl)amino)thiazol-4-yl)methyl)piperazine-2,5-dione; 4-((2-(bis(3-methoxybenzyl)amino)thiazol-4-yl)methyl)piperazin-2-one; N,N-bis(3-methoxybenzyl)-4-methyloxazol-2-amine; N-(3-(dimethylamino)benzyl)-N-(3-methoxybenzyl)-4-methyloxazol-2-amine; N-(3-methoxybenzyl)-4-methyl-N-(4-(pyrrolidin-1-yl)benzyl)oxazol-2-amine; N-(3-methoxybenzyl)-4-methyl-N-(3-morpholinobenzyl)oxazol-2-amine; N-(3-methoxybenzyl)-4-methyl-N-(3-(pyrrolidin-1-yl)benzyl)oxazol-2-amine; N-(3-(1H-imidazol-1-yl)benzyl)-N-(3-methoxybenzyl)-4-methyloxazol-2-amine; N-(3-(1H-pyrazol-1-yl)benzyl)-N-(3-methoxybenzyl)-4-methyloxazol-2-amine; N-((1H-indazol-5-yl)methyl)-N-(3-methoxybenzyl)-4-methyloxazol-2-amine; N-((1H-benzo[d]imidazol-5-yl)methyl)-N-(3-methoxybenzyl)-4-methyloxazol-2-amine; N-(3-methoxybenzyl)-4-methyl-N-(quinolin-6-ylmethyl)oxazol-2-amine; N-(3-methoxybenzyl)-4-((2-(3-methoxybenzyloxy)ethoxy)methyl)-N-(quinolin-6-ylmethyl)oxazol-2-amine; 4-((2-(3-(dimethylamino)phenoxy)ethoxy)methyl)-N-(3-methoxybenzyl)-N-(quinolin-6-ylmethyl)oxazol-2-amine; N-(3-(dimethylamino)benzyl)-N-(3-methoxybenzyl)-4-((2-(3-methoxybenzyloxy)ethoxy)methyl)oxazol-2-amine; N-(3-methoxybenzyl)-4-((2-(3-methoxybenzyloxy)ethoxy)methyl)-N-(3-(pyrrolidin-1-yl)benzyl)oxazol-2-amine; N-(3-(1H-imidazol-1-yl)benzyl)-N-(3-methoxybenzyl)-4-((2-(3-methoxybenzyloxy)ethoxy)methyl)oxazol-2-amine; N-(3-(1H-pyrazol-1-yl)benzyl)-N-(3-methoxybenzyl)-4-((2-(3-methoxybenzyloxy)ethoxy)methyl)oxazol-2-amine; N-(3-methoxybenzyl)-4-((2-(3-methoxybenzyloxy)ethoxy)methyl)-N-(3-morpholinobenzyl)oxazol-2-amine; N-(3-methoxybenzyl)-4-((2-(3-methoxybenzyloxy)ethoxy)methyl)-N-(3-(4-methylpiperazin-1-yl)benzyl)oxazol-2-amine; N-((1H-benzo[d]imidazol-5-yl)methyl)-N-(3-methoxybenzyl)-4-((2-(3-methoxybenzyloxy)ethoxy)methyl)oxazol-2-amine; N-((1H-indazol-5-yl)methyl)-N-(3-methoxybenzyl)-4-((2-(3-methoxybenzyloxy)ethoxy)methyl)oxazol-2-amine; N-(3-methoxybenzyl)-4-((2-(3-methoxybenzyloxy)ethoxy)methyl)-N-(quinolin-6-ylmethyl)thiazol-2-amine; N-(3-methoxybenzyl)-4-((2-morpholinoethoxy)methyl)-N-(quinolin-6-ylmethyl)oxazol-2-amine; N-(4-(1H-imidazol-1-yl)benzyl)-N-(3-methoxybenzyl)-4-((2-morpholinoethoxy)methyl)oxazol-2-amine; N-(4-(1H-pyrazol-1-yl)benzyl)-N-(3-methoxybenzyl)-4-((2-morpholinoethoxy)methyl)oxazol-2-amine; N-(3-methoxybenzyl)-N-(4-morpholinobenzyl)-4-((2-morpholinoethoxy)methyl)oxazol-2-amine; N-(3-methoxybenzyl)-4-((2-morpholinoethoxy)methyl)-N-(4-(pyrrolidin-1-yl)benzyl)oxazol-2-amine; N-(3-methoxybenzyl)-N-(4-(4-methylpiperazin-1-yl)benzyl)-4-((2-morpholinoethoxy)methyl)oxazol-2-amine; N-((1H-benzo[d]imidazol-6-yl)methyl)-N-(3-methoxybenzyl)-4-((2-morpholinoethoxy)methyl)oxazol-2-amine; N-((1H-indazol-6-yl)methyl)-N-(3-methoxybenzyl)-4-((2-morpholinoethoxy)methyl)oxazol-2-amine; N-(3-(dimethylamino)benzyl)-N-(3-methoxybenzyl)-4-methylthiazol-2-amine; N-(3-methoxybenzyl)-4-methyl-N-(3-morpholinobenzyl)thiazol-2-amine; N-(3-methoxybenzyl)-4-methyl-N-(3-morpholinobenzyl)oxazol-2-amine; N-(3-(1H-imidazol-1-yl)benzyl)-N-(3-methoxybenzyl)-4-methyloxazol-2-amine; N-(3-(1H-pyrazol-1-yl)benzyl)-N-(3-methoxybenzyl)-4-methyloxazol-2-amine; N-(3-(1H-imidazol-1-yl)benzyl)-N-(3-methoxybenzyl)-4-methylthiazol-2-amine; N-(3-(1H-pyrazol-1-yl)benzyl)-N-(3-methoxybenzyl)-4-methylthiazol-2-amine; N-(3-methoxybenzyl)-4-methyl-N-(4-(pyrrolidin-1-yl)benzyl)thiazol-2-amine; N-(4-(1H-imidazol-1-yl)benzyl)-N-(3-methoxybenzyl)-4-methylthiazol-2-amine; N-(4-(1H-pyrazol-1-yl)benzyl)-N-(3-methoxybenzyl)-4-methylthiazol-2-amine; N-(4-(1H-imidazol-1-yl)benzyl)-N-(3-methoxybenzyl)-4-methyloxazol-2-amine; N-(3-methoxybenzyl)-4-methyl-N-(4-(4-methylpiperazin-1-yl)benzyl)thiazol-2-amine; N-(3-methoxybenzyl)-4-methyl-N-(4-morpholinobenzyl)thiazol-2-amine; N-((1H-benzo[d]imidazol-6-yl)methyl)-N-(3-methoxybenzyl)-4-methylthiazol-2-amine; N-((1H-indazol-6-yl)methyl)-N-(3-methoxybenzyl)-4-methylthiazol-2-amine; N-(3-methoxybenzyl)-4-methyl-N-(quinolin-6-ylmethyl)thiazol-2-amine; N,N-bis(3-methoxybenzyl)-4-((2-(2-(3-methoxyphenoxy)ethoxy)ethoxy)methyl)oxazol-2-amine; 4-((2-(2-(benzyloxy)ethoxy)ethoxy)methyl)-N,N-bis(3-methoxybenzyl)oxazol-2-amine; 4-((2-(3-(dimethylamino)phenoxy)ethoxy)methyl)-N,N-bis(3-methoxybenzyl)oxazol-2-amine; N,N-bis(3-methoxybenzyl)-4-((2-(2-(3-methoxybenzyloxy)ethoxy)ethoxy)methyl)oxazol-2-amine; N,N-bis(3-methoxybenzyl)-4-((2-(3-methoxybenzyloxy)ethoxy)methyl)oxazol-2-amine; N,N-bis(3-methoxybenzyl)-4-(morpholinomethyl)oxazol-2-amine; N,N-bis(3-(2-methoxyethoxy)benzyl)-4-methyloxazol-2-amine; N,N-bis(3-methoxybenzyl)-4-((2-(2-morpholinoethoxy)ethoxy)methyl)oxazol-2-amine; N,N-bis(3-methoxybenzyl)-4-((2-morpholinoethoxy)methyl)oxazol-2-amine; N-(3-methoxybenzyl)-4-((2-(3-methoxybenzyloxy)ethoxy)methyl)-N-(4-(pyrrolidin-1-yl)benzyl)oxazol-2-amine; N-(4-(1H-imidazol-1-yl)benzyl)-N-(3-methoxybenzyl)-4-((2-(3-methoxybenzyloxy)ethoxy)methyl)oxazol-2-amine; N-(4-(1H-pyrazol-1-yl)benzyl)-N-(3-methoxybenzyl)-4-((2-(3-methoxybenzyloxy)ethoxy)methyl)oxazol-2-amine; N-(3-methoxybenzyl)-4-((2-(3-methoxybenzyloxy)ethoxy)methyl)-N-(4-morpholinobenzyl)oxazol-2-amine; N-(3-methoxybenzyl)-4-((2-(3-methoxybenzyloxy)ethoxy)methyl)-N-(4-(4-methylpiperazin-1-yl)benzyl)oxazol-2-amine; N-(3-methoxybenzyl)-4-((2-(3-methoxybenzyloxy)ethoxy)methyl)-N-(quinolin-6-ylmethyl)thiazol-2-amine; N-(3-methoxybenzyl)-4-((2-(3-methoxybenzyloxy)ethoxy)methyl)-N-(3-(pyrrolidin-1-yl)benzyl)thiazol-2-amine; N-(3-(dimethylamino)benzyl)-N-(3-methoxybenzyl)-4-((2-(3-methoxybenzyloxy)ethoxy)methyl)thiazol-2-amine; N-(3-(1H-imidazol-1-yl)benzyl)-N-(3-methoxybenzyl)-4-((2-(3-methoxybenzyloxy)ethoxy)methyl)thiazol-2-amine; N-(3-(1H-pyrazol-1-yl)benzyl)-N-(3-methoxybenzyl)-4-((2-(3-methoxybenzyloxy)ethoxy)methyl)thiazol-2-amine; N-(3-methoxybenzyl)-4-((2-(3-methoxybenzyloxy)ethoxy)methyl)-N-(3-morpholinobenzyl)thiazol-2-amine; N-(3-methoxybenzyl)-4-((2-(3-methoxybenzyloxy)ethoxy)methyl)-N-(3-(4-methylpiperazin-1-yl)benzyl)thiazol-2-amine; N-((1H-benzo[d]imidazol-5-yl)methyl)-N-(3-methoxybenzyl)-4-((2-(3-methoxybenzyloxy)ethoxy)methyl)thiazol-2-amine; N-((1H-indazol-5-yl)methyl)-N-(3-methoxybenzyl)-4-((2-(3-methoxybenzyloxy)ethoxy)methyl)thiazol-2-amine; N-(3-methoxybenzyl)-4-((2-(3-methoxybenzyloxy)ethoxy)methyl)-N-(4-(pyrrolidin-1-yl)benzyl)thiazol-2-amine; N-(4-(1H-imidazol-1-yl)benzyl)-N-(3-methoxybenzyl)-4-((2-(3-methoxybenzyloxy)ethoxy)methyl)thiazol-2-amine; N-(4-(1H-pyrazol-1-yl)benzyl)-N-(3-methoxybenzyl)-4-((2-(3-methoxybenzyloxy)ethoxy)methyl)thiazol-2-amine; N-(3-methoxybenzyl)-4-((2-(3-methoxybenzyloxy)ethoxy)methyl)-N-(4-morpholinobenzyl)thiazol-2-amine; N-(3-methoxybenzyl)-4-((2-(3-methoxybenzyloxy)ethoxy)methyl)-N-(4-(4-methylpiperazin-1-yl)benzyl)thiazol-2-amine; N-(3-methoxybenzyl)-N-(3-(4-methylpiperazin-1-yl)benzyl)-4-((2-morpholinoethoxy)methyl)oxazol-2-amine; N-(3-methoxybenzyl)-N-(3-morpholinobenzyl)-4-((2-morpholinoethoxy)methyl)oxazol-2-amine; N-(3-methoxybenzyl)-4-((2-morpholinoethoxy)methyl)-N-(3-(pyrrolidin-1-yl)benzyl)oxazol-2-amine; N-(3-(1H-imidazol-1-yl)benzyl)-N-(3-methoxybenzyl)-4-((2-morpholinoethoxy)methyl)oxazol-2-amine; N-(3-(1H-pyrazol-1-yl)benzyl)-N-(3-methoxybenzyl)-4-((2-morpholinoethoxy)methyl)oxazol-2-amine; N-(3-(dimethylamino)benzyl)-N-(3-methoxybenzyl)-4-((2-morpholinoethoxy)methyl)oxazol-2-amine; N-(3-(dimethylamino)benzyl)-N-(3-methoxybenzyl)-4-((2-morpholinoethoxy)methyl)thiazol-2-amine; N-(3-methoxybenzyl)-4-((2-morpholinoethoxy)methyl)-N-(3-(pyrrolidin-1-yl)benzyl)thiazol-2-amine; N-(3-(1H-pyrazol-1-yl)benzyl)-N-(3-methoxybenzyl)-4-((2-morpholinoethoxy)methyl)thiazol-2-amine; N-(3-(1H-imidazol-1-yl)benzyl)-N-(3-methoxybenzyl)-4-((2-morpholinoethoxy)methyl)thiazol-2-amine; N-(3-methoxybenzyl)-4-((2-morpholinoethoxy)methyl)-N-(4-(pyrrolidin-1-yl)benzyl)thiazol-2-amine; N-(3-methoxybenzyl)-N-(4-morpholinobenzyl)-4-((2-morpholinoethoxy)methyl)thiazol-2-amine; N-(3-methoxybenzyl)-N-(4-(4-methylpiperazin-1-yl)benzyl)-4-((2-morpholinoethoxy)methyl)thiazol-2-amine; N-(3-methoxybenzyl)-N-(3-(4-methylpiperazin-1-yl)benzyl)-4-((2-morpholinoethoxy)methyl)thiazol-2-amine; N-(3-methoxybenzyl)-N-(3-morpholinobenzyl)-4-((2-morpholinoethoxy)methyl)thiazol-2-amine; N-(4-(1H-pyrazol-1-yl)benzyl)-N-(3-methoxybenzyl)-4-((2-morpholinoethoxy)methyl)thiazol-2-amine; N-(4-(1H-imidazol-1-yl)benzyl)-N-(3-methoxybenzyl)-4-((2-morpholinoethoxy)methyl)thiazol-2-amine; N-((1H-benzo[d]imidazol-6-yl)methyl)-N-(3-methoxybenzyl)-4-((2-morpholinoethoxy)methyl)thiazol-2-amine; N-((1H-indazol-5-yl)methyl)-N-(3-methoxybenzyl)-4-((2-morpholinoethoxy)methyl)thiazol-2-amine; N-(3-methoxybenzyl)-4-((2-morpholinoethoxy)methyl)-N-(quinolin-6-ylmethyl)thiazol-2-amine; 4-((2-(2-(dimethylamino)ethoxy)ethoxy)methyl)-N,N-bis(3-methoxybenzyl)oxazol-2-amine; 4-((2-(dimethylamino)ethoxy)methyl)-N,N-bis(3-methoxybenzyl)oxazol-2-amine; N,N-bis(3-methoxybenzyl)-4-(2-methyl-5,8,11-trioxa-2-azadodecan-12-yl)oxazol-2-amine; N,N-bis(3-methoxybenzyl)-4-((2-(2-(2-morpholinoethoxy)ethoxy)ethoxy)methyl)thiazol-2-amine; 4-((2-(2-(indolin-1-yl)ethoxy)ethoxy)methyl)-N,N-bis(3-methoxybenzyl)oxazol-2-amine; 4-((2-(2-(3,4-dihydroquinolin-1(2H)-yl)ethoxy)ethoxy)methyl)-N,N-bis(3-methoxybenzyl)oxazol-2-amine; 4-((2-(3,4-dihydroquinolin-1(2H)-yl)ethoxy)methyl)-N,N-bis(3-methoxybenzyl)oxazol-2-amine; 4-((2-(indolin-1-yl)ethoxy)methyl)-N,N-bis(3-methoxybenzyl)oxazol-2-amine; 1-((2-(bis(3-methoxybenzyl)amino)oxazol-4-yl)methyl)piperazin-2-one; 1-((2-(bis(3-methoxybenzyl)amino)oxazol-4-yl)methyl)piperazine-2,5-dione; 4-((2-(bis(3-methoxybenzyl)amino)oxazol-4-yl)methyl)piperazin-2-one; N-(4-(1H-imidazol-1-yl)benzyl)-4-((2-(3-(dimethylamino)phenoxy)ethoxy)methyl)-N-(3-methoxybenzyl)oxazol-2-amine; N-(4-(1H-pyrazol-1-yl)benzyl)-4-((2-(3-(dimethylamino)phenoxy)ethoxy)methyl)-N-(3-methoxybenzyl)oxazol-2-amine; 4-((2-(3-(dimethylamino)phenoxy)ethoxy)methyl)-N-(3-methoxybenzyl)-N-(4-morpholinobenzyl)oxazol-2-amine; 4-((2-(3-(dimethylamino)phenoxy)ethoxy)methyl)-N-(3-methoxybenzyl)-N-(4-(pyrrolidin-1-yl)benzyl)oxazol-2-amine; 4-((2-(3-(dimethylamino)phenoxy)ethoxy)methyl)-N-(3-methoxybenzyl)-N-(4-(4-methylpiperazin-1-yl)benzyl)oxazol-2-amine; N-(3-(dimethylamino)benzyl)-4-((2-(3-(dimethylamino)phenoxy)ethoxy)methyl)-N-(3-methoxybenzyl)oxazol-2-amine; 4-((2-(3-(dimethylamino)phenoxy)ethoxy)methyl)-N-(3-methoxybenzyl)-N-(3-(pyrrolidin-1-yl)benzyl)oxazol-2-amine; N-(3-(1H-imidazol-1-yl)benzyl)-4-((2-(3-(dimethylamino)phenoxy)ethoxy)methyl)-N-(3-methoxybenzyl)oxazol-2-amine; N-(3-(1H-pyrazol-1-yl)benzyl)-4-((2-(3-(dimethylamino)phenoxy)ethoxy)methyl)-N-(3-methoxybenzyl)oxazol-2-amine; 4-((2-(3-(dimethylamino)phenoxy)ethoxy)methyl)-N-(3-methoxybenzyl)-N-(3-morpholinobenzyl)oxazol-2-amine; 4-((2-(3-(dimethylamino)phenoxy)ethoxy)methyl)-N-(3-methoxybenzyl)-N-(3-(4-methylpiperazin-1-yl)benzyl)oxazol-2-amine; N-((1H-benzo[d]imidazol-6-yl)methyl)-4-((2-(3-(dimethylamino)phenoxy)ethoxy)methyl)-N-(3-methoxybenzyl)oxazol-2-amine; N-((1H-indazol-5-yl)methyl)-4-((2-(3-(dimethylamino)phenoxy)ethoxy)methyl)-N-(3-methoxybenzyl)oxazol-2-amine; 4-((2-(3-(dimethylamino)phenoxy)ethoxy)methyl)-N-(3-methoxybenzyl)-N-(quinolin-6-ylmethyl)oxazol-2-amine; N-(4-(1H-imidazol-1-yl)benzyl)-4-((2-(3-(dimethylamino)phenoxy)ethoxy)methyl)-N-(3-methoxybenzyl)thiazol-2-amine; N-(4-(1H-pyrazol-1-yl)benzyl)-4-((2-(3-(dimethylamino)phenoxy)ethoxy)methyl)-N-(3-methoxybenzyl)thiazol-2-amine; 4-((2-(3-(dimethylamino)phenoxy)ethoxy)methyl)-N-(3-methoxybenzyl)-N-(4-morpholinobenzyl)thiazol-2-amine; 4-((2-(3-(dimethylamino)phenoxy)ethoxy)methyl)-N-(3-methoxybenzyl)-N-(4-(pyrrolidin-1-yl)benzyl)thiazol-2-amine; 4-((2-(3-(dimethylamino)phenoxy)ethoxy)methyl)-N-(3-methoxybenzyl)-N-(4-(4-methylpiperazin-1-yl)benzyl)thiazol-2-amine; N-(3-(dimethylamino)benzyl)-4-((2-(3-(dimethylamino)phenoxy)ethoxy)methyl)-N-(3-methoxybenzyl)thiazol-2-amine; 4-((2-(3-(dimethylamino)phenoxy)ethoxy)methyl)-N-(3-methoxybenzyl)-N-(3-(pyrrolidin-1-yl)benzyl)thiazol-2-amine; N-(3-(1H-imidazol-1-yl)benzyl)-4-((2-(3-(dimethylamino)phenoxy)ethoxy)methyl)-N-(3-methoxybenzyl)thiazol-2-amine; N-(3-(1H-pyrazol-1-yl)benzyl)-4-((2-(3-(dimethylamino)phenoxy)ethoxy)methyl)-N-(3-methoxybenzyl)thiazol-2-amine; 4-((2-(3-(dimethylamino)phenoxy)ethoxy)methyl)-N-(3-methoxybenzyl)-N-(3-morpholinobenzyl)thiazol-2-amine; 4-((2-(3-(dimethylamino)phenoxy)ethoxy)methyl)-N-(3-methoxybenzyl)-N-(3-(4-methylpiperazin-1-yl)benzyl)thiazol-2-amine; N-((1H-benzo[d]imidazol-5-yl)methyl)-4-((2-(3-(dimethylamino)phenoxy)ethoxy)methyl)-N-(3-methoxybenzyl)thiazol-2-amine; N-((1H-indazol-5-yl)methyl)-4-((2-(3-(dimethylamino)phenoxy)ethoxy)methyl)-N-(3-methoxybenzyl)thiazol-2-amine; 4-((2-(3-(dimethylamino)phenoxy)ethoxy)methyl)-N-(3-methoxybenzyl)-N-(quinolin-6-ylmethyl)thiazol-2-amine; N-(3-methoxybenzyl)-4-(morpholinomethyl)-N-(3-(pyrrolidin-1-yl)benzyl)oxazol-2-amine; N-(3-(1H-pyrazol-1-yl)benzyl)-N-(3-methoxybenzyl)-4-(morpholinomethyl)oxazol-2-amine; N-(3-(1H-imidazol-1-yl)benzyl)-N-(3-methoxybenzyl)-4-(morpholinomethyl)oxazol-2-amine; N-(3-methoxybenzyl)-N-(3-morpholinobenzyl)-4-(morpholinomethyl)oxazol-2-amine; N-(3-methoxybenzyl)-N-(3-(4-methylpiperazin-1-yl)benzyl)-4-(morpholinomethyl)oxazol-2-amine; N-(3-methoxybenzyl)-4-(morpholinomethyl)-N-(4-(pyrrolidin-1-yl)benzyl)oxazol-2-amine; N-(3-methoxybenzyl)-N-(4-morpholinobenzyl)-4-(morpholinomethyl)oxazol-2-amine; N-(3-methoxybenzyl)-N-(4-(4-methylpiperazin-1-yl)benzyl)-4-(morpholinomethyl)oxazol-2-amine; N-(4-(1H-pyrazol-1-yl)benzyl)-N-(3-methoxybenzyl)-4-(morpholinomethyl)oxazol-2-amine; N-(4-(1H-imidazol-1-yl)benzyl)-N-(3-methoxybenzyl)-4-(morpholinomethyl)oxazol-2-amine; N-((1H-benzo[d]imidazol-5-yl)methyl)-N-(3-methoxybenzyl)-4-(morpholinomethyl)oxazol-2-amine; N-((1H-indazol-6-yl)methyl)-N-(3-methoxybenzyl)-4-(morpholinomethyl)oxazol-2-amine; N-(3-methoxybenzyl)-4-(morpholinomethyl)-N-(quinolin-6-ylmethyl)oxazol-2-amine; N-(3-(dimethylamino)benzyl)-N-(3-methoxybenzyl)-4-(morpholinomethyl)oxazol-2-amine; N-(3-methoxybenzyl)-4-(morpholinomethyl)-N-(3-(pyrrolidin-1-yl)benzyl)thiazol-2-amine; N-(3-(1H-pyrazol-1-yl)benzyl)-N-(3-methoxybenzyl)-4-(morpholinomethyl)thiazol-2-amine; N-(3-(1H-imidazol-1-yl)benzyl)-N-(3-methoxybenzyl)-4-(morpholinomethyl)thiazol-2-amine; N-(3-methoxybenzyl)-4-(morpholinomethyl)-N-(4-(pyrrolidin-1-yl)benzyl)thiazol-2-amine; N-(3-methoxybenzyl)-N-(4-morpholinobenzyl)-4-(morpholinomethyl)thiazol-2-amine; N-(3-methoxybenzyl)-N-(4-(4-methylpiperazin-1-yl)benzyl)-4-(morpholinomethyl)thiazol-2-amine; N-(4-(1H-pyrazol-1-yl)benzyl)-N-(3-methoxybenzyl)-4-(morpholinomethyl)thiazol-2-amine; N-(4-(1H-imidazol-1-yl)benzyl)-N-(3-methoxybenzyl)-4-(morpholinomethyl)thiazol-2-amine; N-(3-methoxybenzyl)-N-(3-morpholinobenzyl)-4-(morpholinomethyl)thiazol-2-amine; N-(3-methoxybenzyl)-N-(3-(4-methylpiperazin-1-yl)benzyl)-4-(morpholinomethyl)thiazol-2-amine; N-((1H-benzo[d]imidazol-6-yl)methyl)-N-(3-methoxybenzyl)-4-(morpholinomethyl)thiazol-2-amine; N-((1H-indazol-6-yl)methyl)-N-(3-methoxybenzyl)-4-(morpholinomethyl)thiazol-2-amine; N-(3-methoxybenzyl)-4-(morpholinomethyl)-N-(quinolin-6-ylmethyl)thiazol-2-amine; N-(3-(dimethylamino)benzyl)-N-(3-methoxybenzyl)-4-(morpholinomethyl)thiazol-2-amine; N-(3-methoxybenzyl)-4-(piperidin-1-ylmethyl)-N-(3-(pyrrolidin-1-yl)benzyl)oxazol-2-amine; N-(3-(1H-pyrazol-1-yl)benzyl)-N-(3-methoxybenzyl)-4-(piperidin-1-ylmethyl)oxazol-2-amine; N-(3-(1H-imidazol-1-yl)benzyl)-N-(3-methoxybenzyl)-4-(piperidin-1-ylmethyl)oxazol-2-amine; N-(3-methoxybenzyl)-N-(3-morpholinobenzyl)-4-(piperidin-1-ylmethyl)oxazol-2-amine; N-(3-methoxybenzyl)-N-(3-(4-methylpiperazin-1-yl)benzyl)-4-(piperidin-1-ylmethyl)oxazol-2-amine; N-(3-methoxybenzyl)-4-(piperidin-1-ylmethyl)-N-(4-(pyrrolidin-1-yl)benzyl)oxazol-2-amine; N-(3-methoxybenzyl)-N-(4-morpholinobenzyl)-4-(piperidin-1-ylmethyl)oxazol-2-amine; N-(3-methoxybenzyl)-N-(4-(4-methylpiperazin-1-yl)benzyl)-4-(piperidin-1-ylmethyl)oxazol-2-amine; N-(4-(1H-pyrazol-1-yl)benzyl)-N-(3-methoxybenzyl)-4-(piperidin-1-ylmethyl)oxazol-2-amine; N-(4-(1H-imidazol-1-yl)benzyl)-N-(3-methoxybenzyl)-4-(piperidin-1-ylmethyl)oxazol-2-amine; N-((1H-benzo[d]imidazol-5-yl)methyl)-N-(3-methoxybenzyl)-4-(piperidin-1-ylmethyl)oxazol-2-amine; N-((1H-indazol-6-yl)methyl)-N-(3-methoxybenzyl)-4-(piperidin-1-ylmethyl)oxazol-2-amine; N-(3-methoxybenzyl)-4-(piperidin-1-ylmethyl)-N-(quinolin-6-ylmethyl)oxazol-2-amine; N-(3-(dimethylamino)benzyl)-N-(3-methoxybenzyl)-4-(piperidin-1-ylmethyl)oxazol-2-amine; N-(3-methoxybenzyl)-4-(piperidin-1-ylmethyl)-N-(3-(pyrrolidin-1-yl)benzyl)thiazol-2-amine; N-(3-(1H-pyrazol-1-yl)benzyl)-N-(3-methoxybenzyl)-4-(piperidin-1-ylmethyl)thiazol-2-amine; N-(3-(1H-imidazol-1-yl)benzyl)-N-(3-methoxybenzyl)-4-(piperidin-1-ylmethyl)thiazol-2-amine; N-(3-methoxybenzyl)-4-(piperidin-1-ylmethyl)-N-(4-(pyrrolidin-1-yl)benzyl)thiazol-2-amine; N-(3-methoxybenzyl)-N-(4-morpholinobenzyl)-4-(piperidin-1-ylmethyl)thiazol-2-amine; N-(3-methoxybenzyl)-N-(4-(4-methylpiperazin-1-yl)benzyl)-4-(piperidin-1-ylmethyl)thiazol-2-amine; N-(4-(1H-pyrazol-1-yl)benzyl)-N-(3-methoxybenzyl)-4-(piperidin-1-ylmethyl)thiazol-2-amine; N-(4-(1H-imidazol-1-yl)benzyl)-N-(3-methoxybenzyl)-4-(piperidin-1-ylmethyl)thiazol-2-amine; N-(3-methoxybenzyl)-N-(3-morpholinobenzyl)-4-(piperidin-1-ylmethyl)thiazol-2-amine; N-(3-methoxybenzyl)-N-(3-(4-methylpiperazin-1-yl)benzyl)-4-(piperidin-1-ylmethyl)thiazol-2-amine; N-((1H-benzo[d]imidazol-6-yl)methyl)-N-(3-methoxybenzyl)-4-(piperidin-1-ylmethyl)thiazol-2-amine; N-((1H-indazol-6-yl)methyl)-N-(3-methoxybenzyl)-4-(piperidin-1-ylmethyl)thiazol-2-amine; N-(3-methoxybenzyl)-4-(piperidin-1-ylmethyl)-N-(quinolin-6-ylmethyl)thiazol-2-amine; N-(3-(dimethylamino)benzyl)-N-(3-methoxybenzyl)-4-(piperidin-1-ylmethyl)thiazol-2-amine; N-(3-methoxybenzyl)-4-((4-methylpiperazin-1-yl)methyl)-N-(3-(pyrrolidin-1-yl)benzyl)oxazol-2-amine; N-(3-(1H-pyrazol-1-yl)benzyl)-N-(3-methoxybenzyl)-4-((4-methylpiperazin-1-yl)methyl)oxazol-2-amine; N-(3-(1H-imidazol-1-yl)benzyl)-N-(3-methoxybenzyl)-4-((4-methylpiperazin-1-yl)methyl)oxazol-2-amine; N-(3-methoxybenzyl)-4-((4-methylpiperazin-1-yl)methyl)-N-(3-morpholinobenzyl)oxazol-2-amine; N-(3-methoxybenzyl)-N-(3-(4-methylpiperazin-1-yl)benzyl)-4-(piperidin-1-ylmethyl)oxazol-2-amine; N-(3-methoxybenzyl)-4-((4-methylpiperazin-1-yl)methyl)-N-(4-(pyrrolidin-1-yl)benzyl)oxazol-2-amine; N-(3-methoxybenzyl)-4-((4-methylpiperazin-1-yl)methyl)-N-(4-morpholinobenzyl)oxazol-2-amine; N-(3-methoxybenzyl)-N-(4-(4-methylpiperazin-1-yl)benzyl)-4-((4-methylpiperazin-1-yl)methyl)oxazol-2-amine; N-(4-(1H-pyrazol-1-yl)benzyl)-N-(3-methoxybenzyl)-4-((4-methylpiperazin-1-yl)methyl)oxazol-2-amine; N-(4-(1H-imidazol-1-yl)benzyl)-N-(3-methoxybenzyl)-4-((4-methylpiperazin-1-yl)methyl)oxazol-2-amine; N-((1H-benzo[d]imidazol-5-yl)methyl)-N-(3-methoxybenzyl)-4-((4-methylpiperazin-1-yl)methyl)oxazol-2-amine; N-((1H-indazol-6-yl)methyl)-N-(3-methoxybenzyl)-4-((4-methylpiperazin-1-yl)methyl)oxazol-2-amine; N-(3-methoxybenzyl)-4-((4-methylpiperazin-1-yl)methyl)-N-(quinolin-6-ylmethyl)oxazol-2-amine; N-(3-(dimethylamino)benzyl)-N-(3-methoxybenzyl)-4-((4-methylpiperazin-1-yl)methyl)oxazol-2-amine; N-(3-methoxybenzyl)-4-((4-methylpiperazin-1-yl)methyl)-N-(3-(pyrrolidin-1-yl)benzyl)thiazol-2-amine; N-(3-(1H-pyrazol-1-yl)benzyl)-N-(3-methoxybenzyl)-4-((4-methylpiperazin-1-yl)methyl)thiazol-2-amine; N-(3-(1H-imidazol-1-yl)benzyl)-N-(3-methoxybenzyl)-4-((4-methylpiperazin-1-yl)methyl)thiazol-2-amine; N-(3-methoxybenzyl)-4-((4-methylpiperazin-1-yl)methyl)-N-(4-morpholinobenzyl)thiazol-2-amine; N-(3-methoxybenzyl)-N-(4-(4-methylpiperazin-1-yl)benzyl)-4-((4-methylpiperazin-1-yl)methyl)thiazol-2-amine N-(4-(1H-pyrazol-1-yl)benzyl)-N-(3-methoxybenzyl)-4-((4-methylpiperazin-1-yl)methyl)thiazol-2-amine; N-(4-(1H-imidazol-1-yl)benzyl)-N-(3-methoxybenzyl)-4-((4-methylpiperazin-1-yl)methyl)thiazol-2-amine; N-(3-methoxybenzyl)-4-((4-methylpiperazin-1-yl)methyl)-N-(3-morpholinobenzyl)thiazol-2-amine; N-(3-methoxybenzyl)-N-(3-(4-methylpiperazin-1-yl)benzyl)-4-((4-methylpiperazin-1-yl)methyl)thiazol-2-amine; N-((1H-benzo[d]imidazol-6-yl)methyl)-N-(3-methoxybenzyl)-4-((4-methylpiperazin-1-yl)methyl)thiazol-2-amine; N-((1H-indazol-6-yl)methyl)-N-(3-methoxybenzyl)-4-((4-methylpiperazin-1-yl)methyl)thiazol-2-amine; N-(3-methoxybenzyl)-4-((4-methylpiperazin-1-yl)methyl)-N-(quinolin-6-ylmethyl)thiazol-2-amine; N-(3-(dimethylamino)benzyl)-N-(3-methoxybenzyl)-4-((4-methylpiperazin-1-yl)methyl)thiazol-2-amine; 4-((dimethylamino)methyl)-N-(3-methoxybenzyl)-N-(3-(pyrrolidin-1-yl)benzyl)oxazol-2-amine; N-(3-(1H-pyrazol-1-yl)benzyl)-4-((dimethylamino)methyl)-N-(3-methoxybenzyl)oxazol-2-amine; N-(3-(1H-imidazol-1-yl)benzyl)-4-((dimethylamino)methyl)-N-(3-methoxybenzyl)oxazol-2-amine; 4-((dimethylamino)methyl)-N-(3-methoxybenzyl)-N-(3-morpholinobenzyl)oxazol-2-amine 4-((dimethylamino)methyl)-N-(3-methoxybenzyl)-N-(3-(4-methylpiperazin-1-yl)benzyl)oxazol-2-amine; 4-((dimethylamino)methyl)-N-(3-methoxybenzyl)-N-(4-(pyrrolidin-1-yl)benzyl)oxazol-2-amine; 4-((dimethylamino)methyl)-N-(3-methoxybenzyl)-N-(4-morpholinobenzyl)oxazol-2-amine; 4-((dimethylamino)methyl)-N-(3-methoxybenzyl)-N-(4-(4-methylpiperazin-1-yl)benzyl)oxazol-2-amine; N-(4-(1H-pyrazol-1-yl)benzyl)-4-((dimethylamino)methyl)-N-(3-methoxybenzyl)oxazol-2-amine; N-(4-(1H-imidazol-1-yl)benzyl)-4-((dimethylamino)methyl)-N-(3-methoxybenzyl)oxazol-2-amine; N-((1H-indazol-6-yl)methyl)-4-((dimethylamino)methyl)-N-(3-methoxybenzyl)oxazol-2-amine; N-((1H-benzo[d]imidazol-6-yl)methyl)-4-((dimethylamino)methyl)-N-(3-methoxybenzyl)oxazol-2-amine; 4-((dimethylamino)methyl)-N-(3-methoxybenzyl)-N-(quinolin-6-ylmethyl)oxazol-2-amine; N-(3-(dimethylamino)benzyl)-4-((dimethylamino)methyl)-N-(3-methoxybenzyl)oxazol-2-amine; 4-((dimethylamino)methyl)-N-(3-methoxybenzyl)-N-(3-(pyrrolidin-1-yl)benzyl)thiazol-2-amine; N-(3-(1H-pyrazol-1-yl)benzyl)-4-((dimethylamino)methyl)-N-(3-methoxybenzyl)thiazol-2-amine; N-(3-(1H-imidazol-1-yl)benzyl)-4-((dimethylamino)methyl)-N-(3-methoxybenzyl)thiazol-2-amine 4-((dimethylamino)methyl)-N-(3-methoxybenzyl)-N-(4-(pyrrolidin-1-yl)benzyl)thiazol-2-amine; 4-((dimethylamino)methyl)-N-(3-methoxybenzyl)-N-(4-(4-methylpiperazin-1-yl)benzyl)thiazol-2-amine; N-(4-(1H-pyrazol-1-yl)benzyl)-4-((dimethylamino)methyl)-N-(3-methoxybenzyl)thiazol-2-amine; N-(4-(1H-imidazol-1-yl)benzyl)-4-((dimethylamino)methyl)-N-(3-methoxybenzyl)thiazol-2-amine; 4-((dimethylamino)methyl)-N-(3-methoxybenzyl)-N-(3-morpholinobenzyl)thiazol-2-amine; 4-((dimethylamino)methyl)-N-(3-methoxybenzyl)-N-(3-(4-methylpiperazin-1-yl)benzyl)thiazol-2-amine; N-((1H-indazol-6-yl)methyl)-4-((dimethylamino)methyl)-N-(3-methoxybenzyl)thiazol-2-amine; 4-((dimethylamino)methyl)-N-(3-methoxybenzyl)-N-(quinolin-6-ylmethyl)thiazol-2-amine;
N-(3-(dimethylamino)benzyl)-4-((dimethylamino)methyl)-N-(3-methoxybenzyl)thiazol-2-amine; 4-((dimethylamino)methyl)-N,N-bis(3-methoxybenzyl)oxazol-2-amine; 4-((dimethylamino)methyl)-N,N-bis(3-methoxybenzyl)thiazol-2-amine; N,N-bis(3-methoxybenzyl)-4-((2-(2-(3-methoxybenzyloxy)ethoxy)ethoxy)methyl)aniline; 4-((2-(2-(benzyloxy)ethoxy)ethoxy)methyl)-N,N-bis(3-methoxybenzyl)aniline; N,N-bis(3-methoxybenzyl)-4-((2-(2-(3-methoxyphenoxy)ethoxy)ethoxy)methyl)aniline; N,N-bis(3-methoxybenzyl)-4-((2-(2-morpholinoethoxy)ethoxy)methyl)aniline; N,N-bis(3-methoxybenzyl)-4-((2-(3-methoxybenzyloxy)ethoxy)methyl)aniline; 3-(2-(4-(bis(3-methoxybenzyl)amino)benzyloxy)ethoxy)-N,N-dimethylaniline; N,N-bis(3-methoxybenzyl)-4-((2-morpholinoethoxy)methyl)aniline; N,N-bis(3-methoxybenzyl)-4-(morpholinomethyl)aniline; N,N-bis(3-methoxybenzyl)-4-(piperidin-1-ylmethyl)aniline; N,N-bis(3-methoxybenzyl)-4-((4-methylpiperazin-1-yl)methyl)aniline; 4-((dimethylamino)methyl)-N,N-bis(3-methoxybenzyl)aniline; 1-(4-(bis(3-methoxybenzyl)amino)benzyl)piperazin-2-one; 4-(4-(bis(3-methoxybenzyl)amino)benzyl)piperazin-2-one; 1-(4-(bis(3-methoxybenzyl)amino)benzyl)piperazine-2,5-dione; 3-(((3-methoxybenzyl)(4-((2-(2-(3-methoxybenzyloxy)ethoxy)ethoxy)methyl)phenyl)amino)methyl)-N,N-dimethylaniline; 3-(((4-((2-(2-(benzyloxy)ethoxy)ethoxy)methyl)phenyl)(3-methoxybenzyl)amino)methyl)-N,N-dimethylaniline 3-(((3-methoxybenzyl)(4-((2-(2-(3-methoxyphenoxy)ethoxy)ethoxy)methyl)phenyl)amino)methyl)-N,N-dimethylaniline; 3-(((3-methoxybenzyl)(4-((2-(2-morpholinoethoxy)ethoxy)methyl)phenyl)amino)methyl)-N,N-dimethylaniline; 3-(((3-methoxybenzyl)(4-((2-(3-methoxybenzyloxy)ethoxy)methyl)phenyl)amino)methyl)-N,N-dimethylaniline; 3-(2-(4-((3-(dimethylamino)benzyl)(3-methoxybenzyl)amino)benzyloxy)ethoxy)-N,N-dimethylaniline; 3-(((3-methoxybenzyl)(4-((2-morpholinoethoxy)methyl)phenyl)amino)methyl)-N,N-dimethylaniline; 3-(((3-methoxybenzyl)(4-(morpholinomethyl)phenyl)amino)methyl)-N,N-dimethylaniline; 3-(((3-methoxybenzyl)(4-(piperidin-1-ylmethyl)phenyl)amino)methyl)-N,N-dimethylaniline; 3-(((3-methoxybenzyl)(4-((4-methylpiperazin-1-yl)methyl)phenyl)amino)methyl)-N,N-dimethylaniline; 1-(4-((3-(dimethylamino)benzyl)(3-methoxybenzyl)amino)benzyl)piperazin-2-one; 1-(4-((3-(dimethylamino)benzyl)(3-methoxybenzyl)amino)benzyl)piperazine-2,5-dione; 4-(4-((3-(dimethylamino)benzyl)(3-methoxybenzyl)amino)benzyl)piperazin-2-one; 3-(((4-((dimethylamino)methyl)phenyl)(3-methoxybenzyl)amino)methyl)-N,N-dimethylaniline; N-(3-methoxybenzyl)-4-((2-(2-morpholinoethoxy)ethoxy)methyl)-N-(3-(pyrrolidin-1-yl)benzyl)aniline; N-(3-methoxybenzyl)-4-((2-(3-methoxybenzyloxy)ethoxy)methyl)-N-(3-(pyrrolidin-1-yl)benzyl)aniline; 3-(2-(4-((3-methoxybenzyl)(3-(pyrrolidin-1-yl)benzyl)amino)benzyloxy)ethoxy)-N,N-dimethylaniline; N-(3-methoxybenzyl)-4-((2-morpholinoethoxy)methyl)-N-(3-(pyrrolidin-1-yl)benzyl)aniline; N-(3-methoxybenzyl)-4-(morpholinomethyl)-N-(3-(pyrrolidin-1-yl)benzyl)aniline; N-(3-methoxybenzyl)-4-(piperidin-1-ylmethyl)-N-(3-(pyrrolidin-1-yl)benzyl)aniline; N-(3-methoxybenzyl)-4-((4-methylpiperazin-1-yl)methyl)-N-(3-(pyrrolidin-1-yl)benzyl)aniline; 1-(4-((3-methoxybenzyl)(3-(pyrrolidin-1-yl)benzyl)amino)benzyl)piperazin-2-one; 1-(4-((3-methoxybenzyl)(3-(pyrrolidin-1-yl)benzyl)amino)benzyl)piperazine-2,5-dione; 4-(4-((3-methoxybenzyl)(3-(pyrrolidin-1-yl)benzyl)amino)benzyl)piperazin-2-one; 4-((dimethylamino)methyl)-N-(3-methoxybenzyl)-N-(3-(pyrrolidin-1-yl)benzyl)aniline; N-(3-(1H-pyrazol-1-yl)benzyl)-N-(3-methoxybenzyl)-4-((2-(2-morpholinoethoxy)ethoxy)methyl)aniline; N-(3-(1H-pyrazol-1-yl)benzyl)-N-(3-methoxybenzyl)-4-((2-(3 methoxybenzyloxy)ethoxy)methyl)aniline; 3-(2-(4-((3-(1H-pyrazol-1-yl)benzyl)(3-methoxybenzyl)amino)benzyloxy)ethoxy)-N,N-dimethylaniline; N-(3-(1H-pyrazol-1-yl)benzyl)-N-(3-methoxybenzyl)-4-((2-morpholinoethoxy)methyl)aniline; N-(3-(1H-pyrazol-1-yl)benzyl)-N-(3-methoxybenzyl)-4-(morpholinomethyl)aniline; N-(3-(1H-pyrazol-1-yl)benzyl)-N-(3-methoxybenzyl)-4-(piperidin-1-ylmethyl)aniline; N-(3-(1H-pyrazol-1-yl)benzyl)-N-(3-methoxybenzyl)-4-((4-methylpiperazin-1-yl)methyl)aniline; 1-(4-((3-(1H-pyrazol-1-yl)benzyl)(3-methoxybenzyl)amino)benzyl)piperazin-2-one; 1-(4-((3-(1H-pyrazol-1-yl)benzyl)(3-methoxybenzyl)amino)benzyl)piperazine-2,5-dione; 4-(4-((3-(1H-pyrazol-1-yl)benzyl)(3-methoxybenzyl)amino)benzyl)piperazin-2-one; N-(3-(1H-pyrazol-1-yl)benzyl)-4-((dimethylamino)methyl)-N-(3-methoxybenzyl)aniline N-(3-(1H-imidazol-1-yl)benzyl)-N-(3-methoxybenzyl)-4-((2-(2-morpholinoethoxy)ethoxy)methyl)aniline; N-(3-(1H-imidazol-1-yl)benzyl)-N-(3-methoxybenzyl)-4-((2-(3-methoxybenzyloxy)ethoxy)methyl)aniline; 3-(2-(4-((3-(1H-imidazol-1-yl)benzyl)(3-methoxybenzyl)amino)benzyloxy)ethoxy)-N,N-dimethylaniline; N-(3-(1H-imidazol-1-yl)benzyl)-N-(3-methoxybenzyl)-4-((2-morpholinoethoxy)methyl)aniline; N-(3-(1H-imidazol-1-yl)benzyl)-N-(3-methoxybenzyl)-4-(morpholinomethyl)aniline; N-(3-(1H-imidazol-1-yl)benzyl)-N-(3-methoxybenzyl)-4-(piperidin-1-ylmethyl)aniline; N-(3-(1H-imidazol-1-yl)benzyl)-N-(3-methoxybenzyl)-4-((4-methylpiperazin-1-yl)methyl)aniline; 1-(4-((3-(1H-imidazol-1-yl)benzyl)(3-methoxybenzyl)amino)benzyl)piperazin-2-one; 1-(4-((3-(1H-imidazol-1-yl)benzyl)(3-methoxybenzyl)amino)benzyl)piperazine-2,5-dione; 4-(4-((3-(1H-imidazol-1-yl)benzyl)(3-methoxybenzyl)amino)benzyl)piperazin-2-one; N-(3-(1H-imidazol-1-yl)benzyl)-4-((dimethylamino)methyl)-N-(3-methoxybenzyl)aniline N-(3-methoxybenzyl)-4-((2-(2-morpholinoethoxy)ethoxy)methyl)-N-(4-(pyrrolidin-1-yl)benzyl)aniline; N-(3-methoxybenzyl)-4-((2-(3-methoxybenzyloxy)ethoxy)methyl)-N-(4-(pyrrolidin-1-yl)benzyl)aniline; 3-(2-(4-((3-methoxybenzyl)(4-(pyrrolidin-1-yl)benzyl)amino)benzyloxy)ethoxy)-N,N-dimethylaniline; N-(3-methoxybenzyl)-4-((2-morpholinoethoxy)methyl)-N-(4-(pyrrolidin-1-yl)benzyl)aniline; N-(3-methoxybenzyl)-4-(morpholinomethyl)-N-(4-(pyrrolidin-1-yl)benzyl)aniline; N-(3-methoxybenzyl)-4-(piperidin-1-ylmethyl)-N-(4-(pyrrolidin-1-yl)benzyl)aniline; N-(3-methoxybenzyl)-4-((4-methylpiperazin-1-yl)methyl)-N-(4-(pyrrolidin-1-yl)benzyl)aniline; 1-(4-((3-methoxybenzyl)(4-(pyrrolidin-1-yl)benzyl)amino)benzyl)piperazin-2-one; 1-(4-((3-methoxybenzyl)(4-(pyrrolidin-1-yl)benzyl)amino)benzyl)piperazine-2,5-dione; 4-(4-((3-methoxybenzyl)(4-(pyrrolidin-1-yl)benzyl)amino)benzyl)piperazin-2-one; 4-((dimethylamino)methyl)-N-(3-methoxybenzyl)-N-(4-(pyrrolidin-1-yl)benzyl)aniline; N-(3-methoxybenzyl)-N-(4-morpholinobenzyl)-4-((2-(2-morpholinoethoxy)ethoxy)methyl)aniline; N-(3-methoxybenzyl)-4-((2-(3-methoxybenzyloxy)ethoxy)methyl)-N-(4-morpholinobenzyl)aniline; 3-(2-(4-((3-methoxybenzyl)(4-morpholinobenzyl)amino)benzyloxy)ethoxy)-N,N-dimethylaniline; N-(3-methoxybenzyl)-N-(4-morpholinobenzyl)-4-((2-morpholinoethoxy)methyl)aniline; N-(3-methoxybenzyl)-N-(4-morpholinobenzyl)-4-(morpholinomethyl)aniline; N-(3-methoxybenzyl)-N-(4-morpholinobenzyl)-4-(piperidin-1-ylmethyl)aniline; N-(3-methoxybenzyl)-4-((4-methylpiperazin-1-yl)methyl)-N-(4-morpholinobenzyl)aniline; 1-(4-((3-methoxybenzyl)(4-morpholinobenzyl)amino)benzyl)piperazin-2-one; 1-(4-((3-methoxybenzyl)(4-morpholinobenzyl)amino)benzyl)piperazine-2,5-dione; 4-(4-((3-methoxybenzyl)(4-morpholinobenzyl)amino)benzyl)piperazin-2-one; 4-((dimethylamino)methyl)-N-(3-methoxybenzyl)-N-(4-morpholinobenzyl)aniline; N-(3-methoxybenzyl)-N-(4-(4-methylpiperazin-1-yl)benzyl)-4-((2-(2-morpholinoethoxy)ethoxy)methyl)aniline; N-(3-methoxybenzyl)-4-((2-(3-methoxybenzyloxy)ethoxy)methyl)-N-(4-(4-methylpiperazin-1-yl)benzyl)aniline; 3-(2-(4-((3-methoxybenzyl)(4-(4-methylpiperazin-1-yl)benzyl)amino)benzyloxy)ethoxy)-N,N-dimethylaniline N-(3-methoxybenzyl)-N-(4-(4-methylpiperazin-1-yl)benzyl)-4-((2-morpholinoethoxy)methyl)aniline; N-(3-methoxybenzyl)-N-(4-(4-methylpiperazin-1-yl)benzyl)-4-(morpholinomethyl)aniline; N-(3-methoxybenzyl)-N-(4-(4-methylpiperazin-1-yl)benzyl)-4-(piperidin-1-ylmethyl)aniline; N-(3-methoxybenzyl)-N-(4-(4-methylpiperazin-1-yl)benzyl)-4-((4-methylpiperazin-1-yl)methyl)aniline; 1-(4-((3-methoxybenzyl)(4-(4-methylpiperazin-1-yl)benzyl)amino)benzyl)piperazin-2-one; 1-(4-((3-methoxybenzyl)(4-(4-methylpiperazin-1-yl)benzyl)amino)benzyl)piperazine-2,5-dione; 4-(4-((3-methoxybenzyl)(4-(4-methylpiperazin-1-yl)benzyl)amino)benzyl)piperazin-2-one; 4-((dimethylamino)methyl)-N-(3-methoxybenzyl)-N-(4-(4-methylpiperazin-1-yl)benzyl)aniline; N-(3-methoxybenzyl)-N-(3-morpholinobenzyl)-4-((2-(2-morpholinoethoxy)ethoxy)methyl)aniline; N-(3-methoxybenzyl)-4-((2-(3-methoxybenzyloxy)ethoxy)methyl)-N-(3-morpholinobenzyl)aniline; 3-(2-(4-((3-methoxybenzyl)(3-morpholinobenzyl)amino)benzyloxy)ethoxy)-N,N-dimethylaniline; N-(3-methoxybenzyl)-N-(3-morpholinobenzyl)-4-((2-morpholinoethoxy)methyl)aniline; N-(3-methoxybenzyl)-N-(3-morpholinobenzyl)-4-(morpholinomethyl)aniline; N-(3-methoxybenzyl)-N-(3-morpholinobenzyl)-4-(piperidin-1-ylmethyl)aniline; N-(3-methoxybenzyl)-4-((4-methylpiperazin-1-yl)methyl)-N-(3-morpholinobenzyl)aniline; 1-(4-((3-methoxybenzyl)(3-morpholinobenzyl)amino)benzyl)piperazin-2-one; 1-(4-((3-methoxybenzyl)(3-morpholinobenzyl)amino)benzyl)piperazine-2,5-dione; 4-(4-((3-methoxybenzyl)(3-morpholinobenzyl)amino)benzyl)piperazin-2-one; 4-((dimethylamino)methyl)-N-(3-methoxybenzyl)-N-(3-morpholinobenzyl)aniline; N-(3-methoxybenzyl)-N-(3-(4-methylpiperazin-1-yl)benzyl)-4-((2-(2-morpholinoethoxy)ethoxy)methyl)aniline; N-(3-methoxybenzyl)-4-((2-(3-methoxybenzyloxy)ethoxy)methyl)-N-(3-(4-methylpiperazin-1-yl)benzyl)aniline; 3-(2-(4-((3-methoxybenzyl)(3-(4-methylpiperazin-1-yl)benzyl)amino)benzyloxy)ethoxy)-N,N-dimethylaniline; N-(3-methoxybenzyl)-N-(3-(4-methylpiperazin-1-yl)benzyl)-4-((2-morpholinoethoxy)methyl)aniline; N-(3-methoxybenzyl)-N-(3-(4-methylpiperazin-1-yl)benzyl)-4-(morpholinomethyl)aniline; N-(3-methoxybenzyl)-N-(3-(4-methylpiperazin-1-yl)benzyl)-4-(piperidin-1-ylmethyl)aniline; N-(3-methoxybenzyl)-N-(3-(4-methylpiperazin-1-yl)benzyl)-4-((4-methylpiperazin-1-yl)methyl)aniline; 1-(4-((3-methoxybenzyl)(3-(4-methylpiperazin-1-yl)benzyl)amino)benzyl)piperazin-2-one; 1-(4-((3-methoxybenzyl)(3-(4-methylpiperazin-1-yl)benzyl)amino)benzyl)piperazine-2,5-dione; 4-(4-((3-methoxybenzyl)(3-(4-methylpiperazin-1-yl)benzyl)amino)benzyl)piperazin-2-one; 4-((dimethylamino)methyl)-N-(3-methoxybenzyl)-N-(3-(4-methylpiperazin-1-yl)benzyl)aniline; N-(4-(1H-pyrazol-1-yl)benzyl)-N-(3-methoxybenzyl)-4-((2-(2-morpholinoethoxy)ethoxy)methyl)aniline; N-(4-(1H-pyrazol-1-yl)benzyl)-N-(3-methoxybenzyl)-4-((2-(3-methoxybenzyloxy)ethoxy)methyl)aniline; 3-(2-(4-((4-(1H-pyrazol-1-yl)benzyl)(3-methoxybenzyl)amino)benzyloxy)ethoxy)-N,N-dimethylaniline; N-(4-(1H-pyrazol-1-yl)benzyl)-N-(3-methoxybenzyl)-4-((2-morpholinoethoxy)methyl)aniline; N-(4-(1H-pyrazol-1-yl)benzyl)-N-(3-methoxybenzyl)-4-(morpholinomethyl)aniline; N-(4-(1H-pyrazol-1-yl)benzyl)-N-(3-methoxybenzyl)-4-(piperidin-1-ylmethyl)aniline; N-(4-(1H-pyrazol-1-yl)benzyl)-N-(3-methoxybenzyl)-4-((4-methylpiperazin-1-yl)methyl)aniline; 1-(4-((4-(1H-pyrazol-1-yl)benzyl)(3-methoxybenzyl)amino)benzyl)piperazin-2-one; 1-(4-((4-(1H-pyrazol-1-yl)benzyl)(3-methoxybenzyl)amino)benzyl)piperazine-2,5-dione; 4-(4-((4-(1H-pyrazol-1-yl)benzyl)(3-methoxybenzyl)amino)benzyl)piperazin-2-one; N-(4-(1H-pyrazol-1-yl)benzyl)-4-((dimethylamino)methyl)-N-(3-methoxybenzyl)aniline; N-(4-(1H-imidazol-1-yl)benzyl)-N-(3-methoxybenzyl)-4-((2-(2-morpholinoethoxy)ethoxy)methyl)aniline; N-(4-(1H-imidazol-1-yl)benzyl)-N-(3-methoxybenzyl)-4-((2-(3-methoxybenzyloxy)ethoxy)methyl)aniline; 3-(2-(4-((4-(1H-imidazol-1-yl)benzyl)(3-methoxybenzyl)amino)benzyloxy)ethoxy)-N,N-dimethylaniline; N-(4-(1H-imidazol-1-yl)benzyl)-N-(3-methoxybenzyl)-4-((2-morpholinoethoxy)methyl)aniline; N-(4-(1H-imidazol-1-yl)benzyl)-N-(3-methoxybenzyl)-4-(morpholinomethyl)aniline; N-(4-(1H-imidazol-1-yl)benzyl)-N-(3-methoxybenzyl)-4-(piperidin-1-ylmethyl)aniline; N-(4-(1H-imidazol-1-yl)benzyl)-N-(3-methoxybenzyl)-4-((4-methylpiperazin-1-yl)methyl)aniline; 1-(4-((4-(1H-imidazol-1-yl)benzyl)(3-methoxybenzyl)amino)benzyl)piperazin-2-one; 1-(4-((4-(1H-imidazol-1-yl)benzyl)(3-methoxybenzyl)amino)benzyl)piperazine-2,5-dione; 4-(4-((4-(1H-imidazol-1-yl)benzyl)(3-methoxybenzyl)amino)benzyl)piperazin-2-one; N-(4-(1H-imidazol-1-yl)benzyl)-4-((dimethylamino)methyl)-N-(3-methoxybenzyl)aniline; N-((1H-benzo[d]imidazol-6-yl)methyl)-N-(3-methoxybenzyl)-4-((2-(2-morpholinoethoxy)ethoxy)methyl)aniline; N-((1H-benzo[d]imidazol-6-yl)methyl)-N-(3-methoxybenzyl)-4-((2-(3-methoxybenzyloxy)ethoxy)methyl)aniline; 3-(2-(4-(((1H-benzo[d]imidazol-6-yl)methyl)(3-methoxybenzyl)amino)benzyloxy)ethoxy)-N,N-dimethylaniline; N-((1H-benzo[d]imidazol-6-yl)methyl)-N-(3-methoxybenzyl)-4-((2-morpholinoethoxy)methyl)aniline; N-((1H-benzo[d]imidazol-6-yl)methyl)-N-(3-methoxybenzyl)-4-(morpholinomethyl)aniline; N-((1H-benzo[d]imidazol-6-yl)methyl)-N-(3-methoxybenzyl)-4-(piperidin-1-ylmethyl)aniline; N-((1H-benzo[d]imidazol-6-yl)methyl)-N-(3-methoxybenzyl)-4-((4-methylpiperazin-1-yl)methyl)aniline; 1-(4-(((1H-benzo[d]imidazol-6-yl)methyl)(3-methoxybenzyl)amino)benzyl)piperazin-2-one; 1-(4-(((1H-benzo[d]imidazol-6-yl)methyl)(3-methoxybenzyl)amino)benzyl)piperazine-2,5-dione; 4-(4-(((1H-benzo[d]imidazol-6-yl)methyl)(3-methoxybenzyl)amino)benzyl)piperazin-2-one; N-((1H-benzo[d]imidazol-6-yl)methyl)-4-((dimethylamino)methyl)-N-(3-methoxybenzyl)aniline; N-((1H-indazol-6-yl)methyl)-N-(3-methoxybenzyl)-4-((2-(2-morpholinoethoxy)ethoxy)methyl)aniline; N-((1H-indazol-6-yl)methyl)-N-(3-methoxybenzyl)-4-((2-(3-methoxybenzyloxy)ethoxy)methyl)aniline; 3-(2-(4-(((1H-indazol-6-yl)methyl)(3-methoxybenzyl)amino)benzyloxy)ethoxy)-N,N-dimethylaniline; N-((1H-indazol-6-yl)methyl)-N-(3-methoxybenzyl)-4-((2-morpholinoethoxy)methyl)aniline; N-((1H-indazol-6-yl)methyl)-N-(3-methoxybenzyl)-4-(morpholinomethyl)aniline; N-((1H-indazol-6-yl)methyl)-N-(3-methoxybenzyl)-4-(piperidin-1-ylmethyl)aniline; N-((1H-indazol-6-yl)methyl)-N-(3-methoxybenzyl)-4-((4-methylpiperazin-1-yl)methyl)aniline; 1-(4-(((1H-indazol-6-yl)methyl)(3-methoxybenzyl)amino)benzyl)piperazin-2-one; 1-(4-(((1H-indazol-6-yl)methyl)(3-methoxybenzyl)amino)benzyl)piperazine-2,5-dione; 4-(4-(((1H-indazol-6-yl)methyl)(3-methoxybenzyl)amino)benzyl)piperazin-2-one; N-((1H-indazol-6-yl)methyl)-4-((dimethylamino)methyl)-N-(3-methoxybenzyl)aniline; N-(3-methoxybenzyl)-4-((2-(2-morpholinoethoxy)ethoxy)methyl)-N-(quinolin-7-ylmethyl)aniline; N-(3-methoxybenzyl)-4-((2-(3-methoxybenzyloxy)ethoxy)methyl)-N-(quinolin-7-ylmethyl)aniline; 3-(2-(4-((3-methoxybenzyl)(quinolin-7-ylmethyl)amino)benzyloxy)ethoxy)-N,N-dimethylaniline; N-(3-methoxybenzyl)-4-((2-morpholinoethoxy)methyl)-N-(quinolin-7-ylmethyl)aniline; N-(3-methoxybenzyl)-4-(morpholinomethyl)-N-(quinolin-7-ylmethyl)aniline; N-(3-methoxybenzyl)-4-((4-methylpiperazin-1-yl)methyl)-N-(quinolin-7-ylmethyl)aniline; 1-(4-((3-methoxybenzyl)(quinolin-7-ylmethyl)amino)benzyl)piperazin-2-one; 1-(4-((3-methoxybenzyl)(quinolin-7-ylmethyl)amino)benzyl)piperazine-2,5-dione; 4-(4-((3-methoxybenzyl)(quinolin-7-ylmethyl)amino)benzyl)piperazin-2-one; 4-((dimethylamino)methyl)-N-(3-methoxybenzyl)-N-(quinolin-7-ylmethyl)aniline; N,N-bis(3-methoxybenzyl)-3-((2-(2-(3-methoxybenzyloxy)ethoxy)ethoxy)methyl)aniline; 3-((2-(2-(benzyloxy)ethoxy)ethoxy)methyl)-N,N-bis(3-methoxybenzyl)aniline; N,N-bis(3-methoxybenzyl)-3-((2-(2-(3-methoxyphenoxy)ethoxy)ethoxy)methyl)aniline; N,N-bis(3-methoxybenzyl)-3-((2-(2-morpholinoethoxy)ethoxy)methyl)aniline; N,N-bis(3-methoxybenzyl)-3-((2-(3-methoxybenzyloxy)ethoxy)methyl)aniline; 3-(2-(3-(bis(3-methoxybenzyl)amino)benzyloxy)ethoxy)-N,N-dimethylaniline; N,N-bis(3-methoxybenzyl)-3-((2-morpholinoethoxy)methyl)aniline; N,N-bis(3-methoxybenzyl)-3-(morpholinomethyl)aniline; N,N-bis(3-methoxybenzyl)-3-(piperidin-1-ylmethyl)aniline; N,N-bis(3-methoxybenzyl)-3-((4-methylpiperazin-1-yl)methyl)aniline; 1-(3-(bis(3-methoxybenzyl)amino)benzyl)piperazin-2-one; 1-(3-(bis(3-methoxybenzyl)amino)benzyl)piperazine-2,5-dione; 4-(3-(bis(3-methoxybenzyl)amino)benzyl)piperazin-2-one; 3-((dimethylamino)methyl)-N,N-bis(3-methoxybenzyl)aniline; N-(3-(dimethylamino)benzyl)-N-(3-methoxybenzyl)-3-((2-(2-(3-methoxybenzyloxy)ethoxy)ethoxy)methyl)aniline; 3-((2-(2-(benzyloxy)ethoxy)ethoxy)methyl)-N-(3-(dimethylamino)benzyl)-N-(3-methoxybenzyl)aniline; N-(3-(dimethylamino)benzyl)-N-(3-methoxybenzyl)-3-((2-(2-(3-methoxyphenoxy)ethoxy)ethoxy)methyl)aniline; N-(3-(dimethylamino)benzyl)-N-(3-methoxybenzyl)-3-((2-(2-morpholinoethoxy)ethoxy)methyl)aniline; N-(3-(dimethylamino)benzyl)-N-(3-methoxybenzyl)-3-((2-(3-methoxybenzyloxy)ethoxy)methyl)aniline; N-(3-(dimethylamino)benzyl)-3-((2-(3-(dimethylamino)phenoxy)ethoxy)methyl)-N-(3-methoxybenzyl)aniline; N-(3-(dimethylamino)benzyl)-N-(3-methoxybenzyl)-3-((2-morpholinoethoxy)methyl)aniline; N-(3-(dimethylamino)benzyl)-N-(3-methoxybenzyl)-3-(morpholinomethyl)aniline; N-(3-(dimethylamino)benzyl)-N-(3-methoxybenzyl)-3-(piperidin-1-ylmethyl)aniline; N-(3-(dimethylamino)benzyl)-N-(3-methoxybenzyl)-3-((4-methylpiperazin-1-yl)methyl)aniline; 1-(3-((3-(dimethylamino)benzyl)(3-methoxybenzyl)amino)benzyl)piperazin-2-one; 1-(3-((3-(dimethylamino)benzyl)(3-methoxybenzyl)amino)benzyl)piperazine-2,5-dione; 4-(3-((3-(dimethylamino)benzyl)(3-methoxybenzyl)amino)benzyl)piperazin-2-one; N-(3-(dimethylamino)benzyl)-3-((dimethylamino)methyl)-N-(3-methoxybenzyl)aniline; N-(3-methoxybenzyl)-3-((2-(2-morpholinoethoxy)ethoxy)methyl)-N-(3-(pyrrolidin-1-yl)benzyl)aniline; N-(3-methoxybenzyl)-3-((2-(3-methoxybenzyloxy)ethoxy)methyl)-N-(3-(pyrrolidin-1-yl)benzyl)aniline; 3-((2-(3-(dimethylamino)phenoxy)ethoxy)methyl)-N-(3-methoxybenzyl)-N-(3-(pyrrolidin-1-yl)benzyl)aniline; N-(3-methoxybenzyl)-3-((2-morpholinoethoxy)methyl)-N-(3-(pyrrolidin-1-yl)benzyl)aniline; N-(3-methoxybenzyl)-3-(morpholinomethyl)-N-(3-(pyrrolidin-1-yl)benzyl)aniline; N-(3-methoxybenzyl)-3-(piperidin-1-ylmethyl)-N-(3-(pyrrolidin-1-yl)benzyl)aniline; N-(3-methoxybenzyl)-3-((4-methylpiperazin-1-yl)methyl)-N-(3-(pyrrolidin-1-yl)benzyl)aniline 1-(3-((3-methoxybenzyl)(3-(pyrrolidin-1-yl)benzyl)amino)benzyl)piperazin-2-one; 1-(3-((3-methoxybenzyl)(3-(pyrrolidin-1-yl)benzyl)amino)benzyl)piperazine-2,5-dione; 4-(3-((3-methoxybenzyl)(3-(pyrrolidin-1-yl)benzyl)amino)benzyl)piperazin-2-one; 3-((dimethylamino)methyl)-N-(3-methoxybenzyl)-N-(3-(pyrrolidin-1-yl)benzyl)aniline; N-(3-(1H-pyrazol-1-yl)benzyl)-N-(3-methoxybenzyl)-3-((2-(2-morpholinoethoxy)ethoxy)methyl)aniline; N-(3-(1H-pyrazol-1-yl)benzyl)-N-(3-methoxybenzyl)-3-((2-(3 methoxybenzyloxy)ethoxy)methyl)aniline; N-(3-(1H-pyrazol-1-yl)benzyl)-3-((2-(3-(dimethylamino)phenoxy)ethoxy)methyl)-N-(3-methoxybenzyl)aniline; N-(3-(1H-pyrazol-1-yl)benzyl)-N-(3-methoxybenzyl)-3-((2-morpholinoethoxy)methyl)aniline; N-(3-(1H-pyrazol-1-yl)benzyl)-N-(3-methoxybenzyl)-3-(morpholinomethyl)aniline; N-(3-(1H-pyrazol-1-yl)benzyl)-N-(3-methoxybenzyl)-3-(piperidin-1-ylmethyl)aniline; N-(3-(1H-pyrazol-1-yl)benzyl)-N-(3-methoxybenzyl)-3-((4-methylpiperazin-1-yl)methyl)aniline; 1-(3-((3-(1H-pyrazol-1-yl)benzyl)(3-methoxybenzyl)amino)benzyl)piperazin-2-one; 1-(3-((3-(1H-pyrazol-1-yl)benzyl)(3-methoxybenzyl)amino)benzyl)piperazine-2,5-dione; 4-(3-((3-(1H-pyrazol-1-yl)benzyl)(3-methoxybenzyl)amino)benzyl)piperazin-2-one; N-(3-(1H-pyrazol-1-yl)benzyl)-3-((dimethylamino)methyl)-N-(3-methoxybenzyl)aniline; N-(3-(1H-imidazol-1-yl)benzyl)-N-(3-methoxybenzyl)-3-((2-(2-morpholinoethoxy)ethoxy)methyl)aniline; N-(3-(1H-imidazol-1-yl)benzyl)-N-(3-methoxybenzyl)-3-((2-(3-methoxybenzyloxy)ethoxy)methyl)aniline; N-(3-(1H-imidazol-1-yl)benzyl)-3-((2-(3-(dimethylamino)phenoxy)ethoxy)methyl)-N-(3-methoxybenzyl)aniline; N-(3-(1H-imidazol-1-yl)benzyl)-N-(3-methoxybenzyl)-3-((2-morpholinoethoxy)methyl)aniline; N-(3-(1H-imidazol-1-yl)benzyl)-N-(3-methoxybenzyl)-3-(morpholinomethyl)aniline; N-(3-(1H-imidazol-1-yl)benzyl)-N-(3-methoxybenzyl)-3-(piperidin-1-ylmethyl)aniline; N-(3-(1H-imidazol-1-yl)benzyl)-N-(3-methoxybenzyl)-3-((4-methylpiperazin-1-yl)methyl)aniline; 1-(3-((3-(1H-imidazol-1-yl)benzyl)(3-methoxybenzyl)amino)benzyl)piperazin-2-one; 1-(3-((3-(1H-imidazol-1-yl)benzyl)(3-methoxybenzyl)amino)benzyl)piperazine-2,5-dione; 4-(3-((3-(1H-imidazol-1-yl)benzyl)(3-methoxybenzyl)amino)benzyl)piperazin-2-one; N-(3-(1H-imidazol-1-yl)benzyl)-3-((dimethylamino)methyl)-N-(3-methoxybenzyl)aniline; 3-((2-(3-(dimethylamino)phenoxy)ethoxy)methyl)-N-(3-methoxybenzyl)-N-(4-(pyrrolidin-1-yl)benzyl)aniline; N-(3-methoxybenzyl)-3-((2-(2-morpholinoethoxy)ethoxy)methyl)-N-(4-(pyrrolidin-1-yl)benzyl)aniline; N-(3-methoxybenzyl)-3-((2-(3-methoxybenzyloxy)ethoxy)methyl)-N-(4-(pyrrolidin-1-yl)benzyl)aniline; N-(3-methoxybenzyl)-3-((2-morpholinoethoxy)methyl)-N-(4-(pyrrolidin-1-yl)benzyl)aniline; N-(3-methoxybenzyl)-3-(morpholinomethyl)-N-(4-(pyrrolidin-1-yl)benzyl)aniline; N-(3-methoxybenzyl)-3-(piperidin-1-ylmethyl)-N-(4-(pyrrolidin-1-yl)benzyl)aniline; N-(3-methoxybenzyl)-3-((4-methylpiperazin-1-yl)methyl)-N-(4-(pyrrolidin-1-yl)benzyl)aniline; 1-(3-((3-methoxybenzyl)(4-(pyrrolidin-1-yl)benzyl)amino)benzyl)piperazin-2-one; 1-(3-((3-methoxybenzyl)(4-(pyrrolidin-1-yl)benzyl)amino)benzyl)piperazine-2,5-dione; 4-(3-((3-methoxybenzyl)(4-(pyrrolidin-1-yl)benzyl)amino)benzyl)piperazin-2-one; 3-((dimethylamino)methyl)-N-(3-methoxybenzyl)-N-(4-(pyrrolidin-1-yl)benzyl)aniline; N-(3-methoxybenzyl)-N-(4-morpholinobenzyl)-3-((2-(2-morpholinoethoxy)ethoxy)methyl)aniline; N-(3-methoxybenzyl)-3-((2-(3-methoxybenzyloxy)ethoxy)methyl)-N-(4-morpholinobenzyl)aniline; 3-((2-(3-(dimethylamino)phenoxy)ethoxy)methyl)-N-(3-methoxybenzyl)-N-(4-morpholinobenzyl)aniline; N-(3-methoxybenzyl)-N-(4-morpholinobenzyl)-3-((2-morpholinoethoxy)methyl)aniline; N-(3-methoxybenzyl)-N-(4-morpholinobenzyl)-3-(morpholinomethyl)aniline; N-(3-methoxybenzyl)-N-(4-morpholinobenzyl)-3-(piperidin-1-ylmethyl)aniline; N-(3-methoxybenzyl)-3-((4-methylpiperazin-1-yl)methyl)-N-(4-morpholinobenzyl)aniline; 1-(3-((3-methoxybenzyl)(4-morpholinobenzyl)amino)benzyl)piperazin-2-one; 1-(3-((3-methoxybenzyl)(4-morpholinobenzyl)amino)benzyl)piperazine-2,5-dione; 4-(3-((3-methoxybenzyl)(4-morpholinobenzyl)amino)benzyl)piperazin-2-one; 3-((dimethylamino)methyl)-N-(3-methoxybenzyl)-N-(4-morpholinobenzyl)aniline; N-(3-methoxybenzyl)-N-(4-(4-methylpiperazin-1-yl)benzyl)-3-((2-(2-morpholinoethoxy)ethoxy)methyl)aniline; N-(3-methoxybenzyl)-3-((2-(3-methoxybenzyloxy)ethoxy)methyl)-N-(4-(4-methylpiperazin-1-yl)benzyl)aniline; 3-((2-(3-(dimethylamino)phenoxy)ethoxy)methyl)-N-(3-methoxybenzyl)-N-(4-(4-methylpiperazin-1-yl)benzyl)aniline; N-(3-methoxybenzyl)-N-(4-(4-methylpiperazin-1-yl)benzyl)-3-((2-morpholinoethoxy)methyl)aniline; N-(3-methoxybenzyl)-N-(4-(4-methylpiperazin-1-yl)benzyl)-3-(morpholinomethyl)aniline; N-(3-methoxybenzyl)-N-(4-(4-methylpiperazin-1-yl)benzyl)-3-(piperidin-1-ylmethyl)aniline; N-(3-methoxybenzyl)-N-(4-(4-methylpiperazin-1-yl)benzyl)-3-((4-methylpiperazin-1-yl)methyl)aniline; 1-(3-((3-methoxybenzyl)(4-(4-methylpiperazin-1-yl)benzyl)amino)benzyl)piperazin-2-one; 1-(3-((3-methoxybenzyl)(4-(4-methylpiperazin-1-yl)benzyl)amino)benzyl)piperazine-2,5-dione; 3-((dimethylamino)methyl)-N-(3-methoxybenzyl)-N-(4-(4-methylpiperazin-1-yl)benzyl)aniline; N-(3-methoxybenzyl)-N-(3-morpholinobenzyl)-3-((2-(2-morpholinoethoxy)ethoxy)methyl)aniline; N-(3-methoxybenzyl)-3-((2-(3-methoxybenzyloxy)ethoxy)methyl)-N-(3-morpholinobenzyl)aniline; 3-((2-(3-(dimethylamino)phenoxy)ethoxy)methyl)-N-(3-methoxybenzyl)-N-(3-morpholinobenzyl)aniline; N-(3-methoxybenzyl)-N-(3-morpholinobenzyl)-3-((2-morpholinoethoxy)methyl)aniline; N-(3-methoxybenzyl)-N-(3-morpholinobenzyl)-3-(morpholinomethyl)aniline; N-(3-methoxybenzyl)-N-(3-morpholinobenzyl)-3-(piperidin-1-ylmethyl)aniline; N-(3-methoxybenzyl)-3-((4-methylpiperazin-1-yl)methyl)-N-(3-morpholinobenzyl)aniline; 1-(3-((3-methoxybenzyl)(3-morpholinobenzyl)amino)benzyl)piperazin-2-one; 1-(3-((3-methoxybenzyl)(3-morpholinobenzyl)amino)benzyl)piperazine-2,5-dione; 4-(3-((3-methoxybenzyl)(3-morpholinobenzyl)amino)benzyl)piperazin-2-one; 3-((dimethylamino)methyl)-N-(3-methoxybenzyl)-N-(3-morpholinobenzyl)aniline; N-(3-methoxybenzyl)-N-(3-(4-methylpiperazin-1-yl)benzyl)-3-((2-(2-morpholinoethoxy)ethoxy)methyl)aniline; N-(3-methoxybenzyl)-3-((2-(3-methoxybenzyloxy)ethoxy)methyl)-N-(3-(4-methylpiperazin-1-yl)benzyl)aniline; 3-((2-(3-(dimethylamino)phenoxy)ethoxy)methyl)-N-(3-methoxybenzyl)-N-(3-(4-methylpiperazin-1-yl)benzyl)aniline; N-(3-methoxybenzyl)-N-(3-(4-methylpiperazin-1-yl)benzyl)-3-((2-morpholinoethoxy)methyl)aniline; N-(3-methoxybenzyl)-N-(3-(4-methylpiperazin-1-yl)benzyl)-3-(morpholinomethyl)aniline; N-(3-methoxybenzyl)-N-(3-(4-methylpiperazin-1-yl)benzyl)-3-(piperidin-1-ylmethyl)aniline; N-(3-methoxybenzyl)-N-(3-(4-methylpiperazin-1-yl)benzyl)-3-((4-methylpiperazin-1-yl)methyl)aniline; 1-(3-((3-methoxybenzyl)(3-(4-methylpiperazin-1-yl)benzyl)amino)benzyl)piperazin-2-one; 1-(3-((3-methoxybenzyl)(3-(4-methylpiperazin-1-yl)benzyl)amino)benzyl)piperazine-2,5-dione; 4-(3-((3-methoxybenzyl)(3-(4-methylpiperazin-1-yl)benzyl)amino)benzyl)piperazin-2-one; 3-((dimethylamino)methyl)-N-(3-methoxybenzyl)-N-(3-(4-methylpiperazin-1-yl)benzyl)aniline; N-(4-(1H-pyrazol-1-yl)benzyl)-N-(3-methoxybenzyl)-3-((2-(2-morpholinoethoxy)ethoxy)methyl)aniline; N-(4-(1H-pyrazol-1-yl)benzyl)-N-(3-methoxybenzyl)-3-((2-(3-methoxybenzyloxy)ethoxy)methyl)aniline; N-(4-(1H-pyrazol-1-yl)benzyl)-3-((2-(3-(dimethylamino)phenoxy)ethoxy)methyl)-N-(3-methoxybenzyl)aniline; N-(4-(1H-pyrazol-1-yl)benzyl)-N-(3-methoxybenzyl)-3-((2-morpholinoethoxy)methyl)aniline; N-(4-(1H-pyrazol-1-yl)benzyl)-N-(3-methoxybenzyl)-3-(morpholinomethyl)aniline; N-(4-(1H-pyrazol-1-yl)benzyl)-N-(3-methoxybenzyl)-3-(piperidin-1-ylmethyl)aniline N-(4-(1H-pyrazol-1-yl)benzyl)-N-(3-methoxybenzyl)-3-((4-methylpiperazin-1-yl)methyl)aniline; 1-(3-((4-(1H-pyrazol-1-yl)benzyl)(3-methoxybenzyl)amino)benzyl)piperazin-2-one; 1-(3-((4-(1H-pyrazol-1-yl)benzyl)(3-methoxybenzyl)amino)benzyl)piperazine-2,5-dione; 4-(3-((4-(1H-pyrazol-1-yl)benzyl)(3-methoxybenzyl)amino)benzyl)piperazin-2-one; N-(4-(1H-pyrazol-1-yl)benzyl)-3-((dimethylamino)methyl)-N-(3-methoxybenzyl)aniline; N-(4-(1H-imidazol-1-yl)benzyl)-N-(3-methoxybenzyl)-3-((2-(2-morpholinoethoxy)ethoxy)methyl)aniline; N-(4-(1H-imidazol-1-yl)benzyl)-N-(3-methoxybenzyl)-3-((2-(3-methoxybenzyloxy)ethoxy)methyl)aniline; N-(4-(1H-imidazol-1-yl)benzyl)-3-((2-(3-(dimethylamino)phenoxy)ethoxy)methyl)-N-(3-methoxybenzyl)aniline; N-(4-(1H-imidazol-1-yl)benzyl)-N-(3-methoxybenzyl)-3-((2-morpholinoethoxy)methyl)aniline; N-(4-(1H-imidazol-1-yl)benzyl)-N-(3-methoxybenzyl)-3-(morpholinomethyl)aniline; N-(4-(1H-imidazol-1-yl)benzyl)-N-(3-methoxybenzyl)-3-(piperidin-1-ylmethyl)aniline; N-(4-(1H-imidazol-1-yl)benzyl)-N-(3-methoxybenzyl)-3-((4-methylpiperazin-1-yl)methyl)aniline; 1-(3-((4-(1H-imidazol-1-yl)benzyl)(3-methoxybenzyl)amino)benzyl)piperazin-2-one; 1-(3-((4-(1H-imidazol-1-yl)benzyl)(3-methoxybenzyl)amino)benzyl)piperazine-2,5-dione; 4-(3-((4-(1H-imidazol-1-yl)benzyl)(3-methoxybenzyl)amino)benzyl)piperazin-2-one; N-(4-(1H-imidazol-1-yl)benzyl)-3-((dimethylamino)methyl)-N-(3-methoxybenzyl)aniline; N-((1H-benzo[d]imidazol-6-yl)methyl)-N-(3-methoxybenzyl)-3-((2-(2-morpholinoethoxy)ethoxy)methyl)aniline; N-((1H-benzo[d]imidazol-6-yl)methyl)-N-(3-methoxybenzyl)-3-((2-(3-methoxybenzyloxy)ethoxy)methyl)aniline; N-((1H-benzo[d]imidazol-6-yl)methyl)-3-((2-(3-(dimethylamino)phenoxy)ethoxy)methyl)-N-(3-methoxybenzyl)aniline; N-((1H-benzo[d]imidazol-6-yl)methyl)-N-(3-methoxybenzyl)-3-((2-morpholinoethoxy)methyl)aniline; N-((1H-benzo[d]imidazol-6-yl)methyl)-N-(3-methoxybenzyl)-3-(morpholinomethyl)aniline; N-((1H-benzo[d]imidazol-6-yl)methyl)-N-(3-methoxybenzyl)-3-(piperidin-1-ylmethyl)aniline; N-((1H-benzo[d]imidazol-6-yl)methyl)-N-(3-methoxybenzyl)-3-((4-methylpiperazin-1-yl)methyl)aniline; 1-(3-(((1H-benzo[d]imidazol-6-yl)methyl)(3-methoxybenzyl)amino)benzyl)piperazin-2-one; 1-(3-(((1H-benzo[d]imidazol-6-yl)methyl)(3-methoxybenzyl)amino)benzyl)piperazine-2,5-dione; 4-(3-(((1H-benzo[d]imidazol-6-yl)methyl)(3-methoxybenzyl)amino)benzyl)piperazin-2-one; N-((1H-benzo[d]imidazol-6-yl)methyl)-3-((dimethylamino)methyl)-N-(3-methoxybenzyl)aniline; N-((1H-indazol-6-yl)methyl)-N-(3-methoxybenzyl)-3-((2-(2-morpholinoethoxy)ethoxy)methyl)aniline; N-((1H-indazol-6-yl)methyl)-N-(3-methoxybenzyl)-3-((2-(3-methoxybenzyloxy)ethoxy)methyl)aniline; N-((1H-indazol-6-yl)methyl)-3-((2-(3-(dimethylamino)phenoxy)ethoxy)methyl)-N-(3-methoxybenzyl)aniline; N-((1H-indazol-6-yl)methyl)-N-(3-methoxybenzyl)-3-((2-morpholinoethoxy)methyl)aniline; N-((1H-indazol-6-yl)methyl)-N-(3-methoxybenzyl)-3-(morpholinomethyl)aniline; N-((1H-indazol-6-yl)methyl)-N-(3-methoxybenzyl)-3-(piperidin-1-ylmethyl)aniline; N-((1H-indazol-6-yl)methyl)-N-(3-methoxybenzyl)-3-((4-methylpiperazin-1-yl)methyl)aniline; 1-(3-(((1H-indazol-6-yl)methyl)(3-methoxybenzyl)amino)benzyl)piperazin-2-one; 1-(3-(((1H-indazol-6-yl)methyl)(3-methoxybenzyl)amino)benzyl)piperazine-2,5-dione; 4-(3-(((1H-indazol-6-yl)methyl)(3-methoxybenzyl)amino)benzyl)piperazin-2-one; N-((1H-indazol-6-yl)methyl)-3-((dimethylamino)methyl)-N-(3-methoxybenzyl)aniline; N-(3-methoxybenzyl)-3-((2-(2-morpholinoethoxy)ethoxy)methyl)-N-(quinolin-7-ylmethyl)aniline; N-(3-methoxybenzyl)-3-((2-(3-methoxybenzyloxy)ethoxy)methyl)-N-(quinolin-7-ylmethyl)aniline; 3-((2-(3-(dimethylamino)phenoxy)ethoxy)methyl)-N-(3-methoxybenzyl)-N-(quinolin-7-ylmethyl)aniline; N-(3-methoxybenzyl)-3-((2-morpholinoethoxy)methyl)-N-(quinolin-7-ylmethyl)aniline N-(3-methoxybenzyl)-3-(morpholinomethyl)-N-(quinolin-7-ylmethyl)aniline; N-(3-methoxybenzyl)-3-(piperidin-1-ylmethyl)-N-(quinolin-7-ylmethyl)aniline; N-(3-methoxybenzyl)-3-((4-methylpiperazin-1-yl)methyl)-N-(quinolin-7-ylmethyl)aniline; 1-(3-((3-methoxybenzyl)(quinolin-7-ylmethyl)amino)benzyl)piperazin-2-one; 1-(3-((3-methoxybenzyl)(quinolin-7-ylmethyl)amino)benzyl)piperazine-2,5-dione; 4-(3-((3-methoxybenzyl)(quinolin-7-ylmethyl)amino)benzyl)piperazin-2-one; 3-((dimethylamino)methyl)-N-(3-methoxybenzyl)-N-(quinolin-7-ylmethyl)aniline; N,N-bis(3-methoxybenzyl)-2-((2-(2-(3-methoxybenzyloxy)ethoxy)ethoxy)methyl)pyridin-4-amine; 2-((2-(2-(benzyloxy)ethoxy)ethoxy)methyl)-N,N-bis(3-methoxybenzyl)pyridin-4-amine; N,N-bis(3-methoxybenzyl)-2-((2-(2-(3-methoxyphenoxy)ethoxy)ethoxy)methyl)pyridin-4-amine; N,N-bis(3-methoxybenzyl)-2-((2-(2-morpholinoethoxy)ethoxy)methyl)pyridin-4-amine; N,N-bis(3-methoxybenzyl)-2-((2-(3-methoxybenzyloxy)ethoxy)methyl)pyridin-4-amine; 2-((2-(3-(dimethylamino)phenoxy)ethoxy)methyl)-N,N-bis(3-methoxybenzyl)pyridin-4-amine; N,N-bis(3-methoxybenzyl)-2-((2-morpholinoethoxy)methyl)pyridin-4-amine; N,N-bis(3-methoxybenzyl)-2-(morpholinomethyl)pyridin-4-amine; N,N-bis(3-methoxybenzyl)-2-(piperidin-1-ylmethyl)pyridin-4-amine; N,N-bis(3-methoxybenzyl)-2-((4-methylpiperazin-1-yl)methyl)pyridin-4-amine; 1-((4-(bis(3-methoxybenzyl)amino)pyridin-2-yl)methyl)piperazin-2-one; 1-((4-(bis(3-methoxybenzyl)amino)pyridin-2-yl)methyl)piperazine-2,5-dione; 4-((4-(bis(3-methoxybenzyl)amino)pyridin-2-yl)methyl)piperazin-2-one; 2-((dimethylamino)methyl)-N,N-bis(3-methoxybenzyl)pyridin-4-amine; N-(3-(dimethylamino)benzyl)-N-(3-methoxybenzyl)-2-((2-(2-(3-methoxybenzyloxy)ethoxy)ethoxy)methyl)pyridin-4-amine; 2-((2-(2-(benzyloxy)ethoxy)ethoxy)methyl)-N-(3-(dimethylamino)benzyl)-N-(3-methoxybenzyl)pyridin-4-amine; N-(3-(dimethylamino)benzyl)-N-(3-methoxybenzyl)-2-((2-(2-(3-methoxyphenoxy)ethoxy)ethoxy)methyl)pyridin-4-amine; N-(3-(dimethylamino)benzyl)-N-(3-methoxybenzyl)-2-((2-(2-morpholinoethoxy)ethoxy)methyl)pyridin-4-amine; N-(3-(dimethylamino)benzyl)-N-(3-methoxybenzyl)-2-((2-(3-methoxybenzyloxy)ethoxy)methyl)pyridin-4-amine; N-(3-(dimethylamino)benzyl)-2-((2-(3-(dimethylamino)phenoxy)ethoxy)methyl)-N-(3-methoxybenzyl)pyridin-4-amine; N-(3-(dimethylamino)benzyl)-N-(3-methoxybenzyl)-2-((2-morpholinoethoxy)methyl)pyridin-4-amine; N-(3-(dimethylamino)benzyl)-N-(3-methoxybenzyl)-3-(morpholinomethyl)aniline; N-(3-(dimethylamino)benzyl)-N-(3-methoxybenzyl)-2-(piperidin-1-ylmethyl)pyridin-4-amine; N-(3-(dimethylamino)benzyl)-N-(3-methoxybenzyl)-2-((4-methylpiperazin-1-yl)methyl)pyridin-4-amine; 1-((4-((3-(dimethylamino)benzyl)(3-methoxybenzyl)amino)pyridin-2-yl)methyl)piperazin-2-one; 1-((4-((3-(dimethylamino)benzyl)(3-methoxybenzyl)amino)pyridin-2-yl)methyl)piperazine-2,5-dione; 4-((4-((3-(dimethylamino)benzyl)(3-methoxybenzyl)amino)pyridin-2-yl)methyl)piperazin-2-one; N-(3-(dimethylamino)benzyl)-2-((dimethylamino)methyl)-N-(3-methoxybenzyl)pyridin-4-amine; N-(3-methoxybenzyl)-2-((2-(2-morpholinoethoxy)ethoxy)methyl)-N-(3-(pyrrolidin-1-yl)benzyl)pyridin-4-amine; N-(3-methoxybenzyl)-2-((2-(3-methoxybenzyloxy)ethoxy)methyl)-N-(3-(pyrrolidin-1-yl)benzyl)pyridin-4-amine; 2-((2-(3-(dimethylamino)phenoxy)ethoxy)methyl)-N-(3-methoxybenzyl)-N-(3-(pyrrolidin-1-yl)benzyl)pyridin-4-amine; N-(3-methoxybenzyl)-2-((2-morpholinoethoxy)methyl)-N-(3-(pyrrolidin-1-yl)benzyl)pyridin-4-amine; N-(3-methoxybenzyl)-2-(morpholinomethyl)-N-(3-(pyrrolidin-1-yl)benzyl)pyridin-4-amine; N-(3-methoxybenzyl)-2-(piperidin-1-ylmethyl)-N-(3-(pyrrolidin-1-yl)benzyl)pyridin-4-amine; N-(3-methoxybenzyl)-2-((4-methylpiperazin-1-yl)methyl)-N-(3-(pyrrolidin-1-yl)benzyl)pyridin-4-amine; 1-((4-((3-methoxybenzyl)(3-(pyrrolidin-1-yl)benzyl)amino)pyridin-2-yl)methyl)piperazin-2-one; 1-((4-((3-methoxybenzyl)(3-(pyrrolidin-1-yl)benzyl)amino)pyridin-2-yl)methyl)piperazine-2,5-dione; 4-((4-((3-methoxybenzyl)(3-(pyrrolidin-1-yl)benzyl)amino)pyridin-2-yl)methyl)piperazin-2-one; 2-((dimethylamino)methyl)-N-(3-methoxybenzyl)-N-(3-(pyrrolidin-1-yl)benzyl)pyridin-4-amine; N-(3-(1H-pyrazol-1-yl)benzyl)-N-(3-methoxybenzyl)-2-((2-(2-morpholinoethoxy)ethoxy)methyl)pyridin-4-amine; N-(3-(1H-pyrazol-1-yl)benzyl)-N-(3-methoxybenzyl)-2-((2-(3-methoxybenzyloxy)ethoxy)methyl)pyridin-4-amine; N-(3-(1H-pyrazol-1-yl)benzyl)-2-((2-(3-(dimethylamino)phenoxy)ethoxy)methyl)-N-(3-methoxybenzyl)pyridin-4-amine; N-(3-(1H-pyrazol-1-yl)benzyl)-N-(3-methoxybenzyl)-2-((2-morpholinoethoxy)methyl)pyridin-4-amine; N-(3-(1H-pyrazol-1-yl)benzyl)-N-(3-methoxybenzyl)-2-(morpholinomethyl)pyridin-4-amine; N-(3-(1H-pyrazol-1-yl)benzyl)-N-(3-methoxybenzyl)-2-(piperidin-1-ylmethyl)pyridin-4-amine; N-(3-(1H-pyrazol-1-yl)benzyl)-N-(3-methoxybenzyl)-2-((4-methylpiperazin-1-yl)methyl)pyridin-4-amine; 1-((4-((3-(1H-pyrazol-1-yl)benzyl)(3-methoxybenzyl)amino)pyridin-2-yl)methyl)piperazin-2-one; 1-((4-((3-(1H-pyrazol-1-yl)benzyl)(3-methoxybenzyl)amino)pyridin-2-yl)methyl)piperazine-2,5-dione; 4-((4-((3-(1H-pyrazol-1-yl)benzyl)(3-methoxybenzyl)amino)pyridin-2-yl)methyl)piperazin-2-one; N-(3-(1H-pyrazol-1-yl)benzyl)-2-((dimethylamino)methyl)-N-(3-methoxybenzyl)pyridin-4-amine; N-(3-(1H-imidazol-1-yl)benzyl)-N-(3-methoxybenzyl)-2-((2-(2-morpholinoethoxy)ethoxy)methyl)pyridin-4-amine; N-(3-(1H-imidazol-1-yl)benzyl)-N-(3-methoxybenzyl)-2-((2-(3 methoxybenzyloxy)ethoxy)methyl)pyridin-4-amine; N-(3-(1H-imidazol-1-yl)benzyl)-2-((2-(3-(dimethylamino)phenoxy)ethoxy)methyl)-N-(3-methoxybenzyl)pyridin-4-amine; N-(3-(1H-imidazol-1-yl)benzyl)-N-(3-methoxybenzyl)-2-((2-morpholinoethoxy)methyl)pyridin-4-amine; N-(3-(1H-imidazol-1-yl)benzyl)-N-(3-methoxybenzyl)-2-(morpholinomethyl)pyridin-4-amine; N-(3-(1H-imidazol-1-yl)benzyl)-N-(3-methoxybenzyl)-2-(piperidin-1-ylmethyl)pyridin-4-amine; N-(3-(1H-imidazol-1-yl)benzyl)-N-(3-methoxybenzyl)-2-((4-methylpiperazin-1-yl)methyl)pyridin-4-amine; 1-((4-((3-(1H-imidazol-1-yl)benzyl)(3-methoxybenzyl)amino)pyridin-2-yl)methyl)piperazin-2-one; 1-((4-((3-(1H-imidazol-1-yl)benzyl)(3-methoxybenzyl)amino)pyridin-2-yl)methyl)piperazine-2,5-dione; 4-((4-((3-(1H-imidazol-1-yl)benzyl)(3-methoxybenzyl)amino)pyridin-2-yl)methyl)piperazin-2-one; N-(3-(1H-imidazol-1-yl)benzyl)-2-((dimethylamino)methyl)-N-(3-methoxybenzyl)pyridin-4-amine; 2-((2-(3-(dimethylamino)phenoxy)ethoxy)methyl)-N-(3-methoxybenzyl)-N-(4-(pyrrolidin-1-yl)benzyl)pyridin-4-amine; N-(3-methoxybenzyl)-2-((2-(2-morpholinoethoxy)ethoxy)methyl)-N-(4-(pyrrolidin-1-yl)benzyl)pyridin-4-amine; N-(3-methoxybenzyl)-2-((2-(3-methoxybenzyloxy)ethoxy)methyl)-N-(4-(pyrrolidin-1-yl)benzyl)pyridin-4-amine; N-(3-methoxybenzyl)-2-((2-morpholinoethoxy)methyl)-N-(4-(pyrrolidin-1-yl)benzyl)pyridin-4-amine; N-(3-methoxybenzyl)-2-(morpholinomethyl)-N-(4-(pyrrolidin-1-yl)benzyl)pyridin-4-amine; N-(3-methoxybenzyl)-2-(piperidin-1-ylmethyl)-N-(4-(pyrrolidin-1-yl)benzyl)pyridin-4-amine; N-(3-methoxybenzyl)-2-((4-methylpiperazin-1-yl)methyl)-N-(4-(pyrrolidin-1-yl)benzyl)pyridin-4-amine; 1-((4-((3-methoxybenzyl)(4-(pyrrolidin-1-yl)benzyl)amino)pyridin-2-yl)methyl)piperazin-2-one; 1-((4-((3-methoxybenzyl)(4-(pyrrolidin-1-yl)benzyl)amino)pyridin-2-yl)methyl)piperazine-2,5-dione; 4-((4-((3-methoxybenzyl)(4-(pyrrolidin-1-yl)benzyl)amino)pyridin-2-yl)methyl)piperazin-2-one; 2-((dimethylamino)methyl)-N-(3-methoxybenzyl)-N-(4-(pyrrolidin-1-yl)benzyl)pyridin-4-amine; N-(3-methoxybenzyl)-N-(4-morpholinobenzyl)-2-((2-(2-morpholinoethoxy)ethoxy)methyl)pyridin-4-amine; N-(3-methoxybenzyl)-2-((2-(3-methoxybenzyloxy)ethoxy)methyl)-N-(4-morpholinobenzyl)pyridin-4-amine; 2-((2-(3-(dimethylamino)phenoxy)ethoxy)methyl)-N-(3-methoxybenzyl)-N-(4-morpholinobenzyl)pyridin-4-amine; N-(3-methoxybenzyl)-N-(4-morpholinobenzyl)-2-((2-morpholinoethoxy)methyl)pyridin-4-amine; N-(3-methoxybenzyl)-N-(4-morpholinobenzyl)-2-(morpholinomethyl)pyridin-4-amine; N-(3-methoxybenzyl)-N-(4-morpholinobenzyl)-2-(piperidin-1-ylmethyl)pyridin-4-amine; N-(3-methoxybenzyl)-2-((4-methylpiperazin-1-yl)methyl)-N-(4-morpholinobenzyl)pyridin-4-amine; 1-((4-((3-methoxybenzyl)(4-morpholinobenzyl)amino)pyridin-2-yl)methyl)piperazin-2-one; 1-((4-((3-methoxybenzyl)(4-morpholinobenzyl)amino)pyridin-2-yl)methyl)piperazine-2,5-dione; 4-((4-((3-methoxybenzyl)(4-morpholinobenzyl)amino)pyridin-2-yl)methyl)piperazin-2-one; 2-((dimethylamino)methyl)-N-(3-methoxybenzyl)-N-(4-morpholinobenzyl)pyridin-4-amine; N-(3-methoxybenzyl)-N-(4-(4-methylpiperazin-1-yl)benzyl)-2-((2-(2 morpholinoethoxy)ethoxy)methyl)pyridin-4-amine; N-(3-methoxybenzyl)-2-((2-(3-methoxybenzyloxy)ethoxy)methyl)-N-(4-(4-methylpiperazin-1-yl)benzyl)pyridin-4-amine; 2-((2-(3-(dimethylamino)phenoxy)ethoxy)methyl)-N-(3-methoxybenzyl)-N-(4-(4-methylpiperazin-1-yl)benzyl)pyridin-4-amine; N-(3-methoxybenzyl)-N-(4-(4-methylpiperazin-1-yl)benzyl)-2-((2-morpholinoethoxy)methyl)pyridin-4-amine; N-(3-methoxybenzyl)-N-(4-(4-methylpiperazin-1-yl)benzyl)-2-(morpholinomethyl)pyridin-4-amine; N-(3-methoxybenzyl)-N-(4-(4-methylpiperazin-1-yl)benzyl)-2-(piperidin-1-ylmethyl)pyridin-4-amine; N-(3-methoxybenzyl)-N-(4-(4-methylpiperazin-1-yl)benzyl)-2-((4-methylpiperazin-1-yl)methyl)pyridin-4-amine; 1-((4-((3-methoxybenzyl)(4-(4-methylpiperazin-1-yl)benzyl)amino)pyridin-2-yl)methyl)piperazin-2-one; 1-((4-((3-methoxybenzyl)(4-(4-methylpiperazin-1-yl)benzyl)amino)pyridin-2-yl)methyl)piperazine-2,5-dione; 4-((4-((3-methoxybenzyl)(4-(4-methylpiperazin-1-yl)benzyl)amino)pyridin-2-yl)methyl)piperazin-2-one; 2-((dimethylamino)methyl)-N-(3-methoxybenzyl)-N-(4-(4-methylpiperazin-1-yl)benzyl)pyridin-4-amine; N-(3-methoxybenzyl)-N-(3-morpholinobenzyl)-2-((2-(2-morpholinoethoxy)ethoxy)methyl)pyridin-4-amine; N-(3-methoxybenzyl)-2-((2-(3-methoxybenzyloxy)ethoxy)methyl)-N-(3-morpholinobenzyl)pyridin-4-amine; 2-((2-(3-(dimethylamino)phenoxy)ethoxy)methyl)-N-(3-methoxybenzyl)-N-(3-morpholinobenzyl)pyridin-4-amine; N-(3-methoxybenzyl)-N-(3-morpholinobenzyl)-2-((2-morpholinoethoxy)methyl)pyridin-4-amine; N-(3-methoxybenzyl)-N-(3-morpholinobenzyl)-2-(morpholinomethyl)pyridin-4-amine; N-(3-methoxybenzyl)-N-(3-morpholinobenzyl)-2-(piperidin-1-ylmethyl)pyridin-4-amine; N-(3-methoxybenzyl)-2-((4-methylpiperazin-1-yl)methyl)-N-(3-morpholinobenzyl)pyridin-4-amine; 1-((4-((3-methoxybenzyl)(3-morpholinobenzyl)amino)pyridin-2-yl)methyl)piperazin-2-one; 1-((4-((3-methoxybenzyl)(3-morpholinobenzyl)amino)pyridin-2-yl)methyl)piperazine-2,5-dione; 4-((4-((3-methoxybenzyl)(3-morpholinobenzyl)amino)pyridin-2-yl)methyl)piperazin-2-one; 2-((dimethylamino)methyl)-N-(3-methoxybenzyl)-N-(3-morpholinobenzyl)pyridin-4-amine; N-(3-methoxybenzyl)-N-(3-(4-methylpiperazin-1-yl)benzyl)-2-((2-(2-morpholinoethoxy)ethoxy)methyl)pyridin-4-amine; N-(3-methoxybenzyl)-2-((2-(3-methoxybenzyloxy)ethoxy)methyl)-N-(3-(4-methylpiperazin-1-yl)benzyl)pyridin-4-amine; 2-((2-(3-(dimethylamino)phenoxy)ethoxy)methyl)-N-(3-methoxybenzyl)-N-(3-(4-methylpiperazin-1-yl)benzyl)pyridin-4-amine; N-(3-methoxybenzyl)-N-(3-(4-methylpiperazin-1-yl)benzyl)-2-((2-morpholinoethoxy)methyl)pyridin-4-amine; N-(3-methoxybenzyl)-N-(3-(4-methylpiperazin-1-yl)benzyl)-2-(morpholinomethyl)pyridin-4-amine; N-(3-methoxybenzyl)-N-(3-(4-methylpiperazin-1-yl)benzyl)-2-(piperidin-1-ylmethyl)pyridin-4-amine; N-(3-methoxybenzyl)-N-(3-(4-methylpiperazin-1-yl)benzyl)-2-((4-methylpiperazin-1-yl)methyl)pyridin-4-amine; 1-((4-((3-methoxybenzyl)(3-(4-methylpiperazin-1-yl)benzyl)amino)pyridin-2-yl)methyl)piperazin-2-one; 1-((4-((3-methoxybenzyl)(3-(4-methylpiperazin-1-yl)benzyl)amino)pyridin-2-yl)methyl)piperazine-2,5-dione; 4-((4-((3-methoxybenzyl)(3-(4-methylpiperazin-1-yl)benzyl)amino)pyridin-2-yl)methyl)piperazin-2-one; 2-((dimethylamino)methyl)-N-(3-methoxybenzyl)-N-(3-(4-methylpiperazin-1-yl)benzyl)pyridin-4-amine; N-(4-(1H-pyrazol-1-yl)benzyl)-N-(3-methoxybenzyl)-2-((2-(2-morpholinoethoxy)ethoxy)methyl)pyridin-4-amine; N-(4-(1H-pyrazol-1-yl)benzyl)-N-(3-methoxybenzyl)-2-((2-(3-methoxybenzyloxy)ethoxy)methyl)pyridin-4-amine; N-(4-(1H-pyrazol-1-yl)benzyl)-2-((2-(3-(dimethylamino)phenoxy)ethoxy)methyl)-N-(3-methoxybenzyl)pyridin-4-amine; N-(4-(1H-pyrazol-1-yl)benzyl)-N-(3-methoxybenzyl)-2-((2-morpholinoethoxy)methyl)pyridin-4-amine; N-(4-(1H-pyrazol-1-yl)benzyl)-N-(3-methoxybenzyl)-2-(morpholinomethyl)pyridin-4-amine; N-(4-(1H-pyrazol-1-yl)benzyl)-N-(3-methoxybenzyl)-2-(piperidin-1-ylmethyl)pyridin-4-amine; 1-((4-((4-(1H-pyrazol-1-yl)benzyl)(3-methoxybenzyl)amino)pyridin-2-yl)methyl)piperazin-2-one; 1-((4-((4-(1H-pyrazol-1-yl)benzyl)(3-methoxybenzyl)amino)pyridin-2-yl)methyl)piperazine-2,5-dione; 4-((4-((4-(1H-pyrazol-1-yl)benzyl)(3-methoxybenzyl)amino)pyridin-2-yl)methyl)piperazin-2-one; N-(4-(1H-pyrazol-1-yl)benzyl)-2-((dimethylamino)methyl)-N-(3-methoxybenzyl)pyridin-4-amine; N-(4-(1H-imidazol-1-yl)benzyl)-N-(3-methoxybenzyl)-2-((2-(2-morpholinoethoxy)ethoxy)methyl)pyridin-4-amine; N-(4-(1H-imidazol-1-yl)benzyl)-N-(3-methoxybenzyl)-2-((2-(3-methoxybenzyloxy)ethoxy)methyl)pyridin-4-amine; N-(4-(1H-imidazol-1-yl)benzyl)-2-((2-(3-(dimethylamino)phenoxy)ethoxy)methyl)-N-(3-methoxybenzyl)pyridin-4-amine; N-(4-(1H-imidazol-1-yl)benzyl)-N-(3-methoxybenzyl)-2-((2-morpholinoethoxy)methyl)pyridin-4-amine; N-(4-(1H-imidazol-1-yl)benzyl)-N-(3-methoxybenzyl)-2-(morpholinomethyl)pyridin-4-amine; N-(4-(1H-imidazol-1-yl)benzyl)-N-(3-methoxybenzyl)-2-(piperidin-1-ylmethyl)pyridin-4-amine; N-(4-(1H-imidazol-1-yl)benzyl)-N-(3-methoxybenzyl)-2-((4-methylpiperazin-1-yl)methyl)pyridin-4-amine; 1-((4-((4-(1H-imidazol-1-yl)benzyl)(3-methoxybenzyl)amino)pyridin-2-yl)methyl)piperazin-2-one; 1-((4-((4-(1H-imidazol-1-yl)benzyl)(3-methoxybenzyl)amino)pyridin-2-yl)methyl)piperazine-2,5-dione; 4-((4-((4-(1H-imidazol-1-yl)benzyl)(3-methoxybenzyl)amino)pyridin-2-yl)methyl)piperazin-2-one; N-(4-(1H-imidazol-1-yl)benzyl)-2-((dimethylamino)methyl)-N-(3-methoxybenzyl)pyridin-4-amine; N-((1H-benzo[d]imidazol-6-yl)methyl)-N-(3-methoxybenzyl)-2-((2-(2-morpholinoethoxy)ethoxy)methyl)pyridin-4-amine; N-((1H-benzo[d]imidazol-6-yl)methyl)-N— (3-methoxybenzyl)-2-((2-(3-methoxybenzyloxy)ethoxy)methyl)pyridin-4-amine; N-((1H-benzo[d]imidazol-6-yl)methyl)-2-((2-(3-(dimethylamino)phenoxy)ethoxy)methyl)-N-(3-methoxybenzyl)pyridin-4-amine; N-((1H-benzo[d]imidazol-6-yl)methyl)-N-(3-methoxybenzyl)-2-((2-morpholinoethoxy)methyl)pyridin-4-amine; N-((1H-benzo[d]imidazol-6-yl)methyl)-N-(3-methoxybenzyl)-2-(morpholinomethyl)pyridin-4-amine; N-((1H-benzo[d]imidazol-6-yl)methyl)-N-(3-methoxybenzyl)-2-(piperidin-1-ylmethyl)pyridin-4-amine; N-((1H-benzo[d]imidazol-6-yl)methyl)-N-(3-methoxybenzyl)-2-((4-methylpiperazin-1-yl)methyl)pyridin-4-amine; 1-((4-(((1H-benzo[d]imidazol-6-yl)methyl)(3-methoxybenzyl)amino)pyridin-2-yl)methyl)piperazin-2-one; 1-((4-(((1H-benzo[d]imidazol-6-yl)methyl)(3-methoxybenzyl)amino)pyridin-2-yl)methyl)piperazine-2,5-dione; 4-((4-(((1H-benzo[d]imidazol-6-yl)methyl)(3-methoxybenzyl)amino)pyridin-2-yl)methyl)piperazin-2-one; N-((1H-benzo[d]imidazol-6-yl)methyl)-2-((dimethylamino)methyl)-N-(3-methoxybenzyl)pyridin-4-amine; N-((1H-indazol-6-yl)methyl)-N-(3-methoxybenzyl)-2-((2-(2-morpholinoethoxy)ethoxy)methyl)pyridin-4-amine; N-((1H-indazol-6-yl)methyl)-N-(3-methoxybenzyl)-2-((2-((3-methoxybenzyl)oxy)ethoxy)methyl)pyridin-4-amine; N-((1H-indazol-6-yl)methyl)-2-((2-(3-(dimethylamino)phenoxy)ethoxy)methyl)-N-(3-methoxybenzyl)pyridin-4-amine; N-((1H-indazol-6-yl)methyl)-N-(3-methoxybenzyl)-2-((2-morpholinoethoxy)methyl)pyridin-4-amine; N-((1H-indazol-6-yl)methyl)-N-(3-methoxybenzyl)-2-(morpholinomethyl)pyridin-4-amine; N-((1H-indazol-6-yl)methyl)-N-(3-methoxybenzyl)-2-(piperidin-1-ylmethyl)pyridin-4-amine; N-((1H-indazol-6-yl)methyl)-N-(3-methoxybenzyl)-2-((4-methylpiperazin-1-yl)methyl)pyridin-4-amine; 1-((4-(((1H-indazol-6-yl)methyl)(3-methoxybenzyl)amino)pyridin-2-yl)methyl)piperazin-2-one; 1-((4-(((1H-indazol-6-yl)methyl)(3-methoxybenzyl)amino)pyridin-2-yl)methyl)piperazine-2,5-dione; 4-((4-(((1H-indazol-6-yl)methyl)(3-methoxybenzyl)amino)pyridin-2-yl)methyl)piperazin-2-one; N-((1H-indazol-6-yl)methyl)-2-((dimethylamino)methyl)-N-(3-methoxybenzyl)pyridin-4-amine; N-(3-methoxybenzyl)-2-((2-(2-morpholinoethoxy)ethoxy)methyl)-N-(quinolin-7-ylmethyl)pyridin-4-amine; N-(3-methoxybenzyl)-2-((2-((3-methoxybenzyl)oxy)ethoxy)methyl)-N-(quinolin-7-ylmethyl)pyridin-4-amine; 2-((2-(3-(dimethylamino)phenoxy)ethoxy)methyl)-N-(3-methoxybenzyl)-N-(quinolin-7-ylmethyl)pyridin-4-amine; N-(3-methoxybenzyl)-2-((2-morpholinoethoxy)methyl)-N-(quinolin-7-ylmethyl)pyridin-4-amine; N-(3-methoxybenzyl)-2-(morpholinomethyl)-N— (quinolin-7-ylmethyl)pyridin-4-amine; N-(3-methoxybenzyl)-2-(piperidin-1-ylmethyl)-N-(quinolin-7-ylmethyl)pyridin-4-amine; N-(3-methoxybenzyl)-2-((4-methylpiperazin-1-yl)methyl)-N-(quinolin-7-ylmethyl)pyridin-4-amine; 1-((4-((3-methoxybenzyl)(quinolin-7-ylmethyl)amino)pyridin-2-yl)methyl)piperazin-2-one; 1-((4-((3-methoxybenzyl)(quinolin-7-ylmethyl)amino)pyridin-2-yl)methyl)piperazine-2,5-dione; 4-((4-((3-methoxybenzyl)(quinolin-7-ylmethyl)amino)pyridin-2-yl)methyl)piperazin-2-one; 2-((dimethylamino)methyl)-N-(3-methoxybenzyl)-N-(quinolin-7-ylmethyl)pyridin-4-amine; 4-((2-(2-((3-methoxybenzyl)oxy)ethoxy)ethoxy)methyl)-N,N-bis(3-(2-methoxyethoxy)benzyl)thiazol-2-amine; 4-((2-(2-(benzyloxy)ethoxy)ethoxy)methyl)-N,N-bis(3-(2-methoxyethoxy)benzyl)thiazol-2-amine; N,N-bis(3-(2-methoxyethoxy)benzyl)-4-((2-(2-(3-methoxyphenoxy)ethoxy)ethoxy)methyl)thiazol-2-amine; 4-((2-((3-methoxybenzyl)oxy)ethoxy)methyl)-N,N-bis(3-(2-methoxyethoxy)benzyl)thiazol-2-amine; 4-((2-(3-(dimethylamino)phenoxy)ethoxy)methyl)-N,N-bis(3-(2-methoxyethoxy)benzyl)thiazol-2-amine; N,N-bis(3-(2-methoxyethoxy)benzyl)-4-((2-(2-morpholinoethoxy)ethoxy)methyl)thiazol-2-amine; N,N-bis(3-(2-methoxyethoxy)benzyl)-4-((2-morpholinoethoxy)methyl)thiazol-2-amine; N,N-bis(3-(2-methoxyethoxy)benzyl)-4-(morpholinomethyl)thiazol-2-amine; 4-((2-(2-((3-methoxybenzyl)oxy)ethoxy)ethoxy)methyl)-N,N-bis(3-(2-methoxyethoxy)benzyl)oxazol-2-amine; 4-((2-(2-(benzyloxy)ethoxy)ethoxy)methyl)-N,N-bis(3-(2-methoxyethoxy)benzyl)oxazol-2-amine; N,N-bis(3-(2-methoxyethoxy)benzyl)-4-((2-(2-(3-methoxyphenoxy)ethoxy)ethoxy)methyl)oxazol-2-amine; 4-((2-((3-methoxybenzyl)oxy)ethoxy)methyl)-N,N-bis(3-(2-methoxyethoxy)benzyl)oxazol-2-amine; 4-((2-(3-(dimethylamino)phenoxy)ethoxy)methyl)-N,N-bis(3-(2-methoxyethoxy)benzyl)oxazol-2-amine; N,N-bis(3-(2-methoxyethoxy)benzyl)-4-((2-(2-morpholinoethoxy)ethoxy)methyl)oxazol-2-amine; N,N-bis(3-(2-methoxyethoxy)benzyl)-4-((2-morpholinoethoxy)methyl)oxazol-2-amine; N,N-bis(3-(2-methoxyethoxy)benzyl)-4-(morpholinomethyl)oxazol-2-amine; N-(3-methoxybenzyl)-4-((2-(2-((3-methoxybenzyl)oxy)ethoxy)ethoxy)methyl)-N-(3-(2-methoxyethoxy)benzyl)thiazol-2-amine; 4-((2-(2-(benzyloxy)ethoxy)ethoxy)methyl)-N-(3-methoxybenzyl)-N-(3-(2-methoxyethoxy)benzyl)thiazol-2-amine; N-(3-methoxybenzyl)-N-(3-(2-methoxyethoxy)benzyl)-4-((2-(2-(3-methoxyphenoxy)ethoxy)ethoxy)methyl)thiazol-2-amine; N-(3-methoxybenzyl)-4-((2-((3-methoxybenzyl)oxy)ethoxy)methyl)-N-(3-(2-methoxyethoxy)benzyl)thiazol-2-amine; 4-((2-(3-(dimethylamino)phenoxy)ethoxy)methyl)-N-(3-methoxybenzyl)-N-(3-(2-methoxyethoxy)benzyl)thiazol-2-amine; N-(3-methoxybenzyl)-N-(3-(2-methoxyethoxy)benzyl)-4-((2-(2-morpholinoethoxy)ethoxy)methyl)thiazol-2-amine; N-(3-methoxybenzyl)-N-(3-(2-methoxyethoxy)benzyl)-4-((2-morpholinoethoxy)methyl)thiazol-2-amine; N-(3-methoxybenzyl)-N-(3-(2-methoxyethoxy)benzyl)-4-(morpholinomethyl)thiazol-2-amine; N-(3-methoxybenzyl)-4-((2-(2-((3-methoxybenzyl)oxy)ethoxy)ethoxy)methyl)-N-(3-(2-methoxyethoxy)benzyl)oxazol-2-amine; 4-((2-(2-(benzyloxy)ethoxy)ethoxy)methyl)-N-(3-methoxybenzyl)-N-(3-(2-methoxyethoxy)benzyl)oxazol-2-amine; N-(3-methoxybenzyl)-N-(3-(2-methoxyethoxy)benzyl)-4-((2-(2-(3-methoxyphenoxy)ethoxy)ethoxy)methyl)oxazol-2-amine; 4-((2-(3-(dimethylamino)phenoxy)ethoxy)methyl)-N-(3-methoxybenzyl)-N-(3-(2-methoxyethoxy)benzyl)oxazol-2-amine; N-(3-methoxybenzyl)-N-(3-(2-methoxyethoxy)benzyl)-4-((2-(2-morpholinoethoxy)ethoxy)methyl)oxazol-2-amine; N-(3-methoxybenzyl)-N-(3-(2-methoxyethoxy)benzyl)-4-((2-morpholinoethoxy)methyl)oxazol-2-amine; N-(3-methoxybenzyl)-N-(3-(2-methoxyethoxy)benzyl)-4-(morpholinomethyl)oxazol-2-amine; N,N-bis(3-methoxybenzyl)-4-(2-morpholinoethyl)thiazol-2-amine; N,N-bis(3-methoxybenzyl)-4-methyl-5-(morpholinomethyl)thiazol-2-amine; N-(3-(dimethylamino)benzyl)-N-(3-methoxybenzyl)-4-(2-morpholinoethyl)thiazol-2-amine; N-(3-(dimethylamino)benzyl)-N-(3-methoxybenzyl)-4-methyl-5-(morpholinomethyl)thiazol-2-amine; N-(3-methoxybenzyl)-4-(2-morpholinoethyl)-N-(3-(pyrrolidin-1-yl)benzyl)thiazol-2-amine; N-(3-methoxybenzyl)-4-methyl-5-(morpholinomethyl)-N-(3-(pyrrolidin-1-yl)benzyl)thiazol-2-amine; N-(3-(1H-pyrazol-1-yl)benzyl)-N-(3-methoxybenzyl)-4-(2-morpholinoethyl)thiazol-2-amine; N-(3-(1H-pyrazol-1-yl)benzyl)-N-(3-methoxybenzyl)-4-methyl-5-(morpholinomethyl)thiazol-2-amine; N-(3-(1H-imidazol-1-yl)benzyl)-N-(3-methoxybenzyl)-4-(2-morpholinoethyl)thiazol-2-amine; N-(3-(1H-imidazol-1-yl)benzyl)-N-(3-methoxybenzyl)-4-methyl-5-(morpholinomethyl)thiazol-2-amine; N-(3-methoxybenzyl)-4-(2-morpholinoethyl)-N-(4-(pyrrolidin-1-yl)benzyl)thiazol-2-amine; N-(3-methoxybenzyl)-4-methyl-5-(morpholinomethyl)-N-(4-(pyrrolidin-1-yl)benzyl)thiazol-2-amine; N-(3-methoxybenzyl)-N-(4-morpholinobenzyl)-4-(2-morpholinoethyl)thiazol-2-amine; N-(3-methoxybenzyl)-4-methyl-N-(4-morpholinobenzyl)-5-(morpholinomethyl)thiazol-2-amine; N-(3-methoxybenzyl)-N-(4-(4-methylpiperazin-1-yl)benzyl)-4-(2-morpholinoethyl)thiazol-2-amine; N-(3-methoxybenzyl)-4-methyl-N-(4-(4-methylpiperazin-1-yl)benzyl)-5-(morpholinomethyl)thiazol-2-amine; N-(3-methoxybenzyl)-N-(3-morpholinobenzyl)-4-(2-morpholinoethyl)thiazol-2-amine; N-(3-methoxybenzyl)-4-methyl-N-(3-morpholinobenzyl)-5-(morpholinomethyl)thiazol-2-amine; N-(3-methoxybenzyl)-N-(3-(4-methylpiperazin-1-yl)benzyl)-4-(2-morpholinoethyl)thiazol-2-amine; N-(3-methoxybenzyl)-4-methyl-N-(3-(4-methylpiperazin-1-yl)benzyl)-5-(morpholinomethyl)thiazol-2-amine; N-(4-(1H-pyrazol-1-yl)benzyl)-N-(3-methoxybenzyl)-4-(2-morpholinoethyl)thiazol-2-amine; N-(4-(1H-pyrazol-1-yl)benzyl)-N-(3-methoxybenzyl)-4-methyl-5-(morpholinomethyl)thiazol-2-amine; N-(4-(1H-imidazol-1-yl)benzyl)-N-(3-methoxybenzyl)-4-(2-morpholinoethyl)thiazol-2-amine; N-(4-(1H-imidazol-1-yl)benzyl)-N-(3-methoxybenzyl)-4-methyl-5-(morpholinomethyl)thiazol-2-amine; N-((1H-benzo[d]imidazol-6-yl)methyl)-N-(3-methoxybenzyl)-4-(2-morpholinoethyl)thiazol-2-amine; N-((1H-benzo[d]imidazol-6-yl)methyl)-N-(3-methoxybenzyl)-4-methyl-5-(morpholinomethyl)thiazol-2-amine; N-((1H-indazol-6-yl)methyl)-N-(3-methoxybenzyl)-4-(2-morpholinoethyl)thiazol-2-amine; N-((1H-indazol-6-yl)methyl)-N-(3-methoxybenzyl)-4-methyl-5-(morpholinomethyl)thiazol-2-amine; N-(3-methoxybenzyl)-4-(2-morpholinoethyl)-N-(quinolin-7-ylmethyl)thiazol-2-amine; N-(3-methoxybenzyl)-4-methyl-5-(morpholinomethyl)-N-(quinolin-7-ylmethyl)thiazol-2-amine; N,N-bis(3-methoxybenzyl)-4-(2-morpholinoethyl)oxazol-2-amine; N,N-bis(3-methoxybenzyl)-4-methyl-5-(morpholinomethyl)oxazol-2-amine; N-(3-(dimethylamino)benzyl)-N-(3-methoxybenzyl)-4-(2-morpholinoethyl)oxazol-2-amine; N-(3-(dimethylamino)benzyl)-N-(3-methoxybenzyl)-4-methyl-5-(morpholinomethyl)oxazol-2-amine; N-(3-methoxybenzyl)-4-(2-morpholinoethyl)-N-(3-(pyrrolidin-1-yl)benzyl)oxazol-2-amine; N-(3-methoxybenzyl)-4-methyl-5-(morpholinomethyl)-N-(3-(pyrrolidin-1-yl)benzyl)oxazol-2-amine; N-(3-(1H-pyrazol-1-yl)benzyl)-N-(3-methoxybenzyl)-4-(2-morpholinoethyl)oxazol-2-amine; N-(3-(1H-pyrazol-1-yl)benzyl)-N-(3-methoxybenzyl)-4-methyl-5-(morpholinomethyl)oxazol-2-amine; N-(3-(1H-imidazol-1-yl)benzyl)-N-(3-methoxybenzyl)-4-(2-morpholinoethyl)oxazol-2-amine; N-(3-(1H-imidazol-1-yl)benzyl)-N-(3-methoxybenzyl)-4-methyl-5-(morpholinomethyl)oxazol-2-amine; N-(3-methoxybenzyl)-4-(2-morpholinoethyl)-N-(4-(pyrrolidin-1-yl)benzyl)oxazol-2-amine; N-(3-methoxybenzyl)-4-methyl-5-(morpholinomethyl)-N-(4-(pyrrolidin-1-yl)benzyl)oxazol-2-amine; N-(3-methoxybenzyl)-N-(4-morpholinobenzyl)-4-(2-morpholinoethyl)oxazol-2-amine; N-(3-methoxybenzyl)-4-methyl-N-(4-morpholinobenzyl)-5-(morpholinomethyl)oxazol-2-amine; N-(3-methoxybenzyl)-N-(4-(4-methylpiperazin-1-yl)benzyl)-4-(2-morpholinoethyl)oxazol-2-amine; N-(3-methoxybenzyl)-4-methyl-N-(4-(4-methylpiperazin-1-yl)benzyl)-5-(morpholinomethyl)oxazol-2-amine; N-(3-methoxybenzyl)-N-(3-morpholinobenzyl)-4-(2-morpholinoethyl)oxazol-2-amine; N-(3-methoxybenzyl)-4-methyl-N-(3-morpholinobenzyl)-5-(morpholinomethyl)oxazol-2-amine; N-(3-methoxybenzyl)-N-(3-(4-methylpiperazin-1-yl)benzyl)-4-(2-morpholinoethyl)oxazol-2-amine; N-(3-methoxybenzyl)-4-methyl-N-(3-(4-methylpiperazin-1-yl)benzyl)-5-(morpholinomethyl)oxazol-2-amine; N-(4-(1H-imidazol-1-yl)benzyl)-N-(3-methoxybenzyl)-4-(2-morpholinoethyl)oxazol-2-amine; N-(4-(1H-imidazol-1-yl)benzyl)-N-(3-methoxybenzyl)-4-methyl-5-(morpholinomethyl)oxazol-2-amine; N-(4-(1H-pyrazol-1-yl)benzyl)-N-(3-methoxybenzyl)-4-(2-morpholinoethyl)oxazol-2-amine; N-(4-(1H-pyrazol-1-yl)benzyl)-N-(3-methoxybenzyl)-4-methyl-5-(morpholinomethyl)oxazol-2-amine; N-((1H-benzo[d]imidazol-6-yl)methyl)-N-(3-methoxybenzyl)-4-(2-morpholinoethyl)oxazol-2-amine; N-((1H-benzo[d]imidazol-6-yl)methyl)-N-(3-methoxybenzyl)-4-methyl-5-(morpholinomethyl)oxazol-2-amine; N-((1H-indazol-6-yl)methyl)-N-(3-methoxybenzyl)-4-(2-morpholinoethyl)oxazol-2-amine; N-((1H-indazol-6-yl)methyl)-N-(3-methoxybenzyl)-4-methyl-5-(morpholinomethyl)oxazol-2-amine; N-(3-methoxybenzyl)-4-(2-morpholinoethyl)-N-(quinolin-7-ylmethyl)oxazol-2-amine; N-(3-methoxybenzyl)-4-methyl-5-(morpholinomethyl)-N-(quinolin-7-ylmethyl)oxazol-2-amine; 5-ethyl-N,N-bis(3-methoxybenzyl)-1,3,4-thiadiazol-2-amine; N,N-bis(3-methoxybenzyl)-5-((2-(2-(3-methoxyphenoxy)ethoxy)ethoxy)methyl)pyridin-2-amine; 5-((2-(2-(benzyloxy)ethoxy)ethoxy)methyl)-N,N-bis(3-methoxybenzyl)pyridin-2-amine; N,N-bis(3-methoxybenzyl)-5-((2-(2-((3-methoxybenzyl)oxy)ethoxy)ethoxy)methyl)pyridin-2-amine; 5-((2-(3-(dimethylamino)phenoxy)ethoxy)methyl)-N,N-bis(3-methoxybenzyl)pyridin-2-amine; N,N-bis(3-methoxybenzyl)-5-((2-((3-methoxybenzyl)oxy)ethoxy)methyl)pyridin-2-amine; N,N-bis(3-methoxybenzyl)-5-((2-(2-morpholinoethoxy)ethoxy)methyl)pyridin-2-amine; N,N-bis(3-methoxybenzyl)-5-((2-morpholinoethoxy)methyl)pyridin-2-amine; N,N-bis(3-methoxybenzyl)-5-(morpholinomethyl)pyridin-2-amine; N-(3-(dimethylamino)benzyl)-N-(3-methoxybenzyl)-5-((2-(2-(3-methoxyphenoxy)ethoxy)ethoxy)methyl)pyridin-2-amine; 5-((2-(2-(benzyloxy)ethoxy)ethoxy)methyl)-N-(3-(dimethylamino)benzyl)-N-(3-methoxybenzyl)pyridin-2-amine; N-(3-(dimethylamino)benzyl)-N-(3-methoxybenzyl)-5-((2-(2-((3-methoxybenzyl)oxy)ethoxy)ethoxy)methyl)pyridin-2-amine; N-(3-(dimethylamino)benzyl)-5-((2-(3-(dimethylamino)phenoxy)ethoxy)methyl)-N-(3-methoxybenzyl)pyridin-2-amine; N-(3-(dimethylamino)benzyl)-N-(3-methoxybenzyl)-5-((2-((3-methoxybenzyl)oxy)ethoxy)methyl)pyridin-2-amine; N-(3-(dimethylamino)benzyl)-N-(3-methoxybenzyl)-5-((2-(2-morpholinoethoxy)ethoxy)methyl)pyridin-2-amine; N-(3-(dimethylamino)benzyl)-N-(3-methoxybenzyl)-5-((2-morpholinoethoxy)methyl)pyridin-2-amine; N-(3-(dimethylamino)benzyl)-N-(3-methoxybenzyl)-5-(morpholinomethyl)pyridin-2-amine; N-(3-methoxybenzyl)-5-((2-(2-(3-methoxyphenoxy)ethoxy)ethoxy)methyl)-N-(4-morpholinobenzyl)pyridin-2-amine; 5-((2-(2-(benzyloxy)ethoxy)ethoxy)methyl)-N-(3-methoxybenzyl)-N-(4-morpholinobenzyl)pyridin-2-amine; N-(3-methoxybenzyl)-5-((2-(2-((3-methoxybenzyl)oxy)ethoxy)ethoxy)methyl)-N-(4-morpholinobenzyl)pyridin-2-amine; 5-((2-(3-(dimethylamino)phenoxy)ethoxy)methyl)-N-(3-methoxybenzyl)-N-(4-morpholinobenzyl)pyridin-2-amine; N-(3-methoxybenzyl)-5-((2-((3-methoxybenzyl)oxy)ethoxy)methyl)-N-(4-morpholinobenzyl)pyridin-2-amine; N-(3-methoxybenzyl)-N-(4-morpholinobenzyl)-5-((2-(2-morpholinoethoxy)ethoxy)methyl)pyridin-2-amine; N-(3-methoxybenzyl)-N-(4-morpholinobenzyl)-5-((2-morpholinoethoxy)methyl)pyridin-2-amine; N-(3-methoxybenzyl)-N-(4-morpholinobenzyl)-5-(morpholinomethyl)pyridin-2-amine; N-((1H-indazol-6-yl)methyl)-N-(3-methoxybenzyl)-5-((2-(2-(3-methoxyphenoxy)ethoxy)ethoxy)methyl)pyridin-2-amine; N-((1H-indazol-6-yl)methyl)-5-((2-(2-(benzyloxy)ethoxy)ethoxy)methyl)-N-(3-methoxybenzyl)pyridin-2-amine; N-((1H-indazol-6-yl)methyl)-N-(3-methoxybenzyl)-5-((2-(2-((3-methoxybenzyl)oxy)ethoxy)ethoxy)methyl)pyridin-2-amine; N-((1H-indazol-6-yl)methyl)-5-((2-(3-(dimethylamino)phenoxy)ethoxy)methyl)-N-(3-methoxybenzyl)pyridin-2-amine; N-((1H-indazol-6-yl)methyl)-N-(3-methoxybenzyl)-5-((2-((3-methoxybenzyl)oxy)ethoxy)methyl)pyridin-2-amine; N-((1H-indazol-6-yl)methyl)-N-(3-methoxybenzyl)-5-((2-(2 morpholinoethoxy)ethoxy)methyl)pyridin-2-amine; N-((1H-indazol-6-yl)methyl)-N-(3-methoxybenzyl)-5-((2-morpholinoethoxy)methyl)pyridin-2-amine; and N-((1H-indazol-6-yl)methyl)-N-(3-methoxybenzyl)-5-(morpholinomethyl)pyridin-2-amine. N,N-bis(3-methoxybenzyl)-4-(2-(2-(3-methoxyphenoxy)ethoxy)ethoxy)aniline; 4-(2-(2-(benzyloxy)ethoxy)ethoxy)-N,N-bis(3-methoxybenzyl)aniline; N,N-bis(3-methoxybenzyl)-4-(2-(2-morpholinoethoxy)ethoxy)aniline; 3-(2-(4-(bis(3-methoxybenzyl)amino)phenoxy)ethoxy)-N,N-dimethylaniline; N,N-bis(3-methoxybenzyl)-4-(2-morpholinoethoxy)aniline; 3-(((3-methoxybenzyl)(4-(2-(2-(3-methoxyphenoxy)ethoxy)ethoxy)phenyl)amino)methyl)-N,N-dimethylaniline; 3-(((4-(2-(2-(benzyloxy)ethoxy)ethoxy)phenyl)(3-methoxybenzyl)amino)methyl)-N,N-dimethylaniline; 3-(((3-methoxybenzyl)(4-(2-(2-morpholinoethoxy)ethoxy)phenyl)amino)methyl)-N,N-dimethylaniline; 3-(2-(4-((3-(dimethylamino)benzyl)(3-methoxybenzyl)amino)phenoxy)ethoxy)-N,N-dimethylaniline; 3-(((3-methoxybenzyl)(4-(2-morpholinoethoxy)phenyl)amino)methyl)-N,N-dimethylaniline; N-(3-methoxybenzyl)-4-(2-(2-(3-methoxyphenoxy)ethoxy)ethoxy)-N-(3-(pyrrolidin-1-yl)benzyl)aniline; 4-(2-(2-(benzyloxy)ethoxy)ethoxy)-N-(3-methoxybenzyl)-N-(3-(pyrrolidin-1-yl)benzyl)aniline; N-(3-methoxybenzyl)-4-(2-(2-morpholinoethoxy)ethoxy)-N-(3-(pyrrolidin-1-yl)benzyl)aniline; 3-(2-(4-((3-methoxybenzyl)(3-(pyrrolidin-1-yl)benzyl)amino)phenoxy)ethoxy)-N,N-dimethylaniline; N-(3-methoxybenzyl)-4-(2-morpholinoethoxy)-N-(3-(pyrrolidin-1-yl)benzyl)aniline; N-(3-(1H-imidazol-1-yl)benzyl)-N-(3-methoxybenzyl)-4-(2-(2-(3-methoxyphenoxy)ethoxy)ethoxy)aniline; N-(3-(1H-imidazol-1-yl)benzyl)-4-(2-(2-(benzyloxy)ethoxy)ethoxy)-N-(3-methoxybenzyl)aniline; N-(3-(1H-imidazol-1-yl)benzyl)-N-(3-methoxybenzyl)-4-(2-(2-morpholinoethoxy)ethoxy)aniline; 3-(2-(4-((3-(1H-imidazol-1-yl)benzyl)(3-methoxybenzyl)amino)phenoxy)ethoxy)-N,N-dimethylaniline; N-(3-(1H-imidazol-1-yl)benzyl)-N-(3-methoxybenzyl)-4-(2-morpholinoethoxy)aniline; N-(3-methoxybenzyl)-4-(2-(2-(3-methoxyphenoxy)ethoxy)ethoxy)-N-(4-morpholinobenzyl)aniline; 4-(2-(2-(benzyloxy)ethoxy)ethoxy)-N-(3-methoxybenzyl)-N-(4-morpholinobenzyl)aniline; N-(3-methoxybenzyl)-N-(4-morpholinobenzyl)-4-(2-(2-morpholinoethoxy)ethoxy)aniline; 3-(2-(4-((3-methoxybenzyl)(4-morpholinobenzyl)amino)phenoxy)ethoxy)-N,N-dimethylaniline; N-(3-methoxybenzyl)-N-(4-morpholinobenzyl)-4-(2-morpholinoethoxy)aniline; N-(4-(1H-pyrazol-1-yl)benzyl)-N-(3-methoxybenzyl)-4-(2-(2-(3-methoxyphenoxy)ethoxy)ethoxy)aniline; N-(4-(1H-pyrazol-1-yl)benzyl)-4-(2-(2-(benzyloxy)ethoxy)ethoxy)-N-(3-methoxybenzyl)aniline; N-(4-(1H-pyrazol-1-yl)benzyl)-N-(3-methoxybenzyl)-4-(2-(2-morpholinoethoxy)ethoxy)aniline; 3-(2-(4-((4-(1H-pyrazol-1-yl)benzyl)(3-methoxybenzyl)amino)phenoxy)ethoxy)-N,N-dimethylaniline; N-(4-(1H-pyrazol-1-yl)benzyl)-N-(3-methoxybenzyl)-4-(2-morpholinoethoxy)aniline; N-((1H-benzo[d]imidazol-6-yl)methyl)-N-(3-methoxybenzyl)-4-(2-(2-(3-methoxyphenoxy)ethoxy)ethoxy)aniline; N-((1H-benzo[d]imidazol-6-yl)methyl)-4-(2-(2-(benzyloxy)ethoxy)ethoxy)-N-(3-methoxybenzyl)aniline; N-((1H-benzo[d]imidazol-6-yl)methyl)-N-(3-methoxybenzyl)-4-(2-(2-morpholinoethoxy)ethoxy)aniline; 3-(2-(4-(((1H-benzo[d]imidazol-6-yl)methyl)(3-methoxybenzyl)amino)phenoxy)ethoxy)-N,N-dimethylaniline; N-((1H-benzo[d]imidazol-6-yl)methyl)-N-(3-methoxybenzyl)-4-(2-morpholinoethoxy)aniline; N,N-bis(3-methoxybenzyl)-3-(2-(2-(3-methoxyphenoxy)ethoxy)ethoxy)aniline; 3-(2-(2-(benzyloxy)ethoxy)ethoxy)-N,N-bis(3-methoxybenzyl)aniline; N,N-bis(3-methoxybenzyl)-3-(2-(2-morpholinoethoxy)ethoxy)aniline; 3-(2-(3-(bis(3-methoxybenzyl)amino)phenoxy)ethoxy)-N,N-dimethylaniline; N,N-bis(3-methoxybenzyl)-3-(2-morpholinoethoxy)aniline; N-(3-(dimethylamino)benzyl)-N-(3-methoxybenzyl)-3-(2-(2-(3-methoxyphenoxy)ethoxy)ethoxy)aniline; 3-(2-(2-(benzyloxy)ethoxy)ethoxy)-N-(3-(dimethylamino)benzyl)-N-(3-methoxybenzyl)aniline; N-(3-(dimethylamino)benzyl)-N-(3-methoxybenzyl)-3-(2-(2-morpholinoethoxy)ethoxy)aniline; N-(3-(dimethylamino)benzyl)-3-(2-(3-(dimethylamino)phenoxy)ethoxy)-N-(3-methoxybenzyl)aniline; N-(3-(dimethylamino)benzyl)-N-(3-methoxybenzyl)-3-(2-morpholinoethoxy)aniline; N-(3-methoxybenzyl)-3-(2-(2-(3-methoxyphenoxy)ethoxy)ethoxy)-N-(3-(pyrrolidin-1-yl)benzyl)aniline; 3-(2-(2-(benzyloxy)ethoxy)ethoxy)-N-(3-methoxybenzyl)-N-(3-(pyrrolidin-1-yl)benzyl)aniline; N-(3-methoxybenzyl)-3-(2-(2-morpholinoethoxy)ethoxy)-N-(3-(pyrrolidin-1-yl)benzyl)aniline; 3-(2-(3-(dimethylamino)phenoxy)ethoxy)-N-(3-methoxybenzyl)-N-(3-(pyrrolidin-1-yl)benzyl)aniline; N-(3-methoxybenzyl)-3-(2-morpholinoethoxy)-N-(3-(pyrrolidin-1-yl)benzyl)aniline; N-(3-(1H-imidazol-1-yl)benzyl)-N-(3-methoxybenzyl)-3-(2-(2-(3-methoxyphenoxy)ethoxy)ethoxy)aniline; N-(3-(1H-imidazol-1-yl)benzyl)-3-(2-(2-(benzyloxy)ethoxy)ethoxy)-N-(3-methoxybenzyl)aniline; N-(3-(1H-imidazol-1-yl)benzyl)-N-(3-methoxybenzyl)-3-(2-(2-morpholinoethoxy)ethoxy)aniline; N-(3-(1H-imidazol-1-yl)benzyl)-3-(2-(3-(dimethylamino)phenoxy)ethoxy)-N-(3-methoxybenzyl)aniline; N-(3-(1H-imidazol-1-yl)benzyl)-N-(3-methoxybenzyl)-3-(2-morpholinoethoxy)aniline; N-(3-methoxybenzyl)-3-(2-(2-(3-methoxyphenoxy)ethoxy)ethoxy)-N-(4-morpholinobenzyl)aniline; N-(3-methoxybenzyl)-3-(2-(2-(3-methoxyphenoxy)ethoxy)ethoxy)-N-(4-morpholinobenzyl)aniline; N-(3-methoxybenzyl)-N-(4-morpholinobenzyl)-3-(2-(2-morpholinoethoxy)ethoxy)aniline; 3-(2-(3-(dimethylamino)phenoxy)ethoxy)-N-(3-methoxybenzyl)-N-(4-morpholinobenzyl)aniline; N-(3-methoxybenzyl)-N-(4-morpholinobenzyl)-3-(2-morpholinoethoxy)aniline; N-(4-(1H-pyrazol-1-yl)benzyl)-N-(3-methoxybenzyl)-3-(2-(2-(3-methoxyphenoxy)ethoxy)ethoxy)aniline; N-(4-(1H-pyrazol-1-yl)benzyl)-3-(2-(2-(benzyloxy)ethoxy)ethoxy)-N-(3-methoxybenzyl)aniline; N-(4-(1H-pyrazol-1-yl)benzyl)-N-(3-methoxybenzyl)-3-(2-(2-morpholinoethoxy)ethoxy)aniline; N-(4-(1H-pyrazol-1-yl)benzyl)-3-(2-(3-(dimethylamino)phenoxy)ethoxy)-N-(3-methoxybenzyl)aniline; N-(4-(1H-pyrazol-1-yl)benzyl)-N-(3-methoxybenzyl)-3-(2-morpholinoethoxy)aniline; N-((1H-benzo[d]imidazol-6-yl)methyl)-N-(3-methoxybenzyl)-3-(2-(2-(3-methoxyphenoxy)ethoxy)ethoxy)aniline; N-((1H-benzo[d]imidazol-6-yl)methyl)-3-(2-(2-(benzyloxy)ethoxy)ethoxy)-N-(3-methoxybenzyl)aniline; N-((1H-benzo[d]imidazol-6-yl)methyl)-N-(3-methoxybenzyl)-3-(2-(2-morpholinoethoxy)ethoxy)aniline; N-((1H-benzo[d]imidazol-6-yl)methyl)-3-(2-(3-(dimethylamino)phenoxy)ethoxy)-N-(3-methoxybenzyl)aniline; N-((1H-benzo[d]imidazol-6-yl)methyl)-N-(3-methoxybenzyl)-3-(2-morpholinoethoxy)aniline; N,N-bis(3-methoxybenzyl)-2-(2-(2-(3-methoxyphenoxy)ethoxy)ethoxy)pyridin-4-amine; 2-(2-(2-(benzyloxy)ethoxy)ethoxy)-N,N-bis(3-methoxybenzyl)pyridin-4-amine; N,N-bis(3-methoxybenzyl)-2-(2-(2-morpholinoethoxy)ethoxy)pyridin-4-amine; 2-(2-(3-(dimethylamino)phenoxy)ethoxy)-N,N-bis(3-methoxybenzyl)pyridin-4-amine; N,N-bis(3-methoxybenzyl)-2-(2-morpholinoethoxy)pyridin-4-amine; N-(3-(dimethylamino)benzyl)-N-(3-methoxybenzyl)-2-(2-(2-(3-methoxyphenoxy)ethoxy)ethoxy)pyridin-4-amine; 2-(2-(2-(benzyloxy)ethoxy)ethoxy)-N-(3-(dimethylamino)benzyl)-N-(3-methoxybenzyl)pyridin-4-amine; N-(3-(dimethylamino)benzyl)-N-(3-methoxybenzyl)-2-(2-(2-morpholinoethoxy)ethoxy)pyridin-4-amine; N-(3-(dimethylamino)benzyl)-2-(2-(3-(dimethylamino)phenoxy)ethoxy)-N-(3-methoxybenzyl)pyridin-4-amine; N-(3-(dimethylamino)benzyl)-N-(3-methoxybenzyl)-2-(2-morpholinoethoxy)pyridin-4-amine; N-(3-methoxybenzyl)-2-(2-(2-(3-methoxyphenoxy)ethoxy)ethoxy)-N-(3-(pyrrolidin-1-yl)benzyl)pyridin-4-amine; 2-(2-(2-(benzyloxy)ethoxy)ethoxy)-N-(3-methoxybenzyl)-N-(3-(pyrrolidin-1-yl)benzyl)pyridin-4-amine; N-(3-methoxybenzyl)-2-(2-(2-morpholinoethoxy)ethoxy)-N-(3-(pyrrolidin-1-yl)benzyl)pyridin-4-amine; 2-(2-(3-(dimethylamino)phenoxy)ethoxy)-N-(3-methoxybenzyl)-N-(3-(pyrrolidin-1-yl)benzyl)pyridin-4-amine; N-(3-methoxybenzyl)-2-(2-morpholinoethoxy)-N-(3-(pyrrolidin-1-yl)benzyl)pyridin-4-amine; N-(3-(1H-imidazol-1-yl)benzyl)-N-(3-methoxybenzyl)-2-(2-(2-(3-methoxyphenoxy)ethoxy)ethoxy)pyridin-4-amine; N-(3-(1H-imidazol-1-yl)benzyl)-2-(2-(2-(benzyloxy)ethoxy)ethoxy)-N-(3-methoxybenzyl)pyridin-4-amine; N-(3-(1H-imidazol-1-yl)benzyl)-N-(3-methoxybenzyl)-2-(2-(2-morpholinoethoxy)ethoxy)pyridin-4-amine; N-(3-(1H-imidazol-1-yl)benzyl)-2-(2-(3-(dimethylamino)phenoxy)ethoxy)-N-(3-methoxybenzyl)pyridin-4-amine; N-(3-(1H-imidazol-1-yl)benzyl)-N-(3-methoxybenzyl)-2-(2-morpholinoethoxy)pyridin-4-amine; N-(3-methoxybenzyl)-2-(2-(2-(3-methoxyphenoxy)ethoxy)ethoxy)-N-(4-morpholinobenzyl)pyridin-4-amine; 2-(2-(2-(benzyloxy)ethoxy)ethoxy)-N-(3-methoxybenzyl)-N-(4-morpholinobenzyl)pyridin-4-amine; N-(3-methoxybenzyl)-N-(4-morpholinobenzyl)-2-(2-(2-morpholinoethoxy)ethoxy)pyridin-4-amine; 2-(2-(3-(dimethylamino)phenoxy)ethoxy)-N-(3-methoxybenzyl)-N-(4-morpholinobenzyl)pyridin-4-amine; N-(3-methoxybenzyl)-N-(4-morpholinobenzyl)-2-(2-morpholinoethoxy)pyridin-4-amine; N-(4-(1H-pyrazol-1-yl)benzyl)-N-(3-methoxybenzyl)-2-(2-(2-(3 methoxyphenoxy)ethoxy)ethoxy)pyridin-4-amine; N-(4-(1H-pyrazol-1-yl)benzyl)-2-(2-(2-(benzyloxy)ethoxy)ethoxy)-N-(3-methoxybenzyl)pyridin-4-amine; N-(4-(1H-pyrazol-1-yl)benzyl)-N-(3-methoxybenzyl)-2-(2-(2-morpholinoethoxy)ethoxy)pyridin-4-amine; N-(4-(1H-pyrazol-1-yl)benzyl)-2-(2-(3-(dimethylamino)phenoxy)ethoxy)-N-(3-methoxybenzyl)pyridin-4-amine; N-(4-(1H-pyrazol-1-yl)benzyl)-N-(3-methoxybenzyl)-2-(2-morpholinoethoxy)pyridin-4-amine; N-((1H-benzo[d]imidazol-6-yl)methyl)-N-(3-methoxybenzyl)-2-(2-(2-(3-methoxyphenoxy)ethoxy)ethoxy)pyridin-4-amine; N-((1H-benzo[d]imidazol-6-yl)methyl)-2-(2-(2-(benzyloxy)ethoxy)ethoxy)-N-(3-methoxybenzyl)pyridin-4-amine; N-((1H-benzo[d]imidazol-6-yl)methyl)-N-(3-methoxybenzyl)-2-(2-(2-morpholinoethoxy)ethoxy)pyridin-4-amine; N-((1H-benzo[d]imidazol-6-yl)methyl)-2-(2-(3-(dimethylamino)phenoxy)ethoxy)-N-(3-methoxybenzyl)pyridin-4-amine; N-((1H-benzo[d]imidazol-6-yl)methyl)-N-(3-methoxybenzyl)-2-(2-morpholinoethoxy)pyridin-4-amine; N,N-bis(3-methoxybenzyl)-5-(2-(2-(3-methoxyphenoxy)ethoxy)ethoxy)pyridin-2-amine; 5-(2-(2-(benzyloxy)ethoxy)ethoxy)-N,N-bis(3-methoxybenzyl)pyridin-2-amine; N,N-bis(3-methoxybenzyl)-5-(2-(2-morpholinoethoxy)ethoxy)pyridin-2-amine; 5-(2-(3-(dimethylamino)phenoxy)ethoxy)-N,N-bis(3-methoxybenzyl)pyridin-2-amine; N,N-bis(3-methoxybenzyl)-5-(2-morpholinoethoxy)pyridin-2-amine; N-(3-(dimethylamino)benzyl)-N-(3-methoxybenzyl)-5-(2-(2-(3-methoxyphenoxy)ethoxy)ethoxy)pyridin-2-amine; 5-(2-(2-(benzyloxy)ethoxy)ethoxy)-N-(3-(dimethylamino)benzyl)-N-(3-methoxybenzyl)pyridin-2-amine; N-(3-(dimethylamino)benzyl)-N-(3-methoxybenzyl)-5-(2-(2-morpholinoethoxy)ethoxy)pyridin-2-amine; N-(3-(dimethylamino)benzyl)-5-(2-(3-(dimethylamino)phenoxy)ethoxy)-N-(3-methoxybenzyl)pyridin-2-amine; N-(3-(dimethylamino)benzyl)-N-(3-methoxybenzyl)-5-(2-morpholinoethoxy)pyridin-2-amine; N-(3-methoxybenzyl)-5-(2-(2-(3-methoxyphenoxy)ethoxy)ethoxy)-N-(3-(pyrrolidin-1-yl)benzyl)pyridin-2-amine; 5-(2-(2-(benzyloxy)ethoxy)ethoxy)-N-(3-methoxybenzyl)-N-(3-(pyrrolidin-1-yl)benzyl)pyridin-2-amine; N-(3-methoxybenzyl)-5-(2-(2-morpholinoethoxy)ethoxy)-N-(3-(pyrrolidin-1-yl)benzyl)pyridin-2-amine; 5-(2-(3-(dimethylamino)phenoxy)ethoxy)-N-(3-methoxybenzyl)-N-(3-(pyrrolidin-1-yl)benzyl)pyridin-2-amine; N-(3-methoxybenzyl)-5-(2-morpholinoethoxy)-N-(3-(pyrrolidin-1-yl)benzyl)pyridin-2-amine; N-(3-(1H-imidazol-1-yl)benzyl)-N-(3-methoxybenzyl)-5-(2-(2-(3-methoxyphenoxy)ethoxy)ethoxy)pyridin-2-amine; N-(3-(1H-imidazol-1-yl)benzyl)-5-(2-(2-(benzyloxy)ethoxy)ethoxy)-N-(3-methoxybenzyl)pyridin-2-amine; N-(3-(1H-imidazol-1-yl)benzyl)-N-(3-methoxybenzyl)-5-(2-(2-morpholinoethoxy)ethoxy)pyridin-2-amine; N-(3-(1H-imidazol-1-yl)benzyl)-5-(2-(3-(dimethylamino)phenoxy)ethoxy)-N-(3-methoxybenzyl)pyridin-2-amine; N-(3-(1H-imidazol-1-yl)benzyl)-N-(3-methoxybenzyl)-5-(2-morpholinoethoxy)pyridin-2-amine; N-(3-methoxybenzyl)-5-(2-(2-(3-methoxyphenoxy)ethoxy)ethoxy)-N-(4-morpholinobenzyl)pyridin-2-amine; 5-(2-(2-(benzyloxy)ethoxy)ethoxy)-N-(3-methoxybenzyl)-N-(4-morpholinobenzyl)pyridin-2-amine; N-(3-methoxybenzyl)-N-(4-morpholinobenzyl)-5-(2-(2-morpholinoethoxy)ethoxy)pyridin-2-amine; 5-(2-(3-(dimethylamino)phenoxy)ethoxy)-N-(3-methoxybenzyl)-N-(4-morpholinobenzyl)pyridin-2-amine; N-(3-methoxybenzyl)-N-(4-morpholinobenzyl)-5-(2-morpholinoethoxy)pyridin-2-amine; N-(4-(1H-pyrazol-1-yl)benzyl)-N-(3-methoxybenzyl)-5-(2-(2-(3 methoxyphenoxy)ethoxy)ethoxy)pyridin-2-amine; N-(4-(1H-pyrazol-1-yl)benzyl)-5-(2-(2-(benzyloxy)ethoxy)ethoxy)-N-(3-methoxybenzyl)pyridin-2-amine; N-(4-(1H-pyrazol-1-yl)benzyl)-N-(3-methoxybenzyl)-5-(2-(2-morpholinoethoxy)ethoxy)pyridin-2-amine; N-(4-(1H-pyrazol-1-yl)benzyl)-5-(2-(3-(dimethylamino)phenoxy)ethoxy)-N-(3-methoxybenzyl)pyridin-2-amine; N-(4-(1H-pyrazol-1-yl)benzyl)-N-(3-methoxybenzyl)-5-(2-morpholinoethoxy)pyridin-2-amine; N-((1H-benzo[d]imidazol-6-yl)methyl)-N-(3-methoxybenzyl)-5-(2-(2-(3-methoxyphenoxy)ethoxy)ethoxy)pyridin-2-amine; N-((1H-benzo[d]imidazol-6-yl)methyl)-5-(2-(2-(benzyloxy)ethoxy)ethoxy)-N-(3-methoxybenzyl)pyridin-2-amine; N-((1H-benzo[d]imidazol-6-yl)methyl)-N-(3-methoxybenzyl)-5-(2-(2-morpholinoethoxy)ethoxy)pyridin-2-amine; N-((1H-benzo[d]imidazol-6-yl)methyl)-5-(2-(3-(dimethylamino)phenoxy)ethoxy)-N-(3-methoxybenzyl)pyridin-2-amine; and N-((1H-benzo[d]imidazol-6-yl)methyl)-N-(3-methoxybenzyl)-5-(2-morpholinoethoxy)pyridin-2-amine.

8. The compound of claim 1, wherein the VLA-4 integrin agonist is selected from the group consisting essentially of: N,N-bis(3-methoxybenzyl)-4-((2-(3-methoxyphenoxy)ethoxy)methyl)thiazol-2-amine; N,N-bis(3-methoxybenzyl)-4-((2-(2-morpholinoethoxy)ethoxy)methyl)thiazol-2-amine; N,N-bis(3-methoxybenzyl)-4-((2-(2-morpholinoethoxy)ethoxy)methyl)thiazol-2-amine; N,N-bis(3-methoxybenzyl)-4-((2-(2-(piperidin-1-yl)ethoxy)ethoxy)methyl)thiazol-2-amine; N,N-bis(3-methoxybenzyl)-4-(morpholinomethyl)thiazol-2-amine; N,N-bis(3-methoxybenzyl)-4-((2-morpholinoethoxy)methyl)thiazol-2-amine; N,N-bis(3-methoxybenzyl)-4-((2-(piperidin-1-yl)ethoxy)methyl)thiazol-2-amine; N-(3-methoxybenzyl)-N-(3-(2-methoxyethoxy)benzyl)-4-(morpholinomethyl)thiazol-2-amine; N-(4-(1H-pyrazol-1-yl)benzyl)-N-(3-methoxybenzyl)-4-(morpholinomethyl)thiazol-2-amine; N-(3-(1H-pyrazol-1-yl)benzyl)-N-(3-methoxybenzyl)-4-(morpholinomethyl)thiazol-2-amine; N,N-bis(3-methoxybenzyl)-4-(2-morpholinoethyl)thiazol-2-amine; N,N-bis(3-methoxybenzyl)-4-((2-morpholinoethoxy)methyl)oxazol-2-amine; N,N-bis(3-methoxybenzyl)-4-((tetrahydro-2H-pyran-4-ylamino)methyl)thiazol-2-amine; N,N-bis(3-methoxybenzyl)-4-((2-(tetrahydro-2H-pyran-4-yloxy)ethoxy)methyl)thiazol-2-amine; and N-(4-(1H-pyrazol-1-yl)benzyl)-N-(3-methoxybenzyl)-4-((3-methoxybenzyloxy)methyl)thiazol-2-amine.

9. A pharmaceutical composition comprising (i) the compound of claim 1, or a pharmaceutically acceptable salt thereof, and (ii) a pharmaceutically acceptable carrier.

10. A pharmaceutical composition comprising (i) the compound of claim 5, or a pharmaceutically acceptable salt thereof, and (ii) a pharmaceutically acceptable carrier.

11. A pharmaceutical composition comprising (i) the compound of claim 6, or a pharmaceutically acceptable salt thereof, and (ii) a pharmaceutically acceptable carrier.

12. A pharmaceutical composition comprising (i) the compound of claim 7, or a pharmaceutically acceptable salt thereof, and (ii) a pharmaceutically acceptable carrier.

13. A method comprising contacting integrin expressing cells with a VLA-4 integrin agonist having formula (I): 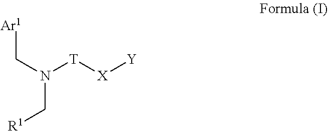
wherein, R¹ is hydrogen, lower alkyl, or aryl; Ar is aryl, provided that when Ar¹ and R¹ are both phenyl, the 2 and 6 positions of each ring are substituted with hydrogen; T is aryl; X is a 1-20 atom linking chain containing any combination of —CH₂—, —C(O)—, —C(O)NR²—, —NR²—, —C(O)O— and —O—, each of which may independently be absent or present one or more times; provided that when X contains —C(O)N(R²)—, and —C(O)N(R²)— directly attaches to T, then R² is hydrogen; Y is selected from a group consisting essentially of hydrogen, (C₁-C₄)alkyl, aryl, cycloalkyl, N,N-dialkylamino, heterocyclyl, N,N-di(aralkyl)aminoaryl, (N-aralkyl)(N-alkyl)aminoaryl and a combination thereof, Ar¹ and R¹ may each be independently substituted or multiply substituted and selected at each occurrence from the group consisting essentially of lower alkyl, alkoxy, hydroxyalkyl, —OH, alkoxyalkyl, (C₁-C₃ alkyl)₂amino, alkoxyalkoxy, cycloalkyl, cycloalkylalkyl, aryl, heterocyclyl, alkylaryl, aralkyl, alkylheterocyclyl, and heterocyclylalkyl, groups; Y may be unsubstituted or substituted with one or more substituents selected from the group consisting essentially of halogen, lower alkyl, alkoxy, hydroxyalkyl, aliphatic acyl, —CF₃, oxo, —CN, —OH, alkoxyalkyl, (C₁-C₃ alkyl)₂amino, alkoxyalkoxy, cycloalkyl, cycloalkylalkyl, aryl, heterocyclyl, alkylaryl, aralkyl, alkylheterocyclyl, heterocyclylalkyl, and aryloxyalkyl groups; T may be unsubstituted or substituted with one or more substituents selected from the group consisting essentially of lower alkyl, alkoxy, hydroxyalkyl, aliphatic acyl, —CN, —OH, halogen, heterocycloyl, alkoxyalkyl, (C₁-C₃ alkyl)₂amino, alkoxyalkoxy, cycloalkyl, cycloalkylalkyl, heterocyclyl, alkylheterocyclyl, heterocyclylalkyl and a combination thereof, provided that when T is thiazolyl group and X and Y taken together form a C₁-C₈ alkyl group, then the 5-position of said thiazole ring is unsubstituted; substituents of T may be taken together with up to two atoms of X to form ring; and R² when present, may be selected independently and at each occurrence from the groups consisting essentially of: hydrogen, lower alkyl, acetyl and methanesulfonyl.

14. The method of claim 13, wherein the VLA-4 integrin agonist is selected from the group consisting essentially of N,N-bis(3-methoxybenzyl)-4-((2-(3-methoxyphenoxy)ethoxy)methyl)thiazol-2-amine; N,N-bis(3-methoxybenzyl)-4-((2-(2-morpholinoethoxy)ethoxy)methyl)thiazol-2-amine; N,N-bis(3-methoxybenzyl)-4-((2-(2-morpholinoethoxy)ethoxy)methyl)thiazol-2-amine; N,N-bis(3-methoxybenzyl)-4-((2-(2-(piperidin-1-yl)ethoxy)ethoxy)methyl)thiazol-2-amine; N,N-bis(3-methoxybenzyl)-4-(morpholinomethyl)thiazol-2-amine; N,N-bis(3-methoxybenzyl)-4-((2-morpholinoethoxy)methyl)thiazol-2-amine; N,N-bis(3-methoxybenzyl)-4-((2-(piperidin-1-yl)ethoxy)methyl)thiazol-2-amine; N-(3-methoxybenzyl)-N-(3-(2-methoxyethoxy)benzyl)-4-(morpholinomethyl)thiazol-2-amine; N-(4-(1H-pyrazol-1-yl)benzyl)-N-(3-methoxybenzyl)-4-(morpholinomethyl)thiazol-2-amine; N-(3-(1H-pyrazol-1-yl)benzyl)-N-(3-methoxybenzyl)-4-(morpholinomethyl)thiazol-2-amine; N,N-bis(3-methoxybenzyl)-4-(2-morpholinoethyl)thiazol-2-amine; N,N-bis(3-methoxybenzyl)-4-((2-morpholinoethoxy)methyl)oxazol-2-amine; N,N-bis(3-methoxybenzyl)-4-((tetrahydro-2H-pyran-4-ylamino)methyl)thiazol-2-amine; N,N-bis(3-methoxybenzyl)-4-((2-(tetrahydro-2H-pyran-4-yloxy)ethoxy)methyl)thiazol-2-amine; and N-(4-(1H-pyrazol-1-yl)benzyl)-N-(3-methoxybenzyl)-4-((3-methoxybenzyloxy)methyl)thiazol-2-amine.

15. The method of claim 13, wherein the integrin expressing cells comprise neutrophils, eosinophils, basophils, mast cells, monocytes, macrophages, dendritic cells, natural killer cells, and lymphocytes, immune cells, embryonic stem cells, adult stem cells, progenitor cells, induced pluripotent stem cells, or a combination thereof.

16. The method of claim 13, wherein the integrin expressing cells express a naturally occurring integrin.

17. The method of claim 13, wherein the integrin expressing cells express a transgenic integrin.

18. A complex formed between (i) an integrin expressing cell and a integrin agonist; and/or (ii) an integrin binding protein wherein the integrin agonist has general formula (I) 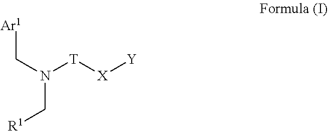
wherein, R¹ is hydrogen, lower alkyl, or aryl; Ar is aryl, provided that when Ar¹ and R¹ are both phenyl, the 2 and 6 positions of each ring are substituted with hydrogen; T is aryl; X is a 1-20 atom linking chain containing any combination of —CH₂—, —C(O)—, —C(O)NR²—, —NR²—, —C(O)O— and —O—, each of which may independently be absent or present one or more times; provided that when X contains —C(O)N(R²)—, and —C(O)N(R²)— directly attaches to T, then R² is hydrogen; Y is selected from a group consisting essentially of hydrogen, (C₁-C₄)alkyl, aryl, cycloalkyl, N,N-dialkylamino, heterocyclyl, N,N-di(aralkyl)aminoaryl, (N-aralkyl)(N-alkyl)aminoaryl and a combination thereof, Ar¹ and R¹ may each be independently substituted or multiply substituted and selected at each occurrence from the group consisting essentially of lower alkyl, alkoxy, hydroxyalkyl, —OH, alkoxyalkyl, (C₁-C₃ alkyl)₂amino, alkoxyalkoxy, cycloalkyl, cycloalkylalkyl, aryl, heterocyclyl, alkylaryl, aralkyl, alkylheterocyclyl, and heterocyclylalkyl, groups; Y may be unsubstituted or substituted with one or more substituents selected from the group consisting essentially of halogen, lower alkyl, alkoxy, hydroxyalkyl, aliphatic acyl, —CF₃, oxo, —CN, —OH, alkoxyalkyl, (C₁-C₃ alkyl)₂amino, alkoxyalkoxy, cycloalkyl, cycloalkylalkyl, aryl, heterocyclyl, alkylaryl, aralkyl, alkylheterocyclyl, heterocyclylalkyl, and aryloxyalkyl groups; T may be unsubstituted or substituted with one or more substituents selected from the group consisting essentially of lower alkyl, alkoxy, hydroxyalkyl, aliphatic acyl, —CN, —OH, halogen, heterocycloyl, alkoxyalkyl, (C₁-C₃ alkyl)₂amino, alkoxyalkoxy, cycloalkyl, cycloalkylalkyl, heterocyclyl, alkylheterocyclyl, heterocyclylalkyl and a combination thereof, provided that when T is thiazolyl group and X and Y taken together form a C₁-C₈ alkyl group, then the 5-position of said thiazole ring is unsubstituted; substituents of T may be taken together with up to two atoms of X to form ring; and R² when present, may be selected independently and at each occurrence from the groups consisting essentially of: hydrogen, lower alkyl, acetyl and methanesulfonyl.

19. The compound of claim 18, wherein the VLA-4 integrin agonist is selected from the group consisting essentially of: N,N-bis(3-methoxybenzyl)-4-((2-(3-methoxyphenoxy)ethoxy)methyl)thiazol-2-amine; N,N-bis(3-methoxybenzyl)-4-((2-(2-morpholinoethoxy)ethoxy)methyl)thiazol-2-amine; N,N-bis(3-methoxybenzyl)-4-((2-(2-morpholinoethoxy)ethoxy)methyl)thiazol-2-amine; N,N-bis(3-methoxybenzyl)-4-((2-(2-(piperidin-1-yl)ethoxy)ethoxy)methyl)thiazol-2-amine; N,N-bis(3-methoxybenzyl)-4-(morpholinomethyl)thiazol-2-amine; N,N-bis(3-methoxybenzyl)-4-((2-morpholinoethoxy)methyl)thiazol-2-amine; N,N-bis(3-methoxybenzyl)-4-((2-(piperidin-1-yl)ethoxy)methyl)thiazol-2-amine; N-(3-methoxybenzyl)-N-(3-(2-methoxyethoxy)benzyl)-4-(morpholinomethyl)thiazol-2-amine; N-(4-(1H-pyrazol-1-yl)benzyl)-N-(3-methoxybenzyl)-4-(morpholinomethyl)thiazol-2-amine; N-(3-(1H-pyrazol-1-yl)benzyl)-N-(3-methoxybenzyl)-4-(morpholinomethyl)thiazol-2-amine; N,N-bis(3-methoxybenzyl)-4-(2-morpholinoethyl)thiazol-2-amine; N,N-bis(3-methoxybenzyl)-4-((2-morpholinoethoxy)methyl)oxazol-2-amine; N,N-bis(3-methoxybenzyl)-4-((tetrahydro-2H-pyran-4-ylamino)methyl)thiazol-2-amine; N,N-bis(3-methoxybenzyl)-4-((2-(tetrahydro-2H-pyran-4-yloxy)ethoxy)methyl)thiazol-2-amine; and N-(4-(1H-pyrazol-1-yl)benzyl)-N-(3-methoxybenzyl)-4-((3-methoxybenzyloxy)methyl)thiazol-2-amine.

20. The complex of claim 18, wherein the integrin binding protein comprises vascular cell adhesion molecule-1 (VCAM 1), fibronectin, mucosal addressin cell adhesion molecule-1 (MAdCAM-1), intercellular adhesion molecule-1 (ICAM-1), intercellular adhesion molecule-2 (ICAM-2) or a combination thereof.

21.-29. (canceled)
